(19) United States
(12) Patent Application Publication
(10) Pub. No.: US 2022/0025364 A1
(43) Pub. Date: Jan. 27, 2022

(54) METHODS FOR STABLE GENOMIC INTEGRATION IN RECOMBINANT MICROORGANISMS

(71) Applicant: BioPlx, Inc., Boulder, CO (US)

(72) Inventors: Dan Rouse, Wheat Ridge, CO (US); Timothy W. Starzl, Boulder, CO (US); Ravi S. V. Starzl, Boulder, CO (US)

(21) Appl. No.: 17/337,033

(22) Filed: Jun. 2, 2021

Related U.S. Application Data
(60) Provisional application No. 63/033,681, filed on Jun. 2, 2020.
(60) Provisional application No. 63/049,544, filed on Jul. 8, 2020.

(51) Int. Cl. C12N 15/11; C12N 9/64; C12N 1/20

(52) U.S. Cl. CPC C12N 15/111; C12N 9/6408; C12R 2001/445; C12Y 304/21026; C12N 1/205

(57) ABSTRACT

Improved methods are provided for preparing synthetic microorganisms, recombinant microorganisms, live biotherapeutic products (rLBPs), and compositions thereof The synthetic microorganisms exhibit functional stability over at least 500 generations and are useful for treatment, prevention, and/or prevention of recurrence of microbial infections.

### CROSS REFERENCES TO RELATED APPLICATIONS

This application claims the benefit of U.S. Provisional Application No. 63/033,681, filed Jun. 2, 2020, and U.S. Provisional Application No. 63/049,544, filed Jul. 8, 2020, the entire contents of each of which are incorporated herein by reference.

### SEQUENCE LISTING

The present application includes a Sequence Listing in electronic format as a txt file entitled “Sequence-Listing,” which was created on 2 Jun. 2021 and which has a size of 245 kilobytes (KB). The contents of txt file “Sequence-Listing” are incorporated by reference herein.

### BACKGROUND

### Field of the Disclosure

Improved methods are provided for preparing synthetic microorganisms, recombinant live biotherapeutic products (rLBPs), and compositions thereof. The synthetic microorganisms exhibit functional stability over at least 500 generations and are useful in the treatment, prevention, or prevention of recurrence of microbial infections.

### Description of the Related Art

Bacterial interference can be an effective therapeutic strategy in the management of the microbiome to prevent infectious disease. In response to a methicillin-resistant Staphylococcus aureus (MRSA) outbreak in the 1960s, Shinefield et al. used a strain of Staphylococcus aureus (SA) called 502a and clinically demonstrated its ability to exclude MRSA from infant microbiomes, given the right conditions. However, during those trials, an infant was accidentally injected with the bacteria resulting in its death. Houck et al., “Fatal septicemia due to Staphylococcus aureus 502A.” American Journal of Diseases of Children 123 (1972): 45-48.

Methods and compositions for resisting microbial infection and reducing recurrence of microbial infection by decolonizing and replacing with a drug susceptible microorganism are in development.

WO 2019/113096 A1 (Starzl et al.) discloses a synthetic microorganism having a molecular modification comprising genomic insertion of an inducible promoter operably associated with a cell death gene. The synthetic microorganism exhibits good growth in dermal or mucosal environments, and desirably exhibits self-destruction by inducing expression of the cell death gene upon exposure to blood or serum. However, design and production of the genetic modifications can be laborious. For example, by using stitch PCR and Gibson assembly, full operons were constructed including the promoter region responsible for upregulating serum/blood genes in Staphylococcus aureus to drive the expression of the sprA1 toxin, and optionally using the promoter regions responsible for downregulating serum/blood genes in Staphylococcus aureus to drive the expression of the sprA1_{AS}.

WO 2017/123676 (Falb et al.) discloses recombinant E. coli Nissle strain comprising a heterologous gene encoding an amino acid catabolic enzyme operably linked to, e.g., a fumarate and nitrate reductase regulator responsive (FNR)-inducible promoter, which is amenable to growth in the human gut. Optionally, the cell may include auxotrophic and/or delayed kill switch modifications to prevent long-term colonization of the subject.

It is desirable to provide improved, efficient methods for making stable recombinant microorganisms comprising minimal genomic modifications that are capable of safely and durably replacing an undesirable microorganism, for example, under dermal or mucosal conditions.

### SUMMARY OF THE DISCLOSURE

The disclosure provides methods for making synthetic microbial strains comprising stable, genomically incorporated kill switch (KS) modifications as safety mechanisms to ensure that the resultant synthetic microorganisms and biotherapeutic compositions thereof are incapable of becoming accidental pathogens.

The present disclosure provides numerous strains of genetically-modified bacteria to ensure their safety and efficacy. Generally, though not exclusively, these engineered microorganisms including kill switch (KS) genomic modification have been designed to possess two key attributes. First, they are designed to durably occupy exterior epithelial niches (skin, nares) of the host's microbiome. Second, once introduced to internal systemic body fluid environments (plasma, serum, synovial fluid) genomically-modified KS strains have been designed to promptly initiate artificially-programmed cell death. Synthetic microorganisms comprising a kill switch were originally developed in Staphylococcus aureus to combat hospital-acquired MRSA infections, via the “Suppress and Replace” type paradigm of bacterial interference. In short, potentially harmful SA strains are first decolonized, or removed, from the host's microbiome, and then pathologically-inert KS strains of SA are introduced to the microbiome to fill the now vacant ecological niches that were once filled by potential pathogens.

As provided herein, synthetic Staph aureus KS strains have shown good efficacy in human plasma, human serum, human synovial fluid, and rabbit cerebrospinal fluid assays in vitro. Synthetic Staph aureus KS strains provided herein have shown good efficacy in in vivo mouse bacteremia and SSTI studies. In addition, synthetic Staph aureus KS strains are provided which are incapable of causing bacteremia or skin and soft tissue infection in vivo.

Recombinant microorganisms are provided comprising minimal genomic modifications that exhibit functional and genomic stability over time.

In some embodiments, recombinant microorganisms are provided having minimum molecular modification comprising genomic insertion of an action gene operably associated with an endogenous inducible gene or promoter, or comprising genomic insertion of an inducible promoter operably associated with an endogenous action gene.

Improved pass through microbial strains are provided for efficiently producing plasmids comprising an action gene, optionally a control arm, and homology arms for use in targeted insertion of the action gene behind an endogenous promoter gene in a target strain, for example, by homologous recombination. The pass through strain may comprise genetic modifications, for example, an epigenetic adaptation (e.g., DNA methylation pattern of target microorganism) and an antitoxin gene specific for the action gene to improve efficiency of plasmid preparation, and improve integration of the action gene into the genome of the target strain.

Methods are provided for preparing safe synthetic microorganisms that grow in dermal or mucosal environments, but will self-destruct upon exposure to systemic conditions, for example, upon exposure to blood, serum, plasma, contaminated cerebral spinal fluid, or synovial fluid.

For example, the synthetic microorganisms may contain an action gene that is a cell death gene operably associated with an inducible promoter gene that is not induced under dermal or mucosal conditions, but will be induced causing expression of the cell death gene upon exposure to systemic conditions, causing self-destruction of the synthetic microorganism.

Safe synthetic microorganisms are provided comprising minimal genomic disruption that may safely and durably replace an undesirable microorganism under, for example, dermal or mucosal conditions.

Synthetic microorganisms are provided that exhibit evolutionary stability of the genomic integration into the target strain over at least 500 generations. The synthetic microorganisms exhibit genetic stability and functional stability over at least 500 generations.

The synthetic microorganisms may be designed to durably occupy exterior epithelial niches (e.g., skin, nares) of the host subject's microbiome.

For example, safe synthetic microorganisms have been designed to durably occupy exterior epithelial niches (skin, nares) of the host subject's microbiome, but once introduced into interior body fluid (systemic) environments of the host subject, the safe synthetic strains initiate programmed cell death causing self-destruction to significantly decrease, or prevent bacteremia in the host subject.

The synthetic microorganism may be prepared by a method comprising genomic insertion of a first recombinant nucleotide into a target microorganism. The first recombinant nucleotide may comprise, consist essentially of, or consist of an action gene and optionally a control arm. The synthetic microorganism may comprise a genomic integration of a first recombinant nucleotide comprising a control arm and an action gene. The control arm may be located 5′ to the action gene. The control arm may be located immediately adjacent to the start codon of the action gene. The control arm may be located 3′ to the action gene. The control arm may be located immediately adjacent to the stop codon of the action gene. The control arm may be designed to be transcribed but not translated. The control arm may be complementary to an antisense nucleotide which may be used to tune the expression of the action gene.

The action gene may be a toxin gene. The toxin gene may be, for example, a sprA1, sma1, rsaE, relF, 187/lysK, Holin, lysostaphin, SprG1, SprA2, mazF, or Yoeb gene.

The disclosure provides a method of preparing a synthetic microorganism comprising transforming a target microorganism in the presence of a plasmid comprising a synthetic nucleic acid sequence comprising an action gene flanked by an upstream homology arm and a downstream homology arm, wherein the upstream and downstream homology arms comprise a first and a second complementary nucleic sequence, respectively, for targeting insertion of the action gene behind a native inducible promoter gene in the genome of the target microorganism.

The method may further comprise selecting a native inducible promoter gene in the target strain for targeted insertion of the synthetic nucleic acid sequence comprising the action gene, comprising comparing the relative RNA transcription levels of a native inducible gene in the target microorganism when grown in a first environmental condition compared to a second environmental condition, wherein the target microorganism exhibits at least a 10-fold increase in RNA transcription level when grown in the second environmental condition compared to the first for a comparable period of time. The period of time may be selected from the group consisting of at least about 15 min, 20 min, 30 min, 40 min, 45 min, 50 min, 60 min, 75 min, 90 min, 120 min, 180 min, 210 min, 240 min, 270 min, 300 min, 330 min, and 360 min, or any time point in between, and optionally wherein the RNA transcription levels in the target microorganism are assessed using an RNA-seq assay.

The target microorganism may be a bacterial species capable of colonizing a first environmental niche and may be a member of a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas. The first environmental condition may be a complete media or a dermal, gastrointestinal, genitourinary, or mucosal niche in a subject.

The second environmental condition may comprise exposure to or an increase in concentration of blood, plasma, serum, interstitial fluid, synovial fluid, contaminated cerebral spinal fluid, lactose, glucose, or phenylalanine in the subject.

In some embodiments, the synthetic microorganism may comprises a first molecular modification inserted to the genome of the target microorganism, the molecular modification comprising a first recombinant nucleotide comprising the action gene, wherein the first recombinant nucleotide is operatively associated with an endogenous first regulatory region comprising a native inducible first promoter gene, and wherein the native inducible first promoter imparts conditionally high level gene transcription of the first recombinant nucleotide in response to exposure to the second environmental condition of at least 10-fold increase, at least 20-fold increase, at least 50-fold increase, at least 75-fold increase, or at least a 100-fold increase, compared to the first environmental condition.

The action gene may be selected from the group consisting of a cell death action gene, virulence block action gene, metabolic modification action gene, nanofactory action gene, transcriptional regulator TetR-family gene, lacZ gene which codes for β-galactosidase (lactase or β-gal), or a gene which encodes an enzyme or hormone selected from the group consisting of sortase A (e.g., srt A), aerobic glycerol-3-phosphate dehydrogenase gene (e.g., glpD), thymidine kinase (tdk), glutenase, endopeptidase, prolyl endopeptidase (PEP), endopeptidase 40, and insulin. In some embodiments, the action gene is a cell death gene.

The plasmid may be derived from a shuttle vector suitable for use in both a pass through microorganism and the target microorganism.

In some embodiments, a synthetic pass through strain is provided comprising (a) a first genomic modification comprising a first synthetic nucleic acid sequence encoding a DNA methylation enzyme and/or acetylation enzyme derived from the target microorganism; and (b) a second genomic modification comprising a second synthetic nucleic acid sequence comprising an antitoxin gene encoding an antisense RNA sequence capable of hybridizing with at least a portion of the cell death gene. The presence of the antisense genomic modification in the pass through strain may allow the pass through strain to propagate the plasmid comprising the cell death gene, and allows the pass through strain to survive leaky expression of the toxin gene in the plasmid. The presence of the genomic modification encoding the methylation enzyme and/or acetylation enzyme in the pass through strain may allow the pass through strain to impart a methylation pattern and/or acetylation pattern on the plasmid DNA similar enough to the methylation pattern and/or acetylation pattern of the target microorganism, to enable or enhance efficiency of transformation of the target strain with the plasmid propagated in the pass through strain. The pass through strain may be an Escherichia coli strain or a yeast strain.

In some embodiments, the target microorganism may have the same genus and species as an undesirable microorganism capable of causing bacteremia or SSTI in the subject. In some embodiments, the undesirable microorganism may be capable of causing bacteremia or SSTI in the subject.

A synthetic microorganism prepared according to methods of the disclosure may exhibit measurable average cell death of the synthetic microorganism within at least a preset period of time following exposure to a second environmental condition. The measurable average cell death may occur within the preset period of time selected from the group consisting of within at least about 15, 30, 60, 90, 120, 180, 240, 300, or 360 min minutes following exposure to the second environmental condition. The first environmental condition may be a complete media or a dermal, or mucosal niche in a subject. The second environmental condition may comprise exposure to or an increase in concentration of blood, plasma, serum, interstitial fluid, synovial fluid, or contaminated cerebral spinal fluid.

In some embodiments, the measurable average cell death is a cfu count reduction of at least 50%, at least 70%, at least 80%, at least 90%, at least 95%, at least 99%, at least 99.5%, at least 99.8%, or at least 99.9% cfu count reduction following the preset period of time.

In some embodiments, the synthetic microorganism is incapable of causing bacteremia or SSTI in a subject.

In some embodiments, the target microorganism is derived from a Staphylococcus aureus strain.

In some embodiments, the action gene is a cell death gene selected from or derived from the group consisting of sprA1, sprA2, sprG, mazF, relE, relF, hokB, hokD, yafQ, rsaE, yoeB, yefM, kpn1, sma1, or lysostaphin toxin gene. In some embodiments, the action gene comprises a nucleotide sequence selected from the group consisting of SEQ ID NOs: BP_DNA_003 (SEQ ID NO: 3), BP_DNA_008 (SEQ ID NO: 8), BP_DNA_0032, BP_DNA_035 (SEQ ID NO:25), BP_DNA_045 (SEQ ID NO: 29), BP_DNA_065 (SEQ ID NO: 34), BP_DNA_067 (SEQ ID NO: 35), BP_DNA_068 (SEQ ID NO: 36), BP_DNA 069 (SEQ ID NO: 37), BP_DNA 070 (SEQ ID NO: 38), BP_DNA_71 (SEQ ID NO: 39), or a substantially identical nucleotide sequence.

In some embodiments, the target microorganism is a S. aureus strain, and the inducible first promoter gene is selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosyntheis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrichrome transport ATP-binding protein fhuA), fhuB (ferrichrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

In some embodiments, the inducible first promoter gene comprises a nucleotide sequence complementary to an upstream or downstream homology arm having a nucleic acid sequence selected from the group consisting of BP_DNA_001(SEQ ID NO: 1), BP_DNA_002 (SEQ ID NO: 2), BP_DNA_004 (SEQ ID NO: 4), BP_DNA_006 (SEQ ID NO: 6), BP_DNA_007 (SEQ ID NO: 7), BP_DNA_010 (SEQ ID NO: 9), BP_DNA_BP_DNA_012 (SEQ ID NO: 10), BP_DNA_013 (SEQ ID NO: 11), BP_DNA_014 (SEQ ID NO: 12), BP_DNA_016 (SEQ ID NO: 13), BP_DNA_017 (SEQ ID NO: 14), BP_DNA_029 (SEQ ID NO: 20), BP_DNA_031 (SEQ ID NO: 22), BP_DNA_033 (SEQ ID NO: 24), BP_DNA_041 (SEQ ID NO: 27), and BP_DNA_057 (SEQ ID NO: 31), or a substantially identical nucleotide sequence thereof.

The method for preparing a synthetic microorganism may further comprise inserting at least a second molecular modification (expression clamp) into the genome of the target microorganism, the second molecular modification comprising a (anti-action) regulator gene encoding a small noncoding RNA (sRNA) specific for the control arm or action gene, wherein the regulator gene is operably associated with an second regulatory region comprising a second promoter gene which is transcriptionally active (constitutive) when the synthetic microorganism is grown in the first environmental condition, but is not induced, induced less than 1.5-fold, or is repressed after exposure to the second environmental condition for a period of time of at least 120 minutes.

In some embodiments, the regulator gene may encode an sRNA sequence capable of hybridizing with at least a portion of the action gene.

In some embodiments, the synthetic microorganism comprises a second molecular modification comprising or derived a toxin gene selected from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively. In some embodiments, the second molecular modification comprises a nucleotide sequence comprising BP_DNA_005 (SEQ ID NO: 5), or a substantially identical nucleotide sequence.

The second promoter may comprises or be derived from a gene selected from the group consisting of PsprA1as (sprA1as native promoter), cfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR faimly transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

In some embodiments, a synthetic microorganism is provided comprising a first molecular modification inserted to the genome of a target microorganism, the molecular modification comprising a first recombinant nucleotide comprising an action gene, wherein the first recombinant nucleotide is operatively associated with an endogenous first regulatory region comprising a native inducible first promoter gene, and wherein the native inducible first promoter imparts conditionally high level gene transcription of the first recombinant nucleotide in response to exposure to a change in state of at least three fold increase compared to basal productivity.

In some embodiments, a synthetic microorganism is provided comprising a first molecular modification inserted to the genome of a target microorganism, the molecular modification comprising a recombinant nucleotide comprising a first regulatory region comprising an inducible first promoter gene, wherein the inducible first promoter gene is operably associated with an endogenous action gene, and wherein the inducible first promoter imparts conditionally high level gene transcription of the endogenous action gene in response to a change in state of at least three fold increase of basal productivity.

The basal productivity may be determined by gene transcription level of the inducible first promoter gene and/or action gene when the synthetic microorganism is grown under a first environmental condition over a period of time.

In some embodiments, the inducible first promoter gene is upregulated by at least 10-fold within a period of time of at least 120 min following the change in state comprising an exposure to a second environmental condition.

In some embodiments, the target microorganism has the same genus and species as an undesirable microorganism.

In some embodiments, the target microorganism is an isolated wild-type microorganism, commercially available microorganism, or a synthetic microorganism.

In some embodiments, the synthetic microorganism comprising the first promoter gene is not induced, induced less than 1.5 fold, or is repressed when the synthetic microorganism is grown under the first environmental condition.

The first recombinant gene may further comprise a control arm immediately adjacent to the action gene. The control arm may include a 5′ untranslated region (UTR) and/or a 3′ UTR relative to the action gene. The control arm may be complementary to an antisense oligonucleotide encoded by the genome of the synthetic microorganism. The antisense oligonucleotide may be encoded by a gene that is endogenous or inserted to the genome of the synthetic microorganism.

The first promoter gene may induce conditionally high level gene expression of the action gene in response to exposure to the second environmental condition of at least three-fold, five-fold, at least ten-fold, at least 20-fold, at least 50-fold, or at least 100-fold increase of basal productivity.

The synthetic microorganism comprises the action gene and the first promoter gene within the same operon.

The action gene may be integrated between the stop codon and the transcriptional terminator of any gene located in the same operon as the first promoter gene.

The synthetic microorganism may comprise at least a second molecular modification (expression clamp) comprising a (anti-action) regulator gene encoding a small noncoding RNA (sRNA) specific for the control arm or action gene, wherein the regulator gene is operably associated with an endogenous second regulatory region comprising a second promoter gene which is transcriptionally active (constitutive) when the synthetic microorganism is grown in the first environmental condition, but is not induced, induced less than 1.5-fold, or is repressed after exposure to the second environmental condition for a period of time of at least 120 minutes.

In some embodiments, the transcription of the regulator gene produces the sRNA in an effective amount to prevent or suppress the expression of the action gene when the microorganism is grown under the first environmental condition.

The first molecular modification may be selected from the group consisting of kill switch molecular modification, virulence block molecular modification, metabolic molecular modification, and nano factory molecular modification.

The synthetic microorganism according to the disclosure may exhibit genomic stability of the first molecular modification and functional stability of the action gene over at least 500 generations, at least 1,000 generations, at least 1,500 generations, at least 3,000 generations, or more.

The synthetic microorganism may comprise a kill switch molecular modification comprising an action gene including a first cell death gene operatively associated with a native inducible first promoter gene, wherein the cell death gene and the native inducible first promoter are not operably associated in nature.

The synthetic microorganism may further comprise a deletion of at least a portion of a native action gene, optionally wherein the deleted native action gene is a toxin gene or portion thereof. The deletion of at least a portion of the native action (toxin) gene may comprise a deletion of a native nucleic acid sequence selected from the group consisting of the Shine-Dalgarno sequence, ribosomal binding site, and the transcription start site of the native toxin gene.

The synthetic microorganism may comprise a deletion of at least a portion of a native antitoxin gene specific for the native toxin gene, optionally wherein the native antitoxin gene encodes an mRNA or sRNA antisense or antitoxin peptide specific for the native toxin gene.

A synthetic microorganism is provided prepared according to a method of the disclosure, wherein a measurable average cell death of the synthetic microorganism occurs within at least a preset period of time following change of state when the synthetic microorganism is exposed to the second environmental condition. The measurable average cell death may occur within at least a preset period of time selected from the group consisting of within at least 1, 5, 15, 30, 60, 90, 120, 180, 240, 300, or 360 min minutes following exposure to the second environmental condition. The measurable average cell death may be a cfu count reduction of at least 50%, at least 70%, at least 80%, at least 90%, at least 95%, at least 99%, at least 99.5%, at least 99.8%, or at least 99.9% cfu count reduction following the preset period of time.

A synthetic microorganism is provided according to the disclosure comprising a kill switch molecular modification that is capable of reducing or preventing infectious growth of the synthetic microorganism within the second environmental condition.

The first environmental condition may be selected from the group consisting of dermal, mucosal, genitourinary, gastrointestinal in a subject, or a complete media.

The second environmental condition may be selected from the group consisting of exposure to or an increase in concentration of blood, plasma, serum, interstitial fluid, synovial fluid, contaminated cerebral spinal fluid, lactose, glucose, or phenylalanine.

The target microorganism may be susceptible to at least one antimicrobial agent.

The target microorganism may be selected from the group consisting of bacteria and yeast target microorganisms.

The target microorganism may be a bacterial species having a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

The target microorganism may be selected from the group consisting of Staphylococcus aureus, Escherichia co/i, and Streptococcus spp.

The action gene may be a cell death gene selected from or derived from the group consisting of sprA1, sprA2, sprG, mazF, relE, relF, hokB, hokD, yafQ, rsaE, yoeB, yefM, kpn1, sma1, or lysostaphin toxin gene. The cell death gene may comprise a nucleotide sequence selected from the group consisting of SEQ ID NOs: BP_DNA_003 (SEQ ID NO: 3), BP_DNA_008 (SEQ ID NO: 8), BP_DNA_0032, BP_DNA_035 (SEQ ID NO:25), BP_DNA_045 (SEQ ID NO: 29), BP_DNA_065 (SEQ ID NO: 34), BP_DNA_067 (SEQ ID NO: 35), BP_DNA_068 (SEQ ID NO: 36), BP_DNA_069 (SEQ ID NO: 37), BP_DNA 070 (SEQ ID NO: 38), BP_DNA 071 (SEQ ID NO: 39), or a substantially identical nucleotide sequence.

The target microorganism may be a S. aureus strain, wherein the inducible first promoter gene is selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosyntheis protein), sbnD, sbnI, sbnE (lucA/lucC family siderophore biosynthesis protein), isdI, IrgA (murein hydrolase regulator A), IrgB (murein hydrolase regulator B), ear (Ear protein), fhuA (ferrichrome transport ATP-binding protein fhuA), fhuB (ferrichrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

The target microorganism may be a S. aureus strain, wherein the inducible first promoter gene comprises a nucleotide sequence complementary to an upstream or downstream homology arm having a nucleic acid sequence selected from the group consisting of BP_DNA_001 (SEQ ID NO: 1), BP_DNA_002 (SEQ ID NO: 2), BP_DNA_004 (SEQ ID NO: 4), BP_DNA_006 (SEQ ID NO: 6), BP_DNA_007 (SEQ ID NO: 7), BP_DNA_010 (SEQ ID NO: 9), BP_DNA_BP_DNA_012 (SEQ ID NO: 10), BP_DNA_013 (SEQ ID NO: 11), BP_DNA_014 (SEQ ID NO: 12), BP_DNA_016 (SEQ ID NO: 13), BP_DNA_017 (SEQ ID NO: 14), BP_DNA_029 (SEQ ID NO: 20), BP_DNA_031 (SEQ ID NO: 22), BP_DNA_033 (SEQ ID NO: 24), BP_DNA_041 (SEQ ID NO: 27), and BP_DNA_057 (SEQ ID NO: 31), or a substantially identical nucleotide sequence thereof.

The synthetic microorganism may comprise a second molecular modification encoding an sRNA sequence capable of hybridizing with at least a portion of the action gene, or encoding an peptide specific for at least a portion of a protein encoded by the action gene. The second molecular modification may comprises or be derived from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively. The second molecular modification comprises a nucleotide sequence comprising BP_DNA_005 (SEQ ID NO: 5), or a substantially identical nucleotide sequence.

The second promoter gene may comprise or be derived from a gene selected from the group consisting of PsprA1as (sprA1as native promoter), clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR faimly transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

A method for preparing a synthetic microorganism comprising an exogenous action gene is provided, the method comprising selecting a target microorganism of interest; selecting a fluid of interest for activation of the exogenous action gene; identifying a native inducible gene in the target microorganism of interest that exhibits increased expression in the presence of the fluid of interest of at least 3-fold compared to a complete media or the target microorganisms niche environment; and inserting the action gene into the genome of the target microorganism in the same operon as the inducible gene such that the action gene and the inducible gene are operably associated to provide the synthetic microorganism. The target microorganism may be of the same genus and species as an undesirable microorganism. The target microorganism may be an isolated target microorganism, a commercially-available target microorganism, or a synthetic target microorganism. The fluid of interest may be blood, serum, plasma, cerebrospinal fluid, synovial fluid, or milk. The synthetic microorganism may be genetically stable for at least 500 generations in complete media or the target microorganisms niche environment. The target microorganisms niche environment may be complete media or a dermal, gastrointestinal, genitourinary, or mucosal niche in a subject.

In some embodiments, a live biotherapeutic composition is provided comprising one or more, two or more, three of more, four or more, five or more, six or more, seven or more or 1 to 20, 2 to 10, 3 to 5 different synthetic microorganisms prepared from a target microorganism having a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

In some embodiments, a live biotherapeutic composition is provided comprising one or more, two or more, three of more, four or more, five or more, six or more, seven or more or 1 to 20, 2 to 10, 3 to 5 different synthetic microorganisms selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcushyicus, Acinetobacter baumannii, Acinetobacter calcoaceticus, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa.

A composition is provided for use in the manufacture of a medicament for eliminating and preventing the recurrence of a skin and soft tissue infection (SSTI) in a subject, optionally comprising two or more, three or more, four or more, five or more, six or more, seven or more, eight or more, nine or more, or ten or more, or 1 to 20, 2 to 10, 3 to 5 different synthetic microorganisms.

In some embodiments, a live biotherapeutic composition is provided comprising a mixture of synthetic microorganisms comprising at least a Staphylococcus sp., a Escherichia sp., and a Streptococcus sp. synthetic strains.

In a particular embodiment, a live biotherapeutic composition is provided comprising three or more synthetic microorganisms derived from target microorganisms including each of a Staphylococci species, a Streptococci species, and an Escherichia coli species.

The target Staphylococcus species may be selected from the group consisting of a catalase-positive Staphylococcus species and a coagulase-negative Staphylococcus species. The target Staphylococcus species may be selected from the group consisting of Staphylococcus aureus, S. epidermidis, S. chromogenes, S. simulans, S. saprophyticus, S. sciuri, S. haemolyticus, and S. hyicus. The target Streptococci species may be a Group A, Group B or Group C/G species. The target Streptococci species may be selected from the group consisting of Streptococcus uberis, Streptococcus agalactiae, Streptococcus dysgalactiae, and Streptococcus pyogenes. The E. coli species may be a Mammary Pathogenic Escherichia coli (MPEC) species.

A method is provided for treating, preventing, or preventing the recurrence of a skin or soft tissue infection associated with an undesirable microorganism in a subject hosting a microbiome, comprising: (a) decolonizing the host microbiome; and (b) durably replacing the undesirable microorganism by administering to the subject a biotherapeutic composition comprising a synthetic microorganism comprising at least one element imparting a non-native attribute, wherein the synthetic microorganism is capable of durably integrating to the host microbiome, and occupying the same niche in the host microbiome as the undesirable microorganism.

The decolonizing may be performed on at least one site in the subject to substantially reduce or eliminate the detectable presence of the undesirable microorganism from the at least one site.

The niche may be a dermal or mucosal environment that allows stable colonization of the undesirable microorganism at the at least one site.

Methods and compositions are provided for safely and durably influencing microbiological ecosystems (microbiomes) in a subject to perform a variety of functions, for example, including reducing the risk of infection by an undesirable microorganism such as virulent, pathogenic and/or drug-resistant microorganism.

Methods are provided herein to prevent or reduce the risk of colonization, infection, recurrence of colonization, or recurrence of a pathogenic infection by an undesirable microorganism in a subject, comprising: decolonizing the undesirable microorganism on at least one site in the subject to reduce or eliminate the presence of the undesirable microorganism from the site; and durably replacing the undesirable microorganism by administering a synthetic microorganism to the at least one site in the subject, wherein the synthetic microorganism can durably integrate with a host microbiome by occupying the niche previously occupied by the undesirable microorganism; and optionally promoting colonization of the synthetic microorganism within the subject.

The disclosure provides a method for eliminating and preventing the recurrence of a undesirable microorganism in a subject hosting a microbiome, comprising (a) decolonizing the host microbiome; and (b) durably replacing the undesirable microorganism by administering to the subject a synthetic microorganism comprising a kill switch molecular modification, wherein the synthetic microorganism is capable of durably integrating to the host microbiome, and occupying the same niche in the host microbiome as the undesirable microorganism.

In some embodiments, the decolonizing is performed on at least one site in the subject to substantially reduce or eliminate the detectable presence of the undesirable microorganism from the at least one site.

In some embodiments, the detectable presence of an undesirable microorganism or a synthetic microorganism is determined by a method comprising a phenotypic method and/or a genotypic method, optionally wherein the phenotypic method is selected from the group consisting of biochemical reactions, serological reactions, susceptibility to anti-microbial agents, susceptibility to phages, susceptibility to bacteriocins, and/or profile of cell proteins. In some embodiments, the genotypic method is selected a hybridization technique, plasmids profile, analysis of plasmid polymorphism, restriction enzymes digest, reaction and separation by Pulsed-Field Gel Electrophoresis (PFGE), ribotyping, polymerase chain reaction (PCR) and its variants, Ligase Chain Reaction (LCR), and Transcription-based Amplification System (TAS).

In some embodiments, the niche is a dermal or mucosal environment that allows stable colonization of the undesirable microorganism at the at least one site in the subject.

In some embodiments, the ability to durably integrate to the host microbiome is determined by detectable presence of the synthetic microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the ability to durably replace the undesirable microorganism is determined by the absence of detectable presence of the undesirable microorganism at the at least one site for a period of at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the ability to occupy the same niche is determined by absence of co-colonization of the undesirable microorganism and the synthetic microorganism at the at least one site after the administering step. In some embodiments, the absence of co-colonization is determined at least two weeks, at least four weeks, at least six weeks, at least eight weeks, at least ten weeks, at least 12 weeks, at least 16 weeks, at least 26 weeks, at least 30 weeks, at least 36 weeks, at least 42 weeks, or at least 52 weeks after the administering step.

In some embodiments, the synthetic microorganism comprises at least one element imparting the non-native attribute that is durably incorporated to the synthetic microorganism. In some embodiments, the at least one element imparting the non-native attribute is durably incorporated to the host microbiome via the synthetic microorganism.

In some embodiments, the at least one element imparting the non-native attribute is a kill switch molecular modification, virulence block molecular modification, or nanofactory molecular modification. In some embodiments, the synthetic microorganism comprises molecular modification that is integrated to a chromosome of the synthetic microorganism. In some embodiments, the synthetic microorganism comprises a virulence block molecular modification that prevents horizontal gene transfer of genetic material from the undesirable microorganism.

In some embodiments, the measurable average cell death of the synthetic microorganism comprising a kill switch molecular modification occurs within at least a preset period of time following induction of the first promoter after the change in state. In some embodiments, the measurable average cell death occurs within at least a preset period of time selected from the group consisting of within at least 1, 5, 15, 30, 60, 90, 120, 180, 240, 300, or 360 min minutes following the change of state. In some embodiments, the measurable average cell death is at least a 50% cfu, at least 70%, at least 80%, at least 90%, at least 95%, at least 99%, at least 99.5%, at least 99.8%, or at least 99.9% cfu count reduction following the preset period of time. In some embodiments, the change in state is selected from one or more of pH, temperature, osmotic pressure, osmolality, oxygen level, nutrient concentration, blood concentration, plasma concentration, serum concentration, metal concentration, chelated metal concentration, change in composition or concentration of one or more immune factors, mineral concentration, and electrolyte concentration. In some embodiments, the change in state is a higher concentration of and/or change in composition of blood, serum, or plasma compared to normal physiological (niche) conditions at the at least one site in the subject.

The biotherapeutic composition comprising a synthetic microorganism may be administered pre-partum, early, mid-, or late lactation phase or in the dry period to the cow, goat sheep, or sow in need thereof.

In some embodiments, the undesirable microorganism is a Staphyloccoccus aureus strain, and wherein the detectable presence is measured by a method comprising obtaining a sample from the at least one site of the subject, contacting a chromogenic agar with the sample, incubating the contacted agar and counting the positive cfus of the bacterial species after a predetermined period of time.

In some embodiments, a method is provided comprising a decolonizing step comprising topically administering a decolonizing agent to at least one site in the subject to reduce or eliminate the presence of the undesirable microorganism from the at least one site.

In some embodiments, the decolonizing step comprises topical administration of a decolonizing agent, wherein no systemic antimicrobial agent is simultaneously administered. In some embodiments, no systemic antimicrobial agent is administered prior to, concurrent with, and/or subsequent to within one week, two weeks, three weeks, one month, two months, three months, six months, or one year of the first topical administration of the decolonizing agent or administration of the synthetic microorganism. In some embodiments, the decolonizing agent is selected from the group consisting of a disinfectant, bacteriocide, antiseptic, astringent, and antimicrobial agent.

In some embodiments, the decolonizing agent is selected from the group consisting of alcohols (ethyl alcohol, isopropyl alcohol), aldehydes (glutaraldehyde, formaldehyde, formaldehyde-releasing agents (noxythiolin=oxymethylenethiourea, tauroline, hexamine, dantoin), o-phthalaldehyde), anilides (triclocarban=TCC=3,4,4′-triclorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), chloroxylenol (PCMX), 8-hydroxyquinoline, dodecyl benzene sulfonic acid, nisin, chlorine, glycerol monolaurate, C₈-C₁₄ fatty acids, quaternary ammonium compounds (cetrimide, benzalkonium chloride, cetyl pyridinium chloride), silver compounds (silver sulfadiazine, silver nitrate), peroxy compounds (hydrogen peroxide, peracetic acid, benzoyl peroxide), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), isotretinoin, sulfur compounds, botanical extracts (peppermint, calendula, eucalyptus, Melaleuca spp. (tea tree oil), (Vaccinium spp. (e.g., A-type proanthocyanidins), Cassia fistula Linn, Baekea frutesdens L., Melia azedarach L., Muntingia calabura, Vitis vinifera L, Terminalia avicennioides Guill & Perr., Phylantus discoideus muel. Muel-Arg., Ocimum gratissimum Linn., Acalypha wilkesiana Muell-Arg., Hypericum pruinatum Boiss. & Bal., Hypericum olimpicum L. and Hypericum sabrum L., Hamamelis virginiana (witch hazel), Clove oil, Eucalyptus spp., Rosemarinus officinalis spp. (rosemary), Thymus spp. (thyme), Lippia spp. (oregano), Lemongrass spp., Cinnamomum spp., Geranium spp., lavendula spp., Calendula spp.,), aminolevulonic acid, topical antibiotic compounds (bacteriocins; mupirocin, bacitracin, neomycin, polymyxin B, gentamicin).

In some embodiments, the antimicrobial agent is selected from the group consisting of cephapirin, amoxicillin, trimethoprim-sulfonamides, sulfonamides, oxytetracycline, fluoroquinolones, enrofloxacin, danofloxacin, marbofloxacin, cefquinome, ceftiofur, streptomycin, oxytetracycline, vancomycin, cefazolin, cepahalothin, cephalexin, linezolid, daptomycin, clindamycin, lincomycin, mupirocin, bacitracin, neomycin, polymyxin B, gentamicin, prulifloxacin, ulifloxacin, fidaxomicin, minocyclin, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norftoxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, and fluconazole.

In some embodiments, the decolonizing comprises topically administering the decolonizing agent at least one, two, three, four, five or six or more times prior to the replacing step. In some embodiments, the decolonizing step comprises administering the decolonizing agent to the at least one host site in the subject from one to six or more times or two to four times at intervals of between 0.5 to 48 hours apart, and wherein the replacing step is performed after the final decolonizing step.

The replacing step may be performed after the final decolonizing step, optionally wherein the decolonizing agent is in the form of a spray, dip, lotion, foam, cream, balm, or intramammary infusion.

In some embodiments, a method is provided comprising decolonizing an undesirable microorganism, and replacing with a synthetic microorganism comprising topical administration of a composition comprising at least 10⁵, at least 10⁶, at least 10⁷, at least 10⁸, at least 10⁹, at least 10¹⁰, or at least 10¹¹ CFU of the synthetic strain and a pharmaceutically acceptable carrier to at least one host site in the subject. In some embodiments, the initial replacing step is performed within 12 hours, 24 hours, 36 hours, 2 days, 3 days, 4 days, 5 days, 6 days, 7 days, 8 days, 9 days or 10 days, or between 0.5-10 days, 1-7 days, or 2 to 5 days of the decolonizing step. In some embodiments, the replacing step is repeated at intervals of no more than once every two weeks to six months following the final decolonizing step. In some embodiments, the decolonizing step and the replacing step is repeated at intervals of no more than once every two weeks to six months, or three weeks to three months. In some embodiments, the replacing comprises administering the synthetic microorganism to the at least one site at least one, two, three, four, five, six, seven, eight, nine, or ten times. In some embodiments, the replacing comprises administering the synthetic microorganism to the at least one site no more than one, no more than two, no more than three times, or no more than four times per month.

In some embodiments, the method of decolonizing the undesirable microorganism and replacing with a synthetic microorganism further comprises promoting colonization of the synthetic microorganism in the subject. In some embodiments, the promoting colonization of the synthetic microorganism in the subject comprises administering to the subject a promoting agent, optionally where the promoting agent is a nutrient, prebiotic, commensal, stabilizing agent, humectant, and/or probiotic bacterial species. In some embodiments, the promoting comprises administering a probiotic species at from 10⁵ to 10¹⁰ cfu, 10⁶ to 10⁹ cfu, or 10⁷ to 10⁸ cfu to the subject after the initial decolonizing step.

In some embodiments, the nutrient is selected from sodium chloride, lithium chloride, sodium glycerophosphate, phenylethanol, mannitol, tryptone, peptide, and yeast extract. In some embodiments, the prebiotic is selected from the group consisting of short-chain fatty acids (acetic acid, propionic acid, butyric acid, isobutyric acid, valeric acid, isovaleric acid), glycerol, pectin-derived oligosaccharides from agricultural by-products, fructo-oligosaccarides (e.g., inulin-like prebiotics), galacto-oligosaccharides (e.g., raffinose), succinic acid, lactic acid, and mannan-oligosaccharides.

In some embodiments, the probiotic is selected from the group consisting of Bifidobacterium breve, Bifidobacterium bifidum, Bifidobacterium lactis, Bifidobacterium infantis, Bifidobacterium breve, Bifidobacterium longum, Lactobacillus reuteri, Lactobacillus paracasei, Lactobacillus plantarum, Lactobacillus johnsonii, Lactobacillus rhamnosus, Lactobacillus acidophilus, Lactobacillus salivarius, Lactobacillus casei, Lactobacillus plantarum, Lactococcus lactis, Streptococcus thermophiles, and Enterococcus fecalis.

In some embodiments, the undesirable microorganism is an antimicrobial agent-resistant microorganism. In some embodiments, the antimicrobial agent-resistant microorganism is an antibiotic resistant bacteria. In some embodiments, the antibiotic-resistant bacteria is a Gram-positive bacterial species selected from the group consisting of a Streptococcus spp., Cutibacterium spp., and a Staphylococcus spp. In some embodiments, the Streptococcus spp. is selected from the group consisting of Streptococcus pneumoniae, Steptococcus mutans, Streptococcus sobrinus, Streptococcus pyogenes, and Streptococcus agalactiae. In some embodiments, the Cutibacterium spp. is selected from the group consisting of Cutibacterium acnes subsp. acnes, Cutibacterium acnes subsp. defendens, and Cutibacterium acnes subsp. elongatum. In some embodiments, the Staphylococcus spp. is selected from the group consisting of Staphylococcus aureus, Staphylococcus epidermidis, and Staphylococcus saprophyticus. In some embodiments, the undesirable microorganism is a methicillin-resistant Staphylococcus aureus (MRSA) strain that contains a staphylococcal chromosome cassette (SCCmec types I-III), which encode one (SCCmec type I) or multiple antibiotic resistance genes (SCCmec type II and III), and/or produces a toxin. In some embodiments, the toxin is selected from the group consisting of a Panton-Valentine leucocidin (PVL) toxin, toxic shock syndrome toxin-1 (TSST-1), staphylococcal alpha-hemolysin toxin, staphylococcal beta-hemolysin toxin, staphylococcal gamma-hemolysin toxin, staphylococcal delta-hemolysin toxin, enterotoxin A, enterotoxin B, enterotoxin C, enterotoxin D, enterotoxin E, and a coagulase toxin.

In some embodiments, the subject treated with a method according to the disclosure does not exhibit recurrence or colonization of the undesirable microorganism as evidenced by swabbing the subject at the at least one site for at least two weeks, at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

The disclosure provides a synthetic microorganism for durably replacing an undesirable microorganism in a subject. The synthetic microorganism comprises a molecular modification designed to enhance safety by reducing the risk of systemic infection. In one embodiment, the molecular modification causes a significant reduction in growth or cell death of the synthetic microorganism in response to blood, serum, plasma, or interstitial fluid. The synthetic microorganism may be used in methods and compositions for preventing or reducing recurrence of dermal or mucosal colonization or recolonization of an undesirable microorganism in a subject.

The disclosure provides a synthetic microorganism for use in compositions and methods for treating or preventing, reducing the risk of, or reducing the likelihood of colonization, or recolonization, systemic infection, bacteremia, or endocarditis caused by an undesirable microorganism in a subject.

The disclosure provides a synthetic microorganism comprising a recombinant nucleotide comprising at least one kill switch molecular modification comprising a first cell death gene operatively associated with a first regulatory region comprising an inducible first promoter, wherein the first inducible promoter exhibits conditionally high level gene expression of the recombinant nucleotide in response to exposure to blood, serum, or plasma of at least three fold increase of basal productivity. In some embodiments, the inducible first promoter exhibits, comprises, is derived from, or is selected from a gene that exhibits upregulation of at least 5-fold, at least 10-fold, at least 20-fold, at least 50-fold, or at least 100-fold within at least 30 min, 60 min, 90 min, 120 min, 180 min, 240 min, 300 min, or at least 360 min following exposure to blood, serum, or plasma.

In some embodiments, the synthetic microorganism comprises a kill switch molecular modification comprising a first cell death gene operably linked to a first regulatory region comprising a inducible first promoter, wherein the first promoter is activated (induced) by a change in state in the microorganism environment in contradistinction to the normal physiological (niche) conditions at the at least one site in the subject.

In some embodiments, the synthetic microorganism further comprises an expression clamp molecular modification comprising an antitoxin gene specific for the first cell death gene or a product thereof, wherein the antitoxin gene is operably associated with a second regulatory region comprising a second promoter which is constitutive or active upon dermal or mucosal colonization or in a complete media, but is not induced, induced less than 1.5-fold, or is repressed after exposure to blood, serum or plasma for at least 30 minutes. In some embodiments, the second promoter is active upon dermal or mucosal colonization or in TSB media, but is repressed by at least 2 fold upon exposure to blood, serum or plasma after a period of time of at least 30 min, 60 min, 90 min, 120 min, 180 min, 240 min, 300 min, or at least 360 min.

In some embodiments, the synthetic microorganism exhibits measurable average cell death of at least 50% cfu reduction within at least 1, 5, 15, 30, 60, 90, 120, 180, 240, 300, or 360 minutes following exposure to blood, serum, or plasma. In some embodiments, the synthetic microorganism exhibits measurable average cell death of at least 70%, at least 80%, at least 90%, at least 95%, at least 99%, at least 99.5%, at least 99.8%, or at least 99.9% cfu count reduction within at least 1, 5, 15, 30, 60, 90, 120, 180, 240, 300, or 360 minutes following exposure to blood, serum, or plasma.

In some embodiments, the synthetic microorganism comprises a kill switch molecular modification that reduces or prevents infectious growth of the synthetic microorganism under systemic conditions in a subject.

In some embodiments, the synthetic microorganism comprises at least one molecular modification that is integrated to a chromosome of the synthetic microorganism.

In some embodiments, the synthetic microorganism is derived from a target microorganism having the same genus and species as an undesirable microorganism. In some embodiments, the target microorganism is susceptible to at least one antimicrobial agent. In some embodiments, the target microorganism is selected from a bacterial or yeast target microorganism. In certain embodiments, the target microorganism is capable of colonizing a intramammary, dermal and/or mucosal niche.

In some embodiments, the target microorganism has the ability to biomically integrate with the decolonized host microbiome. In some embodiments, the synthetic microorganism is derived from a target microorganism isolated from the host microbiome.

The target microorganism may be a bacterial species capable of colonizing a dermal and/or mucosal niche and may be a member of a genus selected from the group consisting of Staphylococcus, Streptococcus, Escherichia, Acinetobacter, Bacillus, Mycobacterium, Mycoplasma, Enterococcus, Corynebacterium, Klebsiella, Enterobacter, Trueperella, and Pseudomonas.

The target microorganism may be selected from the group consisting of Staphylococcus aureus, coagulase-negative staphylococci (CNS), Streptococci Group A, Streptococci Group B, Streptococci Group C, Streptococci Group C & G, Staphylococcus aureus, Staphylococcus epidermidis, Staphylococcus chromogenes, Staphylococcus simulans, Staphylococcus saprophyticus, Staphylococcus haemolyticus, Staphylococcushyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa, optionally wherein the target strain is a Staphylococcus aureus 502a strain or RN4220 strain.

In some embodiments, the synthetic microorganism comprises a kill switch molecular modification comprising a cell death gene selected from the group consisting of sprA1, sprA2, kpn1, sma1, sprG, relF, rsaE, yoeB, mazF, yefM, or lysostaphin toxin gene.

In some embodiments, the inducible first promoter is a blood, serum, and/or plasma responsive promoter. In some embodiments, the first promoter is upregulated by at least 1.5 fold, at least 3-fold, at least 5-fold, at least 10-fold, at least 20-fold, at least 50-fold, or at least 100-fold within a period of time selected from the group consisting of at least 30 min, 60 min, 90 min, 120 min, 180 min, 240 min, 300 min, and at least 360 min following exposure to human blood, serum or plasma. In some embodiments, the first promoter is not induced, induced less than 1.5 fold, or is repressed in the absence of the change of state. In some embodiments, the first promoter is induced at least 1.5, 2, 3, 4, 5 or at least 6 fold within a period of time in the presence of serum, blood or plasma. In some embodiments, the first promoter is not induced, induced less than 1.5 fold, or repressed under the normal physiological (niche) conditions at the at least one site.

In some embodiments, the inducible first promoter comprises or is derived from a gene selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosyntheis protein), sbnD, sbnI, sbnE (lucA/lucC family siderophore biosynthesis protein), isdI, IrgA (murein hydrolase regulator A), lrgB (murein hydrolase regulator B), ear (Ear protein), fhuA (ferrichrome transport ATP-binding protein fhuA), fhuB (ferrichrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

The disclosure provides a live biotherapeutic composition comprising an effective amount of a synthetic microorganism according to the disclosure and a pharmaceutically acceptable carrier, optionally further comprising one or more of a diluent, surfactant, emollient, binder, excipient, sealant, barrier teat dip, lubricant, sweetening agent, flavoring agent, wetting agent, preservative, buffer, or absorbent, or a combination thereof. In some embodiments, the composition further comprises a promoting agent. In some embodiments, the promoting agent is selected from a nutrient, prebiotic, sealant, barrier teat dip, commensal, and/or probiotic bacterial species.

The disclosure provides a single dose unit comprising a live biotherapeutic composition or synthetic microorganism of the disclosure. In some embodiments, the single dose unit comprises at least at least about 10⁵, at least 10⁶, at least 10⁷, at least 10⁸, at least 10⁹, at least 10¹⁰ CFU, or at least 10¹¹, or about 10⁵ to about 10¹¹, or about 10⁶ to about 10⁹, or about 10⁷ to about 10⁸, of the synthetic strain, and a pharmaceutically acceptable carrier. In some embodiments, the single dose unit is formulated for topical administration. In some embodiments, the single dose unit is formulated for dermal or mucosal administration to at least one site of the subject.

The disclosure provides a synthetic microorganism, composition according to the disclosure for use in the manufacture of a medicament for use in a method eliminating, preventing, or reducing the risk of the recurrence of a undesirable microorganism in a subject. In some embodiments, the subject may be a mammalian subject such as a human, bovine, caprine, porcine, ovine, canine, feline, equine or other mammalian subject. In some embodiments, the subject is a human subject.

A pharmaceutical composition is provided comprising an effective amount of the synthetic microorganism of the disclosure, and a pharmaceutically acceptable excipient. The pharmaceutically acceptable excipient may include a carrier, diluent, emollient, binder, excipient, lubricant, sweetening agent, flavoring agent, wetting agent, preservative, buffer, or absorbent, or a combination thereof.

The pharmaceutical composition may comprise an effective amount of the synthetic microorganism of the disclosure, a nutrient, prebiotic, commensal, and/or probiotic bacterial species, and a pharmaceutically acceptable excipient.

A single dose unit comprising a pharmaceutical composition of the disclosure is provided, comprising at least at least 10⁵, at least 10⁶, at least 10⁷, at least 10⁸, at least 10⁹, at least 10¹⁰ CFU, or at least 10¹¹ of the synthetic microorganism and a pharmaceutically acceptable excipient. The single dose unit may be formulated for topical administration.

A composition is provided comprising the synthetic microorganism or the composition of the disclosure for use in the manufacture of a medicament for eliminating and preventing the recurrence of a undesirable microorganism in a subject.

### BRIEF DESCRIPTION OF THE DRAWINGS

FIG. 1A shows a strain design flow chart for providing a synthetic microorganism comprising a genomically stable, genomically incorporated kill switch (KS) molecular modification.

FIG. 1B shows a linear map of genomic insertion of a toxin using a piggyback method (A), compared to wild type Staphylococcus aureus target strain, BP_001 (B). In the synthetic microorganism produced using the piggyback method (A), the sprA1 gene was inserted directly after the endogenous isdB gene, with an optional intervening control arm, to obtain a synthetic Staphyloccocus aureus comprising isdB::sprA1. The isdB mRNA transcript has been extended in the synthetic microorganism to include the sprA1 gene, and will terminate downstream of the sprA1 gene, instead of right after the isdB gene as it does for the wild type strain, BP_001 (B).

FIG. 1C shows a partial sequence alignment of the insertion sequences to target strain Staphyloccocus aureus BP_001 (502a) comprising isdB::sprA1 in three synthetic strains. The serum inducible promoter is isdB. The toxin gene is sprA1. Sequence A is the mutation free sequence for BP_118, sequence B is the frame shifted mutant which shows how the isdB reading frame is impacted for BP_088, and sequence C contains two extra STOP codons after isdB in different frames for BP_115 (triple stop).

FIG. 2 shows a graph of growth curves for synthetic S. aureus strain BP_088 isdB::sprA1 in human serum (dashed lines) or tryptic soy broth (TSB) complete media (solid lines) in colony forming units per mL (cfu/mL) of culture over time (8 hours)(n=3, each condition). BP_088 growth in TSB increased from about 1×10⁷ to about 1×10⁹ cfu/ml over 4 hrs. In contrast, BP_088 exhibited significantly decreased growth in human serum from about 1×10⁷ to about 1×10³ cfu/ml over 2 hrs or less. BP_088 was unable to grow when exposed to serum, despite frame shift in isdB gene extending the reading frame by 30 bp or 10 amino acids.

FIG. 3 shows a graph of growth curves for synthetic S. aureus strain BP_115 isdB::sprA1 (n=3) and target strain wt 502a (BP_001) in human serum (dashed lines) or TSB (solid lines) in cfu/mL of culture over time (8 hours). BP_115 and wt 502a growth in TSB increased from about 1×10⁷ to about 1×10⁹ cfu/ml over about 4-6 hrs. In serum, wt 502a growth increased from about 1×10⁷ to about 6×10⁷ over about 6 hrs. In contrast, BP_115 exhibited significantly decreased growth in human serum from about 1×10⁷ to about 1×10³ cfu/ml over 2 hrs or less. Parent target strain wt 502a was able to grow when exposed to serum, but S. aureus synthetic strain BP_115 with isdB::sprA1 was unable to grow when exposed to serum.

FIG. 4 shows a graph of growth curves for BP_118 (n=3) and BP_001 (wt 502a) (n=1) in human serum and TSB. Both BP_0118 and wt502a exhibit increased growth in TSB over 8 hr. wt502a exhibits some increased growth in human serum over 8 hr. However, BP_0118 exhibits significantly decreased growth over 2 hrs or less in human serum

FIG. 5 shows a graph of average CFU/mL for S. aureus synthetic strains BP_088, BP_115, and BP_118 in TSB vs. human serum. Each of the strains is able to grow in TSB over 2-8 hr. Each of the strains exhibits significantly decreased growth when exposed to human serum for 2 hrs or less.

FIG. 6A shows Table 7B with additional plasmids and generated Staphylococcus aureus synthetic strains.

FIG. 6B shows a photographic image of a 1% agarose gel that was run to analyze the PCR from 14 Staphylococcus aureus colonies screened for the spa gene using Q5 PCR master mix. All lanes showed a positive band indicating the presence of the spa gene.

FIG. 7 shows a graph of induced and uninduced growth curves for the E. coli strain IM08B (BPEC_023) harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Within 2 hours of induction, the BPEC_023 E. coli culture growth rate slowed significantly for each following time point.

FIG. 8 shows a graph of the growth curves for the Staph aureus strain BP_001 harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Overexpression of the truncated sprA1 gene BP_DNA_090 (SEQ ID NO: 47) (encoding BP_AA_014 (SEQ ID NO: 84)) had an effect on the growing E. coli and Staph aureus cultures. The growth curves for the uninduced cultures began diverging from the induced cultures within 2 hrs following the addition of ATc, where the uninduced cultures continued to grow in log phase and the growth of the induced cultures slowed dramatically directly after the addition of ATc.

FIG. 9 shows a drawing of pIMAY plasmid used for making insertions in the genome of Staph aureus cells. The figure was taken from Monk et al. 2012.

FIG. 10 shows serum-induced fluorescence production by Staph aureus synthetic strains BP_151 (PsbnA::GFP) and BP_152 (isdB::GFP) compared to parent stain BP_001 after being cultured in human serum (dashed lines) and TSB (solid lines) over 4 hours.

FIG. 11 shows a graph of RFP mKATE2 concentration (ng/well) vs. time (hr) for serum-responsive fluorescence production by BP_157 (PsbnA::mKATE2) and BP_158 (isdB::mKATE2) in human serum (dashed lines) and TSB (solid lines). BP_001 (lacking mKATE2) was included as a wild type control.

FIG. 12 shows a graph of the average (n=6) of viable CFU/mL of Staph aureus synthetic strain BP_088 (0 and 500 generation strains) when grown in human serum (dashed lines) or TSB (solid lines). BP_001 (n=6) in TSB and serum was plotted as a wild type control. Error bars represent one standard deviation of all six replicates. The BP_088-500 generation sample is represented by solid squares (▪) and the 0 generation sample (▴). Parent strain BP_001 is represented by a solid circle. Synthetic strain BP_088 exhibits functional stability over at least 500 generations as evidenced by its retained inability to grow when exposed to human serum compared to BP_088 at 0 generations. After 2 hrs in human serum, BP_088 exhibited significantly decreased cfu/mL by about 4 orders of magnitude after about 500 generations.

FIG. 13 shows an alignment of a reference sequence for integrated sprA1 kill switch integration behind the isdB gene and the sanger sequencing results from BP_088 at 0 and 500 generation strains. The top DNA sequence is the reference sequence from a DNA map in Benchling, the middle sequence is from the BP_088 500 generation strain, and the lower sequence is from the BP_088 0 generation strain. The alignment shows no mutations or changes in the bottom two strains when compared to each other or the top reference sequence. Synthetic strain BP_088 exhibits genomic stability over at least 500 generations as evidenced by Sanger sequencing results.

FIG. 14 shows a map of the p262 plasmid made in the Benchling program (Benchling, San Francisco, Calif.). The plasmid features a pIMAYz backbone with the integration of a sprA1 gene fragment flanked by isdB homology arms.

FIG. 15 shows a bar graph of candidate promoter gene activity in serum compared to TSB at 15 min, 30 min or 45 min time points. Upregulated genes at 45 min in human serum include hlgA2, hrtAB, isdA, isdB, isdG, sbnE, ear, splD, and SAUSA300_2617.

FIG. 16 shows a bar graph of candidate promoter activity in human blood compared to TSB at 15 min, 30 min or 45 min time points. Upregulated genes at 45 min in human blood include isdA, isdB, isdG, sbnE, and SAUSA300_2617.

FIG. 17 shows a bar graph of several serum-responsive candidate genes that are upregulated after 90 minutes of incubation in serum. Gene expression at 90 minutes in both TSB and human serum were normalized to values at T=0. Specifically, genes in the isd, sbn, and fhu families are upregulated to varying degrees.

FIG. 18 shows a bar graph of the fold change in expression of 25 genes from Staph aureus at 30 and 90 minute time points in TSB and human serum. The number of reads for each gene was converted to transcripts per million (TPM), the replicates were averaged for each condition (n=3), normalized to the expression of the housekeeping gene gyrB, subtracted from the initial expression levels at t=0, and sorted for the most differentially expressed between the two media conditions at the 90 minute time point. The gene on the bottom of the chart (CH52_00245) had a value of 175 fold upregulation, but was cut short on this figure in order to enlarge the chart and maximize the clarity of the rest of the data.

FIG. 19 shows a graph of kill switch activity over 4 hours as average CFU/mL of 4 Staph aureus synthetic strains with different kill switch integrations in human serum compared to parent target strain BP_001. Strains BP_118 (isdB::spra1), BP_092 (PsbnA::sprA1) and BP_128 (harA::sprA1) each exhibited a decrease in CFU/mL at both the 2 and 4 hour time points. BP_118 (isdB::spra1) exhibited strongest kill switch activity as largest decrease in CFU/mL.

FIG. 20A shows a photograph of an Agarose gel for PCR confirmation of isdb::sprA1 in BP_118 showing the PCR products of from the secondary recombination PCR screen with primers DR_534 and DR_254. Primer DR_534 binds to the genome outside of the homology arm, and the primer DR_254 binds to the sprA1 gene making size of the amplicon is 1367 bp for s strain with the integration and making no PCR fragment if the integration is not present. BP_001 was run as a negative control to show the integration is not present in the parent strain.

FIG. 20B shows a map of the genome of Staph aureus synthetic BP_118 where the sprA1 gene was inserted. It was created with the Benchling program.

FIG. 20C shows a graph of Staph aureus synthetic strain BP_118 and parent target strain BP_001 in kill switch assay in TSB or human serum over 4 hrs. The points plotted on the graph represent an average of 3 biological replicates and the error bars represent the standard deviation for triplicate samples. The solid lines represent the cultures grown in TSB and the dashed lines represent cultures grown in human serum. The human serum assay suggested the kill switch was effective with dramatic reduction in viable cfu/mL for strain BP_118 in serum with no difference in growth in complex media (TSB) compared to the parent strain BP_001.

FIG. 21 shows a graph of an assay of the average CFU/mL for BP_112 (ΔsprA1-sprA1(AS), Site_2::PgyrB-sprA1(AS)(long), isdB::sprA1)(n=3) and BP_001 (n=1) when they are grown in serum (dashed lines) and TSB (solid lines) over an 8-hour period. The error bars represent the standard deviation of the averaged values. The human serum assay suggested kill switch was effective with dramatic reduction in viable CFU/mL for strain BP_112, with no difference in growth in complex media (TSB) compared to the wild-type parent strain BP_001

FIG. 22 shows a bar graph of the concentration of cfu/mL for all of the strains tested human plasma or TSB, at both t=0 and after 3.5 hours of growth (t=3.5). The viable cfu/mL of strains BP_088, BP_101, BP_108, and BP_109 showed over a 99% reduction after 3.5 hours in human plasma. BP_092 showed a 95% reduction in viable cfu/mL after 3.5 hours in human plasma. BP_001 showed very little difference in viable cfu/mL after 3.5 hours in human plasma. All strains grew in TSB media.

FIG. 23 shows a graph of the growth curves as OD600 values of four synthetic E. coli (sprA1) strains 1, 2, 15, 16 grown for 5 hrs in LB (+/−ATc) and induced at t=1 hr. Two different types of target E. coli strains were employed: BPEC_006 strains 1, 2, and 15 are from E. coli K12-type target strain IM08B, and strain 16 is from the bovine E. coli target strain obtained from Udder Health Systems. All induced strains (dashed lines) showed significant decrease in growth over 2-5 hr time points.

FIG. 24 shows a graph of the growth curves as OD600 values over 5 hrs with of (4) different synthetic E. coli isolates grown in LB with an inducible hokB or hokD gene integrated in the genome of K12-type E. coli target strain IM08B. Samples were induced by adding ATc to the culture 1 h post inoculation. The dashed line represents the cultures that were spiked with ATc to induce expression of the putative toxin genes and the solid line represents cultures that did not get induced by ATc. The hokD sample exhibited a diverging curve between the induced and uninduced samples. The hokB_1 is the bovine E. coli strain from Udder Health Systems and the spiked and unspiked samples grew much faster than the other 3 strains tested here

FIG. 25 shows a graph of the average (n=3) growth curves as OD600 values over 5 hrs of two synthetic E. coli strains with relE or yafQ gene integrated in the genome (n=3) grown in LB (+/−ATc). The dashed lines represent the cultures that were spiked with ATc to induce expression of the putative toxin genes and the solid lines represent cultures that did not get induced by ATc. The error bars represent one standard deviation for the averaged OD600 values for each strain. The relE gene showed diverging curves between the cultures that were induced and the uninduced cultures, where the induced cultures had significantly lower OD600 readings. The induced yafQ cultures showed a slightly slower growth between hours 2 and 4 than the uninduced cultures, but at 5 hours the two groups had nearly identical OD600 values.

FIG. 26 shows a graph the concentrations of synthetic S. aureus BP_109 and BP_121 cells grown in in TSB and human synovial fluid over the course of a 4 hour growth assay. Both BP_121 (control) and BP_109 (kill switch) cultures grew in TSB. BP_109 showed a rapid decrease in viable cfu/mL in the synovial fluid condition.

FIG. 27 shows a graph of the concentration of synthetic Staph aureus BP_109 (kill switch) and BP_121 (control) cells in TSB and Serum Enriched CSF over the course of a 6 hour assay. Both BP_121 (control) and BP_109 (kill switch) cultures grew in TSB. BP_121 also grew in CSF enriched with 2.5% human serum; however, BP_109 showed a rapid decrease in cfu/mL in the CSF condition.

FIG. 28 shows a graph of an in vivo bacteremia study in mice after tail vein injection of 10\hat{ }7 wild-type Staphylococcus aureus strains BP_001 killed (2), BP_001 WT (3), CX_001 WT(5) or synthetic Staphylococcus aureus strains comprising a kill switch BP109(4), CX_013 (6) showing avg. health, body weight, and survival over 7 days. Groups receiving BP_001 WT (3) and CX_001 WT (5) exhibited adverse clinical observations starting at day 1, greater than 15% reduction in avg body weight and death starting at day 2. By day 7, all 5 mice in CX_001 WT (5) group had died and 3 of 5 mice in BP_001 WT (3) group had died as shown at the bottom of chart. In contrast, mice receiving synthetic kill switch strains BP109 (4) and CX_013 (6), and BP_001 killed (2) all survived and exhibited no more than 10% weight loss compared to initial weight.

FIG. 29 shows a graph of animal health in an in vivo mouse SSTI study as measured by abscess formation, or lack thereof, following single SC injection of 10\hat{ }7 synthetic Staph aureus KS microorganisms or wild type Staph aureus parent strains over 10 days. Mice in KS Groups 4 (BP_109, n=5) and 6 (CX_013, n=5), respectively, maintained health over the course of this study, as compared to absess formation present in about half of the wild type parent strains Group 3 (BP_001, n=5) and Group 6 (CX_013, n=5), respectively. Animals in the negative control Groups 1 (vehicle, n=5) and 2 (killed WT BP_001, n=5) all remained healthy throughout the study and are not shown.

FIG. 30 shows health, weight and survival of mice in high dose bacteremia study after Staph aureus high dose 10\hat{ }9 injection in Groups 1-7. All mice injected with high dose modified KS strains BP_123 (group 5, n=5) and CX_013 (group 6, n=5) did not develop bacteremia and only experienced minor adverse reactions were on Day 0, the same day as injection. A graphic at the bottom of FIG. 30 represents adverse clinical observations and mortality. Both WT parent strains-BP_001 (group 1, n=5) and CX_001 (group 2, n=5)-caused severe illness and mortality in all 5 mice at 10\hat{ }9 CFU/mouse by day 5. Test group BP_092 (group 3, n=5) exhibited atypical mortality by day 1. Two mice in BP_109 (group 4, n=5) also exhibited mortality by day 4.

FIGS. 31A and 31B show graphs of cell growth assays comparing average CFU/mL (n≥3) during a 4-hour growth period in RPMI 1640 liquid media spiked with different levels of Fe(III) using Staph aureus KS strains (A) BP_109, and (B) BP_144 to determine the iron concentration levels where kill switch activation occurs.

FIG. 31(A) shows a graph of a cell growth assay comparing growth of SA KS synthetic strain BP_109, as the levels of iron in the media increases from 0 to 3 μM Fe(III), at which the growth pattern between the wild-type BP_001 and BP_109 look very similar and have overlapping error bars.

FIG. 31(B) shows a graph of a cell growth assay comparing growth of SA KS synthetic strain BP_144 having extra copy of antisense, as the levels of iron in the media increases from 0 to 1 μM Fe(III) the number of viable cells/mL also increases. The growth curves at both 1 and 3 μM Fe(III) overlap with the wild type BP_001 for the BP_144 strain. The error bars represent one standard deviation for the averaged replicates (n=2-4).

FIG. 32 shows a graph of a cell growth assay comparing the average (n≥3) CFU/mL for Staph aureus strains BP_001 (WT), BP_109 (KS) and BP_144 (KS+AS) performed in RPMI with 0.00 μM Fe(III). The viable cell counts of BP_109 decreased over the four-hour period. The error bars represent one standard deviation from the averaged replicates.

FIG. 33 shows a graph of a cell growth assay comparing average CFU/mL for BP_109 to BP_144 in Fe Spiked RPMI 1640 using with different levels of Fe(III) (0, 0.25, 0.38, and 0.60 μM) over 4 hours. BP_144 had increased viable CFU/mL compared to its parent strain BP_109 in each level of iron tested during the 4-hour growth period.

FIG. 34 shows a graph cell growth assays comparing Staph aureus strains BP_121 (no kill switch) and BP_109 (iron sensitive kill switch) in CSF and BP_109 in rabbit CSF spiked with 1.0% and 2.5% human serum. Strains were cultured in CSF or CSF+serum at a total volume of 500 μL (n=1). BP_121+2.5% human serum was analyzed in a separate assay (n=3). A trend can be seen where BP_109 loses viability as the concentration of human serum in the CSF increases. Conversely, BP_121 increases in viable cell counts upon introduction of serum to the CSF.

FIG. 35 shows a plasmid map for plasmid p306 comprising Ptet::sprG3 DNA on pRAB11 Vector. It is also representative of the plasmid map for p305 comprising Ptet::sprG2, as the only difference is the action gene sprG2 is present as opposed to sprG3.

FIG. 36 shows a graph of a growth curve as OD600 vs time in a 6-hour growth assay used to test the efficacy of action gene sprG2* (*V1M, I2L) to cause bacteriostasis in S. aureus and E. coli. Overexpression of the sprG2* gene halted the growth of both S. aureus (BP_165) and E. coli (BPEC_025), which can be seen by the lines for the induced strains (+ATc) diverging from the uninduced strains (−ATc). BPEC_025 is represented by p305 in E. coli.

FIG. 37 shows a graph of a growth curve as OD600 vs time in a 6-hour growth assay used to test the efficacy of action gene sprG3 to cause bacteriostasis in S. aureus (BP_164) and E. coli (BPEC_024). The overexpression of the sprG3 gene following induction (+ATc) halted the growth of S. aureus; however, it was only able to temporarily and less effectively inhibit the growth of E. coli. BPEC_024 is represented by p306 in E. coli.

FIG. 38 shows a graph of OD600 growth curves over 3 hours for Streptocccus agalactiae (BPST_002) transformed with plasmids p174 (sprA1) or p229 (GFP). The starting cultures were inoculated at a 1:10 dilution from stationary phase cultures. The t=0 hr OD was taken before ATc induction. The dashed line represents the cultures that were induced with ATc and the solid line represents control cultures. All data points represent single cultures. Overexpression of sprA1 toxin gene was able to inhibit S. agalactiae cell growth in exponential phase.

FIG. 39 shows a bar graph of fluorescence values at 3 hours after induction of Streptococccus agalactiae (BPST_002) transformed with plasmid p229 (GFP). The starting cultures were inoculated at a 1:10 dilution from stationary phase cultures. Cultures were grown in duplicate and fluorescence readings were performed in triplicate. Significantly increased fluorescent values of induced p229 cultures indicate the ability of the P_{XYL/Tet} promoter system of pRAB11 to function as an ATc inducible promoter in S. agalactiae.

FIG. 40 shows a bar graph calculated from the CFU/mL data of Stability Suspension D containing BP_123, BPST_002, BPEC_006 at 0 and 24 hours. All dilutions were plated in duplicate on TSB plates. CFU/mL data was calculated from the 10⁴ dilution. The observed CFU/mL at t=0 and 24 h supports the stability of cell suspensions containing a mixture of S. aureus, S. agalactiae and E. coli.

FIG. 41 shows a schematic diagram of an additional lacZ gene integrated into a native lac operon pathway in a cell.

### DETAILED DESCRIPTION

Improved methods are provided for producing stable recombinant microorganisms.

The disclosure provides strategies and methods to efficiently and stably insert specific DNA sequences to a target microorganism to create synthetic microorganisms comprising an action gene utilizing the cells native machinery to provide all of the necessary components to create the desired expression and phenotypic response, but employing minimal genomic modification.

In order to stably express native or heterologous genes over a long period of time in an organism, they may be located in the genome and not merely on a self-replicating plasmid. In addition to the location of the gene, multiple other components are required to be properly expressed, such as a regulated promoter with a transcription start site, ribosome binding site (RBS) if the gene codes for a protein, and transcription terminators. These components combine to produce a phenotypic response in the organism, and traditionally all of the required components are designed, synthesized, and inserted into a non-coding region of the genome together.

The disclosure provides methods and synthetic microorganisms having tailored toxin-antitoxin (TA) systems to engineer numerous strains of bacteria with kill switch (KS) action genes. Several kill switch strains have been designed to behave as phenotypically wild-type strains while occupying exterior niches (skin, nares) of the mammalian microbiome. However, once introduced to internal body fluid environments (plasma, serum, synovial fluid, CSF), which may be iron deficient, these modified KS strains are designed to promptly initiate artificially programmed cell death.

Toxin-antitoxin (TA) systems are biological regulatory programs utilized by most prokaryotes. Sayed et al., Nature structural & molecular biology 19.1 (2012): 105. doi:10.1038/nsmb.2193; Schuster et al., Toxins 8.5 (2016): 140. doi:10.3390/toxins8050140. In Staphylococcus aureus (S. aureus), as in many other species, these living algorithms are used for proteomic regulation in response to environmental signaling. Three types of TA systems have been identified and studied in S. aureus. The sprA1 sprA1_{AS} is a type I TA system, where the synthesis of the protein encoded by the sprA1 gene, peptide A1 (PepA1), is post-transcriptionally regulated by concomitantly transcribed antisense sprA1 (sprA1_{AS}) small non-coding RNA (sRNA). In this system, sprA1_{AS} sRNA binds to the 5′ untranslated region of sprA1 messenger RNA (mRNA) transcripts, covering the ribosome binding site, thus blocking translation of PepA1. PepA1 is a membrane porin toxin. Under normal cellular conditions, the synthesis of PepA1 is inhibited by sprA1_{AS}, which is transcribed at a 35- to 90-fold molar excess compared to sprA1. Several bacterial KS strains are provided using the sprA1 toxin gene as an initiator of cell death by inserting the toxin gene into operons involved in iron acquisition by the cells. For example, the genes isdB and sbnA are involved in iron acquisition and are highly upregulated in human blood, plasma and serum.

For example, the iron-regulated surface determinant (Isd) system binds hemoglobin, removes and transfers heme into the cytoplasm where it is degraded, releasing iron into the cell. Muryoi et al., “Demonstration of the iron-regulated surface determinant (Isd) heme transfer pathway in Staphylococcus aureus.” Journal of Biological Chemistry 2.83.42. (2008): 28125-28136. doi:10.1074/jbc.m802171200.

As another example, the sbn operon encodes the genes to biosynthesize staphyloferrin B which scavenges extracellular iron complexed to host proteins, such as transferrin. Dale et al. “Role of siderophore biosynthesis in virulence of Staphylococcus aureus: identification and characterization of genes involved in production of a siderophore.” Infection and immunity 72.1 (2004): 29-37. doi:10.1128/iai.72.1.29-37.2004.

Synthetic microorganisms are provided that when exposed to blood, plasma, or serum, are designed to activate kill switches, e.g., isdB::sprA1 and/or PsbnA::sprA1, to initiate self-destructive bacteriocidal action within 1, 2, 3, 4, 5, or 6 hrs, or 1-4, or 2-3 hours. Upon activation of one, or both, of these pathways, sprA1 mRNA transcript levels surge beyond the inhibitory threshold of sprA1_{AS}, and translation of the PepA1 protein can no longer be repressed by sprA1_{AS} sRNA. In these instances, we posit that excessive levels of the PepA1 toxin result in cell death for the KS S. aureus strains. Several earlier studies by the present inventors supported the existence of these putative KS mechanisms, both in vivo and in vitro, as provided in the present examples.

Synthetic microorganisms are provided comprising a kill switch minimal genomic modification comprising a toxin gene operably associated with a native inducible gene or promoter sensitive to, e.g., blood, serum, plasma, interstitial fluid, CSF, synovial fluid, or iron concentration. A series of experiments disclosed herein evaluated the effect of iron concentration on the viability of different synthetic S. aureus KS strains, and the ability to “tune” the efficacy of the KS with additional copies of the antitoxin integrated into the genome. The addition of a second sprA1_{AS} expression cassette into the genome may result in increased copies of sprA1_{AS} sRNA transcripts in the cytoplasm. It was hypothesized that this increase in sprA1_{AS} sRNA could be exploited to inhibit PepA1 peptide toxin expression, and thus “tune” the KS to withstand lower levels of available iron than strains harboring only one copy of sprA1_{AS}.

Iron is an essential mineral for the majority of living organisms, and it is often a growth-limiting nutrient for microorganisms. Within the human body, iron mainly exists in complex forms bound to proteins. Abbaspour et al., “Review on iron and its importance for human health.” Journal of research in medical sciences: the official journal of Isfahan University of Medical Sciences 19.2 (2014): 164. https://www.ncbi.nlm.nih.gov/pmc/articles/PMC3999603/. Host sequestration of iron is an innate immune response used to prevent infection from invading microorganisms. Hammer et al., “Molecular mechanisms of Staphylococcus aureus iron acquisition.” Annual review of microbiology 65 (2011): 129-147. doi/full/10.1146/annurev-micro-090110-102851

S. aureus can be a highly pathogenic organism with the ability to acquire iron from its host using a multitude of virulence factors including siderophores, heme acquisition pathways, and secreted enzymes. The present inventors attempted to exploit this natural tug-of-war by utilizing native iron seeking genes as transcriptional promotion sites for KS toxins.

Iron-regulated pathways are typically only highly upregulated when scavenging iron from within its host. The Staph aureus strains comprising KS integrations were strategically placed in these iron regulated pathways to minimize the effect during normal growth conditions and to maximize the effect during infection conditions. When the kill switched cell enters the blood, plasma, serum, or CSF, the iron-regulated genes will be induced along with the sprA1 KS gene, causing apoptosis in the cell and preventing possible infection.

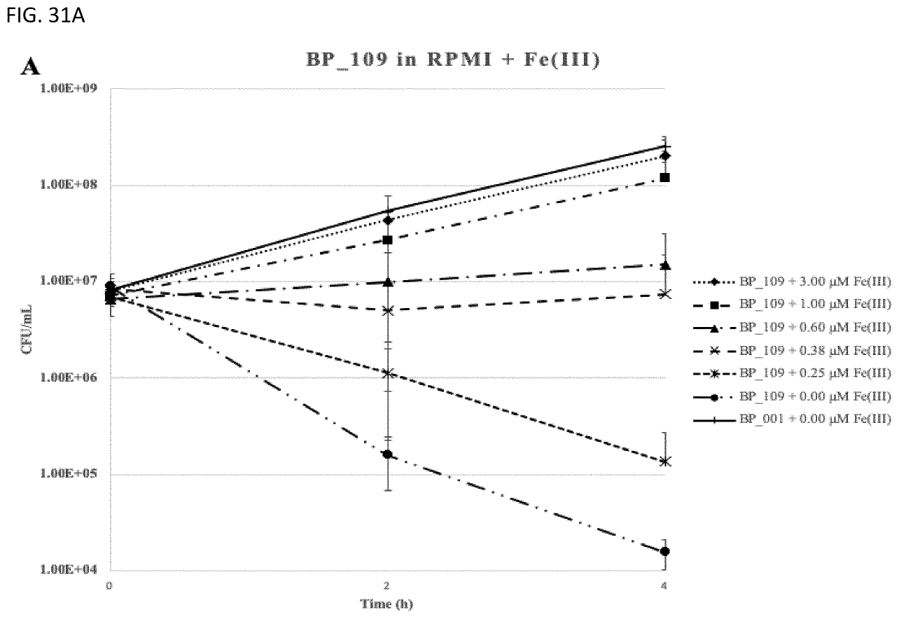

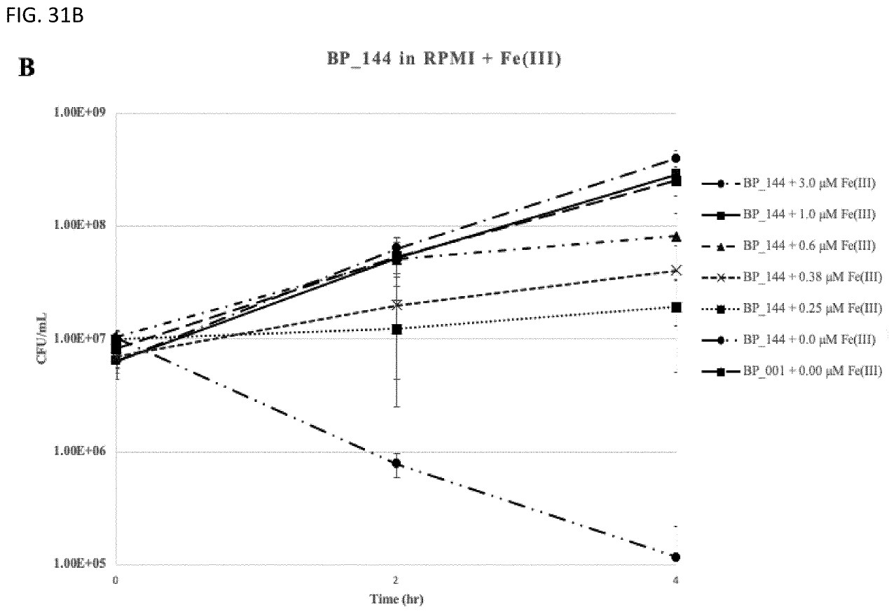

Example 20 provided herein investigated growth patterns of synthetic KS strains of S. aureus compared to WT in response to varying levels of iron availability, for example, in serum and RPMI growth assays. BP_001, a wild type Staph aureus strain, was tested at the same levels of Fe(III) spiked into RPMI media, with no difference or toxicity observed for any level tested. This indicates that any deviation from the wild type growth curve is associated with the integrated kill switches and not a toxic effect seen from too much iron in the media. The data from these assays are shown in FIGS. 31 to 34. In engineered kill switch strains modified with an additional copy of the native sprA1_{AS} expression cassette (e.g., BP_144), viable cell counts were higher at the termination of growth assays in iron deficient media, as compared to their parent strains, e.g., BP_109. Therefore, increasing the number of sprA1_{AS} expression cassettes in a genome can change the efficacy of the sprA1 kill switches when the cells are grown in iron-limiting media. As shown in FIGS. 31 to 34, a linear relationship was demonstrated for a specific range of available iron in the media to the number of viable CFU/mL in a culture in the synthetic Staph aureus strains.

Two observations were made based on tenability experiments.

First, the iron sensitive kill switches in BP_109 and BP_144 appear to activate in an iron dose dependent manner across a limited “action range.” That is to say, within a certain concentration range of available iron, the efficacy of the KS in decreasing bacterial cell viability is negatively correlated to the iron concentration of the media. Conversely, the viability of the KS strains is highly correlated to the concentration of available iron. The linear relationship between cell viability and iron concentration definitively demonstrates the reliance of the KS on iron availability (See FIGS. 31-34). This correlation empirically corroborates the proposed mechanism of action of the synthetic KS strains possessing iron sensitive kill switches.

Second, as an extension of the first finding, the apparent linear correlation between iron availability and KS activation supports the possibility that the isdB::sprA1 and PsbnA::sprA1 kill switches can be tuned. The Isd and sbn iron acquisition pathways are regulated by the transcriptional ferric uptake regulator (fur) which allows partial or variable expression. It seems that the activation of these kill switches is not an “all or nothing” response, but rather a gradient-based system affected by multiple factors, one of which is the level of available iron.

Data from example 20 for SA synthetic strains BP_109 and BP_144 iron spike assays in RPMI 1640 indicate that additional sprA1_{AS} can potentiate the threshold of KS activation. Strains modified with an extra antisense insertion cassette consistently produced more viable cells compared to their parent strain within each condition in the “action range” of iron availability, further suggesting tunability of the KS. The additional copy of the sprA1_{AS} expression cassette was inserted into the genome of certain KS strains (e.g., BP_144) in a non-coding region named Site2. As the extra copy of the sprA1_{AS} appears to help regulate the sprA1 kill switches in other regions of the genome, we can conclude that in order for the antitoxin to be effective, it does not have to be located adjacent to the toxin gene it is suppressing.

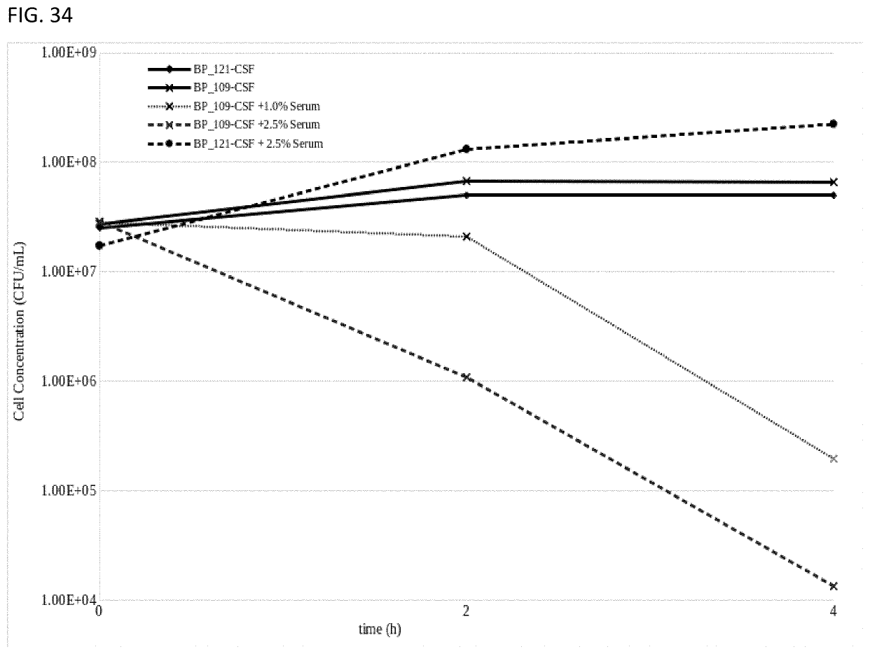

In the CSF assay shown in FIG. 34, a trend can be seen where BP_109 loses viability as the concentration of human serum in the CSF increases. The wild-type control, BP_121, was not grown in the CSF+1.0% serum spiked condition, due to limited CSF availability; however, BP_121 readily grows in human serum and has been demonstrated to show increased viability when cultured in serum-enriched CSF conditions. The data shown here indicate that the level of KS activation in CSF may be linked to the nutrient levels in the environment and the corresponding levels of metabolic activity in the cell.

Tunability of the KS in vivo allows future strains to be designed to thrive in various environments while retaining functionality of the kill switch in desired states. On average, metabolites in the blood and serum of humans may drastically vary in concentration (+/−50%). The ecology of the skin microbiome is dependent on topographical location, endogenous host factors and exogenous environmental factors. The ability to “tune” the kill switch depending on differences in host environments may be exploited to build a generation of a library of KS strains designed to be patient or geographically specific.

Methods for identifying native inducible genes or promoters in a target microorganism are provided. Through RNA seq or qPCR, the transcriptome in a target microorganism strain may be analyzed to identify differentially expressed endogenous promoter gene candidates under various growth conditions. The top endogenous promoter gene candidates demonstrating the appropriate levels of expression under different conditions may be located on the genome. The required elements in the operon may also be identified. For example, if the endogenous candidate promoter genes for genetic insertion are unregulated in the target strain or in a passthrough strain, the action gene may be integrated between the stop codon and the transcriptional terminator of any gene located in an operon. This allows for the inserted action gene to “piggyback” off of the native regulation of the operon by the cell.

### Definitions

The singular forms “a”, “an” and “the” are intended to include the plural forms as well, unless the context clearly indicates otherwise.

The term “and/or” refers to and encompasses any and all possible combinations of one or more of the associated listed items.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The term “pathogen” or “pathogenic microorganism” refers to a microorganism that is capable of causing disease. A pathogenic microorganism may colonize a site on a subject and may subsequently cause systemic infection in a subject. The pathogenic microorganism may have evolved the genetic ability to breach cellular and anatomic barriers that ordinarily restrict other microorganisms. Pathogens may inherently cause damage to cells to forcefully gain access to a new, unique niche that provides them with less competition from other microorganisms, as well as with a ready new source of nutrients. Falkow, Stanley, 1998 Emerging Infectious Diseases, Vol. 4, No. 3, 495-497. The pathogenic microorganism may be a drug-resistant microorganism.

The term “virulent” or “virulence” is used to describe the power of a microorganism to cause disease.

The term “commensal” refers to a form of symbioses in which one organism derives food or other benefits from another organism without affecting it. Commensal bacteria are usually part of the normal flora.

The term “suppress” or “decolonize” means to substantially reduce or eliminate the original undesired pathogenic microorganism by various means (frequently referred to as “decolonization”). Substantially reduce refers to reduction of the undesirable microorganism by greater than 90%, 95%, 98%, 99%, or greater than 99.9% of original colonization by any means known in the art.

The term “replace” refers to replacing the original pathogenic microorganism by introducing a new microorganism (frequently referred to as “recolonization”) that “crowds out” and occupies the niche(s) that the original microorganism would ordinarily occupy, and thus preventing the original undesired microorganism from returning to the microbiome ecosystem (frequently referred to as “interference” and “non-co-colonization”).

The term “durably replace”, “durably exclude”, “durable exclusion”, or “durable replacement”, refers to detectable presence of the new synthetic microorganism for a period of at least 30 days, 60 days, 84 days, 120 days, 168 days, or 180 days after introduction of the new microorganism to a subject, for example, as detected by swabbing the subject. In some embodiments, “durably replace”, “durably exclude”, “durable exclusion”, or “durable replacement” refers to absence of the original pathogenic microorganism for a period of at least 30 days, 60 days, 84 days, 120 days, 168 days, or 180 days after introduction of the new synthetic microorganism to the subject, for example, absence as detected over at least two consecutive plural sample periods, for example, by swabbing the subject.

The term “rheostatic cell” refers to a synthetic microorganism that has the ability to durably occupy a native niche, or naturally occurring niche, in a subject, and also has the ability to respond to change in state in its environment.

The term “promote”, or “promoting”, refers to activities or methods to enhance the colonization and survival of the new organism, for example, in the subject. For example, promoting colonization of a synthetic bacteria in a subject may include administering a nutrient, prebiotic, and/or probiotic bacterial species.

The terms “prevention”, “prevent”, “preventing”, “prophylaxis” and as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The disclosure provides methods and compositions comprising a synthetic microorganism useful for eliminating and preventing the recurrence of a undesirable microorganism in a subject hosting a microbiome, comprising (a) decolonizing the host microbiome; and (b) durably replacing the undesirable microorganism by administering to the subject the synthetic microorganism comprising at least one element imparting a non-native attribute, wherein the synthetic microorganism is capable of durably integrating to the host microbiome, and occupying the same niche in the host microbiome as the undesirable microorganism.

In some embodiments, a method is provided comprising a decolonizing step comprising topically administering a decolonizing agent to at least one site in the subject to reduce or eliminate the presence of an undesirable microorganism from the at least one site.

In some embodiments, the decolonizing step comprises topical administration of a decolonizing agent, wherein no systemic antimicrobial agent is simultaneously administered. In some embodiments, no systemic antimicrobial agent is administered prior to, concurrent with, and/or subsequent to within one week, two weeks, three weeks, one month, two months, three months, six months, or one year of the first topical administration of the decolonizing agent or administration of the synthetic microorganism. In some embodiments, the decolonizing agent is selected from the group consisting of a disinfectant, bacteriocide, antiseptic, astringent, and antimicrobial agent.

The disclosure provides a synthetic microorganism for durably replacing an undesirable microorganism in a subject. The synthetic microorganism comprises a molecular modification designed to enhance safety by reducing the risk of systemic infection. In one embodiment, the molecular modification causes a significant reduction in growth or cell death of the synthetic microorganism in response to blood, serum, plasma, or interstitial fluid. The synthetic microorganism may be used in methods and compositions for preventing or reducing recurrence of dermal or mucosal colonization or recolonization of an undesirable microorganism in a subject.

The disclosure provides a synthetic microorganism for use in compositions and methods for treating or preventing, reducing the risk of, or reducing the likelihood of colonization, or recolonization, systemic infection, bacteremia, or endocarditis caused by an undesirable microorganism in a subject.

In some embodiments, the subject treated with a method according to the disclosure does not exhibit recurrence or colonization of an undesirable microorganism as evidenced by swabbing the subject at the at least one site for at least two weeks, at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to a mammal such as a human being, a companion animal, a service animal, or a food chain mammal, such as cattle, goats, sheep, rabbits, hogs, camel, yak, buffalo, horse, donkey, zebu, reindeer, or giraffe. In particular, the term may specify male or female. In one embodiment, the subject is a female cow, goat, or sheep. The companion animal may be a dog, cat, pleasure horse, bird, rat, gerbil, mouse, guinea pig, or ferret. The food chain animal may be a chicken, turkey, goose, or duck. In another embodiment, both female and male animals may be subjects. In one aspect, the patient is an adult human or animal. In another aspect, the patient is a non-neonate human or animal. In some embodiments, the subject is a female or male human found to be colonized with an undesirable or pathogenic strain of a microorganism.

The term “neonate”, or newborn, refers to an infant in the first 28 days after birth. The term “non-neonate” refers to an animal older than 28 days.

The term “effective amount” as used herein refers to an amount of an agent, either alone or as a part of a pharmaceutical composition, that is capable of having any detectable, positive effect on any symptom, aspect, or characteristics of a disease state or condition. Such effect need not be absolute to be beneficial.

The term “measurable average cell death” refers to the inverse of survival percentage for a microorganism determined at a predefined period of time after introducing a change in state compared to the same microorganism in the absence of a change in state under defined conditions. The survival percentage may be determined by any known method for quantifying live microbial cells. For example, survival percentage may be calculated by counting cfus/mL for cultured synthetic microorganism cells and counting cfus/mL of uninduced synthetic microorganism cells at the predefined period of time, then dividing cfus induced/mL by cfus/mL uninduced×100=x % survival percentage. The measurable average cell death may be determined by 100%−x % survival percentage=y % measurable average cell death. For example, wherein the survival percentage is 5%, the measurable average cell death is 100%−5%=95%. Any method for counting cultured live microbial cells may be employed for calculation of survival percentage including cfu, OD600, flow cytometry, or other known techniques. Likewise, an induced synthetic strain may be compared to a wild-type target microorganism exposed to the same conditions for the same period of time, using similar calculations to determine a “survival rate” wherein 100%−survival rate=z % “reduction in viable cells”.

In some embodiments, the measurable average cell death of the synthetic microorganism occurs within at least a preset period of time following induction of the first promoter after a “change in state”, for example exposure to a second environment. In some embodiments, the measurable average cell death occurs within at least a preset period of time selected from the group consisting of within at least 1, 5, 15, 30, 60, 90, 120, 180, 240, 300, or 360 min minutes following the change of state. In some embodiments, the measurable average cell death is at least a 50% cfu, at least 70%, at least 80%, at least 90%, at least 95%, at least 99%, at least 99.5%, at least 99.8%, or at least 99.9% cfu count reduction following the preset period of time.

In some embodiments, the change in state is a change in the cell environment which may be, for example, selected from one or more of pH, temperature, osmotic pressure, osmolality, oxygen level, nutrient concentration, blood concentration, plasma concentration, serum concentration, metal concentration, iron concentration, chelated metal concentration, change in composition or concentration of one or more immune factors, mineral concentration, and electrolyte concentration. In some embodiments, the change in state is a higher concentration of and/or change in composition of blood, serum, plasma, cerebral spinal fluid (CSF), contaminated CSF, synovial fluid, or interstitial fluid, compared to normal physiological (niche) conditions at the at least one site in the subject. In some embodiments, “normal physiological conditions” may be dermal or mucosal conditions, or cell growth in a complete media such as TSB.

The term “including” as used herein is non-limiting in scope, such that additional elements are contemplated as being possible in addition to those listed; this term may be read in any instance as “including, but not limited to.”

The term “shuttle vector” as used herein refers to a vector constructed so it can propagate in two different host species. Therefore, DNA inserted into a shuttle vector can be tested or manipulated in two different cell types.

The term “plasmid” as used herein refers to a double-stranded DNA, typically in a circular form, that is separate from the chromosomes, for example, which may be found in bacteria and protozoa.

The term “expression vector”, also known as an “expression construct”, is generally a plasmid that is used to introduce a specific gene into a target cell.

The term “transcription” refers to the synthesis of RNA under the direction of DNA.

The term “transformation” or “transforming” as used herein refers to the alteration of a bacterial cell caused by transfer of DNA. The term “transform” or “transformation” refers to the transfer of a nucleic acid fragment into a parent bacterial cell, resulting in genetically-stable inheritance. Synthetic bacterial cells comprising the transformed nucleic acid fragment may also be referred to as “recombinant” or “transgenic” or “transformed” organisms.

As used herein, “stably maintained” or “stable” synthetic bacterium is used to refer to a synthetic bacterial cell carrying non-native genetic material, e.g., a cell death gene, and/or other action gene, that is incorporated into the cell genome such that the non-native genetic material is retained, and propagated. The stable bacterium is capable of survival and/or growth in vitro, e.g., in medium, and/or in vivo, e.g., in a dermal, mucosal, or other intended environment.

The term “operon” as used herein refers to a functioning unit of DNA containing a cluster of genes under the control of a single promoter. The genes are transcribed together into an mRNA strand and either translated together in the cytoplasm, or undergo splicing to create monocistronic mRNAs that are translated separately, i.e. several strands of mRNA that each encode a single gene product. The result of this is that the genes contained in the operon are either expressed together or not at all. Several genes must be co-transcribed to define an operon.

The term “operably linked” refers to an association of nucleic acid sequences on a single nucleic acid sequence such that the function of one is affected by the other. For example, a regulatory element such as a promoter is operably linked with an action gene when it is capable of affecting the expression of the action gene, regardless of the distance between the regulatory element such as the promoter and the action gene. More specifically, operably linked refers to a nucleic acid sequence, e.g., comprising an action gene, that is joined to a regulatory element, e.g., an inducible promoter, in a manner which allows expression of the action gene(s).

The term “regulatory region” refers to a nucleic acid sequence that can direct transcription of a gene of interest, such as an action gene, and may comprise various regulatory elements such as promoter sequences, enhancer sequences, response elements, protein recognition sites, inducible elements, promoter control elements, protein binding sequences, 5 and 3′ untranslated regions, transcriptional start sites, termination sequences, polyadenylation sequences, and introns.

The term “promoter” or “promoter gene” as used herein refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. In some cases, promoters may regulate expression of a coding sequence or gene in response to a particular stimulus, e.g., in a cell- or tissue-specific manner, in response to different environmental or physiological conditions, or in response to specific compounds. Prokaryotic promoters may be classified into two classes: inducible and constitutive.

An “inducible promoter” or “inducible promoter gene” refers to a regulatory element within a regulatory region that is operably linked to one or more genes, such as an action gene, wherein expression of the gene(s) is increased in response to a particular environmental condition or in the presence of an inducer of said regulatory region. An “inducible promoter” refers to a promoter that initiates increased levels of transcription of the coding sequence or gene under its control in response to a stimulus or an exogenous environmental condition. The inducible promoter may be induced upon exposure to a change in environmental condition. The inducible promoter may be a blood or serum inducible promoter, inducible upon exposure to a protein, inducible upon exposure to a carbohydrate, or inducible upon a pH change.

The blood or serum inducible promoter may be selected from the group consisting of isdB, leuA, hlgA, hlgA2, isdG, sbnC, sbnE, hlgB, SAUSA300_2616, splF, fhuB, hlb, hrtAB, IsdG, LrgA, SAUSA300_2268, SAUSA200_2617, SbnE, IsdI, LrgB, SbnC, HlgB, IsdG, SplF, IsdI, LrgA, HlgA2, CH52_04385, CH52_05105, CH52_06885, CH52_10455, PsbnA, and sbnA.

The term “constitutive promoter” refers to a promoter that is capable of facilitating continuous transcription of a coding sequence or gene under its control and/or to which it is operably linked under normal physiological conditions.

The term “animal” refers to the animal kingdom definition.

The term “substantial identity” or “substantially identical,” when referring to a nucleotide or fragment thereof, indicates that, when optimally aligned with appropriate nucleotide insertions or deletions with another nucleotide (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleotide molecule having substantial identity to a reference nucleotide molecule may, in certain instances, encode a polypeptide having the same or substantially similar amino acid sequence as the polypeptide encoded by the reference nucleotide molecule.

The term “derived from” when made in reference to a nucleotide or amino acid sequence refers to a modified sequence having at least 50% of the contiguous reference nucleotide or amino acid sequence respectively, wherein the modified sequence causes the synthetic microorganism to exhibit a similar desirable attribute as the reference sequence of a genetic element such as promoter, cell death gene, antitoxin gene, virulence block, or nanofactory, including upregulation or downregulation in response to a change in state, or the ability to express a toxin, antitoxin, or nanofactory product, or a substantially similar sequence, the ability to transcribe an antisense RNA antitoxin, or the ability to prevent or diminish horizontal gene transfer of genetic material from the undesirable microorganism. The term “derived from” in reference to a nucleotide sequence also includes a modified sequence that has been codon optimized for a particular microorganism to express a substantially similar amino acid sequence to that encoded by the reference nucleotide sequence. The term “derived from” when made in reference to a microorganism, refers to a target microorganism that is subjected to a molecular modification to obtain a synthetic microorganism.

The term “substantial similarity” or “substantially similar” as applied to polypeptides means that two peptide or protein sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions.

The term “conservative amino acid substitution” refers to wherein one amino acid residue is substituted by another amino acid residue having a side chain (R group) with similar chemical properties, such as charge or hydrophobicity. In general, a conservative amino acid substitution will not substantially change the functional properties of the, e.g., toxin or antitoxin protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Polypeptide sequences may be compared using FASTA using default or recommended parameters, a program in GCG Version 6.1. FASTA (e.g., FASTA2 and FASTA3) provides alignments and percent sequence identity of the regions of the best overlap between the query and search sequences (see, e.g., Pearson, W. R., Methods Mol Biol 132: 185-219 (2000), herein incorporated by reference). Another preferred algorithm when comparing a sequence of the disclosure to a database containing a large number of sequences from different organisms is the computer program BLAST, especially BLASTP or TBLASTN, using default parameters. See, e. g., Altschul et al., J Mol Biol 215:403-410 (1990) and Altschul et al., Nucleic Acids Res 25:3389-402 (1997).

Unless otherwise indicated, nucleotide sequences provided herein are presented in the 5′-3′ direction.

All pronouns are intended to be given their broadest meaning. Unless stated otherwise, female pronouns encompass the male, male pronouns encompass the female, singular pronouns encompass the plural, and plural pronouns encompass the singular.

The term “systemic administration” refers to a route of administration into the circulatory system so that the entire body is affected. Systemic administration can take place through enteral administration (absorption through the gastrointestinal tract, e.g. oral administration) or parenteral administration (e.g., injection, infusion, or implantation).

The term “topical administration” refers to application to a localized area of the body or to the surface of a body part regardless of the location of the effect. Typical sites for topical administration include sites on the skin or mucous membranes. In some embodiments, topical route of administration includes enteral administration of medications or compositions.

The term “undesirable microorganism” refers to a microorganism which may be a pathogenic microorganism, drug-resistant microorganism, antibiotic-resistant microorganism, irritation-causing microorganism, odor-causing microorganism and/or may be a microorganism comprising an undesirable virulence factor. The undesirable microorganism may be a bacterial species having a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

The “undesirable microorganism” may be selected from the group consisting of Staphylococcus aureus, coagulase-negative staphylococci (CNS), Streptococci Group A, Streptococci Group B, Streptococci Group C, Streptococci Group C & G, Staphylococcus spp., Staphylococcus epidermidis, Staphylococcus chromogenes, Staphylococcus simulans, Staphylococcus saprophyticus, Staphylococcus haemolyticus, Staphylococcushyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, and Pseudomonas aeruginosa.

In some embodiments, the undesirable microorganism is an antimicrobial agent-resistant microorganism. In some embodiments, the antimicrobial agent-resistant microorganism is an antibiotic resistant bacteria. In some embodiments, the antibiotic-resistant bacteria is a Gram-positive bacterial species selected from the group consisting of a Streptococcus spp., Cutibacterium spp., and a Staphylococcus spp. In some embodiments, the Streptococcus spp. is selected from the group consisting of Streptococcus pneumoniae, Steptococcus mutans, Streptococcus sobrinus, Streptococcus pyogenes, and Streptococcus agalactiae. In some embodiments, the Cutibacterium spp. is selected from the group consisting of Cutibacterium acnes subsp. acnes, Cutibacterium acnes subsp. defendens, and Cutibacterium acnes subsp. elongatum. In some embodiments, the Staphylococcus spp. is selected from the group consisting of Staphylococcus aureus, Staphylococcus epidermidis, and Staphylococcus saprophyticus. In some embodiments, the undesirable microorganism is a methicillin-resistant Staphylococcus aureus (MRSA) strain that contains a staphylococcal chromosome cassette (SCCmec types I-III), which encode one (SCCmec type I) or multiple antibiotic resistance genes (SCCmec type II and III), and/or produces a toxin. In some embodiments, the toxin is selected from the group consisting of a Panton-Valentine leucocidin (PVL) toxin, toxic shock syndrome toxin-1 (TSST-1), staphylococcal alpha-hemolysin toxin, staphylococcal beta-hemolysin toxin, staphylococcal gamma-hemolysin toxin, staphylococcal delta-hemolysin toxin, enterotoxin A, enterotoxin B, enterotoxin C, enterotoxin D, enterotoxin E, and a coagulase toxin.

In some embodiments, the undesirable microorganism is a Staphyloccoccus aureus strain, and wherein the detectable presence is measured by a method comprising obtaining a sample from at least one site of the subject, contacting a chromogenic agar with the sample, incubating the contacted agar and counting the positive cfus of the bacterial species after a predetermined period of time.

The term “synthetic microorganism” refers to an isolated microorganism modified by any means to comprise at least one element imparting a non-native attribute. For example, the synthetic microorganism may be a “recombinant microorganism” engineered to include a molecular modification comprising an addition, deletion and/or modification of genetic material to incorporate a non-native attribute. In some embodiments, the synthetic microorganism is not an auxotroph.

The term “auxotroph”, “auxotrophic strain”, or “auxotrophic mutant”, as used herein refers to a strain of microorganism that requires a growth supplement that the organism from nature (wild-type strain) does not require. For example, auxotrophic strains of Staphylococcus epidermidis that are dependent on D-alanine for growth are disclosed in US 20190256935, Whitfill et al., which is incorporated herein by reference.

The term “biotherapeutic composition” or “live biotherapeutic composition” refers to a composition comprising a synthetic microorganism according to the disclosure.

The term “live biotherapeutic product” (LBP) as used herein refers to a biological product that 1) contains live organisms, such as bacteria; 2) is applicable to prevention, treatment, or cure of a disease or condition in human beings; and 3) is not a vaccine. As described herein, LBPs are not filterable viruses, oncolytic bacteria, or products intended as gene therapy agents, and as a general matter, are not administered by injection.

A “recombinant LBP” (rLBP) as used herein is a live biotherapeutic product comprising microorganisms that have been genetically modified through the purposeful addition, deletion, or modification of genetic material.

A “drug” as used herein includes but is not limited to articles intended for use in the diagnosis, cure, mitigation, treatment, or prevention of disease in man or other animals.

A “drug substance” as used herein is the unformulated active substance that may subsequently be formulated with excipients to produce drug products. The microorganisms contained in an LBP are typically cellular microbes such as bacteria or yeast. Thus the drug substance for an LBP is typically the unformulated live cells.

A “drug product” as used herein is the finished dosage form of the product.

The term “detectable presence” of a microorganism refers to a confirmed positive detection in a sample of a microorganism genus, species and/or strain by any method known in the art. Confirmation may be a positive test interpretation by a skilled practitioner and/or by repeating the method.

The term “microbiome,” or “microbiomic,” or “microbiota” as used herein refers to microbiological ecosystems. These ecosystems are a community of commensal, symbiotic and pathogenic microorganisms found in and on all animals and plants.

The term “microorganism” as used herein refers to an organism that can be seen only with the aid of a microscope and that typically consists of only a single cell. Microorganisms include bacteria, protozoans and fungi.

The term “niche” and “niche conditions” as used herein refers to the ecologic array of environmental and nutritional requirements that are required for a particular species of microorganism. The definitions of the values for the niche of a species defines the places in the particular biomes that can be physically occupied by that species and defines the possible microbial competitors.

The term “colonization” as used herein refers to the persistent detectable presence of a microorganism on a body surface, e.g., a dermal or mucosal surface, without causing disease in the individual.

The term “co-colonization” as used herein refers to simultaneous colonization of a niche in a site on a subject by two or more strains, or variants within the same species of microorganisms. For example, the term “co-colonization” may refer to two or more strains or variants simultaneously and non-transiently occupying the same niche. The term non-transiently refers to positive identification of a strain or variant at a site in a subject over time at two or more time subsequent points in a multiplicity of samples obtained from the subject at least two weeks apart.

The term “target microorganism” as used herein refers to a wild-type microorganism or a parent synthetic microorganism, for example, selected for molecular modification to provide a synthetic microorganism. The target microorganism may be of the same genus and species as the undesirable microorganism, which may cause a pathogenic infection.

The “target microorganism” may be selected from the group consisting of Staphylococcus aureus, coagulase-negative staphylococci (CNS), Streptococci Group A, Streptococci Group B, Streptococci Group C, Streptococci Group C & G, Staphylococcus spp., Staphylococcus epidermidis, Staphylococcus chromogenes, Staphylococcus simulans, Staphylococcus saprophyticus, Staphylococcus haemolyticus, Staphylococcushyicus, Acinetobacter baumannii, Acinetobacter calcoaceticus, Streptococcus pyogenes, Streptococcus agalactiae, Streptococcus dysgalactiae, Streptococcus uberis, Escherichia coli, Mastitis Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, and Pseudomonas aeruginosa.

The “target strain” may be the particular strain of target microorganism selected for molecular modification to provide the synthetic microorganism. Preferably, the target strain is sensitive to one or more antimicrobial agents. For example, if the undesirable microorganism is a Methicillin resistant Staphylococcus aureus (MRSA) strain, the target microorganism may be an antibiotic susceptible target strain, or Methicillin Susceptible Staphylococcus aureus (MSSA) strain, such as WT-502a. In some embodiments, the target microorganism may be of the same species as the undesirable microorganism. In some embodiments, the target microorganism may be a different strain, but of the same species as the undesirable microorganism.

The term “bacterial replacement” or “non-co-colonization” as used herein refers to the principle that only one variant/strain of one species can occupy any given niche within the biome at any given time.

The term “action gene” as used herein refers to a preselected gene to be incorporated to a molecular modification, for example, in a target microorganism. The molecular modification comprises the action gene operatively associated with a regulatory region comprising an inducible promoter. The action gene may include exogenous DNA. The action gene may include endogenous DNA. The action gene may include DNA having the same or substantially identical nucleic acid sequence as an endogenous gene in the target microorganism. The action gene may encode a molecule, such as a protein, that when expressed in an effective amount causes an action or phenotypic response within the cell. The action or phenotypic response may be selected from the group consisting of cell suicide (kill switch molecular modification), prevention of horizontal gene transfer (virulence block molecular modification), metabolic modification (metabolic molecular modification), reporter gene, and production of a desirable molecule (nano factory molecular modification).

The term “kill switch”, or “KS” as used herein refers to an intentional molecular modification of a synthetic microorganism, the molecular modification comprising a cell death gene operably linked to a regulatory region comprising an inducible promoter, genetic element or cassette, wherein induced expression of the cell death gene in the kill switch causes cell death, arrest of growth, or inability to replicate, of the microorganism in response to a specific state change such as a change in environmental condition of the microorganism. For example, in the synthetic microorganism comprising a kill switch, the inducible first promoter may be activated by the presence of blood, serum, plasma, heme, synovial fluid, interstitial fluid, or contaminated cerebrospinal fluid (CSF), wherein the upregulation and transcription/expression of the operably associated cell death gene results in cell death of the microorganism, or arrested growth, of the microorganism so as to improve the safety of the synthetic microorganism.

The target microorganism may be, for example, a Staphylococcus species, Escherichia species, or a Streptococcus species.

The target microorganism may be a Staphylococcus species or an Escherichia species. The target microorganism may be a Staphylococcus aureus target strain. The action gene may be a toxin gene. Toxin genes may be selected from sprA1, sma1, rsaE, relF, 187/lysK, Holin, lysostaphin, SprG1, sprG2, sprG3, SprA2, mazF, Yoeb-sa2. The inducible promoter gene may be a serum, blood, plasma, heme, CSF, interstitial fluid, or synovial fluid inducible promoter gene, for example, selected from isdB, leuA, hlgA, hlgA2, isdG, sbnC, sbnE, hlgB, SAUSA300_2616, splF, fhuB, hlb, hrtAB, IsdG, LrgA, SAUSA300_2268, SAUSA200_2617, SbnE, IsdI, LrgB, SbnC, HlgB, IsdG, SplF, IsdI, LrgA, HlgA2, CH52_04385, CH52_05105, CH52_06885, CH52_10455, PsbnA, or sbnA.

The target microorganism may be a Streptococcus species. The target microorganism may be a Streptococcus agalactiae, Streptococcus pneumonia, or Streptococcus mutans target strain. The action gene may be a toxin gene. The toxin gene may be selected from a RelE/ParE family toxin, ImmA/IrrE family toxin, mazEF, ccd or relBE, Bro, abiGII, HicA, COG2856, RelE, or Fic. The inducible promoter gene may be a serum, blood, plasma, heme, CSF, interstitial fluid, or synovial fluid inducible promoter gene, for example, selected from a Regulatory protein CpsA, Capsular polysaccharide synthesis protein CpsH, Polysaccharide biosynthesis protein CpsL, R3H domain-containing protein, Tyrosine-protein kinase CpsD, Capsular polysaccharide biosynthesis protein CpsC, UDP-N-acetylglucosamine-2-epimerase NeuC, GTP pyrophosphokinase RelA, PTS system transporter subunit IIA, Glycosyl transferase CpsE, Capsular polysaccharide biosynthesis protein CpsJ, NeuD protein, IgA-binding R antigen, Polysaccharide biosynthesis protein CpsG, Polysaccharide biosynthesis protein CpsF, or a Fibrinogen binding surface protein C FbsC.

The term “metabolic molecular modification” refers to an intentional molecular modification of a synthetic microorganism designed to address a genetic disorder of metabolism, wherein a subject produces an abnormal amount of an enzyme that typically regulates a metabolic molecule in the subject.

Metabolism encompasses a complex set of chemical reactions that the body uses to maintain life, including energy production. Certain enzymes break down food or certain chemicals so the body can use them immediately for fuel or store them. Also, certain chemical processes break down substances that the body no longer needs, or make those it lacks.

When these chemical processes do not function properly due to a hormone or enzyme deficiency, a metabolic disorder occurs. Inherited metabolic disorders fall into different categories, depending on the specific substance and whether it builds up in harmful amounts (because it cannot be broken down), it's too low, or it's missing. There are hundreds of inherited metabolic disorders, caused by different genetic defects.

For example, see www.mayoclinic.org/diseases-conditions/inherited-metabolic-disorders/symptoms-causes/syc-20352590.

The subject may suffer from a metabolic disorder such as diabetes mellitus (high blood glucose over prolonged period of time due to low production of insulin), lactose intolerance (inability to metabolize lactose to form glucose and galactose due to reduced lactase production), and phenylketonuria (PKU) (inability to convert phenyalanine into tyrosine due to lack of phenylalanine hydroxylase).

The term “exogenous DNA” as used herein refers to DNA originating outside the target microorganism. The exogenous DNA may be introduced to the genome of the target microorganism using methods described herein. The exogenous DNA may or may not have the same or substantially identical nucleic acid sequence as found in a target microorganism, but may be inserted to a non-natural location in the genome. For example, exogenous DNA may be copied from a different part of the same genome it is being inserted into, since the insertion fragment was created outside the target organism (i.e. PCR, synthetic DNA, etc.) and then transformed into the target organism, it is exogenous.

The term “exogenous gene” as used herein refers to a gene originating outside the target microorganism. The exogenous gene may or may not have the same or substantially identical nucleic acid sequence as found in a target microorganism, but may be inserted to a non-natural location in the genome. Transgenes are exogenous DNA sequences introduced into the genome of a microorganism. These transgenes may include genes from the same microorganism or novel genes from a completely different microorganism. The resulting microorganism is said to be transformed.

The term “endogenous DNA” as used herein refers to DNA originating within the genome of a target microorganism prior to genomic modification.

The term “endogenous gene” as used herein refers to a gene originating within the genome of a target microorganism prior to genomic modification.

As used herein the term “minimal genomic modification” (MGM) refers to a molecular modification made to a target microorganism, wherein the MGM comprises an action gene operatively associated with a regulatory region comprising an inducible promoter gene, wherein the action gene and the inducible promoter are not operably associated in the unmodified target microorganism. Either the action gene or the inducible promoter gene may be exogenous to the target microorganism.

For example, a synthetic microorganism having a first minimal genomic modification may contain a first recombinant nucleic acid sequence consisting of a first exogenous control arm and a first exogenous action gene, wherein the first exogenous action gene is operatively associated with an endogenous regulatory region comprising an endogenous inducible promoter gene.

Inserting an action gene into an operon in the genome will tie the regulation of that gene to the native regulation of the operon into which it was inserted. It is possible to further regulate the transcription or translation of the inserted action gene by adding additional DNA bases to the sequence being inserted into the genome either upstream, downstream, or inside the reading frame of the action gene.

As used herein the term “control arm” refers to additional DNA bases inserted either upstream and/or downstream of the action gene in order to help to control the transcription of the action gene or expression of a protein encoded thereby. The control arm may be located on the terminal regions of the inserted DNA. Synthetic or naturally occurring regulatory elements such as micro RNAs (miRNA), antisense RNA, or proteins can be used to target regions of the control arms to add an additional layer of regulation to the inserted gene.

When the ratio of the regulatory elements to action genes are in sufficient excess, leaky expression of the action gene may be suppressed. When the expression of the operon containing the action gene is induced and/or the expression of the regulatory elements are suppressed, the concentration of action gene mRNA overwhelms the regulatory elements allowing full transcription and translation of the action gene or genes.

For example, a control arm may be employed in a kill switch molecular modification comprising an sprA1 gene, where the control arm may be inserted to the 5′ untranslated region (UTR) in front of the sprA1 gene. When the sprA1 gene from BP_001 was PCR amplified the native sequence just upstream of that (i.e. control arm) was included. The sprA1(AS) binds to the sprA1 mRNA in two places, once right after the start codon, and once in the 5′ UTR blocking the RBS. In order to get maximum efficiency from the sprA1(AS) to suppress the translation of the PepA1 protein, the control arm sequence was retained.

As further examples, the control arm for the kill switch molecular modification comprising an sprA2 gene may also include a 5′ UTR where its antisense binds, and the control arm for the sprG1 gene may include a 3′ UTR where its antisense antitoxin binds, so the control arm is not just limited to regions upstream of the start codon. In some embodiments, the start codon for the action gene may be inserted very close to the stop codon for gene in front of it, or within a few bases behind the previous gene's stop codon and an RBS and then the action gene. In some embodiments, where the molecular modification is a kill switch molecular modification, and the action gene is sprA1, the control arm may be a sprA1 5′ UTR sequence to give better regulation of the action gene with minimal impact on the promoter gene, for example, isdB.

The control arm sequence may be employed as another target to “tune” the expression of the action gene. By making base pair changes, the binding efficiency of the antisense may be used to tweak the level of regulation.

For example, the antitoxin for the sprA1 toxin gene is an antisense sprA1 RNA (sprA1_{AS}) and regulates the translation of the sprA1 toxin (PepA1). When the concentration of sprA1_{AS} RNA is at least 35 times greater than the sprA1 mRNA, PepA1 is not translated and the cell is able to function normally. When the ratio of sprA1_{AS}:sprA1 gets below about 35:1, suppression of sprA1 translation is not complete and the cell struggles to grow normally. At a certain point the ratio of sprA1_{AS}:sprA1 RNA is low enough to allow enough PepA1 translation to induce apoptosis and kill the cells.

The term “cell death gene” or “toxin gene” refers to an action gene that when induced causes a cell to enter a state where it either ceases reproduction, alters regulatory mechanisms of the cell sufficiently to permanently disrupt cell viability, induces senescence, or induces fatal changes in the membrane, genetic, or proteomic systems of the cell. For example, the cell death gene may be a toxin gene encoding a toxin protein or toxin peptide. The toxin gene may be selected from the group consisting of sprA1, sma1, rsaE, relF, 187/lysK, holin, lysostaphin, sprG1, sprA2, sprG2, sprG3, mazF, and yoeb-sa2. The toxin gene may be sprA1. In one embodiment, the toxin gene may encode a toxin protein or toxin peptide. In some embodiments, the toxin protein or toxin peptide may be bactericidal to the synthetic microorganism. In some embodiments, the toxin protein or toxin peptide may be bacteriostatic to the synthetic microorganism.

The term “antitoxin gene” refers to a DNA sequence encoding an antitoxin RNA antisense molecule specific for an action gene, or an antitoxin protein or another antitoxin molecule, for example, specific for a cell death gene or a product encoded thereby. The antitoxin gene may be endogenous and/or exogenous to the target microorganism.

The term “virulence block” or “V-block” refers to a molecular modification of a synthetic microorganism comprising an action gene that results in the organism to have decreased ability to accept foreign DNA from other strains or species. For example, via horizontal gene transfer or other methods. effectively resulting in the organism having decreased ability to acquire exogenous virulence or antibiotic resistance genes.

The term “nanofactory” as used herein refers to the molecular modification of a microorganism comprising an action gene that results in the production of a product—either primary protein, polypeptide, amino acid or nucleic acid or secondary products of these modifications. In some embodiments, the nanofactory product may produce a desirable, beneficial effect the synthetic microorganism, host microbiome, and/or the host subject.

The term “toxin protein” or “toxin peptide” as used herein refers to a substance produced internally within a synthetic microorganism comprising an action gene such as a cell death gene in an effective amount to cause deleterious effects to the microorganism without causing deleterious effects to the subject that it colonizes.

The term “molecular modification” or “molecularly engineered” as used herein refers to an intentional modification of the genes of a microorganism using any gene editing method known in the art, including but not limited to recombinant DNA techniques as described herein below, NgAgo, mini-Cas9, CRISPR-Cpf1, CRISPR-C2c2, Target-AID, Lambda Red, Integrases, Recombinases, or use of phage techniques known in the art. Other techniques for molecular modification may be employed as found in “Molecular Cloning A Laboratory Manual” by Green and Sambrook, Cold Spring Harbor Laboratory Press, 4th Edition 2012, which is incorporated by reference herein in its entirety. The DNA may be sequenced and manipulated chemically or by using molecular biology techniques, for example, to arrange one or more elements, e.g., regulatory regions, promoters, toxin genes, antitoxin genes, or other domains into a suitable configuration, or to introduce codons, delete codons, optimize codons, create cysteine residues, modify, add or delete amino acids, etc. Molecular modification may include, for example, use of plasmids, gene insertion, gene knock-out to excise or remove an undesirable gene, frameshift by adding or subtracting base pairs to break the coding frame, exogenous silencing, e.g., by using inducible promoter or constitutive promoter which may be embedded in DNA encoding, e.g. RNA antisense antitoxin, production of CRISPR-cas9 or other editing proteins to digest, e.g., incoming virulence genes using guide RNA, e.g., linked to an inducible promoter or a constitutive promoter, or a restriction modification/methylation system, e.g., to recognize and destroy incoming virulence genes to increase resistance to horizontal gene transfer. The molecular modification comprising an action gene (e.g. kill switch, expression clamp, and/or v-block) may be durably incorporated to the synthetic microorganism by inserting the modification into the genome of the synthetic microorganism.

The synthetic microorganism may further comprise additional molecular modifications comprising an action gene, (e.g., a nanofactory), which may be incorporated directly into the bacterial genome, or into plasmids, in order to tailor the duration of the effect of, e.g., the nanofactory production, and could range from short term (with non-replicating plasmids for the bacterial species) to medium term (with replicating plasmids without addiction dependency) to long term (with direct bacterial genomic manipulation).

The molecular modifications may confer a non-native attribute desired to be durably incorporated into the host microbiome, may provide enhanced safety or functionality to organisms in the microbiome or to the host microbiome overall, may provide enhanced safety characteristics, including kill switch(s) or other control functions. In some embodiments the safety attributes so embedded may be responsive to changes in state or condition of the microorganism or the host microbiome overall.

The molecular modification may be incorporated to the synthetic microorganism in one or more, two or more, five or more, 10 or more, 30 or more, or 100 or more copies, or no more than one, no more than three, no more than five, no more than 10, no more than 30, or in no more than 100 copies.

The term “genomic stability” or “genomically stable” as used herein in reference to the synthetic microorganism means the molecular modification is stable over at least 500 generations of the synthetic microorganism as assessed by any known nucleic acid sequence analysis technique.

The term “functional stability” or “functionally stable” as used herein in reference to the synthetic microorganism means the phenotypic property imparted by the action gene is stable over at least 500 generations of the synthetic microorganism.

For example, a functionally stable synthetic microorganism comprising a kill switch molecular modification will exhibit cell death within at least about 2 hours, 4 hours, or 6 hours after exposure to blood, serum, or plasma over at least 500 generations of the synthetic microorganism as assessed by any known in vitro culture technique. Functional stability may be assessed, for example, after at least about 500 generations by comparative growth of the synthetic microorganism in a media with or without presence of a change in state. For example, a synthetic microorganism comprising a cell death gene may exhibit cell death following exposure to blood, serum or plasma, for example by comparing cfu/mL over at least about 2 hours, at least about 4 hours, or at least about 6 hours, wherein a decrease in cfu/mL of at least about 3 orders of magnitude, or at least about 4 orders of magnitude compared to starting cfu/mL at t=0 hrs is exhibited. Functional stability of a synthetic microorganism may also be assessed in an in vivo model. For example, a mouse tail vein inoculation bacteremia model may be employed. Mice administered a synthetic microorganism (10\hat{ }7 CFU/mL) having a KS molecular modification, such as a synthetic Staph aureus having a KS molecular modification will exhibit survival over at least about 4 days, 5 days, 6 days, or 7 days, compared to mice administered the same dose of WT Staph aureus exhibiting death or moribund condition over the same time period.

The term “recurrence” as used herein refers to re-colonization of the same niche by a decolonized microorganism.

The term “pharmaceutically acceptable” refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a carrier that is physiologically acceptable to the treated subject while retaining the integrity and desired properties of the synthetic microorganism with which it is administered. Exemplary pharmaceutically acceptable carriers include physiological saline or phosphate-buffered saline (PBS). Sterile Luria broth, tryptone broth, or tryptic soy broth (TSB) may be also employed as carriers. Other physiologically acceptable carriers and their formulations are provided herein, or are known to one skilled in the art and described, for example, in Remington's Pharmaceutical Sciences, (20th edition), ed. A. Gennaro, 2000, Lippincott, Williams & Wilkins, Philadelphia, Pa.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described.

Synthetic Biology and Engineering Organisms

Synthetic biology involves redesigning an organism for a specific purpose by giving it new abilities or retooling the organism's native machinery. Making durable and stable changes to an organism are difficult to engineer, and certain rules must be followed in order to be successful. To stably express native or heterologous genes over a long period of time in an organism, they need to be located in the genome and not on a self replicating plasmid. In addition to the location of the gene, it must have multiple other components to be properly expressed or suppressed, such as a regulated promoter with a transcription start site, a ribosome binding site (RBS) if the gene codes for a protein, and transcription terminators. These components combine to produce a phenotypic response in the organism under certain conditions, and in traditional genetic engineering they are all designed, synthesized, and inserted into a non-coding region of the genome together.

The disclosure provides methods to stably insert DNA sequences to create inducible genetic switches utilizing the cells native machinery to provide most of the necessary components to create the desired expression and phenotypic response. Through RNA seq or qPCR, the transcriptome is analyzed to identify differentially expressed genes under various growth conditions in different environments. The top candidates demonstrating the appropriate levels of expression under the desired conditions and environments are then located on the genome, and the operon in which they are located is characterized.

Through genetic engineering, methods are provided to couple an endogenous or exogenous action gene to the expression of a native gene or operon in an organism's DNA. Targeting genes or operons that are differentially expressed at sufficiently low and high rates in different environments allows the action gene to function in two discrete states, off and on, respectively. This information is exploited to “hide” the action gene from the organism during times of low expression, so it does not get removed from the genome or mutated to be no longer functional when it is needed. Environmental conditions which induce high expression of the native genes also induce high transcription of the integrated action gene leading to the desired phenotypic response.

Native or synthetic small noncoding RNAs (sRNA) can also be used to post transcriptionally regulate endogenous or exogenous genes in an organism. sRNA usually acts to regulate protein expression by binding to a target mRNA molecule creating a double stranded RNA which is sought out and degraded by native systems in the cell. The disclosure provides methods for incorporating sRNA regulation in synthetic switches or genetic circuits to control leaky expression of an action gene, which helps to create very stable genomic integrations.

Small noncoding RNAs (sRNA) found in prokaryotic cells has been determined to regulate gene expression by base pairing with mRNA targets, and fall into two categories called cis- and trans-acting sRNA. Bloch, Sylwia, et al. “Small and smaller—sRNAs and microRNAs in the regulation of toxin gene expression in prokaryotic cells: a mini-review.” Toxins 9.6 (2017): 181.

sRNAs have been shown to regulate a wide range of gene expression including many toxin genes found in the genome of most bacteria. The antitoxins in type I toxin-antitoxin systems in bacteria are sRNAs that post-transcriptionally regulate the expression of the toxins. Schuster, Christopher F., and Ralph Bertram. “Toxin-antitoxin systems of Staphylococcus aureus.” Toxins 8.5 (2016): 140. doi:10.3390/toxins8050140.

The present disclosure demonstrates the ability to re-engineer a cell's toxin-antitoxin system to function as an environment-specific inducible kill switch forcing the cell to induce artificial programmed cell death or halt metabolism under specific conditions. This strategy involves maintaining sufficient concentrations of antitoxin to suppress the translation of the toxin proteins in environments where growth is not to be disrupted, then tipping the ratio of toxin and antitoxin expression the opposite way when the organism gains access to an undesired environment.

Measuring the transcript levels of the toxin gene and the sRNA antitoxin in both the organism's native niche and disease causing environments may be performed in order to predict if a kill switch will be induced or remain dormant in those environments.

There are many tools available to researchers that can quickly preserve and process the RNA from a variety of sample types. Since the sRNA can start degrading within minutes of being synthesized, fast and robust sampling techniques are required to get accurate and reliable data from the samples. Using RNA preservation solutions, such as RNA Shield from Zymo, we can preserve RNA for long periods from many sources such as microbiome swabs or infected tissues.

During the RNA extraction and purification steps, certain RNA kits capture all RNA molecules that are over 20 bases long allowing us to collect the sRNA antitoxin along with the mRNA transcript of the toxin genes that we are interested in. Through qPCR, RNA-seq, northern blots, and other methods, it is possible to quantify the transcript levels of the components of engineered kill switches or native toxin-antitoxin systems. Combining all of the topics discussed above, it is possible to capture and measure the levels of the kill switch components. This allows prediction of the likelihood of kill switched organisms to survive or struggle in a variety of environments, without having to perform costly human or animal trials.

Genomic Modifications

Although other techniques may be employed, DNA sequences can be manipulated in vivo through a method called homologous recombination. This genetic recombination technique revolves around regions of homology between the two DNA sequences and their ability to match up and combine sequences. For making an insertion into a genome of a cell using this technique, a plasmid is constructed with regions of homology (homology arms) to the targeted location in the genome flanking the DNA sequence to be integrated. Typically about 1,000, 1,200, or more base pair long fragments are used for homology arms, which often means that there is likely to be a promoter region upstream of the gene or genes to be inserted.

Staph aureus Genomic Modifications

In the case of editing the Staph aureus genome, an E. coli passthrough strain may be required to produce sufficient quantities of properly methylated plasmid DNA, and if there is a promoter region in the homology arm upstream of the action gene to be inserted, the E. coli passthrough strain will likely transcribe and translate the genes. In the present disclosure, the action gene to be inserted sometimes codes for peptides toxic to the cell producing it, so leaky expression must be kept to a minimum in the passthrough strain.

One method to minimize the leakiness of the expression in the passthrough strain is to target the region for insertion to be behind a large gene in an operon, rather than directly behind the promoter. There is less of a chance for a promoter region to be found in the middle of a gene and adversely affect the expression of the action gene in the passthrough strain. If the site of kill switch integration is chosen to be at the end of a gene, the homology arms required for the integration can be chosen such that the promoter region is not part of the homology arm, reducing the effect the toxin gene located on the plasmid has on the E. coli passthrough strain.

The present disclosure has implemented that strategy for many of the kill switches made with much success. The strategy allows for the inserted gene to piggyback off of the target organism's native regulation of the gene or operon while not killing the passthrough strain or disrupting the expression of the gene it is integrated behind.

Piggyback is Superior to Gene Knock Out (to Control Virulence)

The disclosure provides methods for specializing in the management of mutualistic microbes in the human and animal microbiome in such a way as to not disturb the natural balance in healthy states and yet prevent opportunistic infections from establishing in an individual. To do this, methods and synthetic microorganisms comprising a kill switch have been developed that do not allow an organism to grow and reproduce when it escapes from its natural niche to an environment where it is capable of causing disease.

In order to ensure the viability of an organism within its native niche, while at the same time reducing the ability for symbiotic organisms in the microbiome to cause disease in its host, a method has been developed that identifies genes that are (i) downregulated while the organism occupies its native niche, and (ii) that are significantly upregulated in disease-causing conditions. The method further comprises linking the expression of one or multiple identified differentially regulated gene(s) to the expression of a gene that is toxic to the organism. The toxin gene may be derived from one of the target organism's own toxin-antitoxin systems, which advantageously allows utilization of at least part of its native regulation in the cell. Linking the expression of the differentially regulated native gene and the toxin may comprise inserting the toxin gene in a location in the genome where it will be included on the same transcript as the differentially regulated gene(s), and thus linking the expression of the two.

The synthetic microorganisms comprising a kill switch system of the present disclosure are superior to controlling the viability or virulence of an organism by other traditional methods such as knocking out virulence genes or genes required for causing disease or infections. Knocking out genes in a genome has a greater chance of destabilizing the cell under normal growth conditions than the piggyback method of the disclosure.

Bacterial genomes are generally small and efficient, meaning there is rarely a gene or pathway that is not needed in some respect in all growth conditions. Knocking out the whole gene may give the intended response in the intended environment, but it may also cause changes to the metabolism or viability in the native environment as well. In the case of mutualistic microbes in the microbiome, this may mean that the edited organism will lose its advantage in the niche it usually occupies resulting in decreased stability, decreased durability, which may allow other more virulent strains to take over.

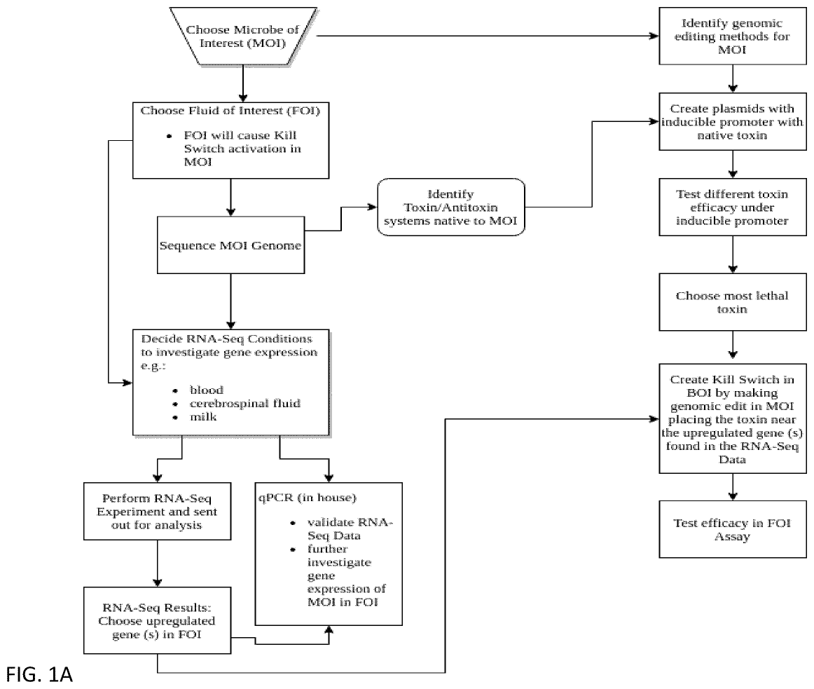

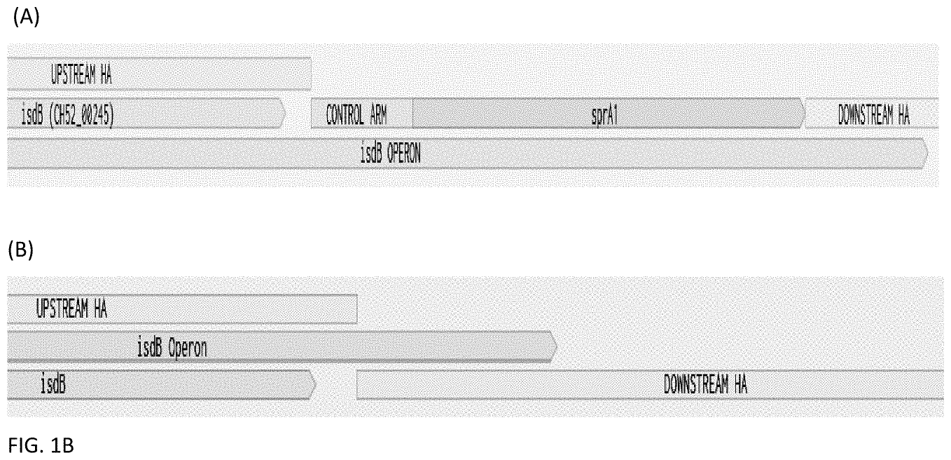

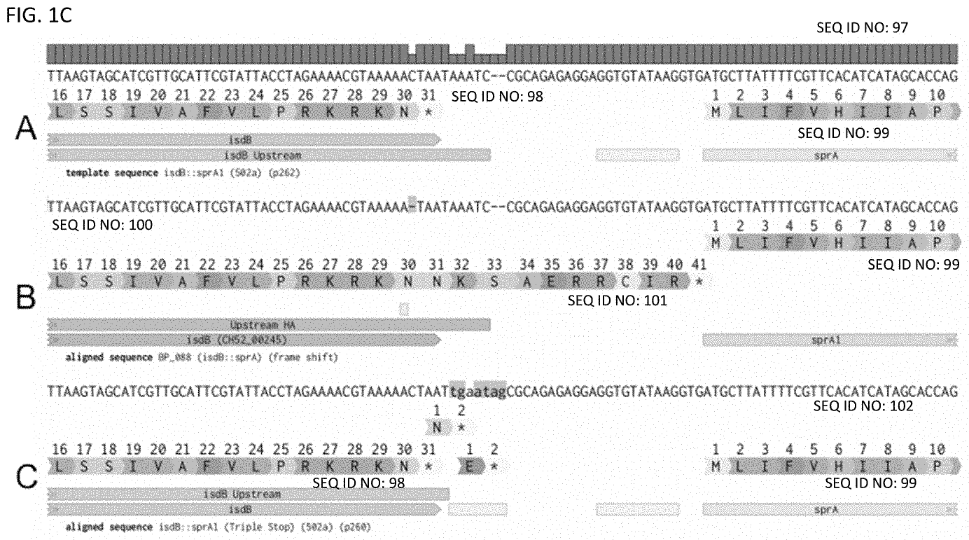

A linear map of genomic insertion of a toxin in a synthetic microorganism designed with a kill switch using a piggyback strategy is shown in FIG. 1B (A), compared to wild type Staphylococcus aureus target strain, BP_001 (B). In the synthetic microorganism (A), the sprA1 gene was inserted directly after the endogenous isdB gene, with an optional intervening control arm, to obtain a synthetic Staphyloccocus aureus comprising isdB::sprA1. The isdB mRNA transcript has been extended in the synthetic microorganism to include the sprA1 gene, and will terminate downstream of the sprA1 gene, instead of right after the isdB gene as it does for the wild type strain, BP_001 (B).

Control Arm

Inserting an action gene into an operon in the genome will tie the regulation of that gene to the native regulation of the operon into which it was inserted. It is possible to further regulate the transcription or translation of the inserted action gene by adding additional DNA bases to the sequence being inserted into the genome either upstream, downstream, or inside the reading frame of the action gene. If the additional DNA bases are either upstream or downstream of the action gene, we refer to it as a control arm because it helps to control the expression of the gene or protein, and is usually found at the terminal regions of the inserted DNA. For example, the control arm may include synthetic or naturally occurring regulatory elements such as microRNAs (miRNA), riboswitches, small noncoding RNAs (sRNA), or proteins to add an additional layer of regulation to the inserted gene.

When the ratio of the regulatory elements to action genes are in sufficient excess, leaky expression of the action gene is suppressed. When the expression of the operon containing the action gene is induced and/or the expression of the regulatory elements are suppressed, the concentration of action gene mRNA overwhelms the regulatory elements allowing full transcription and translation of the action gene or genes.

For example, the antitoxin for the sprA1 toxin gene is an antisense sprA1 sRNA (sprA1_{AS}) and regulates the translation of the sprA1 toxin (PepA1). When the concentration of sprA1_{AS} RNA is at least 35 times greater than the sprA1 mRNA, PepA1 is not translated and the cell is able to function normally. When the ratio of sprA1_{AS}:sprA1 gets below 35:1, suppression of sprA1 translation is not complete and the cell struggles to grow normally. At a certain point, the ratio of sprA1_{AS}:sprA1 RNA is low enough to allow enough PepA1 translation to induce apoptosis and kill the cells.

Synthetic Microorganism Design Methods

Methods are provided for designing a kill switched synthetic microorganism. Kill switching strains may be used to prevent or reduce the risk of opportunistic infection from either endogenous microorganisms or pathogenic microorganisms. The kill switch is not intended to compromise the organism's ability to live within its native niche, but will prevent the organism from reproducing in environments that would cause infection or disease, such as the bloodstream.

In order to design an effective kill switch that is induced only when the synthetic microorganism is in one or more very specific environments, the transcriptional profile of the target organism in intended niche or complete media and in one or more additional specific environments may be investigated.

Some bacteria are known to contain expression systems that either arrest growth or may lead to cell death when overexpressed. Kourtis, MMWR Morb. Mortal. Wkly. Rep. 68, (2019). The bacterial toxin-antitoxin systems and can be manipulated to help create useful kill switch strategies.

The transcriptional profile of the microbe may be used to determine what genes are expressed at low levels while the microbe is living in its normal habitat, and which are significantly up or down regulated while in its disease-causing state. The differentially regulated genes may then be coupled or operably associated with components of the target microorganisms own toxin-antitoxin systems to produce a synthetic microorganism that is capable of living in its normal niche such as a dermal or mucosal niche in the subject, and/or a complete media, but unable to reproduce and cause disease if placed in contact with another environment, such as a systemic environment in the subject's blood, serum, plasma, interstitial fluid, etc.

It is one objective to create a synthetic microorganism comprising a kill switch to ensure it cannot become an accidental pathogen and lead to the diseases they are designed to prevent. Another objective is to provide a safe synthetic microorganism for use in bacterial interference in a subject to prevent colonization or re-colonization of the subject with more virulent strains, such as a pathogenic strain, e.g., an MRSA strain. Thus methods are provided for designing a kill switch genetically inserted into the genome of the synthetic microorganism to cause cell death or bacterial stasis if the strain gains access to unintended regions of the body.

Strain design methods are provided for engineering bacteria to be unable to grow under specific disease causing states without disrupting its behavior in its normal niche. RNA-Seq and qPCR may be employed to identify genes that are differentially regulated in specific disease conditions compared to normal growth conditions. The cell's own toxin-antitoxin systems may be used to control growth and viable cell numbers in specific conditions.

A method for preparing a synthetic microorganism comprising a kill switch according to the disclosure may comprise the steps shown in Table 1A and FIG. 1A. Each step is presented in the context of an exemplary kill switch design; however, alternative or additional action genes may be employed.

TABLE 1A
Strain Design Method

| Sec- | |
|---|---|
| tion | Title |
| 1 | Select Target Microorganism (microbe of interest, MOI) |
| 2 | Select Fluid or Environment for Kill Switch Activation in |
| | Target Microorganism |
| 3 | Target Microorganism Genome |
| 4 | Finding Upregulated Genes using RNA Seq Experiment |
| 5 | Identify Toxin/Antitoxin Systems Native to Target Microorganism |
| 6 | Identify Genomic Editing Methods for Target Microorganism |
| 7 | Create Plasmids with Toxins to Test Toxin Efficacy |
| 8 | Validate Results of RNA Seq with qPCR |
| 9 | Combine Results of RNA-Seq and Plasmid Toxin Screen to Design |
| | Kill Switch |
| 10 | Test Newly Constructed Strain with a fluid of interest (FOI) Assay |

Choosing a Target Microorganism (microbe of interest (MOI))

The criteria for choosing the target microorganism (microbe of interest MOI also known as bug of interest BOI) includes selecting a microorganism that is present or may integrate to a human or animal microbiome. The target microorganism may be of the same species as a pathogenic microorganism capable of causing an opportunistic infection. In some embodiments, the target microorganism may be an antibiotic-susceptible microorganism. For example, the target microorganism may be a methicillin-susceptible Staphylococcus aureus (MSSA), such as a 502a strain. The pathogenic microorganism may be an antibiotic-resistant microorganism. The pathogenic microorganism may be a methicillin-resistant Staphyloccoccus aureus (MRSA). The target microorganism may likely be capable of durably replacing a pathogenic microorganism in the niche of the subject, optionally prior to genomic modification. However, even a relatively benign target microorganism may be capable of causing an opportunistic infection.

Selecting Fluid or Environment for Kill Switch Activation in Target Microorganism

The target microorganism is designed to be able to durably occupy a natural niche, such as a dermal or mucosal niche. The target microorganism will be stably genetically-modified such that it should not be able to survive under systemic conditions in the subject, such as intravenous or subcutaneous physiological environments that can lead to infection. The Fluid of Interest (FOI) may be a bodily fluid where the target microorganism may be capable of causing an opportunistic infection or a food product. Some examples of potential FOI's are blood, serum, cerebrospinal fluid, synovial fluid, and milk. A target microorganism will then be modified to introduce a genomically-integrated kill switch such that the resultant synthetic microorganism be not be able to grow in selected multiple different fluids (FOIs) or environments.

Mapping Target Microorganism Genome
1. A full DNA sequence of the Target Microorganism may be useful to begin investigating the potential of the strain. If no annotated sequence is available on public databases, the Target Microorganism's DNA may be extracted and sequenced. Next gen sequencing techniques may be used to capture most of the genomic sequence (up to 99%), but the technique requires a reference strand to map the short reads onto. Without a reference strand available, nanopore sequencing may be also used to create long sequencing reads that can act as the reference strand for the shorter reads to be mapped onto. Once the genomic sequence is assembled and annotated, it may then be used for genomic mapping, looking for similarity across strains, and editing the genome for kill switch integrations or other applications.

Finding Upregulated Genes Using RNA-Seq Experiment

RNA-Seq (RNA sequencing) or a microarray experiment may be used to capture a profile of the Target Microorganism's transcriptome in different environments to find variable gene expression. Both of these methods can analyze the Target Microorganism's gene/promoter expression by quantifying the levels of RNA transcribed in response to different environments. RNA-Seq may be performed to find a potential kill switch promoter, comprising growing the Target Microorganism in the FOI, and taking samples at predetermined time points, and extracting RNA from the samples, and measuring RNA concentration and purity. After this, the rRNA must be degraded and the remaining mRNA in the sample will be reverse transcribed to create a cDNA library. The resulting cDNA is sequenced on a next generation sequencer. The reads are mapped and aligned to the Target Microorganism reference sequence. The resulting dataset will show the number of reads per gene that were mapped to the annotated reference sequence. Conesa et al. A survey of best practices for RNA-seq data analysis. Genome Biol. 17, 13 (2016).

RNA-Seq (an abbreviation of “RNA sequencing”) is a sequencing technique which uses next-generation sequencing (NGS) to reveal the presence and quantity of RNA in a biological sample at a given moment, analyzing the continuously changing cellular transcriptome. Voelkerding et al., 2009, Clinical Chem 55:4; 641-658-RNA-Seq facilitates the ability to look at alternative gene spliced transcripts, post-transcriptional modifications, gene fusion, mutations/SNPs and changes in gene expression over time, or differences in gene expression in different groups or treatments. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries. Next-generation sequencing (NGS) also known as massive parallel sequencing is a high-throughput approach to DNA sequencing using massively parallel processing. These technologies may include use of minitiarized and parallelized platforms for sequencing of 1 million to 43 million short reads (˜50-400 bases each) per instrument run.

Performing the RNA-Seq may include growing the Target Microorganism in the FOI; taking samples at predetermined time points; extracting RNA from the samples; measuring RNA concentration and purity; degrading rRNA and reverse transcribing remaining mRNA in the sample to create a cDNA library; sequencing the cDNA library to create DNA sequence reads, optionally on a Next-Generation sequencer (ThermoFisher Scientific); mapping and aligning the DNA sequence reads onto a Target Microorganism annotated reference sequence; and calculating number of reads per gene that were mapped to the annotated reference sequence.

An example protocol where the Target Microorganism is grown and sampled in both a control media and a FOI at predetermined time points may include the following steps.

Example Protocol: In an incubated shaker, the Target Microorganism is grown in both a culture media (control) and the FOL. Take samples at t=0, t=30, and t=60 minutes. At each time point, pellet cells and add RNA Protect to preserve RNA. Extract RNA and send out for RNA Seq analysis. RNA Seq Data Analysis may be performed according to Conesa 2016. The dataset is normalized to produce the output of TPM (Transcripts Per Million). Normalize the number of reads per gene to account for gene length (# of reads/length of gene in kilobases, returns RPK=reads per kilobase). Divide the RPK values by per million scaling factor to account for the difference in total reads per sample. This normalizes values that would be artificially inflated/deflated due to an increase/decrease in total reads, not necessarily because they were regulated differently in the different growth states. Finally identify genes that have low level expression in culture media and are upregulated in the FOI (gene/promoter candidates for toxins).

Genes or promoters with high transcript expression levels in the FOI and normal or low expression in the culture media are of interest to use for the engineered kill switch. Genes that “turn on” in the FOI can be used as an area to integrate a toxin gene on the same mRNA transcript so that the toxin is expressed along with the upregulated gene in the FOI.

Identifying Toxin/Antitoxin Systems Native to Target Microorganism

Toxin/antitoxin systems are native to many microbes and can act as a cell growth regulator under stressful conditions. Yamaguchi, Y., Park, J.-H. & Inouye, M. Toxin-antitoxin systems in bacteria and archaea. Annu. Rev. Genet. 45, 61-79 (2011). There are at least six types of toxin/antitoxins systems discovered all of which differ in how the antitoxin regulates the toxin. For example in type I toxin/antitoxin system, the RNA antitoxin inhibits translation of the toxin mRNA. Proteic toxins are small peptides (around 100 bps) that can induce cell death via inhibition of protein, cell wall synthesis and DNA replication, compromising cell wall integrating, and affect mRNA stability. Ideally, the toxins used for the strain design would be native to the Target Strain but other toxin genes from other microbes may be also be employed.

Identifying Genomic Editing Methods for Target Microorganism (MOI).

Genetic editing methods may be identified that are suitable to the target microorganism. Suitable plasmids may be identified that are able to direct homologous recombination to edit the genome of the target microorganism. Thomason et al., Current Protocols in Molecular Biology (eds. Ausubel, F. M. et al.) 1.16.1-1.16.39 (John Wiley & Sons, Inc., 2014). doi:10.1002/0471142727.mb0116s106. Other genomic editing systems may also be used such as the CRISPR/Cas9 system or using ultra competent cells to directly uptake PCR amplicons. Adli, M. The CRISPR tool kit for genome editing and beyond. Nat. Commun. 9; Junges, R. et al. Markerless Genome Editing in Competent Streptococci. Methods Mol. Biol. Clifton N.J. 1537, 233-247 (2017). Thus homologous recombination, CRISPR-Cas9 system, markerless genome editing, or any other suitable method known in the art may be used to create a durable genomic integration of the toxin near the inducible gene or promoter region in the genome of the target microorganism.

Creating Plasmids with Toxins to Test Toxin Efficacy

Native or nonnative candidate toxins may be screened for effectiveness against the target microorganism. This may be done by creating a plasmid containing the candidate toxin underneath the control of an inducible promoter. For example, a plasmid with a tetO operon which can be induced by tetracycline or anhydrotetracycline can be used to induce toxin production. Helle, L. et al. Vectors for improved Tet repressor-dependent gradual gene induction or silencing in Staphylococcus aureus. Microbiology 157, 3314-3323 (2011). When the plasmid is transformed into the target microorganism it can be induced and cell death may be measured, e.g., by CFU plating or measuring the optical density using a spectrophotometer. Nutrition, C. for F. S. and A. BAM: Aerobic Plate Count. FDA (2019). One or more of the most lethal candidate toxins may be selected for genomic integration in the target microorganism under the regulation of the inducible gene or promoter in the FOI, e.g., as found with the RNA-Seq methods mentioned herein.

Validating Results of RNA Seq with qPCR

qPCR or other suitable techniques known in the art may be used to verify RNA-Seq results by using primers that bind to genes of interest and measuring their activity in different environments.¹² The technique can also be used to verify levels of RNA transcripts in kill switch strains to ensure the proper mechanism of the toxin and promoter.

An example growth protocol for qPCR measurement may include the following steps.
a. Grow overnight culture of Target Microorganism (MOI).
b. After 12-16 hours of growth, inoculate a disposable sterile shake flask with 50 mL of overnight culture to an optical density 600 (OD) of 0.1.
c. Grow cells to an OD of 2. At OD 2, remove 500 μl for t=0 minutes RNA sample. Transfer 3×7 mL of the remaining cells to triplicate 50 mL conical tubes. Centrifuge tubes, decant supernatant, wash with 1× phosphate-buffered saline (PBS), centrifuge again, decant supernatant, and resuspend cells in 7 mL of control media (e.g. TSB) and fluid of interest (e.g. blood, serum, milk, etc).
d. Place tubes in 37° C. incubator shaking at 240 rpm.
e. Collect RNA samples at t=0 minutes (sample tubes immediately before placing them into the 37° C. incubator), t=15 minutes and t=45 minutes after exposure to serum or blood. To sample growth samples for RNA, transfer 500 μL to 1.7 mL microfuge tube, spin cells at 13,200 rpm for 1 minute, decant supernatant, and add 100 μL of RNA Protect.
f. Store all samples at −20° C.

An example qPCR sample processing and data analysis protocol may include the following steps, or as found in the literature such as in Taylor et al. Methods 50, S1-S5 (2010).
a. Wash frozen RNA pellets once in PBS.
b. Extract RNA using Ambion RiboPure Bacteria kit and elute in 25 ul.
c. Remove DNA from samples using Ambion Turbo DNase kit.
d. Convert 10 μL of final RNA to cDNA using the Applied Biosystems High-Capacity cDNA Reverse Transcription kit.
e. Perform qPCR measurements using the Applied Biosystems PowerUp SYBR Green Master Mix (10 μl reaction with 1 μl of cDNA).
f. Probe samples to look for changes in gene expression over time and in different media
g. Normalize to housekeeping gene, gyrB, using the ΔΔCt method. Subtract Ct (cycles to threshold) values for gyrB transcripts from Ct values for gene transcripts for each RNA sample. Normalize ΔCt values to t=0 minutes.

Combining Results of RNA-Seq and Plasmid Toxin Screen to Design Kill Switch in Target Microorganism

Using the selected genomic editing method, plasmid or other system may be designed to insert the toxin gene near (before, middle or end) of the inducible gene or promoter region found in the Target Microorganism that is highly upregulated in FOI to create a durable integration of the kill switch. After successful genomic editing has been confirmed via sequencing, the new strain may be tested in the FOI using a FOI assay.

Testing Newly Constructed Synthetic Microorganism in a FOI Assay

A FOI assay (also called kill assay protocol) may be used to demonstrate the lethality of the toxin in the synthetic microorganism. The genetically modified strain may be exposed to or incubated in the FOI and samples taken at predetermined time points. The growth of the samples may be measured by colony forming unit's (CFU) per mL which are measured by plating certain dilutions on an agar plate and counting the number of colonies after an incubation period, or via optical density measurements at OD600, flow cytometry or other type of luminescent assay.

An example kill assay protocol may include the following steps.
a. Grow synthetic strain in cell culture media, spin the cultures down, wash with PBS, spin down again, and resuspend in PBS. Optionally grow target strain similarly.
b. Inoculate the FOI and cell culture media (control) with the washed culture.
c. Sample each culture, t=0 hours, before the inoculated cultures are placed in a 37° C. incubator, shaking at 240 rpm.
d. Perform dilution plating of each sample in PBS to allow the CFU/mL to be calculated the following day.
e. Sample at predetermined time points through the growth of the cultures (e.g. t=2 hours, t=4 hours, t=6 hours, and t=8 hours) and perform dilution plating.

If the kill switch synthetic strain is expressing the toxin at effective levels in the FOI, then the CFU/mL will decrease over the time period of the assay. If the CFU/ml stays the same or is similar to the strain grown in the cell culture media, another strain may be designed by adding a toxin genes to other upregulated genes in FOI or adding a toxin gene to multiple upregulated genes in a single strain. Every synthetic strain construct may be tested using some type of assay that measures cell death in the FOI.

Methods are provided to exploit toxin-antitoxin systems in target microorganisms to create a synthetic microorganism comprising a kill switch that turns on under predetermined environmental conditions to kill the synthetic microorganism. RNA-Seq data may be generated by growing the MOI in the FOI to determine which genes or promoters are upregulated in the FOL. A toxin gene may then be inserted into the genome of the target strain near, and operably associated with, an endogenous inducible gene to produce a synthetic strain comprising a kill switch. When the synthetic strain is exposed to a specific environment, the upregulated region will turn on, therefore producing the newly integrated toxin which kills the strain. This technique allows for creating live biotherapeutic products (LBPs), for example, comprising the synthetic microorganisms for use in bacterial interference without the risk of opportunistic systemic infection in the host subject.

Genomic Integration Site Selection for Optimal Expression of Action Gene: Start Site Optimization for Kill Switch

The disclosure provides methods for inserting action gene DNA fragments into the genome of an organism in order to operably link an inducible promoter to the action gene capable of changing the phenotype of the organism under specific environmental stimuli without compromising the cell's ability to survive in its native niche. Methods comprise making a minimal genomic modification where the cell's native regulatory system sufficiently regulates the transcription and translation of the action gene such that the phenotypic response is either observed or below detectable limits. The exogenous DNA inserted into the genome of the organism can contain either the action gene or an inducible promoter.

There are two main criteria or stages for determining the optimal location for insertion of the exogenous DNA sequence: 1) performing a global search of the target host's transcriptome to find the genes or promoters differentially regulated in the conditions where the action gene is desired to be both “ON” and “OFF”, 2) determining the exact location for integrating the exogenous DNA sequence on a local scale in the genome to optimally express the RNA transcript containing the action gene. For both the global and local scale in the target organism's genome, the location chosen for insertion of the exogenous DNA fragment may have a great effect on the engineered expression of the action gene. In order to achieve optimal performance from the engineered organism, care may be taken when deciding the proper location in the genome to operably link the inducible promoter and action gene.

For the first stage in the development of the environmentally inducible kill switch, an RNA-seq experiment may be performed using samples of a target microorganism (e.g., from Staphylococcus aureus (SA)) in growth assays in different media, such as human serum and tryptic soy broth (TSB). Samples may be taken for RNA extraction at different time points, and the RNA transcripts were sequenced to show the global gene expression at the specific time points in both growth conditions, allowing the identification of differentially expressed genes between the different growth conditions. The differentially regulated genes are identified as potential candidates to further investigate as locations to integrate the exogenous DNA.

Once an inducible gene or promoter has been identified as having the desired expression pattern in the proper environments, it may be investigated further to determine the proper orientation and location for insertion of the exogenous DNA fragment. In order to tether the expression of the action gene to the inducible promoter, the action gene preferably is located in between the transcription start site and the terminator in the RNA transcript in such a way that does not disrupt the transcription or translation of the native genes. Since transcripts for each individual gene, operon, and other regulatory RNAs expressed in a cell vary in a multitude of ways, the optimal location to target the integration is a complex decision. Examples provided herein show that making minor changes to the distance between the stop codon of the gene upstream of the integration in the isdB::sprA1 kill switch had little to no effect on the efficacy of the kill switch when evaluated in serum.

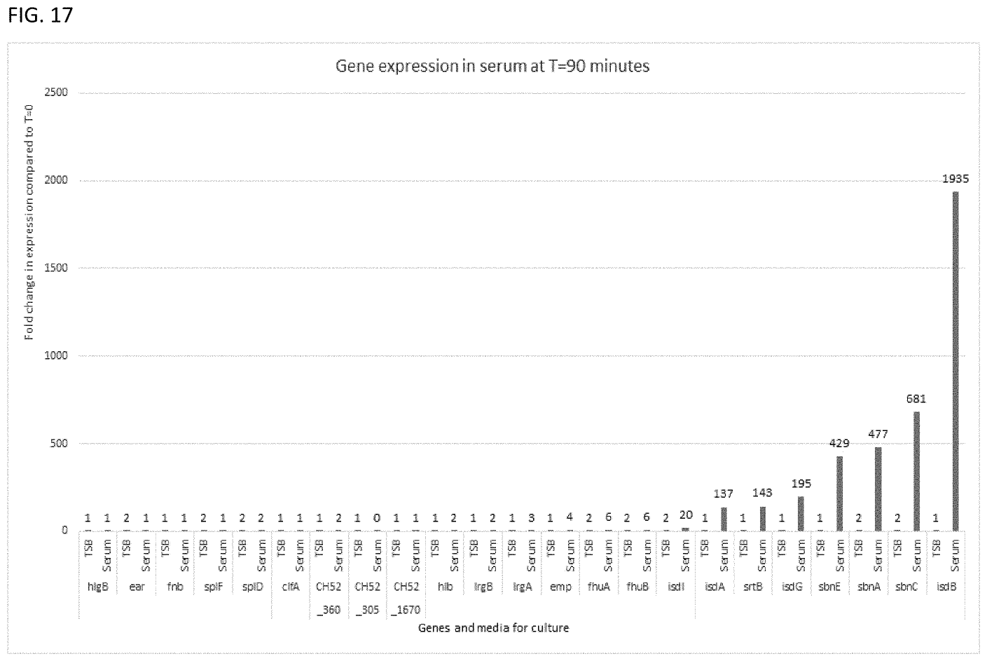

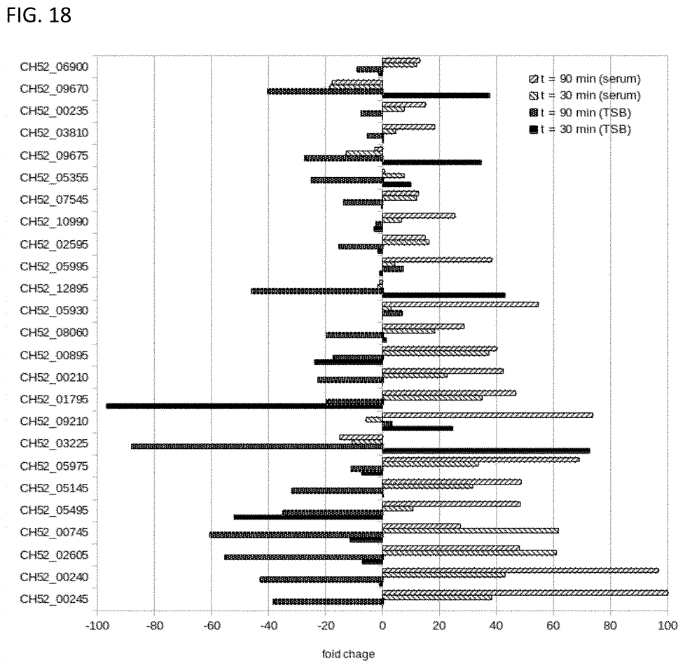

In order to create a durably integrated and operably linked action gene to an inducible promoter system, the location of the genomic insertion plays an important role. As shown in the present examples, the RNA-seq analysis of the Staph aureus strain BP_001 grown in different media conditions showed very different transcript profiles between the different conditions, as shown in FIG. 18 and in the examples. Genes were selected that exhibited very low levels of transcripts present while the target strain was growing in TSB, and very high levels of transcripts while the strain was growing in human serum, such as the isdB, harA, isdC, and sbnA genes to name a few.

The integration of toxin gene sprA1 was targeted into operons of selected genes, including isdB, PsbnA, harA to create synthetic strains BP_118, BP_092, and BP_128, respectively. In one case the native promoter for the sprA1 gene was deleted and replaced with the promoter for the sbnA gene (PsbnA_BP_150). After the serum assay for BP_128 was run, it was found that the strain used for the assay had a frame shifted and truncated sprA1 gene.

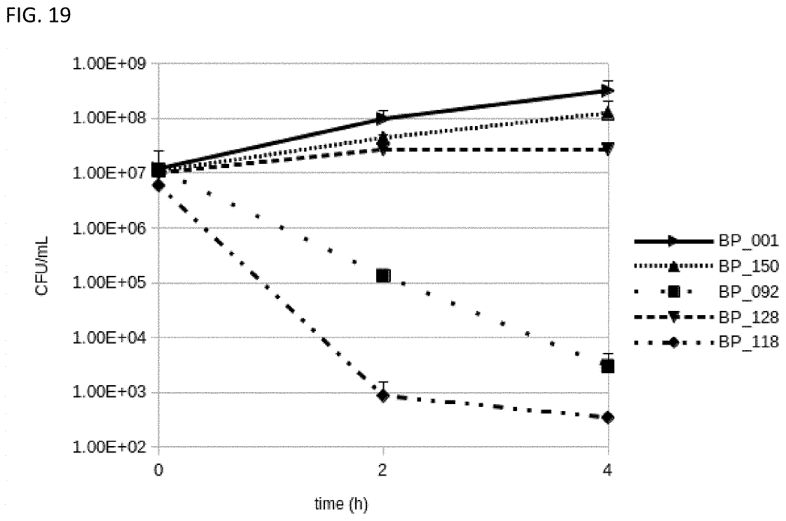

As shown in FIG. 19, when tested for their ability to grow in serum, strains BP_118 (isdB::spra1), BP_092 (PsbnA::sprA1) and BP_128 (harA::sprA1) each exhibited a decrease in CFU/mL at both the 2 and 4 hour time points. BP_118 (isdB::spra1) exhibited strongest kill switch activity as largest decrease in CFU/mL. Strain BP_150 grew only slightly slower than the wild type parent strain, but still maintained a positive growth curve during the 4 hour assay.

None of the strains tested showed a difference in their growth in TSB compared to the wild type strain BP_001, indicating that the expression of the sprA1 action gene was sufficiently suppressed in that growth condition.

Numerous death-inducing kill switches in Staphylococcus aureus (S. aureus) and Escherichia coli (E. coli) are provided herein. These kill switches, contained on a plasmid or integrated within the genome, induce cell death upon sensing certain state changes. These have commonly been designed using the S. aureus toxin gene, sprA1. The overexpression of toxin genes such as sprA1 and sprG1 may lead to cell death in E. coli and Staph aureus. The sprG2 and sprG3 genes found in most Staph aureus strains belong to the Type I toxin-antitoxin family, and their expression can be controlled by their sRNA antitoxins, sprF2 and sprF3, respectively. When the ratio of toxin mRNA to antitoxin sRNA reaches a level at which the translation of the toxin gene can no longer be suppressed by the antitoxin, the toxin proteins are synthesized which leads to bacteriostasis of the transformed cell. Riffaud, Camille, et al. “Functionality and cross-regulation of the four SprG/SprF type I toxin-antitoxin systems in Staphylococcus aureus.” Nucleic acids research 47.4 (2019): 1740-1758. This is demonstrated herein in Example 21. Both action genes sprG2 and sprG3 were tested for their ability to cause bacteriostasis in E. coli and S. aureus using the pRAB11 expression vector. Overexpression of the sprG2 gene on p305 led to bacteriostasis in both E. coli (BPEC_025) and S. aureus (BP_165).

Piggyback Applications Beyond Kill Switch

The present disclosure includes methods for using a cell's machinery to create other inducible genetic switches beyond kill switches. The piggyback method may also be used for the creation and production of “rheostatic” cells. These are cells that can be modified using the piggyback technology to respond in specific manners upon sensing state changes. The kill switch example has been demonstrated with great efficacy; however, the applications beyond an inducible kill switch are vast. Beginning with reporter genes to demonstrate a non-lethal inducible response, several potential piggyback applications are provided that have major impacts on healthcare across the globe.

Reporter Gene Integrations

Reporter genes can be used, such as green fluorescent protein (GFP), to detect specific state changes inside or outside of a cell. By using the same strategy to identify differentially regulated genes and operons described above, we can engineer cells that possess discrete switches that can be used for diagnostic purposes to detect certain environmental conditions, such as pH or temperature changes, the presence or absence of certain chemicals, and other environmental stimuli on a cellular level.

By using the cell's native regulation system, or designing synthetic small noncoding RNA (sRNA) molecules to regulate the reporter gene's transcription and translation rates to be near zero when the switch is “off,” and then removing the gene suppression systems in the presence of the substance or environmental condition to turn the switch “on”, can be a valuable diagnostic tool. The reporter protein could be detected visually by using fluorescent or chromogenic proteins, through smell by using a protein such as alcohol acetyltransferase I which produces isoamyl acetate (banana odor), through changing the phenotypic response of an organism such as inducing catalase production in strains that are normally catalase negative (H₂O₂→2H₂O+O₂), or through molecular biology methods such as qPCR or RNA-seq looking for increased levels of specific mRNA transcripts. The action gene may be a reporter gene, for example a fluorescent reporter gene, such as a green fluorescent protein (GFP) or a red fluorescent protein (RFP), such as mKATE2.

Fluorescent reporter genes may be inserted into the genome of Staph aureus, Strep agalactiae, and E. coli behind serum-responsive promoter genes using the piggyback method. The fluorescence from the reporter proteins may be quantified while the cultures are growing in serum and TSB to obtain quantitative data about the transcription and translation rates of the promoters or mRNA transcripts regulating the expression of the reporter genes. Those transcription and translation rates can be used to gain valuable information about how the cell regulates those pathways in the conditions tested. Green fluorescent protein (GFP) and mKATE2 (red fluorescent protein, RFP) are fluorophores that fluoresce when excited. They were originally isolated from different aquatic animals and both have specific excitation and emission spectra, but have since been engineered and optimized for their specificity and stability. By genomically integrating one of these genes behind tightly controlled promoters, genes, or operons, and then using a fluorescent plate reader to quantify the fluorescence of the cultures, it may be possible to calculate transcription and translation rates of the fluorescent proteins GFP and mKATE2.

The present disclosure demonstrates evidence of functional stability of certain kill switched bacterial strains that have been grown for over 500 generations. As such, this technique should be well suited for the development of organisms that can be used for diagnostic purposes, such as those described above, and has been demonstrated with GFP and mKATE2.

Lactose Intolerance in Humans

Lactose intolerance in humans is caused by the underproduction of the enzyme lactase (also known as β-galactosidase enzyme, encoded by a lacZ gene) in the GI tract leading to the inability to digest the disaccharide sugar compound lactose. This condition affects many people and is as high as 90% among certain populations. High concentrations of lactose are found in mammalian milk, and many individuals lose their ability to generate sufficient lactase for dairy consumption after weaning. Lactose is a disaccharide (4-O-β-galactopyranosyl-D-glucopyranose) composed of galactose and glucose. Campbell et al., “The molecular basis of lactose intolerance.” Science progress 88.3 (2005): 157-202. Lactose intolerant individuals are still able to metabolize galactose and glucose, so if their digestive system was capable of producing enough lactase to sufficiently break down the disaccharide lactose, the symptoms from the condition would be mitigated.

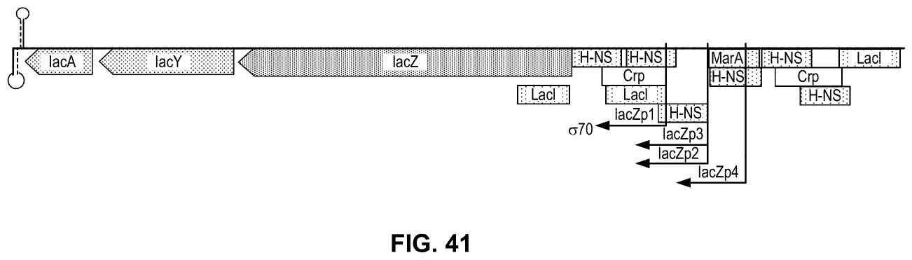

Rheostatic lactase production in synthetic E. coli cells may be performed using the methods provided herein. For example, an additional lacZ gene may be integrated into native lac operon pathways in a cell as illustrated in FIG. 41.

The lactose operon (lac operon) is an operon required for the transport and metabolism of lactose in E. coli and many other enteric bacteria. The lac operon of E. coli contains genes involved in lactose metabolism. The lac operon is expressed only when lactose is present and glucose is absent. The lac operon consists of 3 structural genes, and a promoter, a terminator, regulator, and an operator. The three structural genes are lacZ, lacY, and lacA. lacZ encodes beta-galactosidase (LacZ), an intracellular enzyme that cleaves the disaccharide lactose into glucose and lactose. lacY encodes beta-galactosidase permease (LacY) a transmembrane symporter that pumps beta-galactosides including lactose into the cell using a proton gradient. Permease increases the permeability of the cell to beta-galactosides. LacA encodes beta-galactosidase transacetylase (LacA), an enzyme that transfers an acetyl group from acetyl-CoA to beta-galactosides. Only lacZ and lacY may be necessary for lactose metabolism.

For example, B-galactosidase from Streptococcus thermophiles may be codon optimized for E. coli and inserted to native lactose pathway in E. coli to enhance lactose metabolism. The rates of metabolism of lactose in media is compared between wild-type cells and synthetic strains by measuring loss of lactose in cell media and or an increase in lactose metabolites from lactose in media over time. B-galctosidase genes BP_DNA_152 (SEQ ID NO: 266) and BP_DNA_153 (SEQ ID NO: 267) were prepared after codon optimization by IDT for E. coli (K12), BP_152 being from Streptococcus thermophilus and BP_DNA_153 being from E. coli. These two genes may be integrated separately into one or more, two or more, or several locations in the lac operon in E. coli and tested for their ability to enhance the lactose metabolism of the wild type organism. The Strep therm B-gal may comprise the amino acid sequence of BP_AA_026 (SEQ ID NO: 270). The E. coli B-gal amino acid sequence may comprise BP_AA_024 (SEQ ID NO: 268). In addition, GFP (BP_DNA_077) (SEQ ID NO: 42) may be integrated into the same locations as used above as a reporter gene. GFP integrants may show an increase in fluorescence when grown in the presence of lactose compared to lactose free media.

The piggyback technique may be employed to enhance the activity of lactose metabolism in a subject in need thereof. For example, during lactose metabolism a β-galactosidase enzyme (encoded by a lacZ gene) catalyzes the reaction of cleaving the disaccharide into glucose and galactose. A waning in the activity of this enzymatic step is thought to be responsible for the symptoms experienced by a large majority of humans, the condition referred to as lactose intolerance.

In some embodiments, microbes that are endogenous to the human gut may be engineered to enhance the expression and or activity of a β-galactosidase enzyme when lactose is present. In some embodiments, a β-galactosidase gene from Streptococcus thermophilus will be inserted into the native lac operon found in E. coli. The E. coli lac operon is very well studied and is known to be transcribed only when lactose is present and glucose is absent.

The engineered strains may be tested by growing under conditions where lactose is both present and absent, and taking samples at multiple time points. β-galactosidase activity may be tested by looking at the rate of lactose consumption from the media, or the β-galactosidase activity in crude cell lysates from the same samples. In some embodiments, strains harboring GFP reporter gene integrations will be grown under the same conditions as above, but fluorescence measurements will be taken from each sample which will indicate that the pathway is turned on and that we can use our Piggyback technique to add additional functionality in the E. coli lac operon.

Prokaryotes contain the lac operon which contains the lacZ gene which codes for β-galactosidase (lactase or β-gal). Streptococcus thermophilus contains a similar operon and was demonstrated capable of producing an active β-gal within the digestive tract of mice. Drouault et al. “Streptococcus thermophilus is able to produce a β-galactosidase active during its transit in the digestive tract of germ-free mice.” Appl. Environ. Microbiol. 68.2 (2002): 938-941. The enzyme commission (EC) number for bacterial β-gal is EC 3.2.1.23 (BP_AA_024)(SEQ ID NO: 268). The bacterial lactase shares no amino acid sequence similarity with the lactase produced by the small intestine. The beta-galactosidase encoding gene may comprise the DNA sequence of SEQ ID NO: 266 or 267. The beta-galactosidase enzyme may comprise the amino acid sequence of SEQ ID NO: 94, 268, or 270.

The present disclosure provides a piggyback strategy for integrating environmental kill switches that could be employed to engineer gut microbes (i.e. E. coli, Lactobacilli, Bacteroides) with the addition of a lac operon to produce and secrete lactase when the organisms sense the presence of lactose in the gut. By coupling the production and secretion of lactase to conditions only when the organisms sense that lactose is present should minimally disrupt the metabolism of the cell in its native niche. This provides the engineered organism with the same competitive advantage as other organisms in the gut allowing for durable integration of the engineered organism in the microbiome.

Gluten Intolerance in Humans

Gluten is a heterogenous mixture of insoluble proteins, consisting of gliadins and glutenins present in wheat, barley, and rye. Cavaletti, Linda, et al., 2019 “E40, a novel microbial protease efficiently detoxifying gluten proteins, for the dietary management of gluten intolerance.” Scientific reports 9.1:1-11. It is notoriously difficult to digest by mammalian proteolytic enzymes and therefore allowing proline-rich digestion-resistant peptides to enter the bloodstream and cause an immunologic response. Amador, Maria de Lourdes Moreno, et al. “A new microbial gluten-degrading prolyl endopeptidase: Potential application in celiac disease to reduce gluten immunogenic peptides.” PloS one 14.6 (2019). Over time, the repeated immune response can cause damage to the intestines and surrounding area. Although 30% of the human population has the genetic components which put them at risk for developing celiac disease, a much smaller percentage experience complications associated with this disease, which suggests that there are other components involved. Galipeau, Heather J., and Elena F. Verdu. “Gut microbes and adverse food reactions: Focus on gluten related disorders.” Gut Microbes 5.5 (2014): 594-605. Using effective glutenases (enzymes that degrade the proteins found in gluten), such as a prolyl endopeptidase (PEP), one could attenuate the effects of gluten intolerance in the host by engineering a resident microbe in the gut microbiota using piggyback methods of the present disclosure to excrete the endopeptidases when the organism senses the presence of gluten.

The disclosure provides a piggyback method for engineering organisms, using minimal genomic modifications to tether the expression of an action gene to an inducible promoter system, could produce a microbe that could be durably integrated into the IG tract and capable of sufficiently degrading the gluten proteins before they enter the bloodstream. For instance, engineering a stable resident gut microbe to express and secrete an endopeptidase enzyme such as a prolyl endopeptidase (BP_AA_022) (SEQ ID NO: 92) or the endopeptidase 40 enzyme (BP_AA_023) (SEQ ID NO: 93) only when the organism senses the presence of proline-rich peptides could augment the insufficient protease activity seen in the GI tract of gluten sensitive individuals. Cavaletti et al., 2019. The engineered organisms would have the expression and secretion of the enzymes tethered to promoter systems that are induced, or operons that are upregulated, when the organism is in the presence of gluten proteins.

Promoters and gene operons that are differentially expressed in gut microbiota while in the presence or absence of proline rich proteins, such as gliadins, could be determined by sampling the metatranscriptome and metaproteome of the gut microbiota in a variety of individuals with high and low gluten diets. By sequencing either dataset and mapping the sequences to an annotated reference map, the ideal promoters or gene operons can be sorted and determined. In vitro tests could be run using isolated strains found in the gut and analyzing the cell's individual response to a variety of conditions.

Diabetes Mellitus

Diabetes mellitus is a chronic disease associated with the increased concentration of glucose in the bloodstream. Type I is referred to as insulin dependent diabetes because the body is not able to produce a sufficient amount of insulin, a hormone secreted by the pancreas required for the cells in the body to take up the sugar in the bloodstream. Oral administration of insulin is theoretically possible and the solutions to overcome the many barriers of this treatment technique are a target of research. Wong et al., “Oral delivery of insulin for treatment of diabetes: status quo, challenges and opportunities.” Journal of Pharmacy and Pharmacology 68.9 (2016): 1093-1108. The other treatment solution is injections of purified insulin which lead to a host of problems that arise from the route of administration. By engineering a microbe or microbes in the gut microbiota to produce and secrete insulin that can be absorbed by the epithelial layer in a manner directly proportional to the concentration of glucose in the gut or bloodstream, many effects of the disease could be attenuated.

Glucose is a high energy sugar that many life forms preferentially metabolize. Since its value in nature is high, and many organisms will take up and convert the sugar into energy, there are many systems within cells that are sensitive to the extracellular presence or absence of the sugar. Through metatranscriptomic sequencing projects, promoters and gene operons that are differentially regulated appropriately in all environments could be identified and harnessed to produce insulin capable of being excreted by the microbe and absorbed by the host.

Having a member of the gut microbiota produce the insulin needed by the host bypasses many of the limitations seen with oral administration of insulin, such as limiting the hormone's exposure to harsh acidic conditions in the stomach and long-term stability. It also eliminates other side effects seen from insulin injections like skin diseases, lower patient compliance, and constant monitoring of the blood glucose levels. Tasking the production and secretion of insulin to gut microbiota, and tethering that production to the concentration of glucose being absorbed in the gut would mimic the body's natural response more accurately than the above mentioned treatments as well as bypassing many unfortunate side effects seen by the same treatments. In some embodiments, the action gene may encode insulin or an insulin precursor, for example, comprising the amino acid sequence GIVEQCCTSI CSLYQLENYC NFVNQHLCGS HLVEALYLVC GERGFFYTPK T (SEQ ID NO: 105), or a fragment thereof.

A synthetic microorganism is provided encoding an action gene. In some embodiments, the action gene may encode a toxin, endopeptidase, galactosidase, or an insulin protein. The toxin may be selected from a sprA1, sprA2, truncated sprA1, sprG1, sprG1 truncated, sprG2, sprG2 variant, or sprG3 toxin. The action gene may be a toxin gene encoding a toxin comprising an amino acid sequence selected from SEQ ID NO: 72, 73, 84, 89, 90, 91, or 95. The action gene may be a galactosidase gene encoding a beta-galactosidase enzyme. The gene encoding the beta-galactosidase enzyme may comprise the DNA sequence of SEQ ID NO: 266 or 267. The beta-galactosidase enzyme may comprise the amino acid sequence of SEQ ID NO: 94, 268, or 270. The action gene may encode an endopeptidase gene. The endopeptidase gene may encode a prolyl endopeptidase or endopeptidase 40. The endopeptidase gene may encode an endopeptidase amino acid sequence selected from SEQ ID NO: 92 or 93.

The target microorganism may be a Streptococcus species. In some embodiments, the target microorganism may be Streptococcus agalactiae, Streptococcus pneumonia, or Streptococcus mutans. A method is provided to prepare a safe Streptococcus strain comprising screening a target Streptococcus genes for self-lethality and integrating a lethal gene into the genome in one or more operons that are upregulated in serum. The Streptococcus spp. may be a group B Strep species. The piggyback method may be employed to create a kill switch in, for example, Streptococcus agalactiae.

Strep agalactiae is a pathogenic strain which can cause neonatal sepsis and bovine mastitis. Stoll et al., Pediatrics 2011, 127 (5), 817-826. https://doi.org/10.1542/peds.2010-2217. Keefe, G. P. Streptococcus Agalactiae Mastitis: A Review. Can. Vet. J. 1997, 38 (7), 429-437.

Strep agalactiae can be a part of the normal human microbiome but can also become an opportunistic pathogen if allowed access to certain environments. Using the present technology to create a kill switched Strep agalactiae reduces the risk of infection from that strain without compromising its ability to occupy its native niche. Toxin-antitoxin systems will be harnessed to create a kill switch that is activated in serum to render Strep agalactiae unable to reproduce or induce artificial programmed cell death. This piggyback method allows for design and production of live biotherapeutic products for use as preventative treatments for many opportunistic infections through bacterial interference without the risk of infection.

In the bovine population, Strep agalactiae is a highly contagious pathogen and is well suited to flourishing in the udder environment. Strep agalactiae is one of the major pathogens causing mastitis and a large problem for the dairy industry since the loss of millions of dollars are attributed to mastitis every year. It is also found on up to 30% of pregnant women in the United States which presents a danger to infants since they can become colonized through passage of the birth canal or from infected amniotic fluid.

Strep agalactiae can also be a commensal member of the microbiome and lives causing no adverse symptoms. To prevent opportunistic infections, a kill switch will be designed and integrated into the genome of Strep agalactiae, so if it reaches the bloodstream or other biological fluid, it will not be capable of growing or causing disease. First, toxin/antitoxin systems native to Strep agalactiae will be investigated to find a toxin gene lethal to the Strep strain. The toxin gene may then be integrated into an operon that is highly upregulated in serum. Using genomic editing techniques, the toxin gene will be placed on the same mRNA transcript of the upregulated gene(s) so the expression of the toxin will be tied to the upregulated gene(s). The increased expression of the toxin will induce the cell death of Strep agalactiae in serum.

Vectors and Target Microorganisms

Also described herein are vectors comprising polynucleotide molecules, as well as target cells transformed with such vectors. Polynucleotide molecules described herein may be joined to a vector, which include a selectable marker and origin of replication, for the propagation host of interest. Cells may be are genetically engineered to include these vectors and thereby transcribe RNA and express polypeptides. Vectors herein include polynucleotides molecules operably linked to suitable transcriptional or translational regulatory sequences, such as those for microbial target cells. Examples of regulatory sequences include transcriptional promoters, operators, or enhancers, mRNA ribosomal binding sites, and appropriate sequences which control transcription and translation. Nucleotide sequences as described herein are operably linked when the regulatory sequences herein functionally relate to, e.g., a cell death gene encoding polynucleotide.

Typical vehicles include plasmids, shuttle vectors, baculovirus, inactivated adenovirus, and the like. In certain examples described herein, the vehicle may be a modified pIMAY, pIMAYz, or pKOR integrative plasmid, as discussed herein.

A target microorganism may be selected from any microorganism having the ability to durably replace a specific undesirable microorganism after decolonization. The target microorganism may be a wild-type microorganism that is subsequently engineered to enhance safety by methods described herein. The target microorganism may be selected from a bacterial, fungal, or protozoal target microorganism. The target microorganism may be a strain capable of colonizing a dermal and/or mucosal niche in a subject. The target microorganism may be a wild-type microorganism, or a synthetic microorganism that may be subjected to further molecular modification. The target microorganism may be selected from a genus selected from the group consisting of Staphylococcus, Acinetobacter, Corynebacterium, Streptococcus, Escherichia, Mycobacterium, Enterococcus, Bacillus, Klebsiella, and Pseudomonas. The target microorganism may be selected from the group consisting of Staphylococcus aureus, Staphylococcus epidermidis, Staphylococcus chromogenes, Staphylococcus simulans, Staphylococcus saprophyticus, Staphylococcus haemolyticus, Staphylococcushyicus, E. coli, Acinetobacter baumannii, Streptococcus pyogenes, Streptococcus agalactiae, Streptococcus dysgalactiae, Streptococcus uberis, Escherichia coli, Mammary Pathogenic Escherichia coli (MPEC), Bacillus cereus, Bacillus hemolysis, Mycobacterium tuberculosis, Mycobacterium bovis, Mycoplasma bovis, Enterococcus faecalis, Enterococcus faecium, Corynebacterium bovis, Corynebacterium amycolatum, Corynebacterium ulcerans, Klebsiella pneumonia, Klebsiella oxytoca, Enterobacter aerogenes, Arcanobacterium pyogenes, Trueperella pyogenes, Pseudomonas aeruginosa. The target microorganism may be a species having a genus selected from the group consisting of Candida or Cryptococcus. The target microorganism may be Candida parapsilosis, Candida krusei, Candida tropicalis, Candida albicans, Candida glabrata, or Cryptococcus neoformans.

The target microorganism may be of the same genus and species as the undesirable microorganism, but of a different strain. For example, the undesirable microorganism may be an antibiotic-resistant Staphylococcus aureus strain, such as an MRSA strain. The antibiotic-resistant Staphylococcus aureus stain may be a pathogenic strain, which may be known to be involved in dermal infection, mucosal infection, bacteremia, and/or endocarditis. Where the undesirable microorganism is a Staphylococcus aureus strain, e.g., an MRSA, the target microorganism may be, e.g., a less pathogenic strain which may be an isolated strain such as Staphylococcus aureus target cell such as an RN4220 or 502a strain, and the like. Alternatively, the target cell may be of the same strain as the undesirable microorganism. In another example, the undesirable microorganism is an Escherichia coli strain, for example, a uropathogenic E. coli type 1 strain or p-fimbriated strain, for example, a strain involved in urinary tract infection, bacteremia, and/or endocarditis. In another example, the undesirable strain is a Cutibacterium acnes strain, for example a strain involved in Acnes vulgaris, bacteremia, and/or endocarditis. In another example, the undesirable microorganism is a Streptococcus mutans strain, for example, a strain involved in S. mutans endocarditis, dental caries.

Model Antibiotic-Susceptible Target Microorganism

The target microorganism may be an antibiotic-susceptible microorganism of the same species as the undesirable microorganism. In one embodiment, the undesirable microorganism is an MRSA strain and the replacement target microorganism is an antibiotic susceptible Staphylococcus aureus strain. The antibiotic susceptible microorganism may be Staphylococcus aureus strain 502a (“502a”). 502a is a coagulase positive, penicillin sensitive, nonpenicillinase producing staphylococcus, usually lysed by phages 7, 47, 53, 54, and 77. Serologic type (b)ci. Unusual disc antibiotic sensitivity pattern is exhibited by 502a because this strain is susceptible to low concentrations of most antibiotics except tetracycline; resistant to 5 g, but sensitive to 10 μg of tetracycline. In some embodiments, the 502a strain may be purchased commercially as Staphylococcus aureus subsp. Aureus Rosenbach ATCC®27217™

Methods for Selecting of a Target Microorganism

Selection of the target microorganism may be performed by identification of the undesirable microorganism, and selecting a candidate target microorganism that is of the same genus and species as the undesirable microorganism. Candidate target strains having same genus and species as an undesirable strain may be obtained commercially, e.g., from ATCC®, or may be obtained by isolation from a host subject. The target strain may be a strain that is susceptible to an antimicrobial agent, such as an antibiotic.

Selection of an appropriate target microorganism may be confirmed by effectively decolonizing the undesirable microorganism from a host subject and replacing with a wild-type putative target microorganism, as described in WO 2019113096, Starzl et al., which is incorporated herein by reference. The ability to durably replace an undesirable microorganism with a wild type target microorganism for a period of at least 4 weeks, 8 weeks, 12 weeks, 16 weeks, 20 weeks, or 28 weeks, confirms the selection of the target microorganism. For example, the undesirable microorganism may be Methicillin-Resistant Staphylococcus aureus (MRSA) which is the cause of a disproportionate amount of invasive bacterial infections worldwide. The colonization state for Staphylococcus aureus is regarded as a required precondition for most invasive infections. However, decolonization with standard antiseptic regimens as a method for reducing MRSA colonization and infections provided only mixed results. Starzl et al. studied candidate target strain BP-001 for the feasibility and durability of a novel decolonization approach to undesirable microorganism MRSA by using intentional recolonization with a different Staphylococcus aureus strain as a candidate target microorganism was performed in hopes of improving duration of effect versus standard decolonization. In WO 2019113096, Starzl et al., 765 healthy volunteers were screened for Staphylococcus aureus colonization. The overall MRSA rate for the screened population was 8.5%. A cohort of 53 MRSA colonized individuals participated in a controlled study of a decolonization/recolonization therapy using Staphylococcus aureus 502a WT strain BioPlx-01 vs. a control group of standard decolonization alone. Duration of MRSA absence from the colonization state as well as persistence of the intentional MSSA recolonization was monitored for 6 months. The control group (n=15) for the efficacy portion of the MRSA decolonization protocol showed MRSA recurrence of 60% at the 4 week time point. The test group employing the BioPlx-01WT protocol (n=34) showed 0% MRSA recurrence at the 8 week primary endpoint and continued to show no evidence of MRSA recurrence out to 26 weeks.

The fact that WT target strain Staphylococcus aureus 502a BP-001 when used in a decolonization/recolonization protocol provided good durability of decolonization confirmed the choice of MSSA BP-001 as a target strain. As provided herein below, the spa type of BP_001 assigned by BioNumerics is t010.

Another WT target strain isolated from one of the present inventors is MSSA strain CX_001. As provided herein below, the spa type of CX_001 assigned by BioNumerics is t688.

In some embodiments, the target microorganism and/or the synthetic microorganism comprises (i) the ability to durably colonize a niche in a subject following decolonization of the undesirable microorganism and administering the target or synthetic microorganism to a subject, and (ii) the ability to prevent recurrence of the undesirable microorganism in the subject for a period of at least two weeks, at least four weeks, at least six weeks, at least eight weeks, at least ten weeks, at least 12 weeks, at least 16 weeks, at least 24 weeks, at least 26 weeks, at least 30 weeks, at least 36 weeks, at least 42 weeks, or at least 52 weeks after the administering step.

Unfortunately, even an antimicrobial agent-susceptible target microorganism may cause systemic infection. Therefore, as provided herein, the target microorganism is subjected to molecular modification to incorporate regulatory sequences including, e.g., an inducible first promoter for expression of the cell death gene, v-block, or nanofactory, in order to enhance safety and reduce the likelihood of pathogenic infection as described herein.

Methods for Determining Detectable Presence and Identification of a Microorganism

Any method known in the art may be employed for determination of the detectable presence and identification of an undesirable, target, or synthetic microorganism with respect to genus, species and strain. An overview of methods may be found in Aguilera-Arreola MG. Identification and Typing Methods for the Study of Bacterial Infections: a Brief Review and Mycobacterial as Case of Study. Arch Clin Microbiol. 2015, 7:1, which is incorporated herein by reference.

The detectable presence and/or identification of a genus, species and/or strain of a bacteria may be determined by phenotypic methods and/or genotypic methods. Phenotypic methods may include biochemical reactions, serological reactions, susceptibility to anti-microbial agents, susceptibility to phages, susceptibility to bacteriocins, and/or profile of cell proteins. One example of a biochemical reaction is the detection of extracellular enzymes. For example, staphylococci produce many different extracellular enzymes including DNAase, proteinase and lipases. Gould, Simon et al., 2009, The evaluation of novel chromogenic substrates fro detection of lipolytic activity in clinical isolates of Staphylococcus aureus and MRSA from two European study groups. FEMS Microbiol Let 297; 10-16. Chomogenic substrates may be employed for detection of extracellular enzymes. For example, CHROMager™ MRSA chromogenic media (CHROMagar, Paris, France) may be employed for isolation and differentiation of Methicillin Resistant Staphylococcus aureus (MRSA) including low level MRSA. Samples are obtained from, e.g., nasal, perineal, throat, rectal specimens are obtained with a possible enrichment step. If the agar plate has been refrigerated, it is allowed to warm to room temperature before inoculation. The sample is streaked onto plate followed by incubation in aerobic conditions at 37° C. for 18-24 hours. The appearance of the colonies is read, wherein MRSA colonies appear as rose to mauve colored, Methicillin Susceptible Staphylococcus aureus (MSSA) colonies are inhibited, and other bacteria appear as blue, colorless or inhibited colonies. Definite identification as MRSA requires, in addition, a final identification as Staphylococcus aureus. For example, CHROMagar™ Staph aureus chromogenic media may be employed where S. aurues appears as mauve, S. saprophyticus appears turquoise blue, E. coli, C. albicans and E. faecalis are inhibited. For detection of Group B Streptococcus(GBS) (S. agalactiae), CHROMagar™ StrepB plates may be employed, wherein Streptococcus agalactiae (group B) appear mauve, Enterococcus spp. and E. faecalis appear steel blue, Lactobacilli, leuconostoc and lactococci appear light pink, and other microorganisms are blue, colorless or inhibits. For detection of various Candida spp., CHROMager™ Candida chromogenic media may be employed. Candida species are involved in superficial oropharyngeal and urogenital infections. Although C. albicans remains a major species involved, other types such as C. tropicalis, C. krusai, or C. glabrata have increased as new antifungal agents have worked effectively against C. albicans. Sampling and direct streaking of skin, sputum, urine, vaginal specimens samples and direct streaking or spreading onto plate, followed by incubation in aerobic conditions at 30-37° C. for 48 hours, and reading of plates for colony appearance where C. albicans is green, C. tropicalis is metallic blue, C. krusei is pink and fuzzy, C. kefyr and C. glabrata are mauve-brown, and other species are white to mauve.

Genotypic methods for genus and species identification may include hybridization, plasmids profile, analysis of plasmid polymorphism, restriction enzymes digest, reaction and separation by Pulsed-Field Gel Electrophoresis (PFGE), ribotyping, polymerase chain reaction (PCR) and its variants, phage typing, Ligase Chain Reaction (LCR), Transcription-based Amplification System (TAS), or any of the methods described herein.

Identification of a microbe can be performed, for example, by employing Galileo™ Antimicrobial Resistance (AMR) detection software (Arc Bio LLC, Menlo Park, Calif. and Cambridge, Mass.) that provides annotations for gram-negative bacterial DNA sequences.

The microbial typing method may be selected from genotypic methods including Multilocus Sequence Typing (MLST) which relies on PCR amplification of several housekeeping genes to create allele profiles; PCR-Extragenic Palindromic Repetitive Elements (rep-PCR) which involves PCR amplification of repeated sequences in the genome and comparison of banding patterns; AP-PCR which is Polymerase Chain Reaction using Arbitrary Primers; Amplified Fragment Length Polymorphism (AFLP) which involves enzyme restriction digestion of genomic DNA, binding of restriction fragments and selective amplification; Polymorphism of DNA Restriction Fragments (RFLP) which involves Genomic DNA digestion or of an amplicon with restriction enzymes producing short restriction fragments; Random Amplified Polymorphic DNA (RAPD) which employs marker DNA fragments from PCR amplification of random segments of genomic DNA with single primer of arbitrary nucleotide sequence; Multilocus Tandem Repeat Sequence Analysis (MLVA) which involves PCR amplification of loci VTR, visualizing the polymorphism to create an allele profile; or Pulsed-Fields Gel Electrophoresis (PFGE) which involves comparison of macro-restriction fragments. PFGE method of electrophoresis is capable of separating fragments of various fragment lengths, for example, a length higher than 50 kb up to 10 Mb, which is not possible with conventional electrophoresis, which can separate only fragments of 100 bp to 50 kb. This capacity of PFGE is due to its multidirectional feature, changing continuously the direction of the electrical field, thus, permitting the re-orientation of the direction of the DNA molecules, so that these can migrate through the agarose gel, in addition to this event, the applied electrical pulses are of different duration, fostering the reorientation of the molecules and the separation of the fragments of different size. PFGE is described in Bonness et al., 2008, J Clin Microbiol Vo. 46, No. 2, pp. 456-461, which is incorporated herein by reference. One PFGE apparatus may be the Contour Clamped Homogeneous Electric Fields (CHEF, BioRad). Pulsed-field gel electrophoresis (PFGE) is considered a gold standard technique for MRSA typing, because of its high discriminatory power, but its procedure is complicated and time consuming.

Another method of identifying various S. aureus strains employs sequence-based spa typing. The spa gene encodes a cell wall component of Staphylococcus aureus protein A, and exhibits polymorphism. Single locus DNA-sequencing of the repeat region of the Staphylococcus protein A gene (spa) can be used for reliable, accurate and discriminatory typing. Repeats may be assigned a numerical code and the spa-type may be deduced from the order of specific repeates.

The sequence based-spa typing can be used as a rapid test screen, for example, by the method of Narukawa et al. 2009 Tohoku J Exp Med 2009, 218, 207-213, which is incorporated herein by reference. Spa typing of isolated S. aureus strains was performed as shown in Example 1; eighteen MSSA strains were isolated and spa typed herein as candidate target strains. Results are shown in Table 2. There were at least 10 spa types identified in these SA samples including spa types t010 (strain BP_001), t688 (CX_001), t008 (A1-033N, A1-0905A), t005 (A1-0791N, A1-0940A, A1-0068, A1-1691N), t021 (A1-0915N), t127 (A1-1415N, A1-0609N), t002 (A1-9080A, A1-415), t3841 (A1-1D-915, A1-1618, A1-1235N), t272 (A1-1D-180), and t1328 (A1-0909N).

For example, in order to help predict a microorganism's ability to competitively exclude other similar microorganisms from a specific niche it may be useful to employ a target strain of a known spa type. Understanding the relationship between strain type and durable colonization of a microbiome would also be extremely useful information.

In some embodiments, the target strain is a Staphylococcus aureus strain. The target strain may be an MSSA strain. The target strain may be an S. aureus strain having a spa type selected from t010, t688, t008, t005, t021, t127, t002, t3841, t272, and t1328. The target strain may be an MRSA strain.

Synthetic Microorganisms are Incapable of Causing Bacteremia

A Bacteremia Study was performed in vivo in mice to compare the clinical effects (bacteremia) in mice following tail vein injection of 10\hat{ }7 synthetic Staphylococcus aureus (SA) modified with kill switch (KS) (BP_109, CX_013) technology or wild type (WT) target strains (BP_001, CX_001) and observation over 8 days, as described in the examples herein. The synthetic microorganisms modified with KS technology were designed to initiate artificially programmed cell death upon interacting with blood, serum, or plasma of the mammalian host.

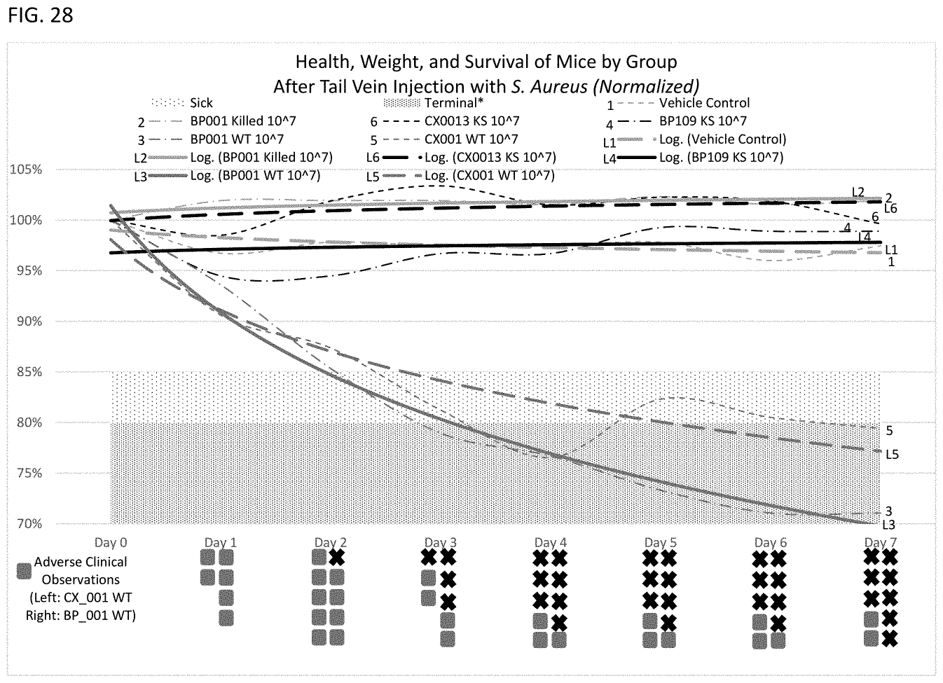

As shown in FIG. 28 and described in the examples, all mice injected intravenously via tail vein injection with KS organisms as well as negative controls were healthy with no adverse clinical symptoms for the duration of the study, excluding one observation of hypoactivity which subsided by next observation. All mice injected with WT organisms experienced a wide variety of abnormal clinical observations, significant morbundity, and were either deceased or were fit for euthanasia by ethical standards. This study demonstrated the efficacy and safety of the KS technology with 100% survival and health of all test subjects. Synthetic Staph aureus strains comprising a kill switch may significantly de-risk protective organisms for use in methods for prevention and treatment of infectious disease.

Methods for Use and Compositions

In some embodiments, synthetic microorganisms provided herein comprising minimal genomic modification may be used in methods comprising decolonization or suppression of an undesirable microorganism, followed by recolonization or replacement with the synthetic microorganism. Expectations for non-co-colonization are important for durability of the present methods for prevention of recurrence of pathogenic colonization or infection.

Suppression/Decolonization

An undesirable microorganism may be suppressed, or decolonized, by topically applying a disinfectant, antiseptic, or biocidal composition directly to the skin or mucosa of the subject, for example, by spraying, dipping, or coating the affected area, optionally the affected area and adjacent areas, or greater than 25%, 50%, 75%, or greater than 90% of the external or mucosal surface area of the subject with the disinfectant, antiseptic, or biocidal composition. In some embodiments, the affected area, or additional surface areas are allowed to air dry or are dried with an air dryer under gentle heat, or are exposed to ultraviolet radiation or sunlight prior to clothing or dressing the subject. In one embodiment, the suppression comprises exposing the affected area, and optionally one or more adjacent or distal areas of the subject, with ultraviolet radiation. In various embodiments, any commonly employed disinfectant, antiseptic, or biocidal composition may be employed. In one embodiment, a disinfectant comprising chlorhexidine or a pharmaceutically acceptable salt thereof is employed.

In some embodiments, the bacteriocide, antiseptic, astringent, and/or antibacterial agent is selected from the group consisting of alcohols (ethyl alcohol, isopropyl alcohol), aldehydes (glutaraldehyde, formaldehyde, formaldehyde-releasing agents (noxythiolin=oxymethylenethiourea, tauroline, hexamine, dantoin), o-phthalaldehyde), anilides (triclocarban=TCC=3,4,4′-triclorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), quaternary ammonium compounds (cetrimide, benzalkonium chloride, cetyl pyridinium chloride), silver compounds (silver sulfadiazine, silver nitrate), peroxy compounds (hydrogen peroxide, peracetic acid), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), botanical extracts (Malaleuca spp. (tea tree oil), Cassia fistula Linn, Baekea frutesdens L., Melia azedarach L., Muntingia calabura, Vitis vinfera L, Terminalia avicennioides Guill & Perr., Phylantus discoideus muel. Muel-Arg., Ocimum gratissimum Linn., Acalypha wilkesiana Muell-Arg., Hypericum pruinatum Boiss. & Bal., Hypericum olimpicum L. and Hypericum sabrum L., Hamamelis virginiana (witch hazel), Eucalyptus spp., Rosemarinus officinalis spp. (rosemary), Thymus spp. (thyme), Lippia spp. (oregano), Cymbopogon spp. (lemongrass), Cinnamomum spp., Geranium spp., Lavendula spp.), and topical antibiotic compounds (bacteriocins; mupirocin, bacitracin, neomycin, polymyxin B, gentamicin).

Suppression of the undesirable microorganism also may be performed by using photosensitizers instead of or in addition to, e.g., topical antibiotics. For example, Peng Zhang et al., Using Photosensitizers Instead of Antibiotics to Kill MRSA, GEN News Highlights, Aug. 20, 2018; 48373, developed a technique using light to activate oxygen, which suppresses to microbial growth. Photosensitizers, such as dye molecules, become excited when illuminated with light. The photosensitizers convert oxygen into reactive oxygen species that kill the microbes, such as MRSA. In order to concentrate the photosensitizers to improve efficacy, water-dispersible, hybrid photosensitizers were developed by Zhang et al., comprising noble metal nanoparticles decorated with amphiphilic polymers to entrap molecular photosensitizers. The hybrid photosensitizers may be applied to a subject, for example, on a dermal surface or wound, in the form of a spray, lotion or cream, then illuminated with red or blue light to reduce microbial growth.

A decolonizing composition may be in the form of a topical solution, lotion, or ointment form comprising a disinfectant, biocide photosensitizer or antiseptic compound and one or more pharmaceutically acceptable carriers or excipients. In one specific example, an aerosol disinfectant spray is employed comprising chlorhexidine gluconate (0.4%), glycerin (10%), in a pharmaceutically acceptable carrier, optionally containing a dye to mark coverage of the spray. In one embodiment, the suppressing step comprises administration to one or more affected areas, and optionally one or more surrounding areas, with a spray disinfectant as disclosed in U.S. Pat. Nos. 4,548,807 and/or 4,716,032, each of which is incorporated herein by reference in its entirety. The disinfectant spray may be commercially available, for example, Fight Bac®, Deep Valley Farm, Inc., Brooklyn, Conn. Other disinfectant materials may include chlorhexidine or salts thereof, such as chlorhexidine gluconate, chlorhexidine acetate, and other diguanides, ethanol, SD alcohol, isopropyl alcohol, p-chloro-o-benzylphenol, o-phenylphenol, quaternary ammonium compounds, such as n-alkyl/dimethyl ethyl benzyl ammonium chloride/n-alkyl dimethyl benzyl ammonium chloride, benzalkonium chloride, cetrimide, methylbenzethonium chloride, benzethonium chloride, cetalkonium chloride, cetylpyridinium chloride, dofanium chloride, domiphen bromide, peroxides and permanganates such as hydrogen peroxide solution, potassium permanganate solution, benzoyl peroxide, antibacterial dyes such as proflavine hemisulphate, triphenylmethane, Brilliant green, Crystal violet, Gentian violet, quinolone derivatives such as hydroxyquinoline sulphate, potassium hydroxyquinoline sulphate, chlorquinaldol, dequalinium chloride, di-iodohydroxyquinoline, Burow's solution (aqueous solution of aluminum acetate), bleach solution, iodine solution, bromide solution. Various Generally Recognized As Safe (GRAS) materials may be employed in the disinfectant or biocidal composition including glycerin, and glycerides, for example but not limited to mono- and diglycerides of edible fat-forming fatty acids, diacetyl tartaric acid esters of mono- and diglycerides, triacetin, acettooleins, acetostearins, glyceryl lactopalmitate, glyceryl lactooleate, and oxystearins.

### Administration and Compositions

In some embodiments, compositions are provided comprising a synthetic microorganism and an excipient, or carrier. The compositions can be administered in any method suitable to their particular immunogenic or biologically or immunologically reactive characteristics, including oral, intravenous, buccal, nasal, mucosal, dermal or other method, within an appropriate carrier matrix. In one embodiment, compositions are provided for topical administration to a dermal site, and/or a mucosal site in a subject. Another specific embodiment involves the oral administration of the composition of the disclosure.

In some embodiments, the replacing step comprises topically administering of the synthetic strain to the dermal or mucosal at least one host subject site and optionally adjacent areas in the subject no more than one, no more than two, or no more than three times. The administration may include initial topical application of a composition comprising at least 10⁶, at least 10⁷, at least 10⁸, at least 10⁹, or at least 10¹⁰ CFU of the synthetic strain and a pharmaceutically acceptable carrier to the at least one host site in the subject. The initial replacing step may be performed within 12 hours, 24 hours, 36 hours, 48 hours, 72 hours, 4 days, 5 days, 6 days, 7 days, 8 days, or 9 days of the final suppressing step.

The composition comprising a synthetic microorganism may be administered to the dermal and/or mucosal at least one site in the subject, and optionally adjacent sites at least once, for example, from one to 30 times, one to 20 times, one to ten times, one to six times, one to five times, one to four times, one to three times, or one to two times, or no more than once, twice, three times, 4 times, 5 times, 6 times, 8 times per month, 10 times, or no more than 12 times per month. Subsequent administration of the composition may occur after a period of, for example, one to 30 days, two to 20 days, three to 15 days, or four to 10 days after the first administration.

Colonization of the synthetic microorganism may be promoted in the subject by administering a composition comprising a promoting agent selected from a nutrient, prebiotic, stabilizing agent, humectant, and/or probiotic bacterial species. The promoting agent may be administered to a subject in a separate promoting agent composition or may be added to the microbial composition.

In some embodiments, the promoting agent may be a nutrient, for example, selected from sodium chloride, lithium chloride, sodium glycerophosphate, phenylethanol, mannitol, tryptone, and yeast extract. In some embodiments, the prebiotic is selected from the group consisting of short-chain fatty acids (acetic acid, propionic acid, butyric acid, isobutyric acid, valeric acid, isovaleric acid), glycerol, pectin-derived oligosaccharides from agricultural by-products, fructo-oligosaccarides (e.g., inulin-like prebiotics), galacto-oligosaccharides (e.g., raffinose), succinic acid, lactic acid, and mannan-oligosaccharides.

In some embodiments, the promoting agent may be a probiotic. The probiotic may be any known probiotic known in the art. Probiotics are live microorganisms that provide a health benefit to the host. In methods provided herein, probiotics may be applied topically to dermal and mucosal microbiomes, and/or probiotics may be orally administered to provide dermal and mucosal health benefits to the subject. Several strains of Lactobacillus have been shown to have systemic anti-inflammatory effects. Studies have shown that certain strains of Lactobacillus reuteri induce systemic anti-inflammatory cytokines, such as interleukin (IL)-10. Soluble factors from Lactobacillus reuteri inhibit production of pro-inflammatory cytokines. Lactobacillus paracasei strains have been shown to inhibit neutrogenic inflammation in a skin model Kober at al., 2015, Int J Women's Dermatol 1(2015) 85-89. In human dermal fibroblasts and hairless mice models, Lactobacillus Plantarum has been shown to inhibit UVB-induced matrix metalloproteinase 1 (MMP-1) expression to preserve procollagen expression in human fibroblasts. Oral administration of L. plantarum in hairless mice histologic samples demonstrated that L. plantarum inhibited MMP-13, MMP-2, and MMP-9 expression in dermal tissue.

Clinically, the topical application of probiotics has also been shown to modify the barrier function of the skin with a secondary increase in antimicrobial properties of the skin. Streptococcus thermophiles when applied topically has been shown to modify the barrier function of the skin with a secondary increase in antimicrobial properties of the skin. Streptococcus thermophiles when applied topically has been shown to increase ceramide production both in vitro and in vivo. Ceramides trap moisture in the skin, and certain ceramide sphingolipids, such as phytosphingosine (PS), exhibit direct antimicrobial activity against P. acnes. Kober at al., 2015, Int J Women's Dermatol 1(2015) 85-89.

Two clinical trials of topical preparations of probiotics have assessed their effect on acne. Enterococcus fecalis lotion applied to the face for 8 weeks resulted in a 50% reduction of inflammatory lesions was noted compared to placebo. A reduction in acne count, size, and associated erythema was noted during a clinical study of Lactobacillus plantarum topical extract. Kober at al., 2015, Int J Women's Dermatol 1(2015) 85-89.

Clinical trials of topical probiotics have evaluated their effect on mucosal systems. In one study, Streptococcus salivarius was administered by nasal spray for the prevention of acute otitis media (AOM). If the nasopharynx was successfully colonized, there was significant effect on reducing AOM. Marchisio et al. (2015). Eur. J. Clin. Microbiol. Infect. Dis. 34, 2377-2383. In another trial, sprayed application of S. sanguinis and L. Rhamnosus decreased middle ear fluid in children with secretory otitis media. Skovbjerg et al. (2008). Arch. Dis. Child. 94, 92-98.

The probiotic may be a topical probiotic or an oral probiotic. The probiotic may be, for example, a different genus and species than the undesirable microorganism, or of the same genus but different species, than the undesirable microorganism. The probiotic species may be a different genus and species than the target microorganism. The probiotic may or may not be modified to comprise a kill switch molecular modification. The probiotic may be selected from a Lactobacillus spp., Bifidobacterium spp. Streptococcus spp., or Enterococcus spp. The probiotic may be selected from Bifidobacterium breve, Bifidobacterium bifadum, Bifidobacterium lactis, Bifidobacterium infantis, Bifidobacterium breve, Bifidobacterium longum, Lactobacillus reuteri, Lactobacillus paracasei, Lactobacillus plantarum, Lactobacillus johnsonii, Lactobacillus rhamnosus, Lactobacillus acidophilus, Lactobacillus salivarius, Lactobacillus casei, Lactobacillus plantarum, Lactococcus lactis, Streptococcus thermophiles, Streptococcus salivarius, or Enterococcus fecalis.

The promoting agent may include a protein stabilizing agent such as those disclosed in an incorporated by reference from U.S. Pat. No. 5,525,336 is included in the composition. Non-limiting examples include glycerol, trehelose, ethylenediaminetetraacetic acid, cysteine, a cyclodextrin such as an alpha-, beta-, or gamma-cyclodextrin, or a derivative thereof, such as a 2-hydroxypropyl beta-cyclodextrin, and proteinase inhibitors such as leupeptin, pepstatin, antipain, and cystatin.

The promoting agent may include a humectant. Non-limiting examples of humectants include glycerin, sorbitol, sodium 2-pyrrolidone-5-carboxylate, soluble collagen, and dibutylphthalate.

### Compositions

Compositions are provided comprising a synthetic microorganism according to the disclosure and a pharmaceutically acceptable carrier, diluent, emollient, binder, excipient, lubricant, sweetening agent, flavoring agent, buffer, thickener, wetting agent, or absorbent.

Pharmaceutically acceptable diluents or carriers for formulating the composition are selected from the group consisting of water, saline, phosphate buffered saline (PBS), PBST, sterile Luria broth, tryptone broth, or tryptic soy broth (TSB), or a solvent. The solvent may be selected from, for example, ethyl alcohol, toluene, isopropanol, n-butyl alcohol, castor oil, ethylene glycol monoethyl ether, diethylene glycol monobutyl ether, diethylene monoethyl ether, dimethyl sulphoxide, dimethyl formamide and tetrahydrofuran. The carrier or diluent may further comprise one or more surfactants such as i) Anionic surfactants, such as metallic or alkanolamine salts of fatty acids for example sodium laurate and triethanolamine oleate; alkyl benzene sulphones, for example triethanolamine dodecyl benzene sulphonate; alkyl sulphates, for example sodium lauryl sulphate; alkyl ether sulphates, for example sodium lauryl ether sulphate (2 to 8 EO); sulphosuccinates, for example sodium dioctyl sulphonsuccinate; monoglyceride sulphates, for example sodium glyceryl monostearate monosulphate; isothionates, for example sodium isothionate; methyl taurides, for example Igepon T; acylsarcosinates, for example sodium myristyl sarcosinate; acyl peptides, for example Maypons and lamepons; acyl lactylates, polyalkoxylated ether glycollates, for example trideceth-7 carboxylic acid; phosphates, for example sodium dilauryl phosphate; Cationic surfactants, such as amine salts, for example sapamin hydrochloride; quartenary ammonium salts, for example Quaternium 5, Quaternium 31 and Quaternium 18; Amphoteric surfactants, such as imidazol compounds, for example Miranol; N-alkyl amino acids, such as sodium cocaminopropionate and asparagine derivatives; betaines, for example cocamidopropylebetaine; Nonionic surfactants, such as fatty acid alkanolamides, for example oleic ethanolamide; esters or polyalcohols, for example Span; polyglycerol esters, for example that esterified with fatty acids and one or several OH groups; Polyalkoxylated derivatives, for example polyoxy:polyoxyethylene stearate; ethers, for example polyoxyethe lauryl ether; ester ethers, for example Tween; amine oxides, for example coconut and dodecyl dimethyl amine oxides. In some embodiments, more than one surfactant or solvent is included.

The composition may include a buffer component to help stabilize the pH. In some embodiments, the pH is between 4.5-8.5. For example, the pH can be approximately 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0, including any value in between. In some embodiments, the pH is from 5.0 to 8.0, 6.0 to 7.5, 6.8 to 7.4, or about 7.0. Non-limiting examples of buffers can include ACES, acetate, ADA, ammonium hydroxide, AMP (2-amino-2-methyl-1-propanol), AMPD (2-amino-2-methyl-1,3-propanediol), AMPSO, BES, BICINE, bis-tris, BIS-TRIS propane, borate, CABS, cacodylate, CAPS, CAPSO, carbonate (pK1), carbonate (pK2), CHES, citrate (pK1), citrate (pK2), citrate (pK3), DIPSO, EPPS, HEPPS, ethanolamine, formate, glycine (pK1), glycine (pK2), glycylglycine (pK1), glycylglycine (pK2), HEPBS, HEPES, HEPPSO, histidine, hydrazine, imidazole, malate (pK1), malate (pK2), maleate (pK1), maleate (pK2), MES, methylamine, MOBS, MOPS, MOPSO, phosphate (pK1), phosphate (pK2), phosphate (pK3), piperazine (pK1), piperazine (pK2), piperidine, PIPES, POPSO, propionate, pyridine, pyrophosphate, succinate (pK1), succinate (pK2), TABS, TAPS, TAPSO, taurine (AES), TES, tricine, triethanolamine (TEA), and Trizma (tris). Excipients may include a lactose, mannitol, sorbitol, microcrystalline cellulose, sucrose, sodium citrate, dicalcium phosphate, phosphate buffer, or any other ingredient of the similar nature alone or in a suitable combination thereof.

The microbial composition may include a binder may, for example, a gum tragacanth, gum acacia, methyl cellulose, gelatin, polyvinyl pyrrolidone, starch, biofilm, or any other ingredient of the similar nature alone or in a suitable combination thereof.

Use of biofilms as a glue or protective matrix in live biotherapeutic compositions in a method of identifying a biologically-active composition from a biofilm is described in U.S. Pat. Nos. 10,086,025; 10,004,771; 9,919,012; 9,717,765; 9,713,631; 9,504,739, each of which is incorporated by reference. Use of biofilms as materials and methods for improving immune responses and skin and/or mucosal barrier functions is described in U.S. Pat. Nos. 10,004,772; and 9,706,778, each of which is incorporated by reference. For example, the compositions may comprise a strain of Lactobacillus fermentum bacterium, or a bioactive extract thereof. In preferred embodiments, extracts of the bacteria are obtained when the bacteria are grown as biofilm. The subject disclosure also provides compositions comprising L. fermentum bacterium, or bioactive extracts thereof, in a lyophilized, freeze dried, and/or lysate form. In some embodiments, the bacterial strain is Lactobacillus fermentum Qi6, also referred to herein as Lf Qi6. In one embodiment, the subject disclosure provides an isolated or a biologically pure culture of Lf Qi6. In another embodiment, the subject disclosure provides a biologically pure culture of Lf Qi6, grown as a biofilm. The pharmaceutical compositions may comprise bioactive extracts of Lf Qi6 biofilm. For example, L. fermentum Qi6 may be grown in MRS media using standard culture methods. Bacteria may be subcultured into 500 ml MRS medium for an additional period, again using proprietary culture methods. Bacteria may be sonicated (Reliance Sonic 550, STERIS Corporation, Mentor, Ohio, USA), centrifuged at 10,000 g, cell pellets dispersed in sterile water, harvested cells lysed (Sonic Ruptor 400, OMNI International, Kennesaw, Ga., USA) and centrifuged again at 10,000 g, and soluble fraction centrifuged (50 kDa Amicon Ultra membrane filter, EMD Millipore Corporation, Darmstadt, Germany, Cat #UFC905008). The resulting fraction may be distributed into 0.5 ml aliquots, flash frozen in liquid nitrogen and stored at −80° C.

The compositions provided herein may optionally contain a single (unit) dose of probiotic bacteria, or lysate, or extract thereof. Suitable doses of probiotic bacteria (intact, lysed or extracted) may be in the range 10\hat{ }4 to 10\hat{ }12 cfu, e.g., one of 10\hat{ }4 to 10\hat{ }10, 10\hat{ }4 to 10\hat{ }8, 10\hat{ }6 to 10\hat{ }12, 10\hat{ }6 to 10\hat{ }10, or 10\hat{ }6 to 10\hat{ }8 cfu. In some embodiments, doses may be administered once or twice daily. In some embodiments, the compositions may comprise one or more each of a binder and or excipient, in at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to 10 about 5%, by weight

The abbreviation cfu refers to a “colony forming unit” that is defined as the number of bacterial cells as revealed by microbiological counts on agar plates.

The composition may include excipients selected from the group consisting of agar-agar, calcium carbonate, sodium carbonate, silicates, alginic acid, corn starch, potato tapioca starch, primogel or any other ingredient of the similar nature alone or in a suitable combination thereof, lubricants selected from the group consisting of a magnesium stearate, calcium stearate, talc, solid polyethylene glycols, sodium lauryl sulfate or any other ingredient of the similar nature alone; glidants selected from the group consisting of colloidal silicon dioxide or any other ingredient of the similar nature alone or in a suitable combination thereof; a stabilizer selected from the group consisting of such as mannitol, sucrose, trehalose, glycine, arginine, dextran, or combinations thereof, an odorant agent or flavoring selected from the group consisting of peppermint, methyl salicylate, orange flavor, vanilla flavor, or any other pharmaceutically acceptable odorant or flavor alone or in a suitable combination thereof; wetting agents selected from the group consisting of acetyl alcohol, glyceryl monostearate or any other pharmaceutically acceptable wetting agent alone or in a suitable combination thereof; absorbents selected from the group consisting of kaolin, bentonite clay or any other pharmaceutically acceptable absorbents alone or in a suitable combination thereof; retarding agents selected from the group consisting of wax, paraffin, or any other pharmaceutically acceptable retarding agent alone or in a suitable combination thereof.

The microbial composition may comprise one or more emollients. Non-limiting examples of emollients include stearyl alcohol, glyceryl monoricinoleate, glyceryl mono stearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, sesame oil, coconut oil, arrachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate.

The microbial composition may include a thickener, for example, where the thickener may be selected from hydroxyethylcelluloses (e.g. Natrosol), starch, gums such as gum arabic, kaolin or other clays, hydrated aluminum silicate, fumed silica, carboxyvinyl polymer, sodium carboxymethyl cellulose or other cellulose derivatives, ethylene glycol monostearate and sodium alginates. The microbial composition may include preservatives, antiseptics, pigments or colorants, fragrances, masking agents, and carriers, such as water and lower alkyl, alcohols, such as those disclosed in an incorporated by reference from U.S. Pat. No. 5,525,336 are included in compositions.

The live biotherapeutic composition may optionally comprise a preservative. Preservatives may be selected from any suitable preservative that does not destroy the activity of the synthetic microorganism. The preservative may be, for example, chitosan oligosaccharide, sodium benzoate, calcium propionate, tocopherols, selected probiotic strains, phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; chelating agents such as EDTA; salt-forming counter-ions such as sodium; metal complexes (e.g. Zn-protein complexes), such as m-cresol or benzyl alcohol. The preservative may be a tocopherol on the list of FDA's GRAS food preservatives. The tocopherol preservative may be, for example, tocopherol, dioleyl tocopheryl methylsilanol, potassium ascorbyl tocopheryl phosphate, tocophersolan, tocopheryl acetate, tocopheryl linoleate, tocopheryl linoleate/oleate, tocopheryl nicotinate, tocopheryl succinate. The composition may include, for example, 0-2%, 0.05-1.5%, 0.5 to 1%, or about 0.9% v/v or wt/v of a preservative.

The compositions of the disclosure may include a stabilizer and/or antioxidant. The stabilizer may be, for example, an amino acid, for example, arginine, glycine, histidine, or a derivative thereof, imidazole, imidazole-4-acetic acid, for example, as described in U.S. Pat. No. 5,849,704. The stabilizer may be a “sugar alcohol” may be added, for example, mannitol, xylitol, erythritol, threitol, sorbitol, or glycerol. In the present context “disaccharide” is used to designate naturally occurring disaccharides such as sucrose, trehalose, maltose, lactose, sepharose, turanose, laminaribiose, isomaltose, gentiobiose, or melibiose. The antioxidant may be, for example, ascorbic acid, glutathione, methionine, and ethylenediamine tetraacetic acid (EDTA). The optional stabilizer or antioxidant may be in an amount from about 0 to about 20 mg, 0.1 to 10 mg, or 1 to 5 mg per mL of the liquid composition.

The microbial compositions for topical administration may be provided in liquid, solution, suspension, cream, lotion, ointment, gel, or in a solid form such as a powder, tablet, or troche for suspension immediately prior to administration. The compositions for topical use may also be provided as hard capsules, or soft gelatin capsules, wherein the benign and/or synthetic microorganism is mixed with water or an oil medium, for example, peanut oil, liquid paraffin, or olive oil. Powders and granulates may be prepared using the ingredients mentioned above under tablets and capsules for dissolution in a conventional manner using, e.g., a mixer, a fluid bed apparatus, lyophilization or a spray drying equipment. A dried microbial composition may administered directly or may be for suspension in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate in a carrier. When the composition is in a powder form, the powders may include chalk, talc, fullers earth, colloidal silicon dioxide, sodium polyacrylate, tetra alkyl and/or trialkyl aryl ammonium smectites and chemically modified magnesium aluminum silicate

The microbial composition may exhibit a stable CFU losing less than 30%, 20%, 10% or 5% cfu over at least one, two, three months, six months, 12 months 18 months, or 24 months when stored at frozen, refrigerated or preferrably at room temperature.

Kits

Any of the above-mentioned compositions or synthetic microorganisms may be provided in the form of a kit. In some embodiments, a kit comprises a container housing live bacteria or a container housing freeze-dried live bacteria. Kits can include a second container including media. Kits may also include one or more decolonizing agents. Kits can also include instructions for administering the composition. In certain embodiments, instructions are provided for mixing the bacterial strains with other components of the composition. In some embodiments, a kit further includes an applicator to apply the microbial composition to a subject.

Dose

In certain embodiments, a composition is provided for topical administration that is a solution composition, or for reconstitution to a solution composition. In one embodiment, composition may include from about 1×10⁵ to 1×10¹² cfu/ml, 1×10⁶ to 1×10¹⁰ cfu/ml, or 1.2×10⁷ to 1.2×10⁹ CFU/mL of the synthetic microorganism in an aqueous solution, such as phosphate buffered saline (PBS). Lower doses may be employed for preliminary irritation studies in a subject.

Preferably, the subject does not exhibit recurrence of the undesirable microorganism as evidenced by swabbing the subject at the at least one site after at least 2, 3, 4, 6, 10, 15, 22, 26, 30 or 52 weeks after performing the initial administering step.

### EXAMPLES

### Example 1. Selection and Identification of Parent Microorganism-Identification of S. aureus Strains by Spa Typing

The rapid and accurate identification of bacterial species is important for disease diagnosis, epidemiology, and understanding the microbiomes across either human or animal populations both locally and on a global scale. Most of the microbial strain typing methods involve lengthy protocols that require growing up the organism to sufficient quantities needed for either genomic analysis such as sequencing or looking at banding patterns, or for analyzing the organism's phenotypic responses to various selective or differential media or reagents. These methods are either very slow, require expensive equipment or kits, or do not lend results that can be compared from one lab to the next.

Spa typing is recommended as a good technique for identification of S. aureus strains on an international level. O'Hara et al., 2016. Spa typing and multilocus sequence typing show comparable performance in a macro epidemiologic study of Staphylococcus aureus in the United States. Microbial Drug Resistance. Vol. 22. No. 1. p. 88-96. It is a technique that analyzes the DNA sequence of the polymorphic region of a protein unique to S. aureus called Staph protein A (spa). Spa typing analyzes the micro and macro variations in 24 base pair repeats that are flanked by well conserved DNA regions and then compares the sequence to a database of known strains. Ridom SpaServer (www.spaserver.ridom.de).

The sequences are retained in an international database which identifies strains based on a number code generated by the number and order of the repeat sequences, and currently contains over 19,000 different spa types and 794 different repeat sequences.⁴⁻⁵ Spa typing is an ideal and cost-effective method to screen for the presence of Staph aureus in various environments, such as bacterial infections where S. aureus is suspected or in the nares of humans. Since the staph protein A is unique to S. aureus, a positive PCR for the presence of spa indicates that S. aureus is present, and the spa type can give details about the strain's prevalence in the region or world, along with the epidemiology of disease involving that strain.

Materials

Equipment and Instrumentation

A thermal cycler for PCR reaction (BioRad #1861096) was employed. Various pipettes, PCR reaction tubes, a microfuge, and Qiaquick PCR Purification Column kit to purify PCR products (Qiagen, 28104) were utilized. Reagents included SA lysis buffer SA lysis buffer for crude gDNA preps of Staph aureus, Protein kinase K used in conjunction with SA lysis buffer to degrade cell wall of Staph aureus (Omega Biotek, AC115), Econotaq® PCR master mix (2×) (Lucigen, 30032), Q5 ® Hot Start High-Fidelity PCR Master Mix (2×), High Fidelity PCR Master Mix (NEB, M0494L), and Molecular Biology grade water, molecular biology grade water DNase-, RNase-, and Protease-free (Light Labs, 80001-04). Table 1B shows the primers used for Econotaq PCR and Q5 PCR reactions.

TABLE 1B
Oligos and Their Sequences

| Name | Sequence (5′→3′) |
|---|---|
| DR_606 | GAACAACGTAACGGCTTCATCC (SEQ ID NO: 106) |
| DR_607 | GTTGCTCGTGCATTTAGATGATTCTTATC (SEQ ID NO: |
| | 107) |

Methods

A single colony of bacteria is isolated by streaking or patching to a fresh agar plate (TSB/MSA/blood agar plate) and incubating the plate at 37° C. for 16-24 hours.

Prepare and lyse the colonies to be screened. 50 μL of cell lysis solution for each colony to be screened (5 μL ProK per 1 mL of SA lysis buffer) was added, and incubated at 55° C. for 1 hour, 95° C. for 10 minutes, then cooled to room temp. Briefly spin the tubes to pellet the cell debris and use 2 μL of the supernatant as the template for the following PCR to amplify a portion of the spa gene. Prepare a High Fidelity Q5 PCR using the primers DR_606 and DR_607. DR_606 binds to the genome in the spa gene upstream of the variable region, and DR_607 binds just downstream of the spa gene.

Run the PCR products on a 1% agarose gel and check the PCR for the right sized band and cleanliness. The band should be about 750 base pairs, but may vary slightly in size between different strains. If the reaction produced one clean band at the correct size, clean up the DNA with a Qiaquick PCR Cleanup kit (Qiagen) per the manufacturer's instructions. The DNA is sent for Sanger sequencing using the primers DR_606 and DR_607.

The DNA sequencing results were used to analyze the variable region of the spa gene using the BioNumerics software per the developer's instructions (Applied Maths). The software assigns the spa type if it finds a match in the database.

Results

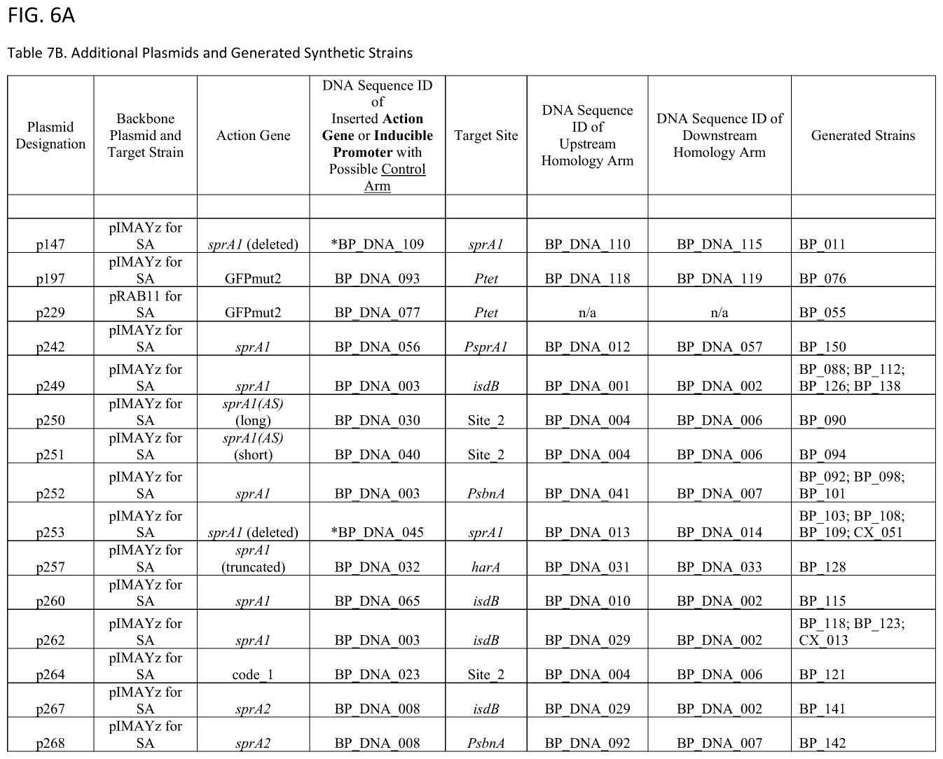

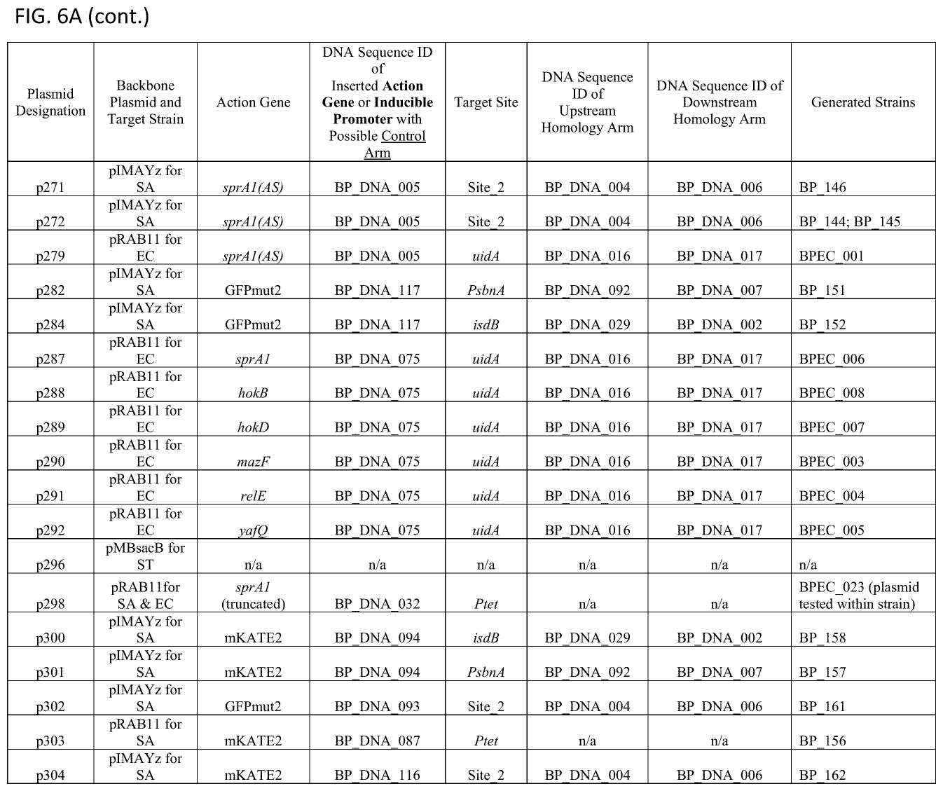

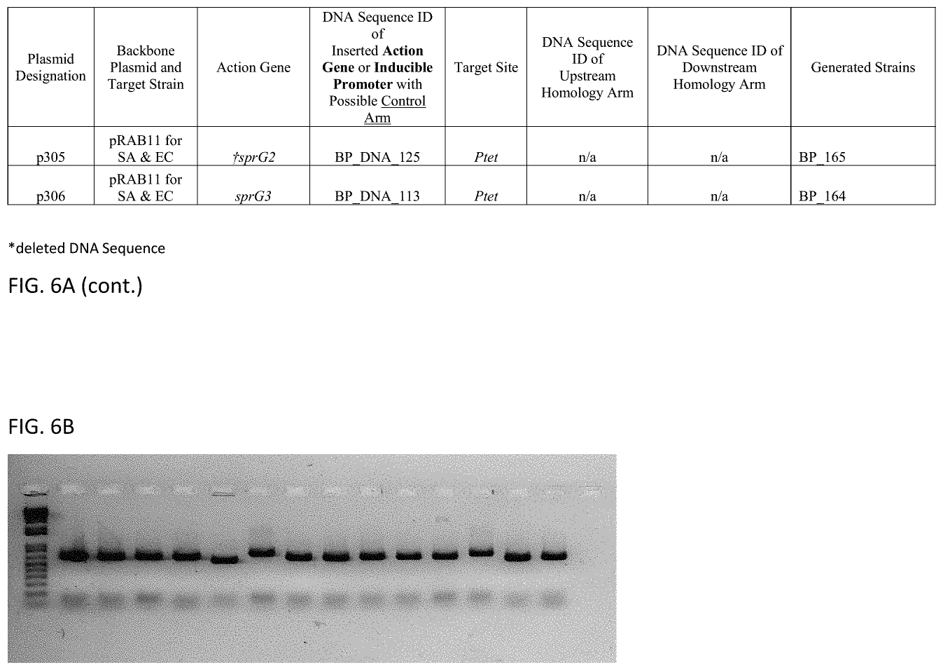

FIG. 6B shows a photographic image of a 1% agarose gel that was run to analyze the PCR from 14 colonies screened for the spa genes using Q5 PCR master mix. All lanes showed a positive band indicating the presence of the spa gene. The differences in the number of repeats in the variable region of the spa gene are the likely cause of the slight differences in the size of the PCR products.

Results of spa typing different S. aureus strains are shown in Table 2.

TABLE 2
Results From spa Typing Strains

| | spa Type | | spa Type |
|---|---|---|---|
| Strain Name in | Assigned by | Strain Name in | Assigned by |
| In house Database | BioNumerics | In house Database | BioNumerics |
| BP_001 | t010 | A1-1D-915 | t3841 |
| CX_001 | t688 | A1-0068 | t005 |
| A1-033N | t008 | A1-0609N | t127 |
| A1-0791N | t005 | A1-0940A | t005 |
| A1-0915N | t021 | A1-9080A | t002 |
| A1-1415N | t127 | A1-1691N | t005 |
| A1-415 | t002 | A1-1235N | t3841 |
| A1-1618 | t3841 | A1-0909N | t1328 |
| A1-1D-180 | t272 | A1-0905A | t008 |

The table above shows the results from spa typing 18 strains collected by BioPlx. There are 10 different spa types identified in these samples.

Spa typing is a quick and accurate test that can be used to type different strains of S. aureus. The Staph Protein A (spa) is unique to S. aureus and contains a hypervariable region at the 3′ end of the coding region of the gene. The test is easy to perform, yields accurate and reproducible results, and with over 19,000 different spa types currently in the database it is able to distinguish a wide variety of different strains. The typing data can be used to track changes in an individual's or population's microbiome, or help to diagnose the potential severity of an infection.

The present inventors performed the Spa typing test on a variety of S. aureus strains that were acquired through sampling human and animal microbiomes. BioNumerics software was utilized to perform the analysis of the repeats and type the strain. It was found that the hypervariable region in the spa protein was easily amplified by PCR from crude gDNA preps, and was easy to locate in the sequencing data. At least 18 different strains were added to the database using this system, having at least 10 separate spa types. Certain of the S. aureus stains were employed as parental target strains in preparation of synthetic microorganisms. Target strains having t010 or t688 were selected for molecular modification.

### Example 2. Multiple sprA1 Kill Switch Designs in Staph aureus

Multiple versions of kill switches using sprA1 toxin gene integrated behind the endogenous serum-inducible isdB gene in genome of Staph aureus strain BP_001 were prepared and evaluated for efficacy.

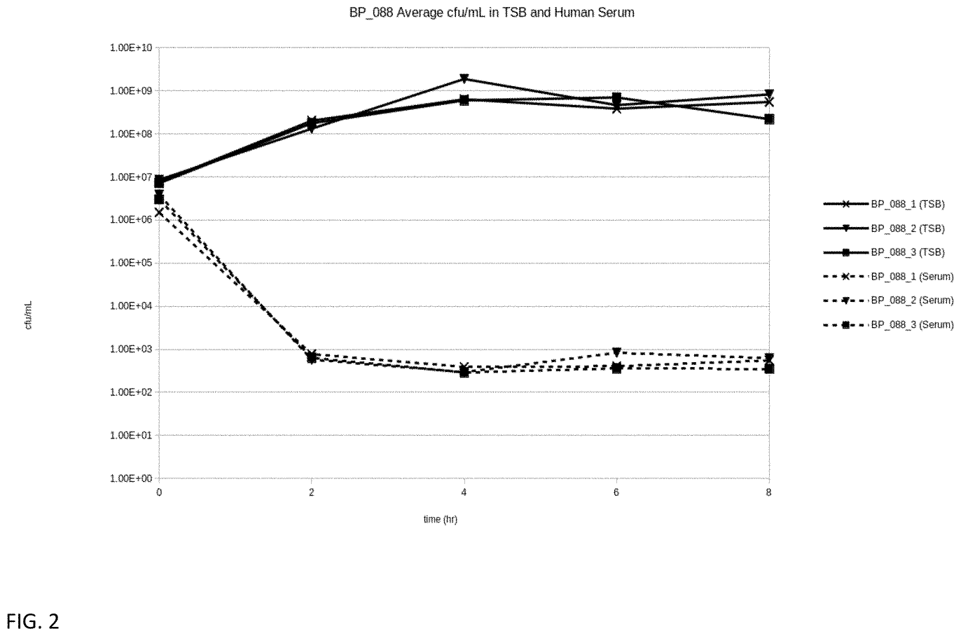

FIG. 1C shows truncated sequence alignment of the isdB::sprA1 sequences inserted to target strain BP_001 (502a) strain. The first synthetic strain BP_088 comprising isdB::sprA1 had a mutation incorporated into the upstream homology arm, which made a frame shift in the isdB gene extending the reading frame by 30 base pairs or 10 amino acids, as shown in FIG. 1C(B). Despite the frame shift, BP_088 comprising isdB::sprA1 exhibited excellent suicidal cell death response (dotted lines) within 2 hours after exposure to human serum as shown in FIG. 2. BP_088 also exhibited good ability to grow in complete media (TSB, solid lines).

Additional insertion vectors were designed to investigate if the phenotypic response that was observed in serum was a result of the frame shifted isdB gene or the integrated toxin gene.

Since at first it was difficult to determine if the mutation was incorporated into the strain BP_088 due to its presence in the original insertion vector, or if the strain mutated the sequence during the recombination event in order to avoid cell death, two new vectors were prepared to test both of these options.

One of the new vectors had the same sequence as the first strain, but without the frame shift in the isdB gene and was used to prepare mutation free synthetic strain BP_118. The other new vector, used to prepare synthetic strain BP_115, added two more stop codons at the end of the isdB gene (triple stop), both in separate frames in case the strain would attempt to mutate the insert during the integration. Both of the new insertion vectors were used to make the edits in the genome of Staph aureus. The ability of synthetic strains BP_088, BP_115, and BP_118 to grow in human serum was evaluated compared to wild type Staph aureus parent strain BP_001 (502a), as shown in FIGS. 2-5.

Materials and Methods

Table 3 shows the different media and other solutions used in the experiment.

TABLE 3
Media and Other Solutions

| | | Manu- | Part |
|---|---|---|---|
| Name | Description | facturer | Number |
| TSB | Tryptic Soy Broth | Teknova | T1395 |
| | (minus glucose) | | |
| TSB agar | Tryptic Soy Agar plates | Teknova | T0144 |
| | (minus glucose) | | |
| Human Serum | Pooled human serum | Sera care | 1830-0005 |
| PBS | 1X Phosphate buffered saline | Teknova | P0200 |

Table 4 shows the oligo names and sequences used to construct the plasmids that were used to insert the kill switches into the genome of BP_001.

TABLE 4
Oligos and Their Sequences

| Name | Sequence (5′→3′) |
|---|---|
| BP_948 | CCCTCGAGGTCGACGGTATCGATAAGCTTGGATGAGCAAGTGA |
| | AATCAGCTATTAC (SEQ ID NO: 108) |
| BP_949 | CACCTCCTCTCTGCGGATTTATTAGTTTTTACGTTTTCTAGGT |
| | AATAC (SEQ ID NO: 109) |
| BP_950 | AAAAACTAATAAATCCGCAGAGAGGAGGTGTATAAGGTGATG |
| | (SEQ ID NO: 110) |
| BP_951 | ATTAAATATAAAGACCTATTTTGTATTGCGTCTACTTAGCCAA |
| | TAAGAAAAAAAC (SEQ ID NO: 111) |
| BP_952 | CGCAATACAAAATAGGTCTTTATATTTAATTATTAAATTAACA |
| | AATTTTAATTG (SEQ ID NO: 112) |
| BP_953 | GTGGCGGCCGCTCTAGAACTAGTGGATCCCGTCAATTACGCAA |
| | TTAAGGAAATATC (SEQ ID NO: 113) |
| DR_511 | CACCTCCTCTCTGCGCTATTCAATTAGTTTTTACGTTTTCTAG |
| | GTAATACGAATGC (SEQ ID NO: 114) |
| DR_512 | CTAATTGAATAGCGCAGAGAGGAGGTGTATAAGGTGATGC |
| | (SEQ ID NO: 115) |

Table 5 shows the plasmid genotypes used to insert the various versions of sprA1 behind the isdB gene in the genome of wild type BP_001 (502a).

TABLE 5
Plasmids Names and Function

| Plasmid Name | DNA to be Inserted Behind isdB Gene |
|---|---|
| p249 | isdB::sprA1(frame shift) |
| p260 | isdB::sprA1(triple stop) |
| p262 | isdB::sprA1 |

Table 6 shows the strains used and created in this study. The bold portion of the sequence represents the sprA1 toxin gene and the underlined sequence represents the 5′ untranslated region of the insert.

TABLE 6
Staphylococcus aureus strains

| | DNA | | |
|---|---|---|---|
| Strain | Seq. ID | Genotype | Sequence of Insert |
| BP_001 | n/a | 502a wild type | N/A |
| BP_088 | BP_DNA_063 | BP_001, | ATAATAAATCCGCAGAGAGGAGGTGTAT |
| | | isdB::sprA1 | AAGGTGATGCTTATTTTCGTTCACATCA |
| | | (frame shift) | TAGCACCAGTCATCAGTGGCTGTGCCA |
| | | | TTGCGTTTTTTTCTTATTGGCTAAGTAG |
| | | | ACGCAATACAAAATAG (SEQ ID NO: 116) |
| BP_115 | BP_DNA_065 | BP_001, | TTGAATAGCGCAGAGAGGAGGTGTATAA |
| | | isdB::sprA1 | GGTGATGCTTATTTTCGTTCACATCATA |
| | | (triple stop) | GCACCAGTCATCAGTGGCTGTGCCATT |
| | | | GCGTTTTTTTCTTATTGGCTAAGTAGAC |
| | | | GCAATACAAAATAG (SEQ ID NO: 34) |
| BP_118 | BP_DNA_003 | BP_001, | CGCAGAGAGGAGGTGTATAAGGTGATGC |
| | | isdB::sprA1 | TTATTTTCGTTCACATCATAGCACCAGT |
| | | | CATCAGTGGCTGTGCCATTGCGTTTTT |
| | | | TTCTTATTGGCTAAGTAGACGCAATAC |
| | | | AAAATAG (SEQ ID NO: 3) |

All of the synthetic strains were constructed in the same manner, which is using a temperature sensitive plasmid (pIMAYz) to facilitate homologous recombination into the host's genome, and subsequent excision leaving behind the desired inserted sequence.

Plasmid Construction
i. p249 (used to make BP_088) Primers for PCR amplification of homology arms and insert.
   1. Upstream homology arm
      a. BP_948/BP_949
   2. Downstream homology arm
      a. BP_952/BP_953
   3. sprA1 insert
      a. BP_950/BP_951
ii. p262 (used to make BP_118) Primers for PCR amplification of homology arms and insert.
   1. Upstream homology arm
      a. BP_948/BP_949
   2. Downstream homology arm
      a. BP_952/BP_953
   3. sprA1 insert
      a. BP_950/BP_951
iii. p260 (used to make BP_115) Primers for PCR amplification of homology arms and insert.
   1. Upstream homology arm
      a. BP_948/DR_511
   2. Downstream homology arm
      a. BP_952/BP_953
   3. sprA1 insert
      a. DR_512/BP_951
iv. For each plasmid, the PCR amplified fragments were combined with a pIMAYz backbone vector and assembled into a circular plasmid using the Gibson Assembly Kit. per the manufacturer's instructions and transformed into electrocompetent E. coli.
v. Colonies were screened and several positive clones were sequenced to confirm proper plasmid sequence.

Strain Construction in Staph aureus
i. Sequence confirmed plasmids were transformed into electrocompetent Staph aureus and plated at 37° C. to force the integration of the plasmid.
ii. Colonies were then screened for the inserted plasmid into the genome.
   1. 3 positive clones were incubated overnight at room temp in 5 mL BHI media and plated on BI (AtC+X-gal).
iii. White colonies were picked and screened for the presence of the plasmid both in the genome or self replicating in the cell.
iv. Colonies showing no sign of residual plasmid were screened for the inserted DNA fragment.
v. Several positive clones were sequenced to confirm the correct sequence was inserted into the genome.
vi. One sequence confirmed clone was stocked in the database and used for a serum assay.

Human Serum Assay
i. Start 3 overnight cultures from 3 separate single colonies of experimental strain in 5 mL TSB. Start one culture of 502a for internal assay control purposes and treat it in the same manner as the experimental samples.
ii. The following morning, cut back the overnight cultures to 0.05 OD600 in 5.5 mL of fresh TSB.
   1. Measure the OD600 by diluting the culture 1:10 in TSB (100 uL culture in 900 uL TSB).
   2. Calculate the necessary volume of overnight culture to inoculate fresh culture tube: (0.05*5.5)/OD600.
   3. Inoculate 5.5 mL of TSB and incubate the culture with agitation (37° C., 240 rpm) for 2 hrs to sync of the metabolism of the cells.
iii. 2 hrs after the fresh cultures in step 2 were inoculated, measure the OD600.
iv. Wash the cultures in sterile PBS.
   1. Centrifuge cultures using swing out rotor (3500 rpm, 5 mins, RT), wash with 5 mL PBS.
   2. Centrifuge again and re-suspend in 1 mL sterile PBS.
v. Calculate amount of re-suspended culture needed to inoculate 5 ml of TSB/Serum at 0.05 OD600.
vi. Inoculate (3 tubes each) of 5 mL of fresh, pre-warmed TSB and human serum at 0.05 OD600.
vii. After addition of inoculum, quickly mix by pulse vortexing and take 100 μL sample for determining cfu/mL. Place remaining cultures in 37° C. shaking incubator.
   1. Sample every two hours for the next 8 hours, and perform serial dilutions to determine cfu/mL.
      a. Serial dilutions are performed by starting with 900 μL of sterile PBS in sterile 1.5 mL tubes. A 100 μL sample is removed from a well-mixed culture and transferred into the first PBS tube.
      b. It is mixed well by pulse vortexing and 100 μL is removed and transferred to the next tube, and so on until the culture has been diluted to a point where 30-300 colonies will grow when 100 μL is spread out on a TSB agar plate. The process is repeated for all culture tubes at every time point.
      c. All plates are incubated 12-16 hours at 37° C., and the colony counts are recorded and used to calculate the cfu/mL of the cultures.

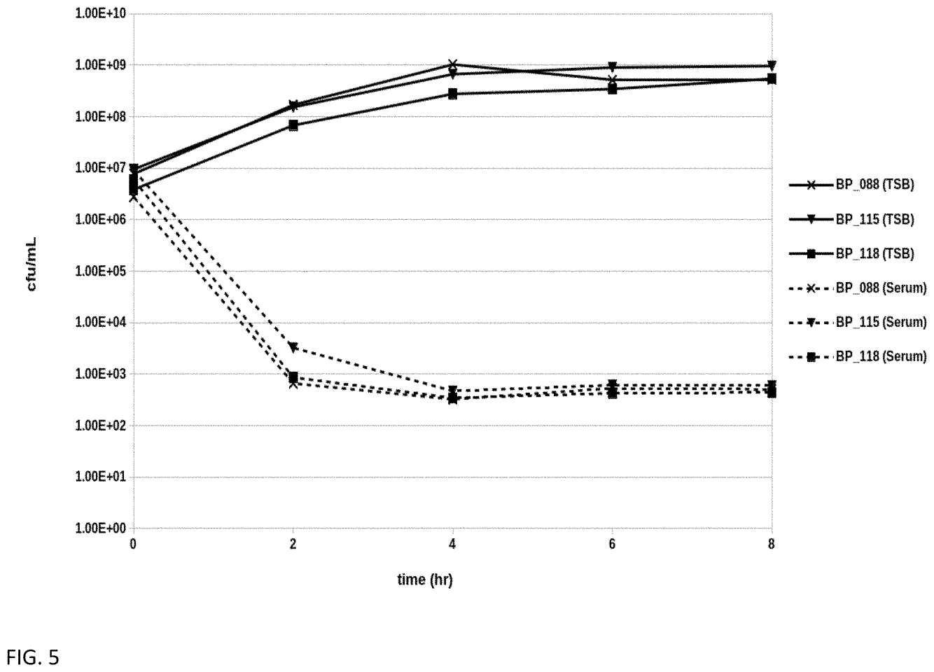

Results are shown in FIGS. 2 to 5 showing graphs of the colony forming units per mL of culture over 8 hours. The dashed lines represent the cultures grown in serum and solid lines represent the cultures grown in TSB. FIG. 5 shows the average (n=3) colony forming units per mL of culture over 8 hours for each of BP_088, BP_115, and BP_118 in TSB or human serum.

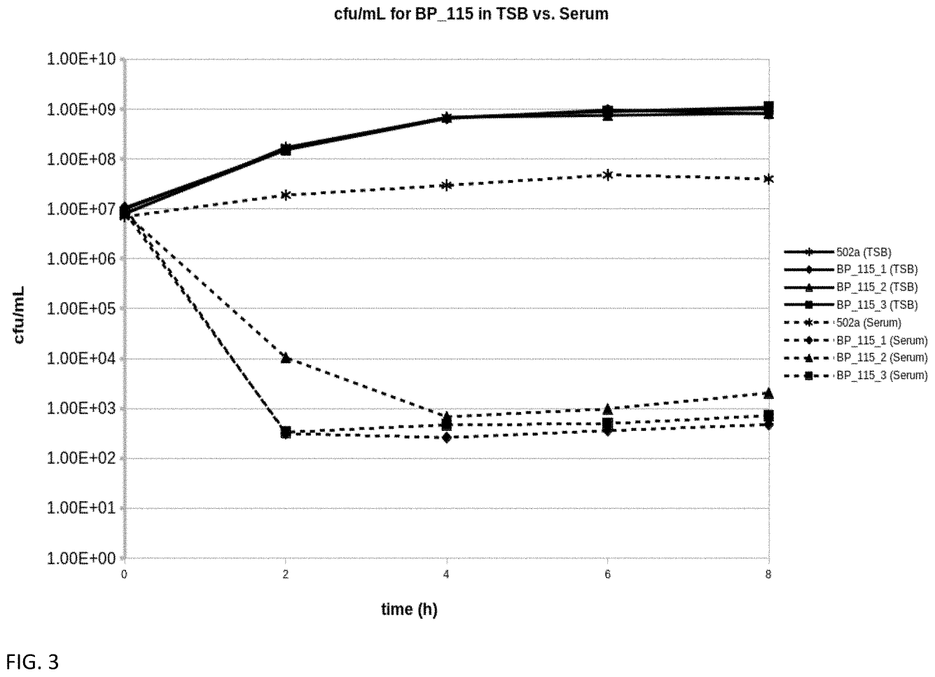

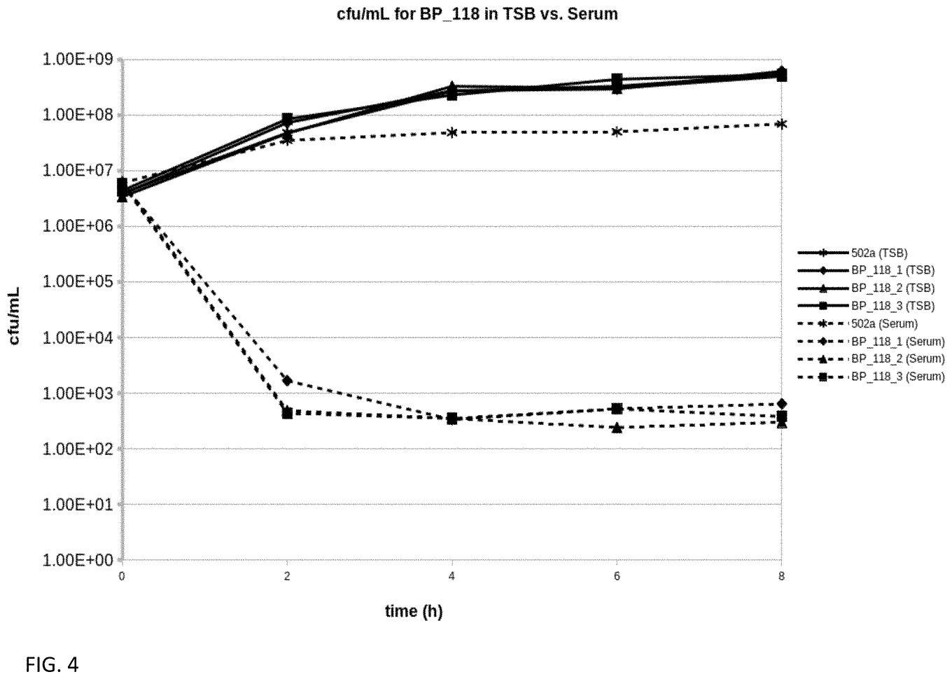

The engineered strains BP_088, BP_115, and BP_118 each comprising isdB::sprA1, and WT parent strain BP_001 each exhibited good cell growth in complete media (TSB, solid lines) as shown in FIGS. 2-5. WT BP_001 also exhibited ability to grow when exposed to human serum, as shown in FIGS. 3 and 4 (dotted lines). However, upon exposure to human serum, all three engineered strains BP_088, BP_115, and BP_118 exhibited significantly decreased growth (dotted lines) within 2 hours after exposure to human serum as shown in FIGS. 2-5.

Conclusion

This series of experiments evaluated the phenotypic response of several engineered strains of Staph aureus while grown in human serum versus TSB. The strains have slightly different kill switch sequences integrated into the same location of the genome. All sequences were inserted directly behind the isdB gene.

One of the integrations resulted in the desired kill switch sequence (BP_118), another integration produced a mutation that resulted in a frame shift in the isdB gene, which is directly before the kill switch and adds 30 more bases to the isdB gene (BP_088), and the third integration introduced multiple STOP codons in different frames directly behind the isdB gene to protect the gene from being disrupted by frameshift mutations.

The three engineered strains were tested for their ability to grow in human serum and TSB versus the wild type (BP_001) strain. For all experimental strains tested (BP_088, BP_115, and BP_118), the phenotypic response showed a significant drop in the cfu/mL when grown in human serum versus TSB. This response was not observed for any WT BP_001 strains in human serum, instead that strain demonstrated the ability to grow in human serum and had multiple doublings in the same time period as the other strains experienced a reduction in population of several orders of magnitude.

A number of additional kill switch cell lines were developed in a similar fashion as shown in Table 7A.

TABLE 7A
Kill Switch Cell Lines and Plasmids

| | | | E. coli | S. aureus |
|---|---|---|---|---|
| Plasmid | Insertion | Description | AbR* | AbR* |
| pTK001 | pCN51-Pcad-sprA1- | sprA1 kill gene and antitoxin under | Amp | Erm |
| | sprA1at | cadmium promoter | | |
| pTK002 | pCN51-Pcad-sprA1- | Reversed SprA1 kill gene and antitoxin | Amp | Erm |
| | sprA1at(rev) | under cadmium promoter | | |
| pTK003 | pCN51-PleuA-sprA1- | SprA1 kill gene and antitoxin under leuA | Amp | Erm |
| | sprA1at | promoter | | |
| pTK004 | pCN51-PleuA-sprA1- | Reversed SprA1 kill gene and antitoxin | Amp | Erm |
| | sprA1at(rev) | under leuA promoter | | |
| pTK005 | pCN51-PleuA- | SprA1 kill gene under leuA promoter, | Amp | Erm |
| | sprA1_PCLFB-sprA1at | with sprA1 antitoxin under CLFB clamp | | |
| | | promoter (opposite orientation of sprA1) | | |
| pTK006 | pCN51-PhlgA-sprA1- | SprA1 kill gene and antitoxin under hlgA | Amp | Erm |
| | sprA1at | promoter | | |
| pTK007 | pCN51-PhlgA- | SprA1 kill gene under hlgA promoter, | Amp | Erm |
| | sprA1_PCLFB-sprA1at | with sprA1 antitoxin under CLFB clamp | | |
| | | promoter (opposite orientation of sprA1) | | |
| pTK008 | pCN51-Pcad-Sma1 | Sma1 restriction enzyme kill gene under | Amp | Erm |
| | | cadmium promoter | | |
| pTK009 | pCN51-PhlgA-Sma1 | Sma1 restriction enzyme kill gene under | Amp | Erm |
| | | hlgA promoter | | |
| pTK010 | pCN51-PleuA-Sma1 | Sma1 restriction enzyme kill gene under | Amp | Erm |
| | | leuA promoter | | |
| pTK011 | pCN51-Pcad-RsaE | RsaE small RNA kill gene under | Amp | Erm |
| | | cadmium promoter | | |
| pTK012 | pCN51-PhlgA-RsaE | RsaE small RNA kill gene under hlgA | Amp | Erm |
| | | promoter | | |
| pTK013 | pCN51-PleuA-RsaE | RsaE small RNA kill gene under leuA | Amp | Erm |
| | | promoter | | |
| p080 | pCN51-Pcad-relF | relF kill gene driven by cadmium- | Amp | Erm |
| | | inducible promoter | | |
| p086 | pCN56-TT-PhlgA2- | SprA1 kill gene and antitoxin driven by | Amp | Erm |
| | sprA1-sprA1at | hlgA2 promoter | | |
| p087 | pCN56-TT-PisdG- | SprA1 kill gene and antitoxin driven by | Amp | Erm |
| | sprA1-sprA1at | isdG promoter | | |
| p088 | pCN56-TT-PsbnC- | SprA1 kill gene and antitoxin driven by | Amp | Erm |
| | sprA1-sprA1at | sbnC promoter | | |
| p089 | pCN56-TT-PsbnE- | SprA1 kill gene and antitoxin driven by | Amp | Erm |
| | sprA1-sprA1at | sbnE promoter | | |
| p090 | pCN56-TT-PhlgB- | SprA1 kill gene and antitoxin driven by | Amp | Erm |
| | sprA1-sprA1at | hlgB promoter | | |
| p091 | pCN56-TT- | SprA1 kill gene and antitoxin driven by | Amp | Erm |
| | PSAUSA300_2616- | SAUSA300_2616 promoter | | |
| | sprA1-sprA1at | | | |
| p092 | pCN56-TT-PlrgA- | SprA1 kill gene and antitoxin driven by | Amp | Erm |
| | sprA1-sprA1at | lrgA promoter | | |
| p096 | pCN56-TT-PhlgA2- | HlgA2 promoter driving sprA1 kill gene | Amp | Enn |
| | sprA1-sprA1at | and antitoxin. Promoter insert synthesized | | |
| | | and cloned into p078_pCN56-TT-sprA1- | | |
| | | sprA1at by GenScript. | | |
| p097 | pCN56-TT-Pcad- | Cadmium-inducible promoter driving | Amp | Erm |
| | sprA1-sprA1at | sprA1 kill gene and antitoxin. Promoter | | |
| | | insert synthesized and cloned into | | |
| | | p078_pCN56-TT-sprA1-sprA1at by | | |
| | | GenScript. | | |
| p098 | pCN56-TT-PhlgB- | HlgB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p099 | pCN56-TT-PsplF- | SplF promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p100 | pCN56-TT-PfhuB- | FhuB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p101 | pCN56-TT-Phlb- | Hlb promoter driving sprA1 kill gene and | Amp | Erm |
| | sprA1-sprA1at | antitoxin. Promoter insert synthesized and | | |
| | | cloned into p078_pCN56-TT-sprA1- | | |
| | | sprA1at by GenScript. | | |
| p102 | pCN56-TT-PhrtAB- | HrtAB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p103 | pCN56-TT-PisdG- | IsdG promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p104 | pCN56-TT-PlrgA- | LrgA promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p105 | pCN56-TT- | SAUSA300_2268 promoter driving | Amp | Erm |
| | PSAUSA300_2268- | sprA1 kill gene and antitoxin. Promoter | | |
| | sprA1-sprA1at | insert synthesized and cloned into | | |
| | | p078_pCN56-TT-sprA1-sprA1at by | | |
| | | GenScript. | | |
| p106 | pCN56-TT- | SAUSA200_2617 promoter driving | Amp | Erm |
| | PSAUSA300_2617- | sprA1 kill gene and antitoxin. Promoter | | |
| | sprA1-sprA1at | insert synthesized and cloned into | | |
| | | p078_pCN56-TT-sprA1-sprA1at by | | |
| | | GenScript. | | |
| p107 | pCN56-TT-PsbnE- | SbnE promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p108 | pCN56-TT-PisdI- | IsdI promoter driving sprA1 kill gene and | Amp | Erm |
| | sprA1-sprA1at | antitoxin. Promoter insert synthesized and | | |
| | | cloned into p078_pCN56-TT-sprA1- | | |
| | | sprA1at by GenScript. | | |
| p109 | pCN56-TT-PlrgB- | LrgB promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p110 | pCN56-TT- | SAUSA300_2616 promoter driving | Amp | Erm |
| | PSAUSA300_2616- | sprA1 kill gene and antitoxin. Promoter | | |
| | sprA1-sprA1at | insert synthesized and cloned into | | |
| | | p178_pCN56-TT-sprA1-sprA1at by | | |
| | | GenScript. | | |
| p111 | pCN56-TT-PsbnC- | SbnC promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p133 | pIMAY-502a-2/3/5HA- | HrtAB promoter driving sprA1 kill gene | Chlor | Chlor |
| | PhrtAB-sprA1-sprA1at | and antitoxin. For genomic integration | | |
| | | into 502a via homologous recombination | | |
| | | (2/3/5 arms). | | |
| p134 | pIMAY-502a-7HA- | HrtAB promoter driving sprA1 kill gene | Chlor | Chlor |
| | Phrt AB-sprA1-sprA1at | and antitoxin. For genomic integration | | |
| | | into 502a via homologous recombination | | |
| | | (7 arms). | | |
| p135 | pIMAY-502a-2/3/5HA- | Hlb promoter driving sprA1 kill gene and | Chlor | Chlor |
| | Phlb-sprA1-sprA1at | antitoxin. For genomic integration into | | |
| | | 502a via homologous recombination | | |
| | | (2/3/5 arms). | | |
| p136 | pIMAY-502a-7HA- | Hlb promoter driving sprA1 kill gene and | Chlor | Chlor |
| | Phlb-sprA1-sprA1at | antitoxin. For genomic integration into | | |
| | | 502a via homologous recombination (7 | | |
| | | arms). | | |
| p137 | pIMAY-502a-2/3/5HA- | SbnC promoter driving sprA1 kill gene | Chlor | Chlor |
| | PsbnC-sprA1-sprA1at | and antitoxin. For genomic integration | | |
| | | into 502a via homologous recombination | | |
| | | (2/3/5 arms). | | |
| p138 | pIMAY-502a-7HA- | SbnC promoter driving sprA1 kill gene | Chlor | Chlor |
| | PsbnC-sprA1-sprA1at | and antitoxin. For genomic integration | | |
| | | into 502a via homologous recombination | | |
| | | (7 arms). | | |
| p139 | pIMAY-502a-2/3/5HA- | HlgB promoter driving sprA1 kill gene | Chlor | Chlor |
| | PhlgB-sprA1-sprA1at | and antitoxin. For genomic integration | | |
| | | into 502a via homologous recombination | | |
| | | (2/3/5 arms). | | |
| p140 | pIMAY-502a-7HA- | HlgB promoter driving sprA1 kill gene | Chlor | Chlor |
| | PhlgB -sprA1-sprA1at | and antitoxin. For genomic integration | | |
| | | into 502a via homologous recombination | | |
| | | (7 arms). | | |
| p141 | pIMAY-502a-7HA- | IsdG promoter driving sprA1 kill gene | Chlor | Chlor |
| | PisdG-sprA1-sprA1at | and antitoxin. For genomic integration | | |
| | | into 502a via homologous recombination | | |
| | | (7 arms). | | |
| p142 | pIMAY-502a-2/3/5HA- | SbnE promoter driving sprA1 kill gene | Chlor | Chlor |
| | PsbnE-sprA1-sprA1at | and antitoxin. For genomic integration | | |
| | | into 502a via homologous recombination | | |
| | | (2/3/5 arms). | | |
| p143 | pIMAY-502a-2/3/5HA- | SplF promoter driving sprA1 kill gene | Chlor | Chlor |
| | PsplF-sprA1-sprA1at | and antitoxin. For genomic integration | | |
| | | into 502a via homologous recombination | | |
| | | (2/3/5 arms). | | |
| p144 | pIMAY-502a-2/3/5HA- | IsdI promoter driving sprA1 kill gene and | Chlor | Chlor |
| | PisdI-sprA1-sprA1at | antitoxin. For genomic integration into | | |
| | | 502a via homologous recombination | | |
| | | (2/3/5 arms). | | |
| p145 | pIMAY-502a-2/3/5HA- | SAUSA300_2616 promoter driving | Chlor | Chlor |
| | P2616-sprA1-sprA1at | sprA1 kill gene and antitoxin. For | | |
| | | genomic integration into 502a via | | |
| | | homologous recombination (2/3/5 arms). | | |
| p148 | pIMAY-502a-2/3/5HA- | LrgA promoter driving sprA1 kill gene | Chlor | Chlor |
| | PlrgA-sprA1-sprA1at | and antitoxin. For genomic integration | | |
| | | into 502a via homologous recombination | | |
| | | (2/3/5 arms). | | |
| p154 | pIMAY-502a-2/3/5HA- | HrtAB promoter driving sprGl kill gene. | Chlor | Chlor |
| | PhrtAB-sprG1 (rev) | For genomic integration into 502a at azlC | | |
| | | locus, on sense strand, via homologous | | |
| | | recombination (2/3/5 arms). Constructed | | |
| | | by GenScript. | | |
| p155 | pIMAY-502a-2/3/5HA- | HlgA2 promoter driving 187/lysK phage | Chlor | Chlor |
| | PhlgA2-187lysK (rev) | lytic chimeric protein kill gene. For | | |
| | | genomic integration into 502a at azlC | | |
| | | locus, on sense strand, via homologous | | |
| | | recombination (2/3/5 arms). Constructed | | |
| | | by GenScript. | | |
| p156 | pIMAY-502a-2/3/5HA- | HrtAB promoter driving 187/lysK phage | Chlor | Chlor |
| | PhrtAB-187lysK (rev) | lytic chimeric protein kill gene. For | | |
| | | genomic integration into 502a at azlC | | |
| | | locus, on sense strand, via homologous | | |
| | | recombination (2/3/5 arms). Constructed | | |
| | | by GenScript. | | |
| p157 | pCN56-TT-PhlgA2- | HlgA2 promoter driving sprA1 kill gene. | Amp | Erm |
| | sprA1 | p086 with sprA1 antitoxin deleted. Kill | | |
| | | switch insert flipped orientation during | | |
| | | cloning (promoter is now closer to Staph | | |
| | | ori than E coli ori). | | |
| p158 | pCN56-TT-Phlb-sprA1 | Hlb promoter driving sprA1 kill gene. | Amp | Erm |
| | | p101 with sprA1 antitoxin deleted. | | |
| p159 | pCN56-TT-PsbnC- | SbnC promoter driving sprA1 kill gene, | Amp | Erm |
| | sprA1 | p111 with sprA1 antitoxin deleted. Kill | | |
| | | switch insert flipped orientation during | | |
| | | cloning (promoter is now closer to Staph | | |
| | | ori than E coli ori). | | |
| p160 | pIMAY-502a-9HA- | HlgA2 promoter driving sprA1 kill gene, | Chlor | Chlor |
| | PhlgA2-sprA1 | p122 with sprA1 antitoxin deleted. For | | |
| | | genomic integration into 502a via | | |
| | | homologous recombination (9 arms). | | |
| p161 | pIMAY-502a-7HA- | HrtAB promoter driving sprA1 kill gene. | Chlor | Chlor |
| | PhrtAB-sprA1 | p134 with sprA1 antitoxin deleted. For | | |
| | | genomic integration into 502a via | | |
| | | homologous recombination (7 arms). | | |
| p162 | pIMAY-502a-7HA- | Hlb promoter driving sprA1 kill gene. | Chlor | Chlor |
| | Phlb-sprA1 | p136 with sprA1 antitoxin deleted. For | | |
| | | genomic integration into 502a via | | |
| | | homologous recombination (7 arms). | | |
| p164 | pIMAY-502a-2/3/5HA- | HlgA2 promoter driving sprG1 kill gene. | Chlor | Chlor |
| | PhlgA2-sprG1 (rev) | For genomic integration into 502a at azlC | | |
| | | locus, on sense strand, via homologous | | |
| | | recombination (2/3/5 arms). Constructed | | |
| | | by GenScript. | | |
| p165 | pIMAY-502a-2/3/5HA- | HrtAB promoter driving holin kill gene. | Chlor | Chlor |
| | PhrtAB-holin (rev) | For genomic integration into 502a at azlC | | |
| | | locus, on sense strand, via homologous | | |
| | | recombination (2/3/5 arms). Constructed | | |
| | | by GenScript. | | |
| p166 | pIMAY-502a-2/3/5HA- | HlgA2 promoter driving holin kill gene. | Chlor | Chlor |
| | PhlgA2-holin (rev) | For genomic integration into 502a at azlC | | |
| | | locus, on sense strand, via homologous | | |
| | | recombination (2/3/5 arms). Constructed | | |
| | | by GenScript. | | |
| p171 | pIMAY-502a-2/3/5HA- | HlgA2 promoter driving lysostaphin kill | Chlor | Chlor |
| | PhlgA2- | gene (mature form). For genomic | | |
| | matureLysostaphin | integration into 502a at azlC locus, on | | |
| | (rev) | sense strand, via homologous | | |
| | | recombination (2/3/5 arms). Constructed | | |
| | | by GenScript. | | |
| p172 | pRAB11-Ptet-1871ysK | 187/lysK phage lytic chimeric kill gene | Amp | Chlor |
| | | under control of tetracycline-inducible | | |
| | | promoter. | | |
| p173 | pRAB11-Ptet-holin | Holin kill gene under control of | Amp | Chlor |
| | | tetracycline-inducible promoter. | | |
| p174 | pRAB11-Ptet-sprA1 | SprA1 kill gene (without antitoxin | Amp | Chlor |
| | | sequence) under control of tetracycline- | | |
| | | inducible promoter. Kill gene includes | | |
| | | some sequence upstream of the start | | |
| | | codon. | | |
| p175 | pRAB11-Ptet- | sprA1 kill gene (without antitoxin | Amp | Chlor |
| | sprA1(ATG) | sequence) under control of tetracycline- | | |
| | | inducible promoter. Kill gene sequence | | |
| | | begins at start codon. | | |
| p176 | pIMAY-502a-2/3/5HA- | HlgA2 promoter driving lysostaphin kill | Chlor | Chlor |
| | PhlgA2- | gene (mature form). For genomic | | |
| | matureLysostaphin | integration into 502a at azlC locus, on | | |
| | | anti-sense strand, via homologous | | |
| | | recombination (2/3/5 arms). Constructed | | |
| | | by GenScript. | | |
| p177 | pIMAY-502a-2/3/5HA- | HrtAB promoter driving lysostaphin kill | Chlor | Chlor |
| | PhrtAB- | gene (mature form). For genomic | | |
| | matureLysostaphin | integration into 502a at azlC locus, on | | |
| | | anti-sense strand, via homologous | | |
| | | recombination (2/3/5 arms). Constructed | | |
| | | by GenScript. | | |
| p178 | pRAB11-Ptet-sprG1 | SprG1 kill gene under control of | Amp | Chlor |
| | | tetracycline-inducible promoter. | | |
| p180 | pCN56-TT-PhrtAB- | HrtAB promoter driving sprA1 kill gene, | Amp | Erm |
| | sprA1 | p102 with sprA1 antitoxin deleted. | | |
| p181 | pIMAY-502a-2/3/5HA- | HrtAB promoter driving lysostaphin kill | Chlor | Chlor |
| | PhrtAB- | gene (mature form). For genomic | | |
| | matureLysostaphin | integration into 502a at azlC locus, on | | |
| | (rev) | sense strand, via homologous | | |
| | | recombination (2/3/5 arms). Constructed | | |
| | | by GenScript. | | |
| p187 | pCN56-TT-PhlgA2- | p086 with His tag | Amp | Erm |
| | sprA1-sprA1at-His | | | |
| p188 | pCN56-TT-Pcad- | p097 with His tag | Amp | Erm |
| | sprA1-sprA1at-His | | | |
| p189 | pRAB11-Ptet-sprA1- | p174 with His tag | Amp | Chlor |
| | His | | | |
| p190 | pRAB11-Ptet- | p175 with His tag | Amp | Chlor |
| | sprA1(ATG)-His | | | |
| p196 | pRAB11-Ptet- | Lysostaphin kill gene under control of | Amp | Chlor |
| | lysostaphin | tetracycline-inducible promoter. | | |
| p232 | pCN56-TT-PhlgA2- | HlgA2 promoter driving sprA1 kill gene. | Amp | Erm |
| | sprA1 | p096 (made by GenScript) with sprA1 | | |
| | | antitoxin deleted. | | |
| p233 | pCN56_TT-P305- | Kill switch using p305 and P360 driving | Amp | Erm |
| | sprA_sprA1-P360-TT | the expression of sprA/sprA(AS) | | |
| p234 | pRAB11-Ptet-noRBS- | Tetracycline-inducible promoter driving | Amp | Chlor |
| | sprG1 | kill gene without an RBS. Serves as a | | |
| | | negative control for Ptet assays. | | |
| p235 | pCN56-TT-PhlgA2- | SprA1 kill gene and antitoxin driven by | Amp | Erm |
| | sprA1-sprA1at | hlgA2 promoter | | |
| p236 | pCN56-TT-PhlgA2- | HlgA2 promoter driving sprA1 kill gene | Amp | Erm |
| | sprA1-sprA1at | and antitoxin. Promoter insert synthesized | | |
| | | and cloned into p078_pCN56-TT-sprA1- | | |
| | | sprA1at by GenScript. | | |
| p238 | pIMAYz_site 2::Pcad- | | Chlor | Chlor |
| | GFP | | | |
| p239 | pIMAYz_site | | Chlor | Chlor |
| | 2::PgyrB-sprA1as | | | |
| p240 | pIMAYz_site 2::Pcad- | | Chlor | Chlor |
| | sprA1 | | | |
| p241 | pIMAYz_site | | Chlor | Chlor |
| | 2::PgyrB-GFP | | | |
| p242 | pZAS_ΔPsprA1::PsbnA | | Chlor | Chlor |
| p244 | Ptet-lysostaphin | Lysostaphin kill gene under control of | | |
| | | tetracycline-inducible promoter. | | |
| p245 | Ptet-sprG1 (short) | SprG1 (short) kill gene (without antitoxin | | |
| | | sequence) under control of tetracycline- | | |
| | | inducible promoter. vector: pRAB11-Ptet | | |
| p246 | Ptet-sprA2 | SprA2 kill gene (without antitoxin | | |
| | | sequence) under control of tetracycline- | | |
| | | inducible promoter. vector: pRAB11-Ptet | | |
| p247 | Ptet-mazF | mazF kill gene (without antitoxin | | |
| | | sequence) under control of tetracycline- | | |
| | | inducible promoter. vector: pRAB11-Ptet | | |
| p248 | Ptet-YoeB-sa2 | Yoeb-sa2 kill gene (without antitoxin | | |
| | | sequence) under control of tetracycline- | | |
| | | inducible promoter. vector: pRAB11-Ptet | | |
| p249 | isdB::sprA1 | sprA with its RBS dropped in behind isdB | Chlor | Chlor |
| | | with a six base spacer. | | |
| p252 | PsbnA::sprA1 | plasmid to insert sprA1 behind the sbnA | Chlor | Chlor |
| | | promoter | | |
| p254 | 04385::sprA1 | integrates sprA1 behind CH52_04385 | Chlor | Chlor |
| p255 | 05105::sprA1 | integrates sprA1 behind CH52_05105 | Chlor | Chlor |
| p256 | 06885::sprA1 | integrates sprA1 behind CH52_06885 | Chlor | Chlor |
| p257 | 10455::sprA1 | integrates sprA1 behind CH52_10455 | Chlor | Chlor |
| p260 | isdb::sprA1(triple stop | sprA with its RBS dropped in behind isdB | Chlor | Chlor |
| | codon) | with a six base spacer, two additional stop | | |
| | | codons added after isdB in different | | |
| | | frames | | |
| p261 | isdB::sprG1 | sprG1 inserted behind isdB | Chlor | Chlor |
| p262 | isdB::sprA1 | sprA1 inserted behind isdB gene (no | Chlor | Chlor |
| | | mutations in homology arms) | | |
| p265 | PsbnA::sprG1 | sprG inserted behind PsbnA | Chlor | Chlor |
| p267 | isdB::sprA2 | sprA2 toxin behind isdB | Chlor | Chlor |
| p268 | PsbnA::sprA2 | sprA2 toxin behind sbnA promoter | Chlor | Chlor |
| | | **Note has point mutation in Right HA | | |

*AbR: Antibiotic Resistance

Additional plasmids with generated synthetic strains are shown in Table 7B shown in FIG. 6A.

### Example 3. Truncated and Frame-Shifted sprA1 Efficacy Assay in E. coli and Staph aureus

When making the plasmid p257 (pIMAYz_harA::sprA1) the sprA1 gene acquired a base pair deletion which resulted in a frameshift and truncated protein (SEQ TD NO: 47) (BP_DNA_090) having amino acid sequence MLIFVHIIIAPVISGCAIAFFLIG (BP_AA_014) (SEQ TD NO: 84) A protein sequence alignment using the BLOSUM62 matrix showed a 64.5% similarity between the mutated protein and native protein having amino acid sequence (BP_AA_002) MLIFVHIIAPVISGCAIAFFSYWLSRRNTK (SEQ ID NO: 72), encoded by BP_DNA_035 (SEQ TD NO:25). In order to test the efficacy of the mutated and truncated protein the mutated sprA1 gene was inserted into the pRAB11 plasmid so it could be regulated by the P_{(xyl/tet)} promoter and induced by anhydrotetracycline (ATc). The new plasmid was named p298 and was tested in E. coli and Staph aureus BP_001 for its effect on the cell culture when overexpressed.

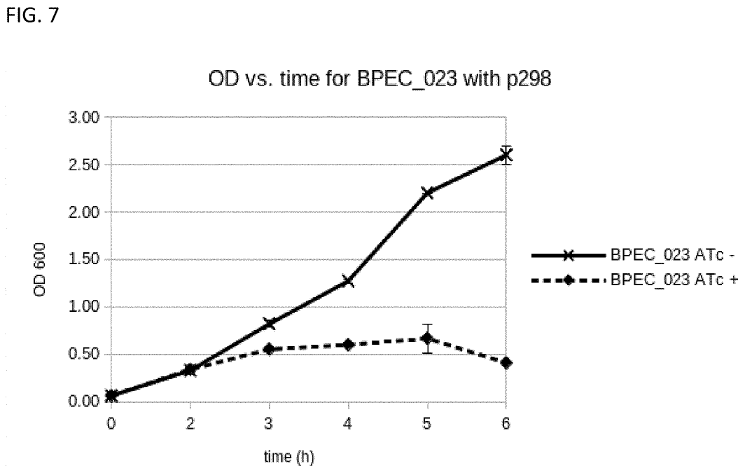

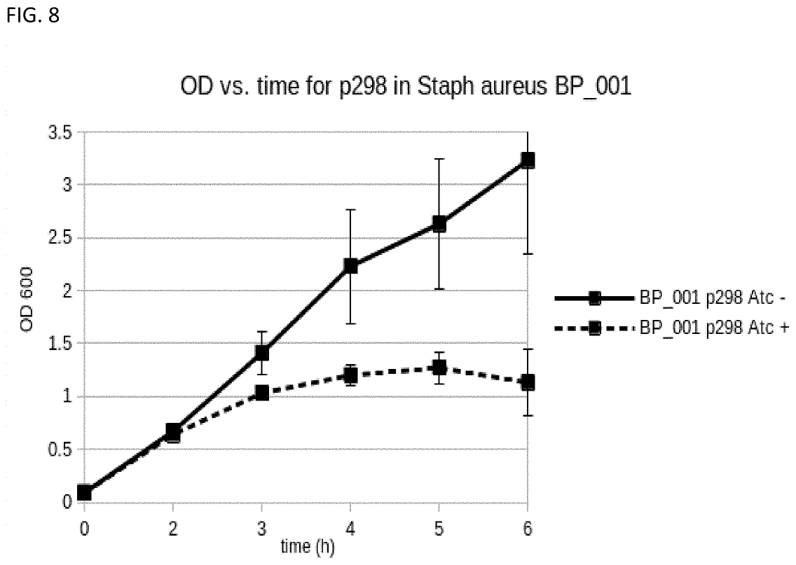

Briefly, three biological replicate overnight cultures for each strain harboring the plasmid were grown in TSB media at 37° C. in a shaking incubator at 240 rpm. The following day the cultures were cut back to an OD of 0.05 and each overnight culture was split into two tubes, grown for 2 hours at 37° C. After two hours of growth, one tube for each strain received a spike of ATc to induce the expression of the truncated sprA1 gene and then placed back in the shaking incubator to continue growing. Samples were taken every hour to measure the density of the culture by measuring the absorbance at 600 nm (OD600). FIGS. 7 and 8 show the average OD measurements plotted against time for the strains tested.

FIG. 7 shows induced and uninduced growth curves for the E. coli strain IM08B (BPEC_023) harboring the p298 plasmid by plotting the OD600 value against time. The solid line represents average values (n=3) for uninduced cultures, and the dashed line represents the average values (n=3) for the induced cultures. The error bars represent the standard deviation of the averaged values. Within 2 hours of induction, the BPEC_023 E. coli culture growth rate slowed for each following time point and eventually went negative before the assay was stopped, whereas uninduced culture exhibited continued growth over 6 hrs of assay.

FIG. 8 shows the growth curves for the Staph aureus strain BP_001 harboring the p298 plasmid by plotting the OD600 value against time. The solid line represents average values (n=3) for uninduced cultures, and the dashed line represents the average values (n=3) for the induced cultures. The error bars represent the standard deviation of the averaged values.

Overexpression of the truncated sprA1 gene (BP_DNA_090, SEQ ID NO: 47) encoding BP_AA_014 (SEQ ID NO: 84) had an effect on the growing E. coli and Staph aureus cultures. The growth curves for the uninduced cultures began diverging from the induced cultures within 2 hrs following the addition of ATc, where the uninduced cultures continued to grow in log phase and the growth of the induced cultures slowed dramatically directly after the addition of ATc. For both strains tested, the growth rate slowed for each following time point and eventually went negative before the assay was stopped. ATc has been shown to be nontoxic and does not inhibit either species tested at the concentrations used in the experiment, so the only variable between the two cultures tested that could have caused the lower culture density in the induced cultures is the overexpressed truncated sprA1 gene.

### Example 4. Group B Strep Kill Switch Design

A piggyback method may be employed to insert action genes behind promoters or differentially regulated genes in bacterial genomes can produce very unique and specific responses to certain stimuli while sufficiently “hiding” the inserted gene or genes from the cell in other environments. We have demonstrated the insertion of an effective kill switch into the genome of Staphylococcus aureus such that the cell induces apoptosis when cultured in biological fluids such as serum, blood, plasma, and cerebrospinal fluid (CSF). These genomic switches have also been shown to be stable for over 500 generations, as provided herein, further indicating that this method of engineering cells can have many uses.

To further demonstrate the usefulness of the piggyback method, the method may be applied to a Streptococcus (Strep) species. The target microorganism may be a Group B Strep, such as Strep agalactiae, a pathogenic strain which can cause SSTI, bovine mastitis, and neonatal sepsis.

Hypothetical toxin/antitoxins of Strep agalactiae may be found in the genome, for example, as provided in Xie et al., 2018. Xie et al., TADB 2.0: An Updated Database of Bacterial Type II Toxin-Antitoxin Loci. Nucleic Acids Res. 2018, 46 (D1), D749-D753. https://doi.org/10.1093/nar/gkx1033. Table 8 shows a list of hypothetical Strep agalactiae toxin genes and their accession numbers. Toxin genes from other Strep species such as Strep pneumonia and Strep mutans may also be screened for potential use. Toxin genes may be PCR amplified out of the genome of Strep agalactiae using specific primer pairs. Toxin genes may also be printed out or synthesized using a DNA printing service. Toxins may be screened for lethality against Strep agalactiae by integrating the toxin gene onto a plasmid with an inducible promoter. For example, a plasmid will be used with a tet inducible promoter system, such as pRAB11, that can be induced (or derepressed) by anhydrotetracycline (ATc), a non-toxic analog of the antibiotic tetracycline. The toxin will be inserted behind the promoter on the plasmid and therefore the expression of the toxin will be induced with the addition ATc. The difference in optical density (OD) between induced and non induced strains will show the effectiveness of the toxin genes added to the plasmid. The most effective toxin genes in the inducible platform may be used to create serum inducible kill switches in Group B Strep. Table 8 shows toxin genes found using the 2.0 Toxin/Antitoxin Database. Xie et al., 2018.

TABLE 8
Potential Toxin Genes for Group B Strep

| | Hypothetical Toxins in | Strep Agalactiae |
|---|---|---|
| Accession Number | Strep agalactiae | Strain |
| WP_000384860.1 | RelE/ParE family toxin | A909 |
| WP_000700104.1 | ImmA/IrrE family toxin | A909 |
| WP_000666489.1 | RelE/ParE family toxin | A909 |
| NP_687263.1 | RelE/ParE family toxin | 2603V/R |
| AAM99341.1 | mazEF, ccd or relBE | 2603V/R |
| NP_687584.1 | Bro | 2603V/R |
| NP_688285.1 | abiGII | 2603V/R |
| NP_688826.1 | HicA | 2603V/R |
| NP_688872.1 | COG2856 | 2603V/R |
| NP_688994.1 | RelE | 2603V/R |
| NP_689104.1 | Fic | 2603V/R |

Selection of inducible promoter gene. Multiple locations in the Strep agalactiae genome may be targeted to integrate a toxin gene or genes. Promoters and genes that are upregulated in serum can be found using RNA-seq or from literature. See Table 9 for a list of Strep agalactiae genes that are necessary for growth or upregulated in serum. One site of interest could be the IgA-binding 3 antigen gene which is upregulated in serum. Hooven et al. The Streptococcus Agalactiae Stringent Response Enhances Virulence and Persistence in Human Blood. Infect. Immun. 2017, 86 (1). https://doi.org/10.1128/IAI.00612-17.

The toxin will be integrated behind the inducible promoter gene in such a way that it will be on the same mRNA transcript as the IgA-binding β antigen gene. The upregulated expression in serum of the IgA-binding β antigen gene will be tied or piggybacked to the toxin gene. This will increase the expression of the toxin gene in serum, creating a kill switch. Table 9 shows candidate serum inducible promoter genes in Strep agalactiae.

TABLE 9
Upregulated or Necessary Genes for
Strep agalactiae in Human Blood

| | Gene Locus | Protein | Purpose |
|---|---|---|---|
| 1 | SAK_1262 | Regulatory protein CpsA | essential for |
| | | | survival in blood |
| 2 | SAK_1255 | Capsular polysaccharide synthesis | essential for |
| | | protein CpsH | survival in blood |
| 3 | SAK_1251 | Polysaccharide biosynthesis protein | essential for |
| | | CpsL | survival in blood |
| 4 | SAK_0483 | R3H domain-containing protein | essential for |
| | | | survival in blood |
| 5 | SAK_1254 | Capsular polysaccharide | essential for |
| | | biosynthesis protein | survival in blood |
| 6 | SAK_1259 | Tyrosine-protein kinase CpsD | essential for |
| | | | survival in blood |
| 7 | SAK_1260 | Capsular polysaccharide | essential for |
| | | biosynthesis protein CpsC | survival in blood |
| 8 | SAK_1249 | UDP-N-acetylglucosamine-2- | essential for |
| | | epimerase NeuC | survival in blood |
| 9 | SAK_1900 | GTP pyrophosphokinase RelA | essential for |
| | | | survival in blood |
| 10 | SAK_1895 | PTS system transporter subunit | essential for |
| | | IIA | survival in blood |
| 11 | SAK_1258 | Glycosyl transferase CpsE | essential for |
| | | | survival in blood |
| 12 | SAK_1253 | Capsular polysaccharide | essential for |
| | | biosynthesis protein CpsJ | survival in blood |
| 13 | SAK_1248 | NeuD protein | essential for |
| | | | survival in blood |
| 14 | SAK_0186 | IgA-binding β antigen | essential for |
| | | | survival in blood |
| 15 | SAK_1256 | Polysaccharide biosynthesis | essential for |
| | | protein CpsG | survival in blood |
| 16 | SAK_1257 | Polysaccharide biosynthesis | essential for |
| | | protein CpsF | survival in blood |
| 17 | gbs0791 | Fibrinogen binding surface | invasion of |
| | | protein C FbsC | epithelial cells |

Table 9 shows genes #1-16 were found to be essential for survival in human blood based on transposon sequencing data. Hooven et al. The Streptococcus Agalactiae Stringent Response Enhances Virulence and Persistence in Human Blood. Infect. Immun. 2017, 86(1). https://doi.org/10.1128/IAI.00612-17. Table 9 shows gene FbsC (#17) was predicted based on whole genome sequencing and characterized as a fibrinogen binding protein. Buscetta et al., 2014, FbsC, a Novel Fibrinogen-binding Protein, Promotes Streptococcus agalactiae-Host Cell Interactions http://www.jbc.org/content/289/30/21003.long. All gene candidates shown should have upregulated expression in blood or epithelial cells which makes them a good target for use in the piggyback method.

To make these insertions into the genome, a plasmid for making the genomic modifications through homologous recombination is selected. The plasmid may be pMBsacB which allows for seamless genomic knockout or integrations using a temperature selective origin of replication and a sucrose counterselection to delete the plasmid out of the genome after the homologous recombination event. Hooven et al. A Counterselectable Sucrose Sensitivity Marker Permits Efficient and Flexible Mutagenesis in Streptococcus Agalactiae. Appl. Environ. Microbiol. 2019, 85 (7). https://doi.org/10.1128/AEM.03009-18.

Homology arms and the toxin gene may be added to the pMBsacB plasmid using Gibson Assembly. Enzymatic assembly of DNA molecules up to several hundred kilobases Nature Methods https://www.nature.com/articles/nmeth.1318/. The plasmid may be transformed into competent Strep agalactiae cells and grown at a permissive temperature to allow for replication of the plasmid. The cells will be switched to a nonpermissive temperature to force the integration of the plasmid into the genome at one of the homology arms. After confirming the integration, the plasmid may be removed from the genome, leaving the edit behind. This will be done with the addition of sucrose which acts as a counterselectant against cells that have retained the plasmid. Colonies may be screened via PCR and sequenced to ensure that the genomic edit is correct and the plasmid has been kicked out. Once the genomic edit is complete the new strain may be tested for its ability to grow in human serum by evaluating it in a serum assay as provided herein. The new kill switched strain will be inoculated into human serum and samples will be taken and plated on agar media at various time points to measure the growth of the culture by calculating colony forming units (CFU) per mL of serum. The new Strep agalactiae kill switched strain should not grow in serum but perform similar to the wild type strain in other complex media.

p296 pMBsacB_colE1. The typical protocol for using this plasmid, as stated above, requires E. coli harboring the plasmid to be grown at 30° C. or lower, which severely reduces the growth rate and extends the overall timeline for making genomic modifications in Strep by several days. In order to speed up the process of assembling plasmids to manipulate DNA in Strep, we added a derivative of the colE1 origin of replication to the pMBsacB plasmid backbone. The colE1 on comes from the plasmid pcolE1, and the modified version we used maintains a copy number around 300-500 plasmids per cell and is not temperature sensitive in E. coli. The promoter should not be recognized by Group B Strep, so it should not interfere with the temperature sensitive in vitro DNA recombination in that strain.

The DNA sequence for the colE1 on was added by linearizing the pMBsacB vector (BP_DNA_086)(SEQ ID NO: 43) by PCR amplification, and adding a PCR amplified DNA fragment containing the colE1 on (BP_DNA_085) from the pRAB11 plasmid. The two PCR products were joined to form one circular plasmid using the Gibson Assembly kit (NEB) per the manufacturer's instructions, transformed into E. coli, and recovered and plated at 37° C. Colonies on the plates were screened for the colE1 insert, and three positive plasmids were purified and sequenced to confirm the correct DNA sequence. The new plasmid was named p296 (BP_DNA_122) and is stocked in the present inventors' plasmid database. Homology arms to target a genomic modification are added to the plasmid and its ability to recombine in the genome to make edits is tested in Group B Strep.

### Example 5. Genetic Engineering of Staphylococcus aureus with pIMAYz

This protocol was designed to make edits to the genome of Staphylococcus aureus and is based on publications by Corvaglia et al. and Ian Monk et al. Genetic manipulation of S. aureus is difficult due to strong endogenous restriction-modification barriers that detect and degrade foreign DNA resulting in low transformation efficiency. The cells identify foreign DNA by the absence of host-specific methylation profiles.¹ Corvaglia, A. R. et al. “A Type III-Like Restriction Endonuclease Functions As A Major Barrier To Horizontal Gene Transfer In Clinical Staphylococcus Aureus Strains”. PNAS vol 107, no. 26, 2010, pp. 11954-11958. doi:10.1073/pnas.1000489107. The E. coli strain IM08B mimics the type I adenine methylation profile of certain S. aureus strains, thus evading the endogenous DNA restriction system.

pIMAYz is an E. coli-Staph aureus shuttle vector, has a chloramphenicol resistance for both strains, and the blue/white screening technique can be used when x-gal is added to the agar plates. The plasmid is not temperature sensitive in E. coli, but is temperature sensitive in Staph aureus meaning the plasmid is able to replicate at 30° C. but is unable to do so at 37° C. The temperature sensitive feature allows for editing a target DNA sequence (genomic DNA) in vivo via homologous recombination.

The homologous recombination technique allows for markerless insertions or deletions in a target sequence using sequences that are homologous between the donor and target DNA sequences. These homologous DNA sequences (homology arms) must first be added to the plasmid backbone. Homology arms correspond to roughly 1000 bases directly upstream and downstream of the location targeted for editing. If an insertion is the end result, the DNA to be inserted should be placed in between the homology arms in the plasmid. If the end result is to be a genomic deletion, the homology arms should be right next to each other on the plasmid.

Once the plasmid is made and transformed into the target organism, the incubation temperature is raised while maintaining chloramphenicol in the media. Since the cell needs the plasmid to maintain resistance to the antibiotic, and the plasmid is unable to replicate at the higher temperatures, the only cells that survive are cells that integrated the plasmid into the target DNA (genome) by matching up the homology arms on the plasmid and target sequence. Once clones that have integrated plasmid are confirmed by PCR, a second crossover event can be allowed to happen by growing the cells with no selection pressure, then plating them on media containing anhydrotetracycline (ATc), a non-toxic analog of the antibiotic tetracycline. The ATc in the media does not directly kill the cells, but induces the secY gene on the plasmid backbone which is toxic to Staph aureus and will kill all of the cells containing the plasmid.

The cells that grow on the ATc plates have either mutated part of the secY gene, or have gone through another recombination event by matching up the homology arms on the plasmid and the genomic DNA again. The plasmid is removed through one of two routes in the second recombination event. If the same homology arms line up to remove the plasmid as did when the plasmid was integrated, there will be no change in the target DNA sequence. If the other set of homology arms line up during the second recombination event, the target molecule will either have the intended insertion or deletion. The multiple outcomes for the second event mean that colonies must be screened both genetically for the insertion/deletion, and phenotypically for their resistance to chloramphenicol and ATc. If a strain has passed all of the QC steps it can be stocked and tested to see the response of the inserted or deleted DNA.

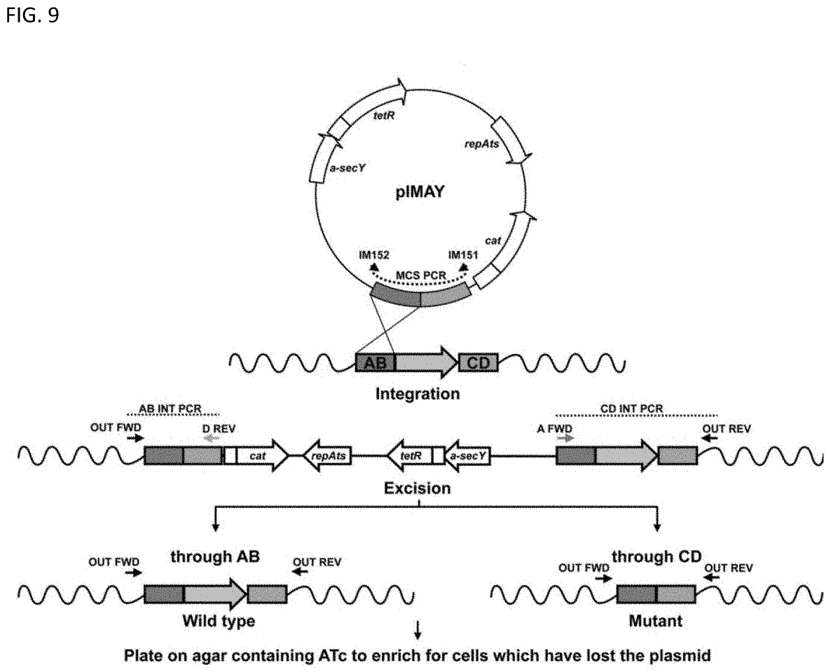

FIG. 9 shows a diagram showing allelic exchange using pIMAY plasmid. The pIMAY plasmid can be used to make insertions in the genome of Staph aureus cells. The figure was taken from Monk et al., Mbio, vol 3, no. 2, 2012. American Society For Microbiology, doi:10.1128/mbio.00277-11.

### Plasmid Prep

Day 1 (PM)—Prepare a highly concentrated pIMAYz integration plasmid (>200 ng/uL).
1. Thoroughly clean surface of biosafety cabinet with 70% alcohol.
2. In the biosafety cabinet, use a 50 mL sterile serological pipet to add 50 mL of LB media to a sterile 250 mL baffled shake flask.
3. Add 100 uL of chloramphenicol (10 mg/mL stock solution).
4. Use a sterile inoculating loop to transfer a colony from a fresh streak plate (less than 5 days old) of the E. coli strain with the desired pIMAYz plasmid into the LB chlor 20 media.
5. Incubate and shake the inoculated culture flask at 37° C. and 240 rpm overnight (12-18 hrs).
6. Clean and sterilize all work spaces and utensils used for the day.

### Day 2

1. Transfer equal volumes of overnight culture into two 50 mL conical tubes.
2. Spin conical tubes in benchtop centrifuge with fixed angle rotor for 4 minutes at 7500×g at room temperature.
3. Carefully pour off the supernatant from both tubes, do not disturb the cell pellet.
4. Add 6 mL of molecular grade water to one of the tubes and resuspend the pellet using the vortex mixer.
5. Transfer the fully resuspended pellet to the second tube and vortex to resuspend the second pellet.
6. Add 600 μL to 10 columns from Zyppy Plasmid Miniprep Kit (Zymo).
7. Perform plasmid extraction according to the manufacturer's instructions.
   a. Elute with 30 uL of warm Zyppy elution buffer (Zymo).
8. Pool elutions, mix, and determine the concentration of extracted plasmid DNA using the Nanodrop.
9. Concentrate extracted plasmid with DNA Clean & Concentrator—25 (Zymo).
   a. Calculate the number of tubes required based on plasmid concentration (each tube can hold 25 ug of DNA).
   b. Perform plasmid concentration according to the manufacturer's instructions.
   c. If multiple tubes are needed, elute into a sterile microfuge tube with 25 uL molecular grade water and pool elutions. If only one tube was required, elute with 40 uL of molecular biology grade water. Initial and date the microfuge tube.
   d. Determine the concentration of the cleaned and concentrated plasmid using the Nanodrop.
   e. Record the concentration on the side of the microfuge tube.
   f. In a separate 1.5 mL microfuge tube, stock 50 μL of 50 ng/L plasmid solution in the −20° C. freezer.
   g. The remaining plasmid can be used immediately for transformation or stored at 4° C. for short term (a couple days) or −20° C. for long term storage. Multiple freeze/thaw cycles will degrade the plasmid over time.
10. Clean and sterilize all work spaces and utensils used for the day.
   Staph aureus Transformation
1. Add small amount of crushed ice to ice bucket. Remove frozen aliquot of electrocompetent Staph aureus cells from −80° C. freezer and incubate on ice for at least 10 minutes.
2. Place 4 BHI (chlor 10/X-gal 100) agar plates in the 37° C. plate incubator and 1 BHI (chlor 10/X-gal 100) agar plate in the 30° C. plate incubator per transformation to begin warming to the appropriate temperature.
3. Set electroporator to the appropriate settings (turn unit on, set volts to 2.5 kV and resistance to 100Ω).
4. Incubate the tubes of electrocompetent Staph aureus cells at room temp for an additional 5 minutes.
5. Pellet the cells in a microcentrifuge at room temp for 1 min at full speed.
6. Use P200 pipette to remove supernatant from pelleted cells. Do not disturb the cell pellet. Resuspend cells in 100-150 μL of sterile cold 500 mM sucrose by gently pipetting up and down. Be sure to break up any chunks if found.
7. Add up to 5 μL of concentrated pIMAYz plasmid to the cell suspension.
8. Transfer cell and plasmid suspension to 2 mM gap electroporation cuvette. Be sure the cell/plasmid suspension goes all the way to the bottom of the cuvette and spreads all the way across the bottom, and that no bubbles are present on top of the cell suspension.
9. Allow the cell and plasmid suspension to rest in the cuvette at room temperature for at least one minute.
10. Preload 1 mL of SA Recovery media (B2 media) in a P1000 pipette and store in a quasi aseptic manner.
11. Place cuvette in the holder of the electroporator and simultaneously press the two red buttons until the low beep is heard from the instrument, then release.
   a. If there is a loud snap or big spark from the cuvette, throw away the cuvette and cells in the biohazard and try the reaction again (also discard the pre-loaded SA recovery buffer).
   b. If there is no snapping or sparking, quickly add the preloaded SA Recovery buffer to the cells in the cuvette and mix by pipetting up and down a couple times, then transfer as much as possible to a sterile 15 mL culture tube.
   c. Incubate the recovering cells in the room temp shaker for 40 minutes at 240 rpm.
   d. After 40 min shaking and recovering at room temp., transfer the culture tube of recovering cells to 37° C. shaking incubator for an additional 30 minutes.
   e. After 30 minutes incubating in the 37° C. shaker, plate aliquots of cells on pre-warmed BHI (chlor 10/X-gal 100) agar plates. Plate 50-200 μL on the prewarmed 37° C. plates, and 150 μL on the 30° C. plate. (Make sure plates are labeled appropriately (strain, plasmid name, date, temperature, volume plated).
12. Incubate the 30° C. plate for up to 48 hours, and the 37° C. plates for up to 24 hours. Colonies should start to be visible around 16-18 hours at 37° C. and 30 hours at 30° C.
   a. Make sure incubator is set to 37° C. or above and put plates in monolayer on the top shelf of the incubator to ensure getting to 37° C. as fast as possible. Once the plates are all sufficiently at 37° C. they can be stacked to conserve room in the incubator.
13. Clean and sterilize all work spaces and utensils used for the day.

### Primary Crossover Selection

1. If blue colonies form on the 37° C. plates, skip to step 2 of this section. If no blue colonies form on the 37° C. plates, toss them in the biohazard and check the 30° C. plate. If there are blue colonies on the 30° C. plate proceed to 1a:
   a. Place 3 BHI (chlor 10/X-gal 100) agar plates in the 37° C. plate incubator to warm for about an hour.
   b. Prefill 6 sterile 1.5 mL tubes per transformation with 900 μL of sterile PBS, TSB, or BHI.
   c. Pick 1-4 blue colonies from the 30° C. plate and fully resuspend them in one of the tubes from the previous step by vortexing.
   d. Perform a serial dilution of the cell suspension by transferring 100 μL of the cell suspension to another tube from step 1.b., and mixing the cells by pulse vortexing at least three times.
      i. Repeat step 1.d until the 10⁻⁵ dilution has been done (4 more transfers).
   e. Appropriately label the prewarmed plates (strain and plasmid name, date, primary cross, dilution), and dispense 100 μL of the 10⁻³-10⁻⁵ dilutions onto the plates and spread using sterile beads or spreaders.
   f. Incubate the plates at 37° C. for 16-24 hours.
2. Screen blue colonies using primer pairs that bind outside the homology arms and inside the plasmid backbone (2 reactions per colony screened). The primers that bind to the gDNA outside the homology arms should be paired with either DR 116, DR_116′, or DR_117, whatever primer has the closest Tm. Getting a band from one (only one) of the primer combinations per sample screened confirms that the plasmid is integrated into the proper location in the genome (no bands=no integration, 2 bands=trouble).
   a. Perform SA lysis in 50 μL of freshly prepared SA lysis buffer (6 μL proK/1 mL of SA buffer).
      i. Prepare sufficient volume of SA lysis solution for the number of colonies that will be screened per SOP_033.
      ii. Use disposable sterile inoculating needle to pick colonies, patch to new BHI (chlor/x-gal) plate, and use remaining cell clump for SA lysis reaction in PCR tube. Incubate the plates at 37° C. for 6-24 hours.
      iii. Use a thermal cycler to perform lysis using SA lysis protocol.
      iv. When thermal cycler SA lysis program has finished, allow the tubes to cool if still warm and pellet the cell debris in the tubes by briefly spinning the tubes in a mini centrifuge with a PCR tube insert.
   b. Screen for the insert by PCR using Econotaq.
      i. 20 μL total reaction volume.
      ii. 2 μL of SA cell lysis for the template.
   c. Run PCR products on 1% agarose gel.
      Optional Step: Screen blue colonies by PCR using same SA lysis for the template, and the primer pair DR_116/DR_117 in Econotaq for the presence of circular plasmid. The 37° C. plates should not produce any band because the plasmid integrated into the host. DR_116/DR_117 are flanking the multiple cloning site in pIMAYz, and for the 30° C. plates will produce a band the same size as the homology arms plus any region being integrated.
3. If the agarose gel shows a band while screening the colonies for integrated plasmid, pick 3 different colonies from the patch plate that had positive PCR bands. Grow overnight (˜16 h) in 5 mL BHI broth in room temperature shaker.
4. The next day, serially dilute cultures (as described above in Step 1 of the Primary Crossover) in PBS to 10⁻⁶ and plate 100 μL of dilutions 10⁻⁴-10⁻⁶ on BHI (ATc 1 μg/mL, X-Gal 100 μg/mL) agar and incubate the plates overnight (16-24 h) at 37° C.

### Secondary Crossover Selection

5. The next day if white colonies are visible, place 3 plates in the 37° C. incubator for an hour to warm: (1) BHI agar (AnhydroTet 1 μg/mL, X-Gal 100 μg/mL) agar plate, (1) BHI (chlor 10 μg/mL, X-Gal 100 μg/mL) agar plate, (1) BHI or TSB agar plate.
6. Place grid stickers on the backs of the plates, label the plates appropriately (media+additives, strain name, secondary cross, date) and patch 50 white colonies to the prewarmed plates.
   a. The patching order of the plates should go
      i. BHI (ATc/x-gal)
      ii. BHI (chlor/x-gal)
      iii. BHI/TSB
   b. If possible take an equal number of colonies from each culture plated.
   c. Incubate all of the plates at 37° C. for 14-24 hours.
7. Screen colonies for resistance to ATC (growth) and sensitivity to chloramphenicol (no growth). Cross out any colonies on the grid that do not satisfy that criteria.
8. Picking from the BHI or TSB agar plate, perform SA lysis in 50 μL of freshly prepared SA lysis buffer (6 L proK/1 mL of SA buffer) on at least 16 patches that show growth on ATc plates and no growth on chloramphenicol plates. Following the SA lysis protocol, spin down the cell debris prior to using the reaction as the template for PCR in the next step.
9. Use the SA lysis from the previous step to screen for the desired genotype by PCR. Use one primer that binds to a unique region in the new genotype (inside inserted DNA or across the deleted sequence) and the other primer will bind outside the homology arm to the genome or target DNA.
   a. PCR screen using Econotaq.
      i. 20 μL total reaction volume.
      ii. 2 μL of SA cell lysis as the template.
   b. Run PCR products on 1% agarose gel.
10. If positive bands are seen from the PCR screen, the strains must be sequenced to confirm the exact sequence of the new strains. Using the same SA lysis template as the previous PCR reaction, use a high fidelity PCR master mix and a primer pair that binds to the genome outside the homology arms to PCR amplify the entire modified region and the homology arms.
   a. Q5 Hot-Start High Fidelity (or Phusion) DNA polymerase.
      i. 50 uL total reaction volume.
      ii. Use up to 5 μL of SA cell lysis as the template (If 2 μL worked previously, use 2 μL).
   b. Run PCR products on 1% agarose gel and view with transilluminator.
      i. If bands look good and the lane is otherwise clean, purify the PCR product with a PCR purification kit per manufacturer's instructions. Elute with 50 μL molecular biology grade water.
      ii. If nonspecific bands or other contaminants are seen on the agarose gel, repeat PCR using more optimized conditions or purify the DNA from the agarose gel using a gel purification kit per the manufacturer's instructions.
   c. Submit purified PCR product for sanger sequencing making sure to get at least 2× coverage of the entire PCR fragment.
11. For each strain that was sequenced, from the same BHI or TSB plate that was used for the SA lysis in the last PCR screen, streak a plate for single colony isolation on BHI or TSB plates and incubate the plates 37° C. overnight.
12. When the sequencing reactions are done and the sequences are emailed back, download the .ab1 files and align them to the reference sequence in Benchling for the desired target DNA modification.
13. If the sequencing alignment comes back with correct sequence, pick at least 3 colonies of the best clone and PCR screen to confirm genotype (just as done in Step 9 above). One primer should bind to a unique region in the new genotype and the other primer should bind outside the homology arm.
   a. If all PCRs show a positive band when run on a 1% agarose gel, a single colony from the streak plate can be used to create strain stocks.
   b. If all PCRs are not positive, restreak the plate and try the PCR again.
14. Clean and sterilize all work spaces and utensils used for the day.

### Stocking Strains

1. After the new strain has been confirmed through sequencing and the final colony screen, give the strain a number and description in the Strain Database.
   a. Create and print out 3 cryolabels per stain.
   b. Include strain number, genotype, and date stocked on label.
2. In biological safety cabinet, pick a single colony from the plate that has been confirmed via PCR (Step 13 above) to inoculate 5 mL of sterile BHI.
3. Incubate in the shaking incubator at 37° C. and 240 rpm overnight (12-20 hrs).
4. The following morning, remove the culture from the shaker and place it in the biological safety cabinet.
   a. Label 3 cryovials prefilled with 750 ul of sterile 50% glycerol with appropriate description.
   b. Using a P1000 pipette, transfer 750 uL of the culture to a cryovial. Repeat transfer step to for remaining cryovials using a new pipette tip each transfer.
   c. Be sure the caps are screwed on tight and vortex or invert the tubes at least 10 times to evenly mix the cells and glycerol.
   d. Store 1 cryovial tube in each of the following boxes in the −80° C. freezer:
      i. Strain Database
      ii. Backup Strain Database
      iii. CU Backup Strain Database

The pIMAYz plasmid allows for efficient and reliable editing the genome of S. aureus. Initial transformations may have low yields due to restriction barriers and other factors, but if the plasmid is designed properly and the Staph aureus strain takes up the plasmid and shows resistance to chloramphenicol, it is highly likely to produce the desired genetic modification if the protocol is followed. The phenotypic and genomic selections allow for timely and reliable results.

Homologous recombination is not known for making edits to DNA outside of the target region, so there is no need to sequence the entire genome after making each modification. Homology arms can be designed to make genomic modifications that can change a single DNA base, delete genes or operons, or make insertions ranging from single bases to tens of thousands of bases. The process can be repeated multiple times in the same strain to make iterative modifications.

### Example 6A. Reporter Genes: GFP and mKATE2 Piggyback Integrations

In this example, two locations in the Staph aureus genome were targeted to integrate reporter genes encoding fluorescent proteins into WT Staph aureus strain BP_001, (i) behind the endogenous isdB gene to prepare synthetic strains BP_0152 isdB::GFP and BP_0158 isdB::mKATE2, and (ii) in between the endogenous promoter for the sbnA gene and the start codon for the sbnA gene to prepare synthetic strains BP_0151 PsbnA::GFP and BP_0157 PsbnA::mKATE2. To make these insertions into the genome, plasmids capable of making the genomic modifications were developed. Homology arms were added to the pIMAYz plasmid backbone and homologous recombination was employed to make the gene edit per the protocol provided herein.

Fluorescent reporter genes may also be inserted to the genomes of E. coli or Streptococcus target microorganisms. For example, based on literature searches for genes in E. coli that are upregulated when the cells are cultured in human serum, several locations were chosen for the integration of GFP and RFP in E. coli. The locations in the genome that were identified for integration are directly behind the genes ybjE, yncE, and ftn. Huja, Sagi, et al. MBio 5.4 (2014): e01460-14. Using the techniques described in the introduction, following successful integration of the fluorescent proteins into the sites identified in the E. coli genome, the level of fluorescence of the engineered cells will be used to calculate the level of expression of the operons in which the integrations were made.

Literature searches for genes that are upregulated in Group B Strep (Streptococcus agalactiae) when they are exposed to human serum showed several genes necessary either for growth in blood or upregulated in that environment, for example, cpsA and IgA-binding β antigen (SAK_0186). Hooven et al. 2018. Infect Immun 86:e00612-17

The Streptococcus agalactiae stringent response enhances virulence and persistence in human blood. Infect Immun 86:e00612417. Through homologous recombination using the pMBsacB plasmid generously donated from Dr. Hooven, GFP and RFP genes may be separately integrated directly behind the genes identified above as necessary or upregulated in human serum as well as multiple other genes in the Streptococcus agalactiae genome.

Fluorescence of the cultures was assayed following their exposure to human serum compared to complete media such as TSB to assess the levels of upregulation in that environment.

Candidate construct strains were designed to harbor serum-responsive reporter genes, and were genotypically confirmed via Sanger sequencing. The strains were grown in human serum or tryptic soy broth (TSB). Culture samples are taken from each growth condition at predetermined time points for phenotypic analysis by fluorescence spectroscopy. Samples were taken at t=0 h, t=1 h, t=2 h, t=3 h, t=4 h, pelleted, and loaded into 96 well microplates, and fluorescence analyses are performed using a plate reader in triplicate. Fluorescence measurements are obtained using a BioTek Synergy II Plate Reader using instrument setting for GFP: Ex λ=485/20, Em λ=530/25, Sensitivity=80 or for mKATE2: Ex λ=575/15, Em λ=645/40, Sensitivity=110. Fluorescence readings obtained from construct strains were then compared to calibration curves generated from certified standards, in order to reliably quantify and rank strain/construct fluorescence output.

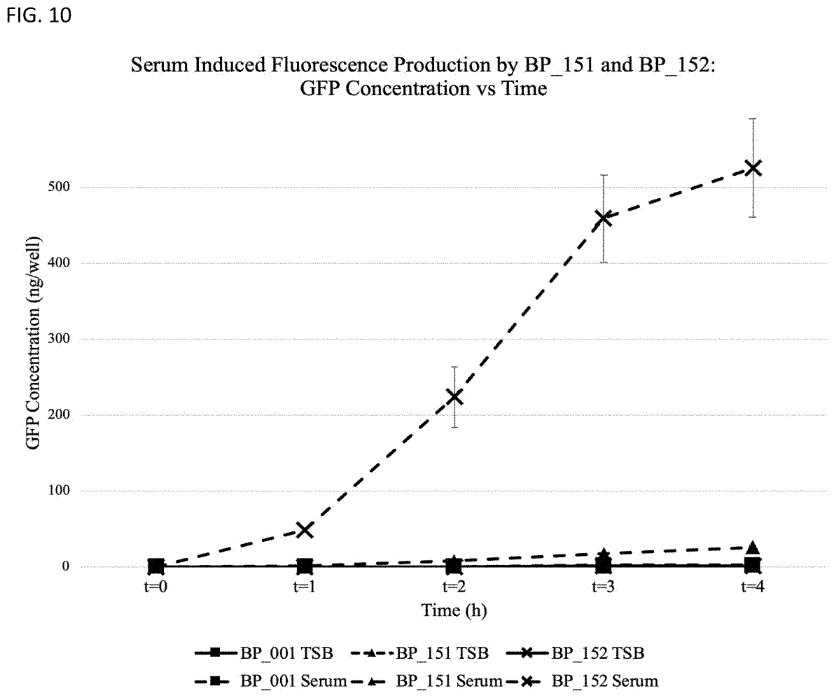

FIG. 10 shows a graph of GFP concentration (ng/well) vs. time (hrs) for serum-induced fluorescence production by Staph aureus synthetic strains BP_151 (PsbnA::GFP) and BP_152 (isdB::GFP) compared to parent stain BP_001 after being cultured in human serum (dashed lines) and TSB (solid lines) over 4 hours. Cultures were prepared as described above and loaded into solid black 96 well microplates in 200 uL technical triplicates. Both strains harbor GFP integrations, which have been placed downstream from serum-inducible promoters using the “piggyback” method. BP_152 exhibited strong fluorescence when cultured in serum. BP_151 also showed emission of fluorescence when cultured in serum. BP_001 was included in the assay as a wild type control—it did not exhibit fluorescence in serum or TSB. Likewise, BP_151 and BP_152 did not fluoresce when cultured in TSB. All strains were prepared and assayed in biological triplicate.

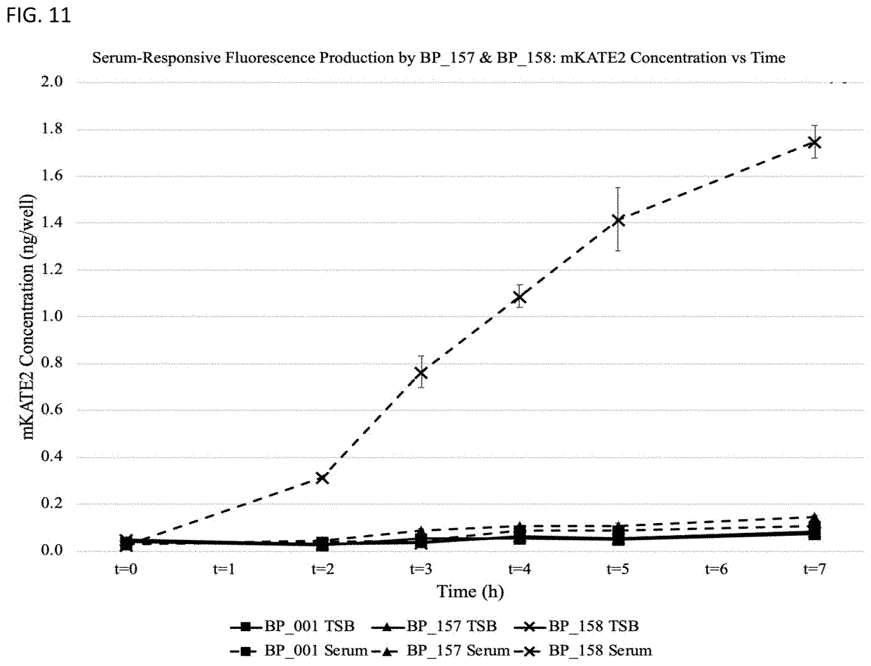

FIG. 11 shows a graph of RFP mKATE2 concentration (ng/well) vs. time (hr) for serum-responsive fluorescence production by BP_157 (PsbnA::mKATE2) and BP_158 (isdB::mKATE2) in human serum (dashed lines) and TSB (solid lines). BP_001 (lacking mKATE2) was included as a wild type control. All strains and conditions were assayed in biological triplicate and were prepared as previously described. Samples were taken at t=0, 2, 3, 4, 5, and 7 h. Technical triplicates were plated in black solid bottom 96 well plates. A strong fluorescence was exhibited by BP_158 when grown in serum. Very slight fluorescence was exhibited by BP_0157. Strains BP_001, BP_157, and BP_158 each showed no fluorescence in TSB.

### Example 6B. Evolutionary Stability of Synthetic Staphylococcus aureus Strain BP_088

In this example, the stability of a synthetic Staph aureus strain prepared according to the disclosure was evaluated over 500 generations. BP_088 (isdB::sprA1) and parent Staph aureus strain BP_001 were grown for an estimated 500 generations by passing growing cultures to fresh media for 250 hours. BP_088 performed the same in human serum prior to and after a 500 generation growth period. No mutations occurred in the DNA sequence of the integrated kill switch region during the 500 generation growth period.

Staph aureus is known to readily undergo genomic changes, and the obstacle of creating a durable genomic integration is always a concern when making edits to an organism's genome. Furthermore, demonstrating the ability to “hide” a genomic edit involving a toxin gene from the organism harboring the edit is important, especially in the Live Biotherapeutic Space (LBP). This has implications for many aspects of genetic engineering wherever there is a concern for the organism to spread once it has left the niche it was intended to inhabit.

Evolutionary stability for the piggyback genomic modification of Staph aureus synthetic strain BP_088 was tested by keeping a culture growing in exponential phase for 250 hours. Since Staph aureus has a generation time of about 30 minutes when grown in rich complex media, it was calculated that after 250 hours of growth the strain should have undergone approximately 500 generations. Maintaining a growing culture was accomplished by diluting a growing culture in a tube with fresh media every 8 to 12 hours, and then testing the strain's response to human serum both before and after the 500 generation growth period. A wild-type Staph aureus (BP_001) was grown alongside a strain containing the isdB::sprA1 integration (BP_088).

The integrations into strains BP_001 to make BP_088 and BP_121 were done using homologous recombination using the pIMAYz plasmid with plasmids p249 and p264 respectively. The edits to the genome of BP_001 to create BP_088 and BP_121 were done following the homologous recombination protocol as provided here.

Tables 10 and 11 show strains employed in the stability assay and the DNA sequence of the genomic edits made, respectively.

TABLE 10
Strains Used in Stability Assay

| | | DNA Sequence ID of |
|---|---|---|
| Strain | Genotype | genomic inserted fragment |
| BP_001 | wild type | n/a |
| BP_088 | BP_001, isdB::spra1 | BP_DNA_003 |
| BP_121 | BP_001, site2::code | BP_DNA_023 |

TABLE 11
DNA Sequences used in this Study

| Se- | | |
|---|---|---|
| quence | | |
| ID | Description | DNA Sequence (5′→3′) |
| BP_{—} | isdB::sprA1 | CGCAGAGAGGAGGTGTATAAGGTGATGCT |
| DNA_003 | | TATTTTCGTTCACATCATAGCACCAGTCA |
| | | TCAGTGGCTGTGCCATTGCGTTTTTTTCT |
| | | TATTGGCTAAGTAGACGCAATACAAAATA |
| | | G (SEQ ID NO: 3) |
| BP_{—} | site2::code | CGATCTTCGACATCGGACCCTAGAACAGA |
| DNA_023 | | ACTA (SEQ ID NO: 19) |

BP_088 for 0 Generation cultures and BP_001 cultures were started in 5 mL of TSB from single colonies on a streak plate. Cultures were grown overnight in a 37° C. incubator, shaking at 240 rpm. The following morning, all cultures were diluted to 0.05 and placed in a 37° C. incubator, shaking at 240 rpm. After 2 hrs the cells were washed once with 5 ml of sterile PBS, and then were used to inoculate 5 mL of prewarmed serum and TSB to 0.05 OD 600. Immediately, the t=0 samples were taken, cultures were placed back into the incubator and serial dilutions were performed and plated. Samples were also taken at t=2, 4, 6, and 8 hrs. BP_088 500 Generation cultures and (1) BP_121 culture were started in 5 mL of TSB from single colonies on a streak plate. Cultures were grown overnight in a 37° C. incubator, shaking at 240 rpm. The following morning, all cultures were diluted to 0.05 and placed in a 37° C. incubator, shaking at 240 rpm. After 2 hrs the cells were washed once with 5 mL of sterile PBS, and then were used to inoculate 5 mL of prewarmed serum and TSB to 0.05 OD. Immediately, the t=0 samples were taken, cultures were placed back into the incubator and serial dilutions were performed and plated. Samples were also taken at t=2, 4, and 9.4 hrs.

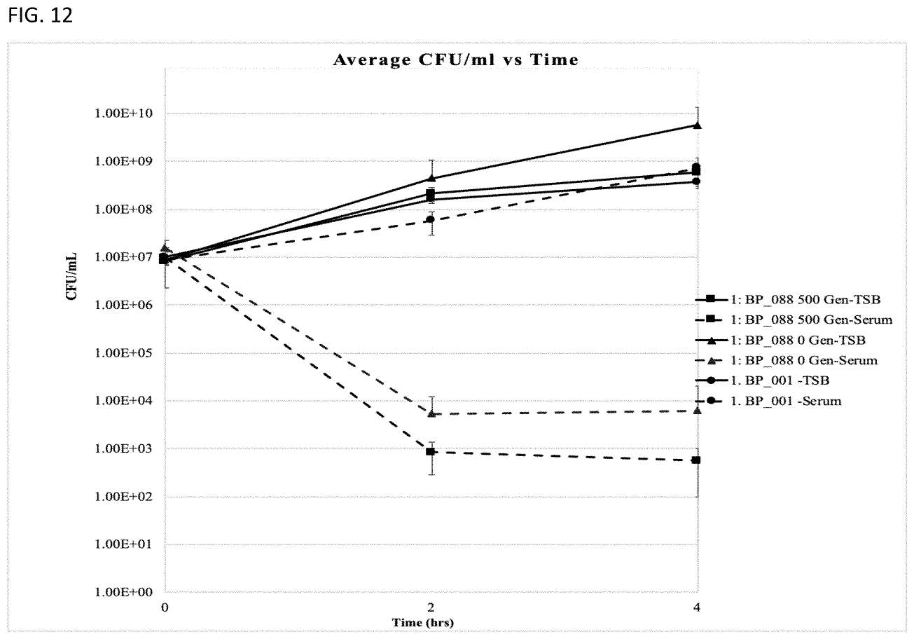

FIG. 12 shows a graph of the average (n=6) of viable CFU/mL of Staph aureus synthetic strain BP_088 (0 and 500 generation strains) when grown in human serum (dashed lines) or TSB (solid lines). BP_001 (n=6) in TSB and serum was plotted as a wild type control. Error bars represent one standard deviation of all six replicates. The BP_088-500 generation sample is represented by solid squares (▪) and the 0 generation sample (▴). Parent strain BP_001 is represented by a solid circle. Synthetic strain BP_088 exhibits functional stability over at least 500 generations as evidenced by its retained inability to grow when exposed to human serum compared to BP_088 at 0 generations. After 2 hrs in human serum, BP_088 exhibited significantly decreased cfu/mL by about 4 orders of magnitude after about 500 generations.

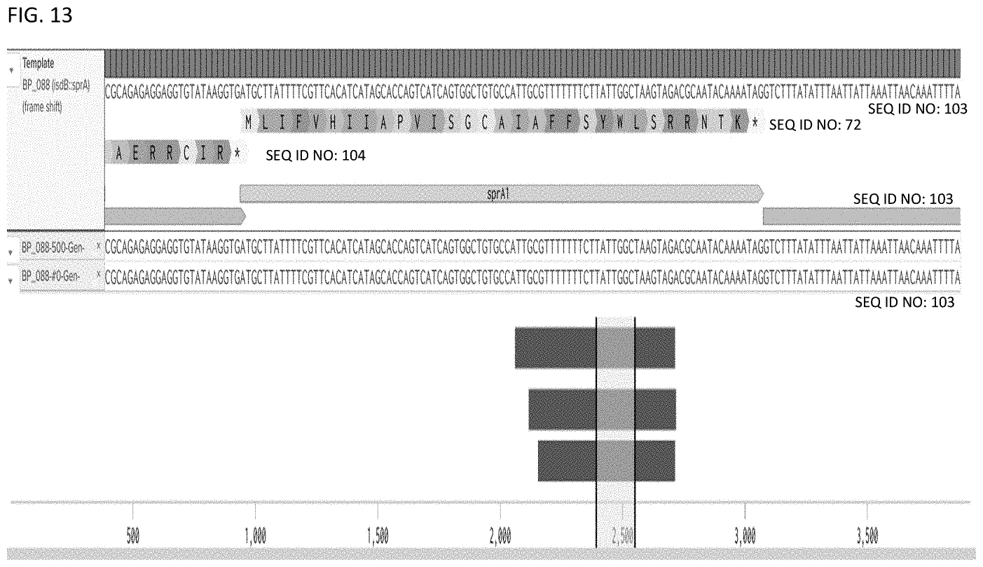

Sanger Sequencing. The isdB::sprA1 insert was PCR amplified from BP_088 for 0 and 500 generation strain streak plates, and sent out for sequencing. The resulting sequencing results were aligned to the BP_088 genomic map. No genetic differences, such as frameshifts or mutations, were seen in the isdB::sprA1 kill switch region. FIG. 13 shows an alignment of a reference sequence for integrated sprA1 kill switch integration behind the isdB gene and the Sanger sequencing results from BP_088 at 0 and 500 generation strains. The top DNA sequence is the reference sequence from a DNA map in Benchling, the middle sequence is from the BP_088 500 generation strain, and the lower sequence is from the BP_088 0 generation strain. The alignment shows no mutations or changes in the bottom two strains when compared to each other or the top reference sequence. Synthetic strain BP_088 exhibits genomic stability over at least 500 generations as evidenced by Sanger sequencing results. Sanger sequencing performed on the isdB::sprA1 integration region revealed there were no genetic differences between BP_088 0 and 500 generation strains in the area sequenced.

De novo sequencing of the entire genome for the BP_088 500 generation strain was also performed. (data not shown).

This example shows that the genomic integration of isdB::sprA1 into BP_001 is stable after roughly 500 generations. This is demonstrated by the serum assay that was run using the BP_088 strain that had been continuously growing for 250 hours. When the assay data is compared to the BP_088 strain that had not undergone the 250 hours of growth, they both had the same response in human serum. Both of the BP_088 strains (0 and 500 generation strains) were unable to grow in human serum in 4 hours, and the viable CFU/mL dropped by over 10⁴ from its starting concentration as shown in FIG. 12.

The stability over at least 500 generations of the integrated kill switch using minimum genomic modification employed in the piggyback method goes far beyond previous publications that attempt to demonstrate evolutionary stability in their integrations. Stirling, Finn, et al. “Rational design of evolutionarily stable microbial kill switches.” Molecular cell 68.4 (2017): 686-697.

### Example 7. Exemplary Plasmid Construction for p262

In this example, preparation of exemplary Plasmid p262 is described. Homology arms were designed to target a sprA1 gene insertion right behind the isdB gene in Staph aureus. Specifically, plasmid p262 is used as an integration vector to insert the sprA1 gene and 24 bases upstream (control arm) in the 5 prime untranslated region into the genome of Staphylococcus aureus directly behind the isdB gene. Plasmid p262 is used to make an isdB::sprA1 integration in Staph aureus genomes, for example, for preparation of synthetic strain BP_118 via the piggyback method.

The backbone of plasmid p262 is pIMAYz, which was designed by Ian Monk et al. to be a shuttle vector for E. coli and Staph aureus epidermidis strains capable of making markerless deletions in both Staph aureus and Staph epidermidis strains. Monk, Ian R. et al., “Complete bypass of restriction systems for major Staphylococcus aureus lineages.” MBio 6.3 (2015): e00308-15; Monk, Ian R. et al., “Transforming the untransformable: application of direct transformation to manipulate genetically Staphylococcus aureus and Staphylococcus epidermidis.” MBio 3.2 (2012): e00277-11.

pIMAYz is temperature sensitive in Staph aureus, meaning it is self replicating when the cells are grown at 30° C., but cannot replicate at temperatures 37° C. or above. The temperature sensitive nature of the plasmid allows for editing of the host's genome using the homologous recombination technique. By adding to the plasmid backbone roughly 1000 base pair (1 kb) regions of homology (homology arms) flanking the DNA location targeted for an insertion or deletion, the plasmid can then be used to make markerless edits to genomic or plasmid DNA in vivo.

In the multiple cloning site on the pIMAYz plasmid, the present inventors added the sprA1 toxin gene and 24 bases upstream of the start codon PCR amplified from genomic DNA of Staph aureus strain BP_001 flanked by homology arms in order to target the integration of the sprA1 fragment directly behind the isdB gene in the genome of Staph aureus. Through a double recombination process, the plasmid is able to fully integrate into the genome of Staph aureus, then remove itself leaving the sprA1 fragment behind the isdB gene in the genome. The present inventors have shown that the sprA1 gene from Staphylococcus aureus is toxic to Staph auerus cells when induced on a high copy plasmid. By analyzing the transcriptome of Staph aureus strain BP_001 during growth in serum and complex media (TSB), it has been shown that the isdB gene has a very low transcript level in TSB media and is highly upregulated in serum.

In order to make a serum inducible kill switch in Staph aureus, the toxic nature of sprA1 expression is operably associated with the highly regulated isdB gene in Staph aureus cells. Plasmid p262 is employed to make this genomic integration.

Materials and Methods

Table 12 shows the single stranded DNA sequences for the primers used during the construction or sequencing of plasmid p262. All of the sequences are in the 5 prime to 3 prime direction.

TABLE 12
Primer sequences used to create and sequence
plasmid p262

| Primer | |
|---|---|
| Name | Primer Sequence (5′→3′) |
| DR_022 | CAAGCTTATCGATACCGTCGACCTC (SEQ ID NO: 117) |
| DR_023 | GGGATCCACTAGTTCTAGAGCGG (SEQ ID NO: 118) |
| DR_116′ | GGGACGTCGTAATACGACTCACTATAGG (SEQ ID |
| | NO: 119) |
| DR_117 | CCAAAGCATAATGGGATAATTAACCCTCACTAAAGGGAA |
| | C (SEQ ID NO: 120) |
| DR_254 | ATGCTTATTTTCGTTCACATCATAGCACCAGTCATCAGTG |
| | (SEQ ID NO: 121) |
| DR_518 | GTGGCGGCCGCTCTAGAACTAGTGGATCCCGTCAATTACG |
| | CAATTAAGGAAATATCAAGG (SEQ ID NO: 122) |
| BP_948 | CCCTCGAGGTCGACGGTATCGATAAGCTTGGATGAGCAAG |
| | TGAAATCAGCTATTAC (SEQ ID NO: 123) |
| BP_949 | CACCTCCTCTCTGCGGATTTATTAGTTTTTACGTTTTCTAG |
| | GTAATAC (SEQ ID NO: 124) |
| BP_950 | AAAAACTAATAAATCCGCAGAGAGGAGGTGTATAAGGTG |
| | ATG (SEQ ID NO: 125) |
| BP_951 | ATTAAATATAAAGACCTATTTTGTATTGCGTCTACTTAGCC |
| | AATAAGAAAAAAAC (SEQ ID NO: 126) |
| BP_952 | CGCAATACAAAATAGGTCTTTATATTTAATTATTAAATTA |
| | ACAAATTTTAATTG (SEQ ID NO: 127) |
| BP_964 | TCAAACTTCAGCAGGTTCTAGC (SEQ ID NO: 128) |
| BP_965 | GTACCAGGTATGACTGAATGCC (SEQ ID NO: 129) |

Table 13 shows the single stranded DNA fragments used in the construction of p262. All fragments used were double stranded DNA. In BP_DNA_003 the sequence in bold is a portion of the reading frame, and the underlined sequence is the control arm.

TABLE 13
DNA Fragments Inserted into pIMAYz Backbone

| Name | Seq. ID | DNA Sequence (5′→3′) |
|---|---|---|
| Upstream | BP_DNA_{—} | GATGAGCAAGTGAAATCAGCTATTACTGAAT |
| Homology | 029 | TCCAAAATGTACAACCAACAAATGAAAAAAT |
| Arm | | GACTGATTTACAAGATACAAAATATGTTGTT |
| | | TATGAAAGTGTTGAGAATAACGAATCTATGA |
| | | TGGATACTTTTGTTAAACACCCTATTAAAAC |
| | | AGGTATGCTTAACGGCAAAAAATATATGGTC |
| | | ATGGAAACTACTAATGACGATTACTGGAAAG |
| | | ATTTCATGGTTGAAGGTCAACGTGTTAGAAC |
| | | TATAAGCAAAGATGCTAAAAATAATACTAGA |
| | | ACAATTATTTTCCCATATGTTGAAGGTAAAA |
| | | CTCTATATGATGCTATCGTTAAAGTTCACGT |
| | | AAAAACGATTGATTATGATGGACAATACCAT |
| | | GTCAGAATCGTTGATAAAGAAGCATTTACAA |
| | | AAGCCAATACCGATAAATCTAACAAAAAAGA |
| | | ACAACAAGATAACTCAGCTAAGAAGGAAGCT |
| | | ACTCCAGCTACGCCTAGCAAACCAACACCAT |
| | | CACCTGTTGAAAAAGAATCACAAAAACAAGA |
| | | CAGCCAAAAAGATGACAATAAACAATTACCA |
| | | AGTGTTGAAAAAGAAAATGACGCATCTAGTG |
| | | AGTCAGGTAAAGACAAAACGCCTGCTACAAA |
| | | ACCAACTAAAGGTGAAGTAGAATCAAGTAGT |
| | | ACAACTCCAACTAAGGTAGTATCTACGACTC |
| | | AAAATGTTGCAAAACCAACAACTGCTTCATC |
| | | AAAAACAACAAAAGATGTTGTTCAAACTTCA |
| | | GCAGGTTCTAGCGAAGCAAAAGATAGTGCTC |
| | | CATTACAAAAAGCAAACATTAAAAACACAAA |
| | | TGATGGACACACTCAAAGCCAAAACAATAAA |
| | | AATACACAAGAAAATAAAGCAAAATCATTAC |
| | | CACAAACTGGTGAAGAATCAAATAAAGATAT |
| | | GACATTACCATTAATGGCATTACTAGCTTTA |
| | | AGTAGCATCGTTGCATTCGTATTACCTAGAA |
| | | AACGTAAAAACTAATAAATC (SEQ ID |
| | | NO: 20) |
| sprA1 | BP_DNA_{—} | CGCAGAGAGGAGGTGTATAAGGTGATGCTTA |
| Fragment | 003 | TTTTCGTTCACATCATAGCACCAGTCATCAG |
| (in- | | TGGCTGTGCCATTGCGTTTTTTTCTTATTGG |
| sertion | | CTAAGTAGACGCAATACAAAATAG (SEQ |
| sequence) | | ID NO: 3) |
| Down- | BP_DNA_{—} | GTCTTTATATTTAATTATTAAATTAACAAAT |
| stream | 002 | TTTAATTGGCGGATGAGGTATCCAGTTACCT |
| Homology | | CGTTCGCCAATTATTTTTCGCAATATAAAAA |
| Arm | | GTCCCACTTAAAACAATCATTTTAAGCGGGA |
| | | CTTTTTATATTGAGTAACTAAAATTATTTAG |
| | | CTGCTACTTCTTCGCCATTGTAAGAACCACA |
| | | GTTTTTACATACACGGTGTGATAATTTGTAT |
| | | TCGCCACAGTTTGGGCATTCAGTCATACCTG |
| | | GTACTGAAATTTTGAAATGCGTACGACGTTT |
| | | GTTTTTTCTAGTTTTAGAAGTTCTTCTTTTT |
| | | GGTACTGCCATGATATATCCTCCTTAGATTA |
| | | TAAACGAAAAATACTAAATGTTAGTTTAATT |
| | | AACAACATTATATCATTAATTAAACTACTTA |
| | | TTGCTCTTTATCATATAATTGTTGTAATTTT |
| | | TGAAGCCTTGGATCAACTTGTCGTGATTCTG |
| | | AATCATCTTGTTCTTGCTGTTTAGCAAGCTC |
| | | ATCTAATTGATCCTCATCGATTACTTCCCAA |
| | | CCATTACCTACTGTCAACATTTGGTCACTTT |
| | | GCTCTGAATAAGCTCTCATTGGTTTCTCAAT |
| | | AATAACTATATCCTCGACAATATCCTGAAGA |
| | | TTAACCATACCATCTTTAATAATGTGATAGT |
| | | GTTCATCTACATCATCTTGATCATCGTTATA |
| | | CTGATTGTACCCTTCTAAATCAAATACTTCT |
| | | GTAGTAGTTACATCTAGTGGGACTTTTACTG |
| | | GTACAAGAGTACGTGCACAAGGCATTGTATA |
| | | CGTTCCAGTAATGTGAATATCCGCAACGACT |
| | | TCTGTTGACTTAATGGTTAACTGACCTTGGA |
| | | TTGTAATTGGAGATAAATCAATTAAATCTAA |
| | | TGATTCTTTTAAATTGTCAAAACTCACCGTT |
| | | TGATCAAATTCAAATGGCTTACCTTGATATT |
| | | TCCTTAATTGCGTAATTGAC (SEQ ID |
| | | NO: 2) |

PCR Fragment Generation
1. The following PCR reactions were performed using Q5 High Fidelity Hot Start 2× Master Mix (NEB) per the manufacturer's instructions:
   1.1. BP_DNA_063—pIMAYz Backbone Fragment
      1.1.1. DR_022/DR_023
   1.2. BP_DNA_010—Upstream Homology Arm
      1.2.1. BP_948/BP_949
   1.3. BP_DNA_003—sprA1 (Inserted sequence)
      1.3.1. BP_950/BP_951
   1.4. BP_DNA_002—Downstream Homology Arms
      1.4.1. BP_952/DR_518
2. The above PCR fragments were checked on a 1% agarose gel to confirm a clean band of proper length, and then purified using a Qiaquick PCR Purification Kit (Qiagen) per the manufacturer's instructions.
3. The pIMAYz fragment was treated with DpnI (NEB) to remove the methylated circular plasmid used as the template for the PCR, and purified again using the Qiaquick PCR Purification Kit (Qiagen).
4. The 4 fragments were used in a Gibson Assembly (NEB) to create a circular plasmid per the manufacturer's instructions.
5. The assembled plasmid was then transformed into our E. coli pass-through strain IMO8B per the transformation protocol in Report_SOP030, plated on LB (chlor/X-gal), and incubated overnight at 37° C.
6. The following day the blue colonies were screened for fully assembled plasmids by colony PCR and was then checked on a 1% agarose gel.
   6.1. DR_116′/DR_254 were used for colony screen.
7. Three positive colonies were chosen as the template for a high fidelity PCR reaction using Q5 Hot Start 2× Master Mix (NEB) using primers DR_116′ and DR_117 which bind to the plasmid backbone and capture the whole insertion region. The PCR was then checked on a 1% agarose gel for correct size and cleanliness. The PCR product was then purified using the Qiaquick PCR Purification Kit (Qiagen), and sent to Quintara Biosciences to be sequenced.
8. The sequencing was aligned in silico using the sequence alignment tool in the molecular biology platform on the Benchling platform.
   8.1. One positive colony that showed optimal alignment was chosen to be stocked in the plasmid database per the protocol in Report_SOP028 Preparing Strain and Plasmid Stocks.

Results

All PCR fragments were amplified successfully from a crude genomic prep of the strain BP_001. After assembling them into a circular plasmid and transforming into IMO8B, several colonies screened showed that the plasmid contained all of the desired fragments.

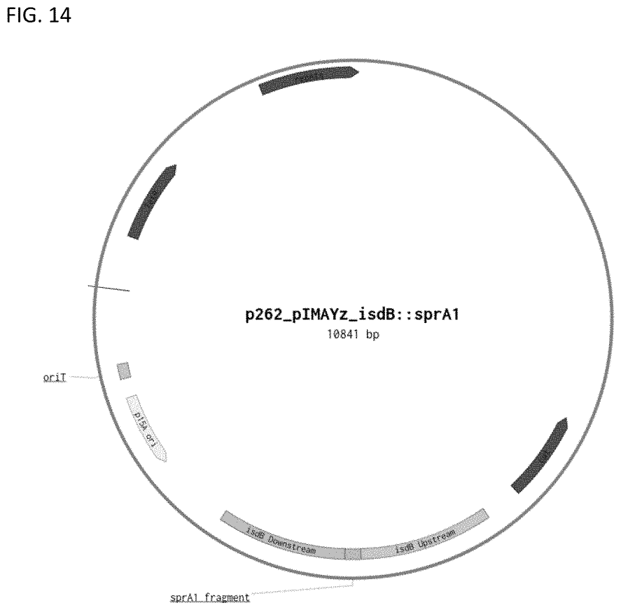

The sequencing results showed no mutations. The data for the sequences and alignment is stored in BioPlx's Benchling account. FIG. 14 shows a map of the p262 plasmid made in the Benchling program. The plasmid features a pIMAYz backbone with the integration of a sprA1 gene fragment flanked by isdB homology arms.

The isdB homology arms and sprA1 gene were PCR amplified from the Staph aureus gDNA (BP_001) and then assembled into the pIMAYz backbone in the multiple cloning site. This allows p262 to be used for markerless insertion of the sprA1 gene right behind the isdB gene in Staph aureus strains that are genetically similar to the BP_001 strain in that region of the genome. The plasmid genotype was successfully confirmed by sequencing. Sanger sequencing of the constructed plasmid indicated no deletions in the homology arms or sprA1 region. The plasmid is named p262 (pIMAYz_isdB::sprA1).

Several other plasmids were constructed in a similar fashion using the pIMAYz backbone, as shown in Table 7.

### Example 8. qRT PCR for Genomic Expression of Serum-Responsive Promoters

In this report, qRT PCR is performed for 20 or 23 S. aureus genes found in the literature to be blood and/or serum responsive. Briefly, 502a (BP_0001) cells were grown in TSB media, blood, or serum, and RNA was extracted at various time points. The results show several genes that are upregulated in blood or serum.

The procedure for investigating gene expression by mRNA level comprises extracting total RNA, removing residual DNA, and converting the single-stranded mRNA to double-stranded DNA (complementary, or cDNA). During this conversion to cDNA, RNA samples from the same experiment are generally normalized to the same concentration, such that each cDNA sample is created from the same amount of RNA.

502a glycerol stock was struck onto a fresh bacterial plate and grown overnight. 3-5 single colonies from the plate were inoculated into a 4 ml culture of BHI media and grown overnight at 37° C. with shaking at 240 rpm. In the morning, the culture was diluted to an optical density (OD) of 0.05 in 5 ml fresh BHI media. Cells were grown at 37° C. with shaking at 150 rpm for several hours to an OD of approximately 1. At this time, samples for RNA were collected for a T=0 time point (1 ml was transferred to a 1.5 ml microcentrifuge tube, centrifuged at 16,000 rpm for 1 minute, supernatant dumped, cells resuspended in 1 ml sterile PBS, centrifuged at 16,000 rpm for 1 minute, supernatant aspirated, cells resuspended in 200 ul RNALater, and stored at −20° C.). The remaining culture was rediluted to an OD of 0.05 in 3 replicate heparinized tubes of 10 ml fresh BHI media or thawed human serum, and incubated at 37° C. with shaking at 150 rpm. Additional samples for RNA were collected at T=90 minutes, and T=180 minutes. For these later samples, one 10 ml tube was centrifuged at 3,000 rpm for 10 minutes, supernatant dumped, cells resuspended in 1 ml PBS, transferred to a 1.5 ml microcentrifuge tube, centrifuged at 16,000 rpm for 1 minute, supernatant aspirated, cells resuspended in 200 ul RNALater, and stored at −20° C.

qPCR Sample Processing and Data Analysis was performed as follows. RNA extraction and cDNA synthesis was performed on 10/8/18. Frozen RNA pellets stored in RNALater were washed once in PBS, extracted using Ambion RiboPure Bacteria kit and eluted in 2×50 ul. RNA samples were DNased using Ambion Turbo DNase kit. Samples with a final concentration less than 50 ng/ul were ethanol precipitated to concentrate DNA. 500 ng of DNased RNA was used in Applied Biosystems High-Capacity cDNA Reverse Transcription kit. qPCR was performed with Applied Biosystems PowerUp SYBR Green Master Mix (10 ul reaction with 1 ul of cDNA).

Samples were probed to look for changes in gene expression over time and in different media, and normalized to housekeeping genes, gyrB, sigB, rho, or an average of the three, using the ΔΔCt method. Table 14 shows qRT-PCR primers used for S. aureus 502A. Ct (cycles to threshold) values for housekeeping gene transcripts were subtracted from Ct values for gene transcripts for each RNA sample. These ΔCt values were then normalized to the initial time point.

TABLE 14
qRT-PCR Primer Table for S. aureus 502a

| Gene | qRT PCR Primers | |
|---|---|---|
| gyrB | BPC802- | BPC803-TCCATCCACATCGGCATCAG |
| | TTGGTACAGGAATCGGTGGC | (SEQ ID NO: 131) |
| | (SEQ ID NO: 130) | |
| isdA | BPC114- | BPC115- |
| | GCAACAGAAGCTACGAACGC | AGAGCCATCTTTTTGCACTTGG (SEQ |
| | (SEQ ID NO: 132) | ID NO: 133) |
| isdB | BPC116- | BPC117- |
| | GCAACAATTTTATCATTATGCCAG | TGGCAACTTTTTGTCACCTTCA (SEQ |
| | C (SEQ ID NO: 134) | ID NO: 135) |
| isdI | BPC764- | BPC765- |
| | ACCGAGGATACAGACGAAGTT | TGCTGTCCATCGICATCACTT (SEQ |
| | (SEQ ID NO: 136) | ID NO: 137) |
| isdG | BPC120- | BPC121-AGGCTTTGATGGCATGTTTG |
| | AACCAATCCGTAAAAGCTTGC | (SEQ ID NO: 139) |
| | (SEQ ID NO: 138) | |
| sbnC | BPC768- | BPC769-TCAGTCCTTCTTCAACGCGA |
| | AGGGAAGGGTGTCTAAGCAAC | (SEQ ID NO: 141) |
| | (SEQ ID NO: 140) | |
| sbnE | BPC124- | BPC125-GCAACTTGTAGCGCATCGTC |
| | ATTCGCTTTAGCCGCAATGG (SEQ | (SEQ ID NO: 143) |
| | ID NO: 142) | |
| IrgA | BPC126- | BPC127- |
| | GATACCGGCTGGTACGAAGAG | TGGTGCTGTTAAGTTAGGCGA (SEQ |
| | (SEQ ID NO: 144) | ID NO: 145) |
| IrgB | BPC128- | BPC129- |
| | ACAAAGACAGGCACAACTGC | GGTGTAGCACCAGCCAAAGA (SEQ |
| | (SEQ ID NO: 146) | ID NO: 147) |
| hlgB | BPC760- | BPC761-GGCATTTGGTGTTGCGCTAT |
| | TGGTTGGGGACCTTATGGAAG | (SEQ ID NO: 149) |
| | (SEQ ID NO: 148) | |
| fhuA | BPC132- | BPC133- |
| | CACGTTGTCTTTGACCACCAC | TGGGCAATGGAAGTTACAGGA |
| | (SEQ ID NO: 150) | (SEQ ID NO: 151) |
| fhuB | BPC134- | BPC135- |
| | CAATACCTGCTGGAACCCCA (SEQ | GGGTCCGCATATTGCCAAAC (SEQ |
| | ID NO: 152) | ID NO: 153) |
| ear | BPC136- | BPC137- |
| | CCACTTGTCAGATCTGCTCCT | GGTTTGGTTACAGATGGACAAACA |
| | (SEQ ID NO: 154) | (SEQ ID NO: 155) |
| fnb | BPC772- | BPC773-TTGGTCCTTGTGCTTGACCA |
| | CGCAGTGAGCGACCATACA (SEQ | (SEQ ID NO: 157) |
| | ID NO: 156) | |
| hlb | BPC140- | BPC141-ACACCTGTACTCGGTCGTTC |
| | CTACGCCACCATCTTCAGCA (SEQ | (SEQ ID NO: 159) |
| | ID NO: 158) | |
| splF | BPC142- | BPC143- |
| | TGCAATTATTCAGCCTGGTAGC | CCTGATGGCTTATTACCGGCAT |
| | (SEQ ID NO: 160) | (SEQ ID NO: 161) |
| splD | BPC144- | BPC145- |
| | AGTGACATCTGATGCGGTTG (SEQ | AACACCAATTGCTTCTCGCTT (SEQ |
| | ID NO: 162) | ID NO: 163) |
| dps | BPC146- | BPC147- |
| | AGCGGTAGGAGGAAACCCTG | GTTCTGCAGAGTAACCTTTCGC |
| | (SEQ ID NO: 164) | (SEQ ID NO: 165) |
| srtB | BPC846- | BPC847- |
| | TGAGCGAGAACATCGACGTAA | CCGACATGGTGCCCGTATAA (SEQ |
| | (SEQ ID NO: 166) | ID NO: 167) |
| emp | BPC854- | BPC855- |
| | TCGCGTGAATGTAGCAACAAA | ACTTCTGGGCCTTTAGCAACA (SEQ |
| | (SEQ ID NO: 168) | ID NO: 169) |
| sbnA | BPC858- | BPC859- |
| | CCTGGAGGCAGCATGAAAGA | CATTGCCAACGCAATGCCTA (SEQ |
| | (SEQ ID NO: 170) | ID NO: 171) |
| CH52_360 | BPC834- | BPC835-TTGCACCCATTGTTGCACCT |
| | TTCAACTCGAACGCTGACGA | (SEQ ID NO: 173) |
| | (SEQ ID NO: 172) | |
| CH52_305 | BPC838- | BPC839- |
| | TTCCTGGAGCAGTACCACCA (SEQ | CAGCGCAATCGCTGTTAAACTA |
| | ID NO: 174) | (SEQ ID NO: 175) |
| CH521670 | BPC842- | BPC843- |
| | GCGATTATGGGACCAAACGG | ACTTCATAGCTTGGGTGTCCC (SEQ |
| | (SEQ ID NO: 176) | ID NO: 177) |
| clfA | BPC850- | BPC851-TAGCTTCACCAGTTACCGGC |
| | TCCAGCACAACAGGAAACGA | (SEQ ID NO: 179) |
| | (SEQ ID NO: 178) | |
| SAUSA300_{—} | BPC778- | BPC779- |
| 2268 | GCTTCTACAGCTTTGCCGAT (SEQ | GATTTGGTGCTTACTGCCACC (SEQ |
| | ID NO: 180) | ID NO: 181) |
| SAUSA300_{—} | BPC774- | BPC775- |
| 2616 | ACAAGCGCAACAAGCAAGAG | TGCGTTTGATACCTTTAACACGG |
| | (SEQ ID NO: 182) | (SEQ ID NO: 183) |
| SAUSA300_{—} | BPC152- | BPC153-ACGCGTTGTTTTTGACCTCC |
| 2617 | GGGCTGAAAAAGTTGGCATGA | (SEQ ID NO: 185) |
| | (SEQ ID NO: 184) | |
| hlgA2 | BPC179- | BPC180-AGCCCCTTTAGCCAATCCAT |
| | TGATTTCTGCACCTTGACCGA | (SEQ ID NO: 187) |
| | (SEQ ID NO: 186) | |
| hrtAB | BPC713- | BPC714-TAACGGTGCTTGCTCTGCTT |
| | ACACAACAACAACGTGATGAGC | (SEQ ID NO: 189) |
| | (SEQ ID NO: 188) | |

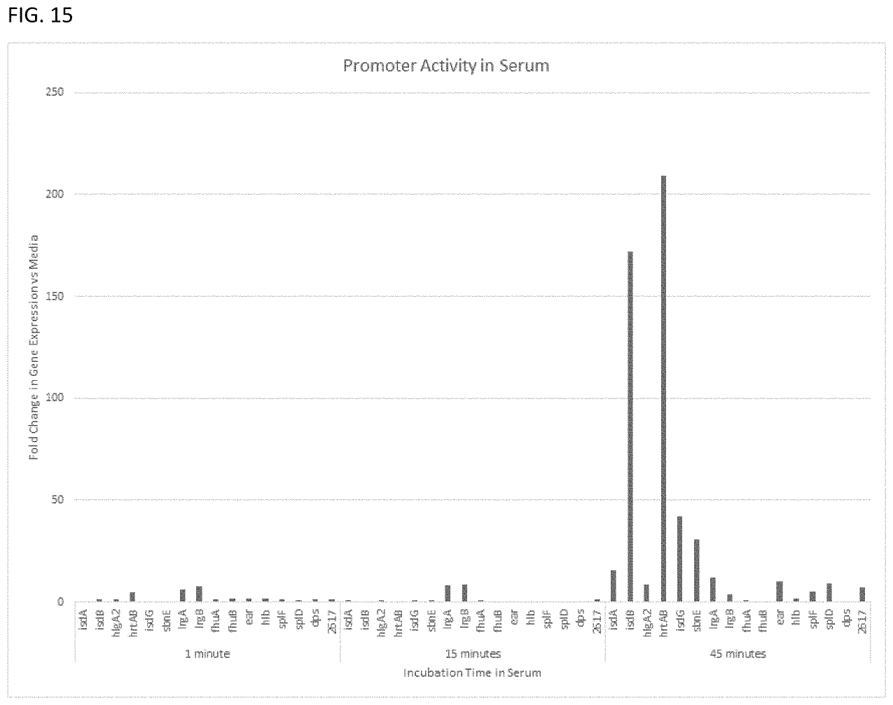

qPCR Results or candidate blood an serum responsive promoter genes are shown in FIGS. 15-17. FIG. 15 shows promoter activity in serum compared to TSB at 15 min, 30 min or 45 min time points. In human serum at 45 min, upregulated genes are shown in Table 15. Upregulated genes at 45 min in human serum include hlgA2, hrtAB, isdA, isdB, isdG, sbnE, ear, splD, and SAUSA300_2617.

TABLE 15
Upregulated serum-responsive S. aureus genes

| Upregulated Gene | Fold Change in Serum at T = 45 |
|---|---|
| hlgA2 | 9 |
| hrtAB | 209 |
| isdA | 15 |
| isdB | 172 |
| isdG | 42 |
| sbnE | 30 |
| ear | 10 |
| splD | 9 |
| SAUSA300_2617 | 7 |

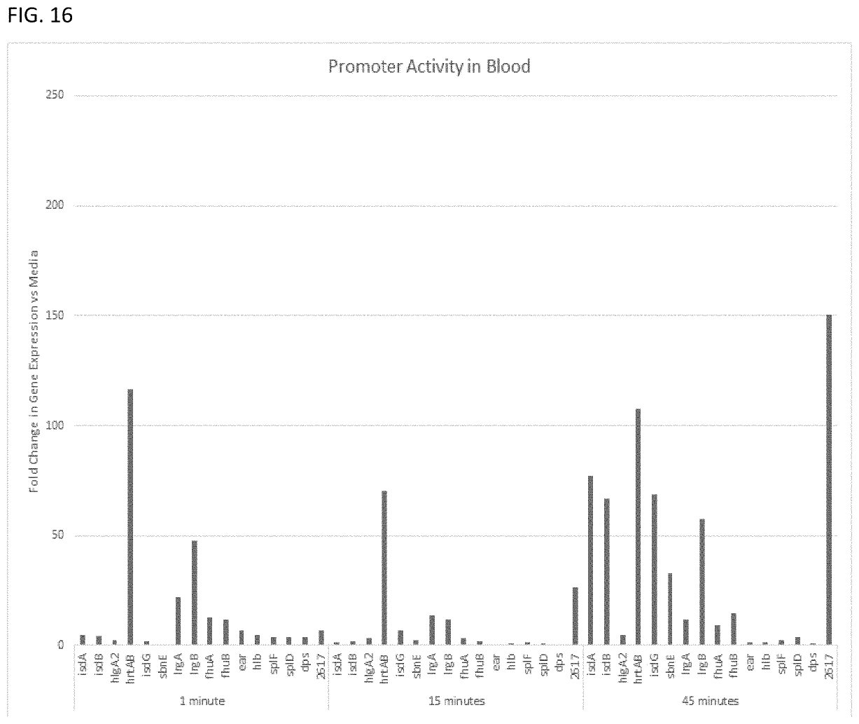

FIG. 16 shows promoter activity in human blood compared to TSB at 15 min, 30 min or 45 min time points. In human blood at 45 min, upregulated genes are shown in Table 16. Upregulated genes at 45 min in human blood include isdA, isdB, isdG, sbnE, and SAUSA300_2617.

TABLE 16
Upregulated blood-responsive S. aureus genes

| Upregulated Gene | Fold Change in Blood at T = 45 |
|---|---|
| isdA | 77 |
| isdB | 66 |
| isdG | 69 |
| sbnE | 33 |
| SAUSA300_2617 | 150 |

FIG. 17 shows several candidate genes that are upregulated after 90 minutes of incubation in serum. Specifically, genes in the isd, sbn, and fhu families are upregulated to varying degrees. Gene expression at 90 minutes in both TSB and serum were normalized to values at T=0. All of the genes surveyed here exhibited stable expression from T=0 to T=90 minutes in TSB.

Several genes from this example show high upregulation in serum, while others show stable expression in serum. Both of these characteristics may be useful to tune kill switch activity. For example, in order to generate a synthetic microorganism that will not have the ability to grow in human systemic conditions, a toxin gene may be placed under control of a promoter that will upregulate in serum, and an antitoxin gene may be placed under control of a promoter that will downregulate or remain stable in serum. In another example, the promoter region for each upregulated or stable gene of interest may be identified and cloned in front of a toxin such as sprA1, or an antitoxin. Promoter genes may also be cloned in front of a reporter gene, such as GFP, to determine expression at the protein level.

### Example 9. Pass Through E. coli Strain

In this example, an E. coli pass through strain is constructed for assembling plasmids that are used for integrating or using toxin genes in Staph aureus strains. The technique of adding antitoxins to E. coli passthrough strains for the purpose of suppressing leaky heterologous toxin gene expression from assembled plasmids in E. coli is described. Plasmids isolated from the pass through strain can be directly transformed into target strains of S. aureus and S. epidermidis.

Challenges often arise when trying to work with wild type bacterial strains. In the case of Staphylococcus aureus, the challenges include bypassing their extra thick peptidoglycan cell wall along with endogenous restriction modification systems that cut up all DNA that is not properly methylated. By making highly concentrated plasmid DNA harvested from an E. coli passthrough strain specially designed to help bypass the restriction systems, the present inventors were able to solve both challenges.

The strong restriction barrier present in Staphylococcus aureus and Staphylococcus epidermidis previously limited functional genomic analysis to a small subset of strains that are amenable to genetic manipulation. A conserved type IV restriction system termed SauUSI which specifically recognizes cytosine methylated DNA was identified as the major barrier to transformation with foreign DNA. Monk et al. 2012 previously constructed a DNA cytosine methyltransferase mutant in the high-efficiency Escherichia co/i K12 cloning strains. Monk et al., 2012, mBio 3(2):e00277-11. doi:10.1128/mBio.00277-11.

Methods of the disclosure comprise genomic integrations via homologous recombination to manipulate toxin genes in the genome of Staph aureus, so the toxin genes are present on the plasmids used to make the edits. This means that the E. coli passthrough strain also needs to be resistant to the toxins present on the plasmids used for homologous recombination in Staph. The toxins may have leaky expression from the plasmid in E. coli, and unless they are able to be controlled they will kill the E. coli cells rendering the cells unable to produce circular plasmid to be harvested and transformed into the Staph strains. To solve this for the sprA1 toxin gene a copy of the sprA1 antitoxin, sprA1 antisense (sprA1_{AS}), was integrated into the genome of the E. coli IM08B strain under the expression of the native promoter in Staph. This allowed transformation of assembled plasmids containing the sprA1 toxin gene in E. coli. Prior to the antisense sprA1 integration in E. coli the present inventors were unable to replicate any useful integration plasmid containing the sprA1 or sprA2 toxin genes. Following the antisense integration the present inventors were able to transform and replicate plasmids with the sprA1 and sprA2 toxin genes, such as p249.

There are three main reasons for constructing this pass-through strain to assemble plasmids that can be used for integrating or using toxin genes in Staph aureus strains. Staph strains are notoriously hard to transform and require large quantities of clean plasmid DNA in order to get transformants. Staph aureus strains have endogenous restriction modification systems that can detect and degrade DNA that is not properly methylated, so the large DNA prep must also be properly methylated before transforming into Staph strains. Monk, Ian R., et al. “Complete bypass of restriction systems for major Staphylococcus aureus lineages.” MBio 6.3 (2015): e00308-15. When assembling plasmids containing genes that are toxic to the pass-through strain, some regulation must be used in order to control the expression of the toxins so they don't kill the pass-through strain under normal growth conditions for producing the plasmids.

To obtain sufficient amounts of plasmid DNA and assure the DNA is properly methylated, Ian Monk created a strain of E. coli (K12) named IM08B that has some of the methylation enzymes integrated into the genome. To overcome the toxin regulation problem, the strain IM08B was given another genomic modification where the antitoxin to the sprA1 toxin peptide PepA1, sprA1 antisense (sprA1_{AS}) along with its native promoter region was taken from the Staph aureus strain BP_001 and integrated into the genome of IM08B. This integration allowed IM08B to be used for the production of plasmid DNA in containing sprA1 and sprA2 toxins to be produced in sufficient quantities to be transformed into Staph aureus strains. The new strain created with this modification is called BPEC_001.

Table 17 shows the plasmids used in this example.

TABLE 17
Plasmids for Pass Through Strain

| | |
|---|---|
| pKD46 | Plasmid containing RED genes under control of the pBAD |
| | promoter, used to make genomic edits in E. coli. |
| pCN51 | Low copy expression shuttle vector (E. coli and |
| | Staph aureus) with kanamycin resistance |

Table 18 shows the primers and their sequences used to build and verify proper construction of the strain BPEC_001.

TABLE 18
Primers for E. coli Pass Through Strain

| Name | Sequence (5′→3′) |
|---|---|
| DR_357 | GAGTTGTTGATGGCTAAGTAGACGCAATACAAAATAGGTG |
| | (SEQ ID NO: 190) |
| DR_410 | CCTGGGTACCAGTCATCAAGCACAGTTTGACTGGAAAG |
| | (SEQ ID NO: 191) |
| DR_359 | GGAACCGATTGAAGGGATTCATTTCGTTG (SEQ ID NO: |
| | 192) |
| DR_409 | CTCGGTTGCTGTGTTGCACACAGTTATCTGTGAG (SEQ |
| | ID NO: 193) |
| DR_361 | TGCGTCTACTTAGCCATCAACAACTCTCCTGGCGCACCATCG |
| | (SEQ ID NO: 194) |
| DR_362 | GTTTCAGGGTTTGCAGACTGATATTCAATGACG (SEQ ID |
| | NO: 195) |
| DR_371 | ACATAGCGCACGTAGAACAACGACG (SEQ ID NO: 196) |
| DR_372 | GCCATCTGTAAATCTTGCGCCATTAGTCC (SEQ ID NO: |
| | 197) |
| DR_407 | GTGTGCAACACAGCAACCGAGCGTTCTGAACAAATCCAG |
| | (SEQ ID NO: 198) |
| DR_408 | GTGCTTGATGACTGGTACCCAGGAAACAGCTATGACCATG |
| | (SEQ ID NO: 199) |
| DR_117 | Ccaaagcataatgggataattaaccctcactaaagggaac |
| | (SEQ ID NO: 200) |
| DR_228 | CTATTTTGTATTGCGTCTACTTAGCCAATAAG (SEQ ID |
| | NO: 201) |
| DR_116 | Ccctgttgataccgggaagccctggg (SEQ ID NO: |
| | 202) |

Table 19 shows DNA sequences used to build the BPEC_001 strain described in this report.

TABLE 19
DNA Sequences for BPEC_001 strain construction

| | Seq. | |
|---|---|---|
| Name | ID | DNA Sequence (5′→3′) |
| kanR | BP_{—} | GTACCCAGGAAACAGCTATGACCATGTAATAC |
| Fragment | DNA_{—} | GACTCACTATACGGGGATATCGTCGGAATTGC |
| | 015 | CAGCTGGGGCGCCCTCTGGTAAGGTTGGGAAG |
| | | CCCTGCAAAGTAAACTGGATGGCTTTCTTGCC |
| | | GCCAAGGATCTGATGGCGCAGGGGATCAAGAT |
| | | CTGATCAAGAGACAGGATGAGGATCGTTTCGC |
| | | ATGATTGAACAAGATGGATTGCACGCAGGTTC |
| | | TCCGGCCGCTTGGGTGGAGAGGCTATTCGGCT |
| | | ATGACTGGGCACAACAGACAATCGGCTGCTCT |
| | | GATGCCGCCGTGTTCCGGCTGTCAGCGCAGGG |
| | | GCGCCCGGTTCTTTTTGTCAAGACCGACCTGT |
| | | CCGGTGCCCTGAATGAACTGCAGGACGAGGCA |
| | | GCGCGGCTATCGTGGCTGGCCACGACGGGCGT |
| | | TCCTTGCGCAGCTGTGCTCGACGTTGTCACTG |
| | | AAGCGGGAAGGGACTGGCTGCTATTGGGCGAA |
| | | GTGCCGGGGCAGGATCTCCTGTCATCTCACCT |
| | | TGCTCCTGCCGAGAAAGTATCCATCATGGCTG |
| | | ATGCAATGCGGCGGCTGCATACGCTTGATCCG |
| | | GCTACCTGCCCATTCGACCACCAAGCGAAACA |
| | | TCGCATCGAGCGAGCACGTACTCGGATGGAAG |
| | | CCGGTCTTGTCGATCAGGATGATCTGGACGAA |
| | | GAGCATCAGGGGCTCGCGCCAGCCGAACTGTT |
| | | CGCCAGGCTCAAGGCGCGCATGCCCGACGGCG |
| | | AGGATCTCGTCGTGACCCATGGCGATGCCTGC |
| | | TTGCCGAATATCATGGTGGAAAATGGCCGCTT |
| | | TTCTGGATTCATCGACTGTGGCCGGCTGGGTG |
| | | TGGCGGACCGCTATCAGGACATAGCGTTGGCT |
| | | ACCCGTGATATTGCTGAAGAGCTTGGCGGCGA |
| | | ATGGGCTGACCGCTTCCTCGTGCTTTACGGTA |
| | | TCGCCGCTCCCGATTCGCAGCGCATCGCCTTC |
| | | TATCGCCTTCTTGACGAGTTCTTCTGAGCGGG |
| | | ACTCTGGGGTTCGAGAGCTCGCTTGGACTCCT |
| | | GTTGATAGATCCAGTAATGACCTCAGAACTCC |
| | | ATCTGGATTTGTTCAGAACGCTCGGTTG |
| | | (SEQ ID NO: 203) |
| ΔuidA | BP_{—} | GGAACCGATTGAAGGGATTCATTTCGTTGACT |
| Upstream | DNA_{—} | ATATGGTCGAGTCCATTGTCTCTCTCACCCAT |
| Homology | 016 | GAAGCCTTTGGACAACGGGCGCTGGTGGTTGA |
| Arm | | AATTATGGCGGAAGGGATGCGTAACCCACAGG |
| | | TCGCCGCCATGCTTAAAAATAAGCATATGACG |
| | | ATCACGGAATTTGTTGCCCAGCGGATGCGTGA |
| | | TGCCCAGCAAAAAGGCGAGATAAGCCCAGACA |
| | | TCAACACGGCAATGACTTCACGTTTACTGCTG |
| | | GATCTGACCTACGGTGTACTGGCCGATATCGA |
| | | AGCGGAAGACCTGGCGCGTGAAGCGTCGTTTG |
| | | CTCAGGGATTACGCGCGATGATTGGCGGTATC |
| | | TTAACCGCATCCTGATTCTCTCTCTTTTCGGC |
| | | GGGCTGGTGATAACTGTGCCCGCGTTTCATAT |
| | | CGTAATTTCTCTGTGCAAAAATTATCCTTCCC |
| | | GGCTTCGGAGAATTCCCCCCAAAATATTCACT |
| | | GTAGCCATATGTCATGAGAGTTTATCGTTCCC |
| | | AATACGCTCGAACGAACGTTCGGTTGCTTATT |
| | | TTATGGCTTCTGTCAACGCTGTTTTAAAGATT |
| | | AATGCGATCTATATCACGCTGTGGGTATTGCA |
| | | GTTTTTGGTTTTTTGATCGCGGTGTCAGTTCT |
| | | TTTTATTTCCATTTCTCTTCCATGGGTTTCTC |
| | | ACAGATAACTGTGTGCAACACAG (SEQ ID |
| | | NO: 13) |
| ΔuidA | BP_{—} | ATCAACAACTCTCCTGGCGCACCATCGTCGGC |
| Down- | DNA_{—} | TACAGCCTCGGTGACGTCGCCAATAACTTCGC |
| stream | 017 | CTTCGCAATGGGGGCGCTCTTCCTGTTGAGTT |
| Homology | | ACTACACCGACGTCGCTGGCGTCGGTGCCGCT |
| Arm | | GCGGCGGGCACCATGCTGTTACTGGTGCGGGT |
| | | ATTCGATGCCTTCGCCGACGTCTTTGCCGGAC |
| | | GAGTGGTGGACAGTGTGAATACCCGCTGGGGA |
| | | AAATTCCGCCCGTTTTTACTCTTCGGTACTGC |
| | | GCCGTTAATGATCTTCAGCGTGCTGGTATTCT |
| | | GGGTGCTGACCGACTGGAGCCATGGTAGCAAA |
| | | GTGGTGTATGCATATTTGACCTACATGGGCCT |
| | | CGGGCTTTGCTACAGCCTGGTGAATATTCCTT |
| | | ATGGTTCACTTGCTACCGCGATGACCCAACAA |
| | | CCACAATCCCGCGCCCGTCTGGGCGCGGCTCG |
| | | TGGGATTGCCGCTTCATTGACCTTTGTCTGCC |
| | | TGGCATTTCTGATAGGACCGAGCATTAAGAAC |
| | | TCCAGCCCGGAAGAGATGGTGTCGGTATACCA |
| | | TTTCTGGACAATTGTGCTGGCGATTGCCGGAA |
| | | TGGTGCTTTACTTCATCTGCTTCAAATCGACG |
| | | CGTGAGAATGTGGTACGTATCGTTGCGCAGCC |
| | | GTCATTGAATATCAGTCTGCAAACCCTGAAAC |
| | | (SEQ ID NO: 14) |
| PsprA1 | BP_{—} | CAGTCATCAAGCACAGTTTGACTGGAAAGAAG |
| (as)- | DNA_{—} | GCATTAACTTTAAAACGAAGGATAATCAAATG |
| sprA1(as) | 018 | GTCCTTTAGAAGGGATAAACAACAAAATAAAA |
| | | TTAATTAAACGTACATCTTTTGGTTAAGGAAG |
| | | TTATAATCATTTGCGAAATCGAATATTATTAT |
| | | GTTCAAAACTTTACGCTCCAAAAAGTAAAAAG |
| | | GAAGCTAAGCAATGTTTAGTTGCCTAACTTCC |
| | | GATATTGAACTCATCAGGCCAATTTGGCATAG |
| | | AGCCTTTTTTAGTTCTTGATGTTTCTCTTTAA |
| | | AACCTTGCATATTTTACAAAGAGAAAGATTAG |
| | | CAGTATAATTGAGATAACGAAAATAAGTATTT |
| | | ACTTATACACCAATCCCCTCACTATTTGCGGT |
| | | AGTGAGGGGATTTTTATTGGTGCGGCTATATG |
| | | TCACCTATTTTGTATTGCGTCTACTTAGCC |
| | | (SEQ ID NO: ) |

Integration Cassette Construction

To make the DNA which will be inserted into the E. coli genome, 4 separate PCR products were generated. Two ˜500 bp homology arms were amplified by PCR from E. coli K12 genomic DNA (gDNA), a fragment containing the kanR gene was amplified from plasmid DNA, and the sprA1_{AS} gene and promoter were amplified by PCR from BP_001 gDNA. The fragment generation and stitch PCR were performed with the Q5 Hot Start Polymerase (NEB) per the manufacturer's instructions and the stitch PCR conditions outlined in Report_SOP036.

PCR Fragment Generation

The primers used to generate the PCR fragments to be stitched together and integrated into the E. coli genome are stated below:
1) Upstream Homology Arm (E. coli gDNA used as template)
   a) DR_359/DR_409
2) PsprA1(as)-sprA1(as) (BP_001 gDNA used as template)
   a) DR_357/DR_410
3) kanR fragment (plasmid pCN51 used as template)
   a) DR_408/DR_407
4) Downstream Homology Arm (E. coli gDNA used as template)
   a) DR_362/DR_361
5) All PCR products were visualized using a blue LED transilluminator and purified using a PCR cleanup kit (Qiagen).

Assembly by Stitch PCR
1) 0.15 pmol of the sprA1_{AS} fragment was combined with 0.15 pmol of the Upstream HA fragment, and 0.15 pmol of the kanR fragment was combined with 0.15 pmol of the Downstream HA fragment.
2) Molecular biology grade water was added to make a total volume of 20 μL.
3) 20 μL of 2× Q5 Hot Start Master Mix (NEB) was added to the PCR fragment cocktail from Steps 1 -2 above.
4) The PCR mixture was put in a thermal cycler using the following conditions
   a) Initial Denaturation @ 98° C. for 30 seconds
   b) DNA Denature @ 98° C. for 5 seconds
   c) Anneal @ 68° C. for 30 seconds
   d) Extension @ 72° C. for 20 seconds
   e) Cycle steps b-d 10 times
   f) Final Extension @ 72° C. for 2 minutes
5) 1 μL from the above reaction was used as the template for a second PCR reaction under normal cycling conditions for Q5 Hot Start Master Mix (NEB).
   a) Downstream HA+sprA1_{AS} Fragment
      i) DR_362/DR_410
   b) Upstream HA+kanR Fragment
      i) DR_3 59/DR_408
6) PCR product was checked on a 1% agarose gel and visualized using a blue LED transilluminator and purified using a PCR cleanup kit (Qiagen).
7) The 2 purified stitched DNA fragments were used in another stitch PCR reaction to create one linear DNA piece containing all 4 original pieces using the same template as described in the previous steps. Following the 10 cycle stitch step the whole fragment was amplified using the primer pair DR_359/DR_362.
8) The stitched PCR product was sequenced (Quintara) and the sequencing results were aligned to the reference sequence using the alignment feature in the Benchling software to verify that there were no mutations introduced during the PCR amplification steps.

Integration of sprA1_{AS} into IM08B Genome
1) Make competent E. coli and transform plasmid pKD46 per the protocol outlined in Report_065. Carbenicillin (Teknova) was used at 100 mg/L working concentration in the media to maintain the plasmid post transformation and recovery.
2) Pick a single colony and restreak for single colonies on a fresh LB carbenicillin plate and incubate at 30° C. overnight or until colonies are visible.
3) Once colonies are visible on the LB carb plate, pick a single colony to start a 5 mL LB+carbenicillin liquid culture, and incubate overnight at 30° C. in a rotary shaker at 240 rpm
4) The following day, transfer 1 mL of the overnight culture to a glass baffled shake flask containing fresh 50 mL LB carb media and continue shaking at 30° C. for 2 hours.
5) After the 2 hour incubation, add 1.75 mL of 10% L-arabinose to the culture and incubate the shake flask at 37° C. shaking at 240 rpm for an additional 45 minutes.
6) After the 45 minute incubation, transfer the shake flask to an ice bath and make prepare electrocompetent E. coli per the protocol outlined in Report_SOPO30.
7) Around 500 ng of the stitched PCR fragment generated above was added to 50 μL of electrocompetent E. coli from step 6 and electroporated.
   a) The DNA/cell solution was transferred to a chilled 0.1 mm gap cuvette.
   b) The settings on the electroporator were 1.8 kV, 200Ω.
   c) 1 ml of SOC broth media was added to the cuvette immediately after the cells were shocked, then placed in a 37° C. incubator shaking at 240 rpm for 3 hours to allow the cells to recover.
8) Following the 3 hour recovery process, the cells were plated on LB kanamycin (50 mg/L) agar plates and incubated at 37° C. overnight.
9) The following day, 8 colonies were screened for the PsprA1(as)-sprA1(as) insert in the E. co/i genome by PCR.
   a) E. coli colonies were patched to a fresh LB+kan agar plate, and the remainder of the colony was suspended in 20 μL of molecular biology grade water in a 0.2 mL PCR tube and heated to 98° C. for 10 minutes in a thermal cycler to lyse the cells, then allowed to slowly cool to room temperature.
   b) The tubes were briefly centrifuged to pellet the cell debris and 1 μL of the solution was used as the template in the PCR reaction screening for the insert.
      i) Primers DR_371 and DR_279 were used to generate a PCR band of 859 base pair.
      ii) The PCR products were then run on a 1% agarose gel to check for the proper band size.
      iii) The gDNA for three of the clones showing a positive band were used as the template for a high fidelity PCR to amplify the entire integrated region with the primers DR_371/DR_372.
      iv) The reactions were then run on an agarose gel to check for proper sized and clean bands. Clean looking reactions were sequenced and aligned to a reference sequence as done previously to verify that the integrations do not contain any mutations. Sequence verified clones were stocked for long term storage.
10) Electrocompetent cells of a sequence verified clone were made per the protocol outlined in Report_SOP030.
11) 0.6 μL of a Gibson Assembly for plasmid p249 was transformed into BPEC_001 electrocomp cells, plated on LB+chloramphenicol (20 μg/mL)/X-gal(100 μg/mL), and incubated overnight at 37° C.
12) The next day blue colonies were screened using primers DR_117 and DR_228, and the reactions were checked on a 1% agarose gel.
13) Genomic DNA from three positive clones was then used as the template for a high fidelity PCR to amplify the sequences inserted into the backbone of the plasmid (homology arms and sprA1).
14) The reactions were then run on an agarose gel to check for proper sized and clean bands. Clean looking reactions were sequenced and aligned to a reference sequence as done previously to verify that the integrations do not contain any mutations. Sequence verified clones were stocked for long term storage.

Results

Following the transformation of the Gibson Assembly of plasmid p249, the LB+chloramphenicol (20 μg/mL)/X-gal(100 μg/mL) agar plates showed many more blue colonies than transformations performed using E. coli K12 or IM08B electrocompetent cells. The colony PCR screen of the blue colonies showed that some of the cells had taken up plasmid DNA containing the sprA1 toxin gene. The following PCR and sequencing confirmed that the plasmids had in fact been assembled and are able to self replicate inside the E. coli host cells.

### Example 10. Strain Construction and Evaluation: Synthetic Microorganism Staph aureus

In this example, synthetic strain BP_118 (isdB::sprA1) was constructed using target strain BP_001 having successful genomic integration of toxin gene sprA1 behind native isdB gene. BP_0118 exhibited dramatic reduction in viable cfu/mL for strain BP_118 in human serum with no difference in growth in complex media (TSB) compared to the parent strain BP_001.

The plasmid p262 was constructed and used to make this edit by transforming it into a Staph aureus strain (BP_001) and integrating it into the genome by homologous recombination. Through a double recombination process, the plasmid was fully integrated into the genome of the Staph aureus strain BP_001, then through a second homologous recombination event the plasmid is removed leaving the sprA1 gene and 5 prime untranslated region directly behind the isdB gene. The efficacy of the genomic integration was evaluated by observing its growth in human serum in vitro.

Materials and Methods

Strain Construction

The plasmid used to make the strain was plasmid p262. The DNA sequences from p262 that are integrated into the strain can be found in Table 21A.

Genomic edits were made to Staph aureus using plasmid constructed from pIMAYz. Briefly, the plasmid was transformed into parent strain, grown at non-permissive temperatures for plasmid replication, screened for primary crossover strains, then grown and replated to screen colonies for the secondary crossover leaving behind the sprA1 gene. The sprA1 insertion was confirmed by Sanger sequencing of a PCR product amplified from gDNA by primers that bind to the genomic DNA outside the homology arms.

Primers used for the screening steps are found in Table 20:
i. Primary screen:
   1. DR 117, DR_533
   2. DR 117, DR_534
ii. Secondary screen:
   1. DR 534, DR_254
iii. Q5 High Fidelity PCR to confirm sprA1 integration:
   1. DR_533/DR_534
iv. Sequencing primers:
   1. DR 533, BP_949, DR 228, BP_965, BP_964, BP_950, DR 534, DR_318
v. Final confirmation:
   1. DR 534, DR_254

Following sequence confirmation of the insert, the new strain, BP_118, was stocked in 50% glycerol and stored at −80° C.

Table 20 shows the sequences for the single stranded primers used in this study. The sequences are all in the 5 prime to 3 prime direction.

TABLE 20
Primers and Their Sequences Used to Screen and
Sequence the Insert

| Primer | |
|---|---|
| Name | Primer Sequence (5′→3′) |
| DR_117 | CCAAAGCATAATGGGATAATTAACCCTCACTAAAGGGAAC |
| | (SEQ ID NO: 205) |
| DR_254 | ATGCTTATTTTCGTTCACATCATAGCACCAGTCATCAGTG |
| | (SEQ ID NO: 206) |
| DR_533 | GATTACGCTTACATTCGCTTCTCTGTTTC (SEQ ID NO: |
| | 207) |
| DR_534 | CAGCTGTTGATAATGCCATTTTTGCACGAG (SEQ ID NO: |
| | 208) |
| BP_964 | TCAAACTTCAGCAGGTTCTAGC (SEQ ID NO: 209) |
| BP_965 | GTACCAGGTATGACTGAATGCC (SEQ ID NO: 210) |
| BP_949 | CACCTCCTCTCTGCGGATTTATTAGTTTTTACGTTTTCTAGG |
| | TAATAC (SEQ ID NO: 211) |
| DR_228 | CTATTTTGTATTGCGTCTACTTAGCCAATAAG (SEQ ID |
| | NO: 212) |
| BP_950 | AAAAACTAATAAATCCGCAGAGAGGAGGTGTATAAGGTGATG |
| | (SEQ ID NO: 213) |
| DR_318 | CGATTACTTCCCAACCATTACCTACTGTCAAC (SEQ ID |
| | NO: 214) |

Table 21A shows the DNA sequences for the homology arms and sprA1 integration. The DNA sequences used were double stranded, but the sequences shown are just one of the strands in the 5 prime to 3 prime direction. For DNA sequence BP_DNA_003, the bold sequence indicates the sprA1 reading frame, and the underlined sequence indicates the 5 prime untranslated region (control arm).

TABLE 21A
DNA Fragments Used for Integration of isdB::sprA1
(p262)

| | Seq. | |
|---|---|---|
| Name | ID | DNA Sequence (5′→3′) |
| Upstream | BP_{—} | GATGAGCAAGTGAAATCAGCTATTACTGAATTC |
| Homology | DNA_{—} | CAAAATGTACAACCAACAAATGAAAAAATGACT |
| Arm | 029 | GATTTACAAGATACAAAATATGTTGTTTATGAA |
| | | AGTGTTGAGAATAACGAATCTATGATGGATACT |
| | | TTTGTTAAACACCCTATTAAAACAGGTATGCTT |
| | | AACGGCAAAAAATATATGGTCATGGAAACTACT |
| | | AATGACGATTACTGGAAAGATTTCATGGTTGAA |
| | | GGTCAACGTGTTAGAACTATAAGCAAAGATGCT |
| | | AAAAATAATACTAGAACAATTATTTTCCCATAT |
| | | GTTGAAGGTAAAACTCTATATGATGCTATCGTT |
| | | AAAGTTCACGTAAAAACGATTGATTATGATGGA |
| | | CAATACCATGTCAGAATCGTTGATAAAGAAGCA |
| | | TTTACAAAAGCCAATACCGATAAATCTAACAAA |
| | | AAAGAACAACAAGATAACTCAGCTAAGAAGGAA |
| | | GCTACTCCAGCTACGCCTAGCAAACCAACACCA |
| | | TCACCTGTTGAAAAAGAATCACAAAAACAAGAC |
| | | AGCCAAAAAGATGACAATAAACAATTACCAAGT |
| | | GTTGAAAAAGAAAATGACGCATCTAGTGAGTCA |
| | | GGTAAAGACAAAACGCCTGCTACAAAACCAACT |
| | | AAAGGTGAAGTAGAATCAAGTAGTACAACTCCA |
| | | ACTAAGGTAGTATCTACGACTCAAAATGTTGCA |
| | | AAACCAACAACTGCTTCATCAAAAACAACAAAA |
| | | GATGTTGTTCAAACTTCAGCAGGTTCTAGCGAA |
| | | GCAAAAGATAGTGCTCCATTACAAAAAGCAAAC |
| | | ATTAAAAACACAAATGATGGACACACTCAAAGC |
| | | CAAAACAATAAAAATACACAAGAAAATAAAGCA |
| | | AAATCATTACCACAAACTGGTGAAGAATCAAAT |
| | | AAAGATATGACATTACCATTAATGGCATTACTA |
| | | GCTTTAAGTAGCATCGTTGCATTCGTATTACCT |
| | | AGAAAACGTAAAAACTAATAAATC (SEQ ID |
| | | NO: 20) |
| sprA1 | BP_{—} | CGCAGAGAGGAGGTGTATAAGGTGATGCTTATT |
| Fragment | DNA_{—} | TTCGTTCACATCATAGCACCAGTCATCAGTGGC |
| (in- | 003 | TGTGCCATTGCGTTTTTTTCTTATTGGCTAAGT |
| sertion | | AGACGCAATACAAAATAG (SEQ ID NO: 3) |
| se- | | |
| quence) | | |
| Down- | BP_{—} | GTCTTTATATTTAATTATTAAATTAACAAATTT |
| stream | DNA_{—} | TAATTGGCGGATGAGGTATCCAGTTACCTCGTT |
| Homology | 002 | CGCCAATTATTTTTCGCAATATAAAAAGTCCCA |
| Arm | | CTTAAAACAATCATTTTAAGCGGGACTTTTTAT |
| | | ATTGAGTAACTAAAATTATTTAGCTGCTACTTC |
| | | TTCGCCATTGTAAGAACCACAGTTTTTACATAC |
| | | TACGGTGTGATAATTGTATTCGCCACAGTTTGG |
| | | GCATTCAGTCATACCTGGTACTGAAATTTTGAA |
| | | ATGCGTACGACGTTTGTTTTTTCTAGTTTTAGA |
| | | AGTTCTTCTTTTTGGTACTGCCATGATATATCC |
| | | TCCTTAGATTATAAACGAAAAATACTAAATGTT |
| | | AGTTTAATTAACAACATTATATCATTAATTAAA |
| | | CTACTTATTGCTCTTTATCATATAATTGTTGTA |
| | | ATTTTTGAAGCCTTGGATCAACTTGTCGTGATT |
| | | CTGAATCATCTTGTTCTTGCTGTTTAGCAAGCT |
| | | CATCTAATTGATCCTCATCGATTACTTCCCAAC |
| | | CATTACCTACTGTCAACATTTGGTCACTTTGCT |
| | | CTGAATAAGCTCTCATTGGTTTCTCAATAATAA |
| | | CTATATCCTCGACAATATCCTGAAGATTAACCA |
| | | TACCATCTTTAATAATGTGATAGTGTTCATCTA |
| | | CATCATCTTGATCATCGTTATACTGATTGTACC |
| | | CTTCTAAATCAAATACTTCTGTAGTAGTTACAT |
| | | CTAGTGGGACTTTTACTGGTACAAGAGTACGTG |
| | | CACAAGGCATTGTATACGTTCCAGTAATGTGAA |
| | | TATCCGCAACGACTTCTGTTGACTTAATGGTTA |
| | | ACTGACCTTGGATTGTAATTGGAGATAAATCAA |
| | | TTAAATCTAATGATTCTTTTAAATTGTCAAAAC |
| | | TCACCGTTTGATCAAATTCAAATGGCTTACCTT |
| | | GATATTTCCTTAATTGCGTAATTGAC (SEQ |
| | | ID NO: 2) |

The sprA1 integration was confirmed by PCR using primers DR_534 and DR_254 (FIG. 1). BP-001 was run as a negative control to show the integration is not present. The strain was then sent for Sanger sequencing (QuintaraBio). The sequencing results showed no mutations. The data for the sequences and alignment is stored in the present inventor's Benchling account.

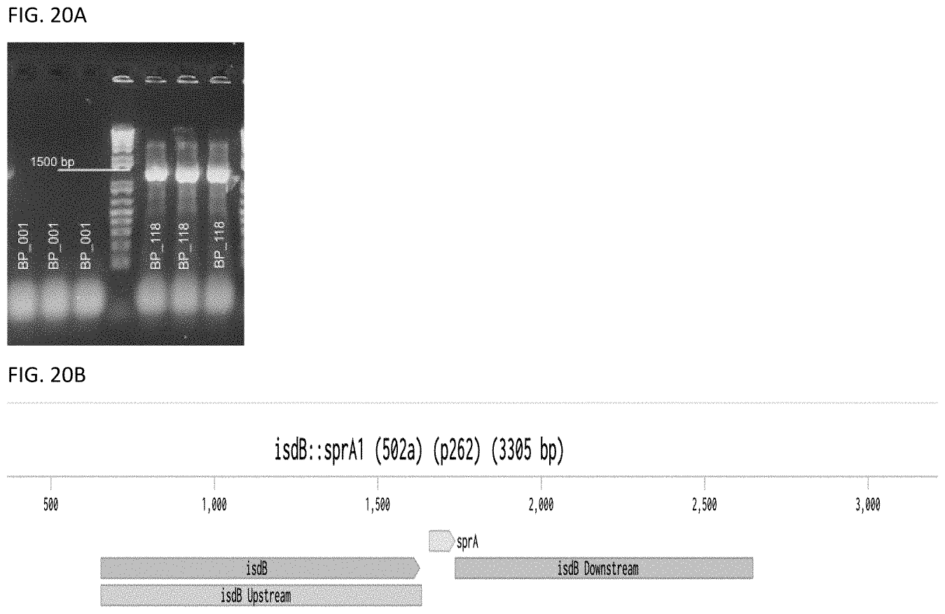

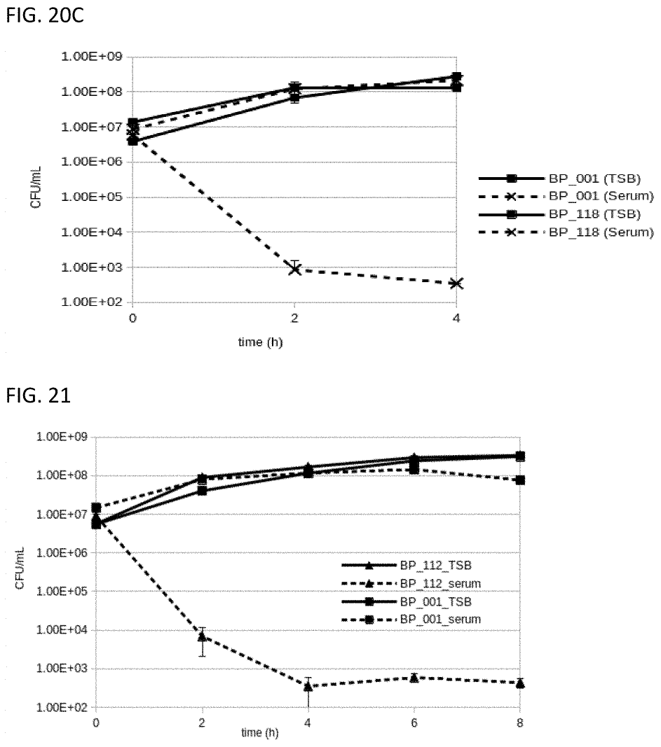

FIG. 20A shows an Agarose gel for PCR confirmation of isdb::sprA1 in BP_118. FIG. 20A shows a photograph of a 100 agarose gel that was run to check the PCR products of from the secondary recombination PCR screen with primers DR_534 and DR_254. Primer DR_534 binds to the genome outside of the homology arm, and the primer DR-254 binds to the sprA1 gene making size of the amplicon is 1367 bp for s strain with the integration and making no PCR fragment if the integration is not present. BP_001 was run as a negative control to show the integration is not present in the parent strain.

FIG. 20B shows a map of the genome of/BP_118 where the sprA1 gene was inserted. It was created with the Benchling program.

FIG. 20C shows the growth curves of 2 strains BP_001 and BP_118 when grown in TSB media and human serum over a 4 hour period. The points plotted on the graph represent an average of 3 biological replicates and the error bars represent the standard deviation for triplicate samples. The solid lines represent the cultures grown in TSB and the dashed lines represent cultures grown in human serum. When BP-118 was evaluated in a serum assay it showed that it was able to grow similar to the wild type strain BP_001 in TSB, but unlike BP_001 cannot sustain growth inhuman serum.

Other Synthetic Staph aureus strains prepared in a similar fashion are shown in Table 21B.

TABLE 21B
Synthetic Staphylococcus aureus Strains

| Strain | Parent | Description/Genetic | | Action | Plasmid for |
|---|---|---|---|---|---|
| Designation | Strain | Modification | Promoter(s) | Gene | Integration |
| BP_001 | n/a | Wild type | n/a | n/a | n/a |
| BP_011 | BP_001 | ΔsprA1-sprA1(AS) | n/a | n/a | p147 |
| BP_055 | BP_001 | Wild type (Plasmid in | n/a | n/a | p229 |
| | | strain - p229) | | | |
| BP_076 | BP_001 | ΔsprA1::Ptet-GFP | Ptet | GFPmut2 | p197 |
| BP_088 | BP_001 | isdB::sprA1 | isdB | sprA1 | p249 |
| BP_090 | BP_011 | ΔsprA1-sprA1(AS), | gyrB | sprA1(AS)(long) | p250 |
| | | Site_2::PgyrB- | | | |
| | | sprA1(AS) (long) | | | |
| BP_092 | BP_001 | PsbnA::sprA1 | sbnA | sprA1 | p252 |
| BP_094 | BP_011 | ΔsprA1-sprA1(AS), | gyrB | sprA1(AS)(long) | p251 |
| | | Site_2::PgyrB- | | | |
| | | sprA1(AS) (short) | | | |
| BP_098 | BP_088 | isdB::sprA1, | isdB, sbnA | sprA1 | p252 |
| | | PsbnA::sprA1 | | | |
| BP_101 | BP_088 | isdB::sprA1, | isdB, sbnA | sprA1 | p252 |
| | | PsbnA::sprA1 | | | |
| BP_103 | BP_001 | ΔsprA1 | n/a | n/a | p253 |
| BP_108 | BP_098 | isdB::sprA1, | isdB, sbnA | sprA1 | p253 |
| | | PsbnA::sprA1, | | | |
| | | ΔsprA1 | | | |
| BP_109 | BP_101 | isdB::sprA1, | isdB, sbnA | sprA1 | p253 |
| | | PsbnA::sprA1, | | | |
| | | ΔsprA1 | | | |
| BP_112 | BP_090 | ΔsprA1-sprA1(AS), | gyrB, isdB | sprA1(AS)(long), | p249 |
| | | Site_2::PgyrB- | | sprA1 | |
| | | sprA1(AS)(long), | | | |
| | | isdB::sprA1 | | | |
| BP_115 | BP_001 | isdB::sprA1 (Triple | isdB | sprA1 | p260 |
| | | STOP) | | | |
| BP_118 | BP_001 | isdB::sprA1 | isdB | sprA1 | p262 |
| BP_121 | BP_001 | Site_2::code_1 | n/a | n/a | p264 |
| BP_123 | BP_103 | ΔsprA1; isdB::sprA1 | isdB | sprA1 | p262 |
| BP_126 | BP_094 | ΔsprA1-sprA1(AS), | gyrB, isdB | sprA1(AS)(short), | p249 |
| | | Site_2::PgyrB- | | sprA1 | |
| | | sprA1(AS)(short), | | | |
| | | isdB::sprA1 | | | |
| BP_128 | BP_001 | harA::sprA1* | harA | sprA1* | p257 |
| BP_138 | BP_001 | isdB::sprA1 (500 | isdB | sprA1 | p249 |
| | | generations) | | | |
| BP_141 | BP_001 | isdB::sprA2 | isdB | sprA2 | p267 |
| BP_142 | BP_001 | PsbnA::sprA2 | sbnA | sprA2 | p268 |
| BP_144 | BP_109 | isdB::sprA1, | isdB, sbnA | sprA1(AS) | p272 |
| | | PsbnA::sprA1, | | | |
| | | ΔsprA1; | | | |
| | | Site_2::PsprA1(AS)- | | | |
| | | sprA1(AS) | | | |
| BP_145 | BP_118 | isdB::sprA1; | isdB | sprA1(AS) | p272 |
| | | Site_2::PsprA1(AS)- | | | |
| | | sprA1(AS) | | | |
| BP_146 | BP_092 | PsbnA::sprA1; | sbnA | sprA1(AS) | p271 |
| | | Site_2::PsprA1(AS)- | | | |
| | | sprA1(AS) | | | |
| BP_150 | BP_001 | ΔPsprA1::PsbnA | sbnA | sprA1 | p242 |
| BP_151 | BP_001 | PsbnA::GFP | sbnA | GFPmut2 | p282 |
| BP_152 | BP_001 | isdB::GFP | isdB | GFPmut2 | p284 |
| BP_156 | BP_001 | Wild type (Plasmid in | n/a | n/a | p303 |
| | | strain - p303) | | | |
| BP_157 | BP_001 | PsbnA::mKATE2 | sbnA | mKATE2 | p301 |
| BP_158 | BP_001 | isdB::mKATE2 | isdB | mKATE2 | p300 |
| BP_161 | BP_001 | Site_2::tetR_Ptet- | Ptet | GFPmut2 | p302 |
| | | GFPmut2 | | | |
| BP_162 | BP_001 | Site_2::tetR_Ptet- | Ptet | mKATE2 | p304 |
| | | mKATE2 | | | |
| CX_001 | n/a | Wild type | n/a | n/a | n/a |
| CX_013 | CX_001 | isdB::sprA1 | isdB | sprA1 | p262 |
| CX_051 | CX_013 | isdB::sprA1, ΔsprA1 | isdB | sprA1 | p253 |

*indicated truncated sprA1

Table 21C shows synthetic E. coli strains.

TABLE 21C
Synthetic E. coli strains

| Strain | Parent | Description/Genetic | | Action | Plasmid for |
|---|---|---|---|---|---|
| Designation | Strain | Modification | Promoter(s) | Gene | Integration |
| BPEC_001 | IM08B | ΔuidA::PsprA1(AS)- | uidA | sprA1(AS) | p279 |
| | | sprA1(AS)_kanR | | | |
| BPEC_002 | IM08B | ΔuidA::PsprA2(AS)- | uidA | sprA2(AS) | |
| | | sprA2(AS)_kanR | | | |
| BPEC_003 | IM08B | ΔuidA::tetR_P_{XYL/tet}- | uidA | mazF | p290 |
| | | mazF_kanR | | | |
| BPEC_004 | IM08B | ΔuidA::tetR_P_{XYL/tet}- | uidA | relE | p291 |
| | | relE_kanR | | | |
| BPEC_005 | IM08B | ΔuidA::tetR_P_{XYL/tet}- | uidA | yafQ | p292 |
| | | yafQ_kanR | | | |
| BPEC_006 | IM08B | ΔuidA::tetR_P_{XYL/tet}- | uidA | sprA1 | p287 |
| | | sprA1_kanR | | | |
| BPEC_007 | IM08B | ΔuidA::tetR_P_{XYL/tet}- | uidA | hokD | p289 |
| | | hokD_kanR | | | |
| BPEC_008 | IM08B | ΔuidA::tetR_P_{XYL/tet}- | uidA | hokB | p288 |
| | | hokB_kanR | | | |
| BPEC_009 | n/a | Wild type | n/a | n/a | n/a |
| BPEC_023 | K12 | Wild type (IM08B) | n/a | n/a | n/a |
| BPEC_024 | IM08B | Wild type (Plasmid in | n/a | n/a | p306 |
| | | strain - p306 - | | | |
| | | pRAB11_Ptet-sprG3) | | | |
| BPEC_025 | IM08B | Wild type (Plasmid in | n/a | n/a | p305 |
| | | strain - p305 - | | | |
| | | pRAB11_Ptet-sprG2†) | | | |

### Example 11. Strain Construction and Evaluation: Synthetic Microorganism Staph Aureus

In this example, a Staph aureus synthetic strain was constructed called BP_112 having genotype BP_001 ΔsprA1-sprA1(AS), Site_2::PgyrB-sprA1 (AS)(long), isdB::sprA1. A human serum assay suggested kill switch was effective with dramatic reduction in viable CFU/mL for strain BP_112, with no difference in growth in complex media (TSB) compared to the wild-type parent strain BP_001.

BP_112 represents a kill switched strain having the expression of antisense sprA1 (sprA1 (AS)) controlled by a promoter other than its native one. To make this strain, the present inventors first deleted the native sprA1 toxin gene along with the sprA1(AS) from the genome of the wild-type Staph aureus strain BP_001 using plasmid p147 (Report_P036). Next, a PgyrB-sprA1(AS)(long) expression cassette was inserted into the non-coding region of the genome referred to as Site_2 using the plasmid p250 (Report_P018). Two versions of the sprA1 (AS) were designed, the version in BP_112 represents the longer of the two versions. Finally, the isdB::sprA1 kill switch was inserted using plasmid p249. The efficacy of the genomic integration was evaluated by observing its growth in human serum in vitro.

The gyrB gene codes for the DNA gyrase subunit B and is constitutively expressed in the cell at reasonably high and stable levels. The promoter for the gene was PCR amplified from the genome of BP_001 and used to drive the expression of the antitoxin for the sprA1 gene, sprA1 (AS). This was placed in the Site_2 location of the genome because we previously demonstrated that this location can be used to insert heterologous DNA without disrupting the phenotype of the cell. In order to properly test the ability of the PgyrB-sprA1(AS) cassette to sufficiently suppress the isdB::sprA1 kill switch, the native sprA1(AS) was deleted from the genome prior to making the modification into Site_2. Studies show that there is no crosstalk between the sprA toxin-antitoxin systems in a Staph cell, so by removing the sprA1(AS) the only regulation of the isdB::sprA1 kill switch will be from the PgyrB-sprA1(AS) expression cassette. Germain-Amiot et al., Nucleic acids research 47.4 (2019): 1759-1773.

Materials and Methods

Table 22 shows the three different strains that were made through multiple rounds of editing the genome to create the final strain BP_112.

TABLE 22
Strain Constructs and Parent Strains in BP_112 Lineage

| | Strain Construct | Parent | Parent Strain's |
|---|---|---|---|
| Construct | Genotype | Strain | Genotype |
| BP_011 | ΔsprA1-sprA1(AS) | BP_001 | Wild type |
| BP_090 | ΔsprA1-sprA1(AS), | BP_011 | ΔsprA1-sprA1(AS) |
| | Site_2::PgyrB- | | |
| | sprA1(AS) (long) | | |
| BP_112 | ΔsprA1-sprA1(AS), | BP_090 | ΔsprA1-sprA1(AS), |
| | Site_2::PgyrB-sprA1(AS) | | Site_2::PgyrB- |
| | (long), isdB::sprA 1 | | sprA1(AS) (long) |

Strain Construction
1. The plasmids p147, p249, and p250 were used to make the strain over three rounds of editing the genome using the protocol outlined herein for genetic engineering of Staph aureus with pIMAYz.
   1.1. Briefly, a plasmid was transformed into parent strain, grown at non-permissive temperatures for plasmid replication, screened for primary crossover strains, then grown and replated to screen colonies for the secondary crossover leaving behind the desired insertion or deletion in the genome. The insertion/deletion was confirmed by Sanger sequencing of a PCR product amplified from gDNA by primers that bind to the genomic DNA outside the homology arms.
2. Following sequence confirmation of the insert, the new strains were stocked in 50% glycerol and stored at −80° C. to prepare strain and plasmid stock.
3. BP_112 was analyzed in an 8-hour human serum assay to assess the phenotypic response of the modified strain. BP_112 was compared to BP_001 and the serum assay was run over 8 hr. The results are included in FIG. 21.

FIG. 21 shows the average CFU/mL for BP_112 (n=3) and BP_001 (n=1) when they are grown in serum (dashed lines) and TSB (solid lines) over an 8-hour period. The error bars represent the standard deviation of the averaged values.

Three genomic modifications were made to the strain BP_001 to create the strain BP_112. First, the sprA1-sprA1(AS) genes were knocked out to remove background expression of either the sprA1 toxin or the antisense (sprA1(AS)). Next, a sprA1(AS) expression cassette was inserted into Site_2 (PgyrB-sprA1(AS)(long)). The final edit was to integrate a kill switch by inserting the sprA1 gene behind the isdB gene. All of these edits were performed successfully and have been stocked in BioPlx's database.

When evaluated in a serum assay, BP_112 (ΔsprA1-sprA1 (AS), Site_2::PgyrB-sprA1 (AS)(long), isdB::sprA1) was able to grow similar to the wild-type strain BP_001 in TSB, but unable to grow in human serum. This demonstrates that BP_112 successfully controlled the sprA1 kill switch using an artificial sprA1 antitoxin expression system.

### Example 12. Genomic Integration Site Selection for Optimal Expression of Action Gene: Start Site Optimization for Kill Switch

The location chosen for integrating an action gene such as a kill switch may affect the efficacy of the toxin. Gene expression can vary widely for each gene within an organism depending on the environmental conditions. As shown in this example, the efficacy of the sprA1 kill switch varies depending on the location in the genome chosen for integration.

In order to test the most optimal site for integrating an exogenous DNA sequence to create a kill switch (KS), a short growth assay was performed in pooled human serum and TSB media with the wild type Staph aureus target strain BP_001.

Briefly, overnight growth cultures of BP_001 in TSB were diluted 1:100 into fresh TSB media and grown for another 2 hours at 37° C. to sync the metabolism of the cells. Following the 2 hours growth period, the OD was taken again as the cells were washed twice and concentrated to 1 mL volumes in phosphate buffered saline (PBS). The concentrated cells were used to inoculate 3 tubes each of TSB and human serum, and grown at 37° C. in the shaking incubator for 90 minutes. Samples were taken at t=0, 30, and 90 minutes after inoculation, and the RNA was extracted and purified using the RiboPure™ RNA Purification Kit, bacteria (ThermoFisher). The RNA samples were then sent to Vertis Biotechnologie AG (Freising, Germany) for removal of the rRNA, creating a cDNA library, sequencing the cDNA library, trimming and processing the sequencing data, and mapping it to an annotated genomic sequence of a Staph aureus 502a strain. The data from the RNA seq experiment was analyzed to highlight the most differentially regulated transcripts which were then used to target the insertion of the action gene sprA1. This gene is part of a native toxin antitoxin system in BP_001 has been shown previously to be toxic when overexpressed.

Several locations in the genome were chosen to integrate the action gene in order to operably link the transcription of the gene and translation of the protein to the cell's native regulatory systems.

The genomic modifications were made using the method described in the examples above for plasmid construction using pIMAYz protocol and homologous recombination. In brief, homology arms were designed both upstream and downstream of the genomic location targeted for integration, and either a DNA fragment containing sprA1 along with a short sequence upstream of the action gene or inducible promoter was inserted into the genome. The efficacy of the integration was then determined by running growth assays in human serum or TSB.

The protocol for this example is similar to that used in the RNA-seq experiment, but after the final serum and TSB cultures were inoculated, the assay was run for 4 hours and samples were taken at t=0, 2, and 4 hours post inoculation, serially diluted by a liquid handling robot, and plated on TSB agar plates to determine the concentration of viable cells in the cultures in colony forming units per mL (CFU/mL). The growth in both TSB and pooled human serum for the engineered strains were compared to the wild type strain BP_001.

Results are shown in FIG. 18 showing the fold change in expression of 25 genes from Staph aureus at 30 and 90 minute time points in TSB and human serum. The genes shown above were most differentially regulated at the 90 minute time point between human serum and TSB broth. The number of reads for each gene was converted to transcripts per million (TPM), the replicates were averaged for each condition (n=3), normalised to the expression of the housekeeping gene gyrB, subtracted from the initial expression levels at t=0, and sorted for the most differentially expressed between the two media conditions at the 90 minute time point. The gene on the bottom of the chart (CH52_00245) had a value of 175 fold upregulation, but was cut short on this figure in order to enlarge the chart maximize the clarity of the rest of the data.

The RNA-seq results revealed many genes in BP_001 that are differentially regulated during growth in TSB and human serum. Many of the most highly differentially regulated genes between TSB and serum involve iron sequestration and acquisition from the environment. The most interesting genes for kill switch design were heavily suppressed in TSB and highly upregulated in human serum.

Table 23 shows the genes or promoters identified as good candidate locations to integrate the action gene. Genes isdB, PsbnA, and isdC are found among the top 25 genes shown in FIG. 18.

TABLE 23
Differentially Regulated Genes Identified and Targeted for Action Gene

| | Promoter or | |
|---|---|---|
| Name (Accession ID) | Gene | Description of Gene/Promoter |
| isdB (CH52_00245) | Gene | iron-regulated surface determinant protein B |
| PsbnA | Promoter | Promoter for siderophore biosynthesis proteins |
| (CH52_05140-05100) | | sbnABCDEFGHI |
| harA (CH52_10455) | Gene | Iron-regulated surface determinant protein H |
| isdC (CH52_00235) | Gene | iron-regulated surface determinant protein C |
| sbnB (CH52_05135) | Gene | 2,3-diaminopropionate biosynthesis protein SbnB |
| isdE (CH52_00225) | Gene | heme uptake system protein IsdE |

Some genes targeted for integration were not present in the top 25 differentially

Some genes targeted for integration were not present in the top 25 differentially regulated genes, but were chosen in order to provide a spectrum of responses from the kill switch. The genes sbnB and isdE were targeted because the PsbnA promoter is a bidirectional promoter and it was hypothesized that it might be regulated in a similar manner for sbnB as it is for sbnA, and isdE is on the same operon as isdC which is among the list of top 25 genes. The harA gene was targeted due to literature claims of the protein being regulated and functionally similar to the isdB gene. Dryla et al. Journal of bacteriology vol. 189, 1 (2007): 254-64. doi:10.1128/JB.01366-06. By choosing candidate gene targets both on and off the list, a tailored spectrum of responses from the kill switch may be explored.

Table 24 shows strains that were made and tested for the sprA1 kill switch's efficacy in human serum and TSB.

Table 24. Strains Made to Test Location of Integration Action Gene or Induced Promoter

| Strain Name | Genotype |
|---|---|
| BP_092 | PsbnA::sprA1 |
| BP_118 | isdB::sprA1 |
| BP_128 | harA::sprA1* |
| BP_150 | ΔPsprA1::PsbnA |

FIG. 19 shows kill switch activity as average CFU/mL of 4 Staph aureus synthetic strains with different kill switch integrations in human serum compared to parent target strain BP_001. FIG. 19 shows the viable CFU/mL of 4 different synthetic SA strains with a sprA1 kill switch integrated into 4 different locations in the genome grown in serum over 4 hours. The data is plotted as CFU/mL at three different time points and the error bars represent the standard deviation of the triplicate samples (except BP_128 which has a n=1). The CFU/mL data for all of the strains grown in TSB overlays with the BP_001 in serum on this chart and was omitted in order to produce a cleaner graph. *The sprA1 gene in BP_128 was found to contain a frameshift mutation that truncates the protein by 7 amino acids, and the last 3 amino acids in the truncated protein have been changed.

As shown in FIG. 19, when tested for their ability to grow in serum, strains BP_118 (isdB::spra1), BP_092 (PsbnA::sprA1) and BP_128 (harA::sprA1) each exhibited a decrease in CFU/mL at both the 2 and 4 hour time points. BP_118 (isdB::spra1) exhibited strongest kill switch activity as largest decrease in CFU/mL. Strain BP_150 grew only slightly slower than the wild type parent strain, but still maintained a positive growth curve during the 4 hour assay.

### Example 13. Human Plasma Kill Assay with BP_088, BP_101, BP_108, and BP_109

Several kill switched Staph aureus strains were tested for efficacy in human plasma. These same strains have been shown to quickly die in human serum, so other biological fluids are being investigated for their ability to induce the integrated kill switch (KS) and reduce the number of viable cells. Table 25 shows the strains employed in the assay.

TABLE 25
Strains Used in the Plasma KS Assay

| Strain Name | Genomic Modifications |
|---|---|
| BP_001 | Wild type Staph aureus |
| BP_088 | isdB::sprA1 |
| BP_092 | PsbnA::sprA1 |
| BP_101 | isdB::sprA1, PsbnA::sprA1 |
| BP_108 | isdB::sprA1, PsbnA::sprA1, ΔsprA1 |
| BP_109 | isdB::sprA1, PsbnA::sprA1, ΔsprA1 |

The serum assay protocol was employed as described herein except exchanging the serum growth condition with human plasma.

Human plasma is the liquid portion of blood. It is acquired by spinning to remove the cells, and still contains proteins, clotting factors, electrolytes, antibodies, antigens and hormones. Since the clotting factors are still present in the liquid, it is a difficult media to use for culturing cells. Clumps of cells and protein form over time and care was taken to homogenize the cultures before sampling. It was found that if assays longer than 3.5 hours are needed, anticoagulants should be added to the plasma prior to inoculation.

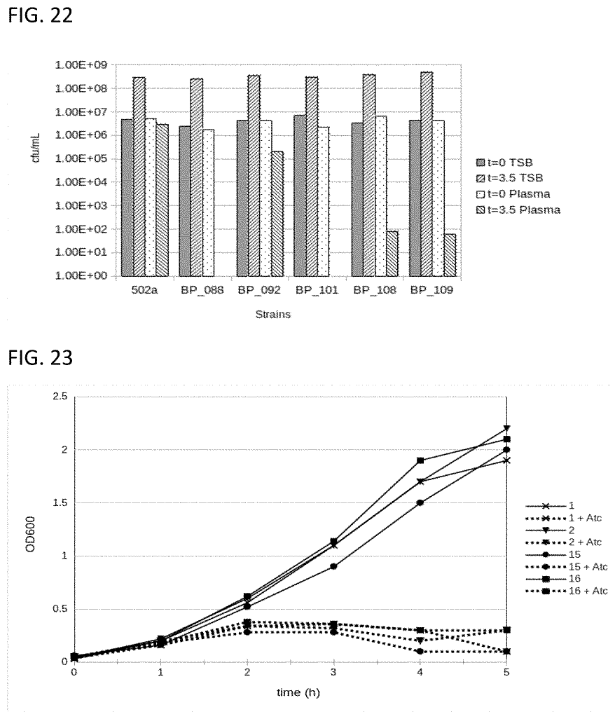

Results are shown in FIG. 22 showing a bar graph of the concentration of cfu/mL for all of the strains tested in both TSB and human plasma, at both t=0 and after 3.5 hours of growth (t=3.5). The viable cfu/mL of strains BP_088, BP_101, BP_108, and BP_109 showed over a 99% reduction after 3.5 hours in human plasma. BP_092 showed a 95% reduction in viable cfu/mL after 3.5 hours in human plasma. BP_001 showed very little difference in viable cfu/mL after 3.5 hours in human plasma. All strains grew in TSB media. The results from the assay show that the Staph aureus strains with integrated KS were unable to grow in human plasma. All of the cultures started around 1*106 cfu/mL in both TSB and human serum, and after 3.5 hours of growth at 37° C. all of the TSB cultures showed an approximate 100-fold increase in cfu/mL. 502a showed a slight decrease in cfu/mL in human plasma, and the kill switched strains (BP_088, BP_092, BP_101, BP_108, BP_109) all showed a decrease in cfu/mL in plasma. The kill switched microorganisms performed well in human plasma. The results from the assay show that the Staph aureus strains with integrated KS were unable to grow in human plasma.

### Example 14. E. coli Toxin Efficacy Test

Two different E. coli strains were genomically modified under the control of the P_{XYL/Tet} promoter to incorporate putative E. coli toxins hokB, hokD, relE, mazF, and yafQ, and known S. aureus toxin sprA1. Overexpression of hokD, sprA1, and relE genes resulted in a decrease in the optical density of the synthetic E. coli cell cultures indicating they function as toxins to the host cells. In contrast, overexpression of E. coli comprising hokB, mazF, and yafQ operably linked to the inducible promoter did not demonstrate a toxic effect towards the host cells under the conditions of this assay.

Putative E. coli toxin genes were incorporated to E. coli genome and resulting strains were tested for their ability to arrest cell growth or kill living cells in a culture. A strong inducible and tightly controlled promoter system P_{XYL/Tet} was selected to perform this assay efficiently and effectively.

E. coli has many genes that have been annotated as a component of endogenous toxin-antitoxin (TA) systems. The present inventors have shown that TA systems can be exploited to develop kill switches in bacteria that are induced by environmental changes. Identifying effective toxin genes across different species and strains is a crucial part of developing such kill switches.

The RED system was used to integrate linear DNA into the genome of two different E. coli strains, a K12 background strain named IM08B (Monk et al., 2015 M Bio 6.3: e00308-15) and a strain purchased from Udder Health Systems which they use as their E. coli bovine standard. Datsenko et al., Proc. Natl. Acad. Sci. U.S.A. 97 (12), 6640-6645 (2000).

The linear DNA integrated into the genome contains a putative toxin gene behind a strong constitutive promoter P_{XYL/Tet} that contains 2 tetO sites where the tet repressor (TetR) protein tightly binds to block transcription of the putative toxin gene, as well as the tetR gene and and a kanamycin resistance gene. Helle et al. “Vectors for improved Tet repressor-dependent gradual gene induction or silencing in Staphylococcus aureus.” Microbiology 157.12 (2011): 3314-3323. When anhydrotetracycline (ATc), a non-toxic form of the antibiotic tetracycline is added to the media it allosterically binds to the tetR protein changing the protein's conformation rendering it the unable to bind to the DNA at the tetO sites and block transcription of the downstream gene or genes. With the TetR proteins deactivated, the constitutive promoter is derepressed and is uninhibited when recruiting RNA polymerase to transcribe the putative toxin gene at a high rate. The effect the toxin has on the culture can be seen by measuring the optical density (OD600) of the cultures over time. By comparing samples that have been spiked with ATc and samples that have not we can see how effective each toxin is. Top candidates will be used in the development of kill switches that are induced or repressed based on environmental conditions.

The integration of the expression cassette and kanamycin resistance gene was made by inserting it in the E. coli genome in place of the uidA gene (also called gusA) which codes for a protein called β-D-glucuronidase. The uidA gene is the first gene a three gene operon, and the integration also removes the first 4 bases in the uidB gene (also called gusB) likely disrupting or disabling the expression of it and the last gene in the operon uidC (gusC). It is nonessential for E. coli growth and its absence will not affect the efficacy of the toxins being tested here, making it a convenient location to make integrations. All of the integrations made in this report used the same homology arms for targeting the location in the genome which means that they were all made in the exact same location.

The list below shows the toxins being tested in this report and a brief description of each one:

sprA1

The sprA1 gene is native to Staph aureus, and is part of a type I toxin antitoxin system. The sprA1 gene codes for a membrane porin protein called PepA1, which accumulates in the cell's membrane and induces apoptosis in dividing cells. Schuster et al., “Toxin-antitoxin systems of Staphylococcus aureus.” Toxins 8.5 (2016): 140. The effectiveness of sprA1 in Staph aureus is provided herein and it was hypothesized it might perform similarly in E. coli. The sprA1 gene used here was PCR amplified from the genome of a 502a-like strain named i BP_001.

hokB

The hokB gene is a member of the type I toxin-antitoxin system in the hok-sok family in E. coli. The protein has been demonstrated to insert itself into the cytoplasmic membrane and form pores that result in leakage of ATP. Wilmaerts et al. 2018. The persistence-inducing toxin HokB forms dynamic pores that cause ATP leakage. mBio 9:e00744-18. https://doi.org/10.1128/mBio 0.00744-18. Sequence analysis has shown that hokB is a homolog of the hok (host killing) gene. The hokB gene used in this report was PCR amplified from the genome of an E. coli K12 strain.

hokD

The hokD gene is a member of the type I toxin-antitoxin system in the hok-sok family in E. coli. The stable mRNA from hokD is post transcriptionally regulated by an sRNA antitoxin sok. The hokD gene codes for a protein that has been shown to be toxic to E. coli, resulting in loss of membrane potential, cell respiration arrest, morphological changes, and host cell death. Gerdes et al., The EMBO journal 5.8 (1986): 2023-2029. Sequence analysis has showed that hokB is a homolog of the hok (host killing) gene. The hokD gene used in this report was PCR amplified from the genome of an E. coli K12 strain.

mazF

The mazF gene is found throughout many species of bacteria, and in combination with the mazE gene, comprise a toxin antitoxin system where mazE functions as the antitoxin and mazF the toxin that has been shown to exhibit ribonuclease activity towards single or double stranded RNA resulting global translation inhibition. Aizenman et al., “An Escherichia coli chromosomal“addiction module” regulated by guanosine 3′,5′-bispyrophosphate: a model for programmed bacterial cell death.” Proceedings of the National Academy of Sciences 93.12 (1996): 6059-6063. The mazF gene used in this report was PCR amplified from the genome of an E. coli K12 strain.

relE

The relE gene is a member of the relE-relB toxin-antitoxin system in E. coli, and has been shown to inhibit protein translation when overexpressed causing reversible cell growth. Translation inhibition occurs from relE catalyzing the cleavage of mRNA in the A site of the ribosome. Pedersen et al., “Rapid induction and reversal of a bacteriostatic condition by controlled expression of toxins and antitoxins.” Molecular microbiology 45.2 (2002): 501-510. The relE gene used in this report was PCR amplified from the genome of an E. coli K12 strain.

Methods

Table 26 shows the primer names and sequences used to construct the linear DNA fragments integrated into the genome of E. coli to test the efficacy of putative toxin genes at killing the host cells.

TABLE 26
Primers Used to Make and Sequence Integration
Fragments

| Primer | |
|---|---|
| Name | DNA Sequence (5′→3′) |
| DR_359 | GGAACCGATTGAAGGGATTCATTTCGTTG (SEQ ID NO: |
| | 192) |
| DR_409 | CTCGGTTGCTGTGTTGCACACAGTTATCTGTGAG (SEQ ID |
| | NO: 193) |
| DR_407 | GTGTGCAACACAGCAACCGAGCGTTCTGAACAAATCCAG |
| | (SEQ ID NO: 198) |
| BM_049 | CGTACTGATTGGGTAGGTGACATATAGCCGCACCAATAAAAAT |
| | TGATAATAGCTG (SEQ ID NO: 215) |
| BM_015 | GGCTATATGTCACCTACCCAATCAGTACGTTAATTTTGGC |
| | (SEQ ID NO: 216) |
| BM_014 | GGTGTATAAGGTGATGGTAAGCCGATACGTACCCGATATG |
| | (SEQ ID NO: 217) |
| BM_013 | TCGGCTTACCATCACCTTATACACCTCCTCTCTGCGG (SEQ |
| | ID NO: 218) |
| DR_634 | CAGGAGAGTTGTTGATGCATGTAACTGGGCAGTGTCTTAAAA |
| | AATCGAC (SEQ ID NO: 219) |
| DR_636 | CAGTTACATGCATCAACAACTCTCCTGGCGCACCATC (SEQ |
| | ID NO: 220) |
| DR_362 | GTTTCAGGGTTTGCAGACTGATATTCAATGACG (SEQ ID |
| | NO: 195) |
| BM_052 | GGTGTATAAGGTGATGATTCAAAGGGATATTGAATACTCGGGA |
| | C (SEQ ID NO: 221) |
| BM_027 | GCTATATGTCACTTACCCAAAGAGCGCCGCG (SEQ ID NO: |
| | 222) |
| BM_025 | CCCTTTGAATCATCACCTTATACACCTCCTCTCTG (SEQ ID |
| | NO: 223) |
| BM_024 | GCTCTTTGGGTAAGTGACATATAGCCGCACCAATAAAAATtg |
| | (SEQ ID NO: 224) |
| BM_018 | GGTGTATAAGGTGATGGCGTATTTTCTGGATTTTGACGAGC |
| | (SEQ ID NO: 225) |
| BM_019 | GGCTATATGTCACTCAGAGAATGCGTTTGACCGCCTCG (SEQ |
| | ID NO: 226) |
| BM_017 | AAAATACGCCATCACCTTATACACCTCCTCTCTGCGG (SEQ |
| | ID NO: 227) |
| BM_016 | CGCATTCTCTGAGTGACATATAGCCGCACCAATAAAAATTG |
| | (SEQ ID NO: 228) |
| DR_244 | CATCACCTTATACACCTCCTCTCTGCGG (SEQ ID NO: |
| | 229) |
| DR_661 | CTGAGGAGTAAGTGACATATAGCCGCACCAATAAAAATTGATA |
| | ATAGCTG (SEQ ID NO: 230) |
| DR_659 | CGCAGAGAGGAGGTGTATAAGGTGATGAAGCAGCAAAAGGCGA |
| | TGTTAATCG (SEQ ID NO: 231) |
| DR_660 | GTGCGGCTATATGTCACTTACTCCTCAGGTTCGTAAGCTGTGA |
| | AGAC (SEQ ID NO: 232) |
| DR_674 | GTCCAGGTAAGTACCCAGGAAACAGCTATGACCATG (SEQ |
| | ID NO: 233) |
| DR_673 | AGCTGTTTCCTGGGTACTTACCTGGACGTGCAGGCCATG |
| | (SEQ ID NO: 234) |
| DR_672 | GGAGGTGTATAAGGTGATGAAGCACAACCCTCTGGTGGTG |
| | (SEQ ID NO: 235) |
| DR_675 | GGTTGTGCTTCATCACCTTATACACCTCCTCTCTGCGG (SEQ |
| | ID NO: 236) |
| DR_280 | GTAGACGCAATACAAAATAGGTGACATATAGCCGCACC (SEQ |
| | ID NO: 237) |
| DR_278 | CGCAGAGAGGAGGTGTATAAGGTGATGCTTATTTTCGTTCACA |
| | TC (SEQ ID NO: 238) |
| DR_228 | CTATTTTGTATTGCGTCTACTTAGCCAATAAG (SEQ ID |
| | NO: 239) |

DNA Fragment Construction

The list below shows the primer pairs (and templates) used to PCR amplify the fragments that were assembled to construct the DNA fragments integrated into the genome of E. coli.
1) ΔuidA::tetR_P_{XYL/Tet}-sprA1_kanR
   a) Upstream HA—DR_359/DR_409 (E. coli gDNA)
   b) kanR—DR_407/DR_637 (pCasSA plasmid)
   c) tetR_P_{XYL/tet}—DR_634/DR_280 (pRAB11 plasmid)
   d) sprA1—DR_278/DR_228 (Staph aureus gDNA)
   e) Downstream HA—DR_362/DR_636 (E. coli gDNA, K12)
2) ΔuidA::tetR_P_{XYL/Tet}-hokB_kanR
   a) Upstream HA—DR_359/DR_409 (E. coli gDNA)
   b) kanR—DR_407/DR_674 (pCasSA plasmid)
   c) tetR_P_{XYL/tet}—DR_634/DR_675 (pRAB11 plasmid)
   d) hokB—DR_672/DR_673 (E. coli gDNA, K12)
   e) Downstream HA—DR_362/DR_636 (E. coli gDNA, K12)
3) ΔuidA::tetR_P_{XYL/Tet}-hokD_kanR
   a) Upstream HA —DR_359/DR_409 (E. coli gDNA)
   b) kanR—DR_407/DR_661 (pCasSA plasmid)
   c) tetR_P_{XYL/tet}—DR_634/DR_244 (pRAB11 plasmid)
   d) hokD—DR_659/DR_660 (E. coli gDNA, K12)
   e) Downstream HA—DR_362/DR_636 (E. coli gDNA, K12)
4) ΔuidA::tetR_P_{XYL/Tet}-relE_kanR
   a) Upstream HA—DR_359/DR_409 (E. coli gDNA, K12)
   b) kanR—DR_407/BM_016 (pCasSA plasmid)
   c) tetR_P_{XYL/tet}—BM_017/DR_634 (pRAB11 plasmid)
   d) relE—BM_018/BM_019 (E. coli gDNA, K12)
   e) Downstream HA—DR_362/DR_636 (E. coli gDNA, K12)
5) ΔuidA::tetR_P_{XYL/tet}-yafQ_kanR
   a) Upstream HA—DR_359/DR_409 (E. coli gDNA, K12)
   b) kanR—BM_024/DR_407 (pCasSA plasmid)
   c) tetR_P_{XYL/tet}—BM_025/DR_634 (pRAB11 plasmid)
   d) yafQ—BM_052/BM_027 (E. coli gDNA, K12)
   e) Downstream HA—DR_362/DR_636 (E. coli gDNA, K12)
6) ΔuidA::tetR_P_{XYL/Tet}-mazF_kanR
   a) Upstream HA—DR_359/DR_409 (E. coli gDNA, K12)
   b) kanR—BM_049/DR_407 (pCasSA plasmid)
   c) tetR_P_{XYL/tet}—BM_013/DR_634 (pRAB11 plasmid)
   d) mazF—BM_015/BM_014 (E. coli gDNA)
   e) Downstream HA—DR_362/DR_636 (E. coli gDNA)

All of the fragments listed above were PCR amplified using Q5 Hot Start DNA polymerase (NEB) per the manufacturer's instructions and run on a 1-2% agarose gel to confirm good amplification from the template DNA. The PCR fragments were then purified using a PCR cleanup kit (Qiagen) and assembled by the stitch PCR protocol outlined in Report_SOP036. The primer pair DR_362/DR_359 was used to create the single linear DNA fragment used to make each integration. This PCR product incorporates the 5 fragments used in the stitch PCR (Upstream HA, kanR, tetR_P_{XYL/tet}, putative toxin gene, Downstream HA).

Table 27 shows the DNA sequences for the putative toxin genes tested and described in this report.

TABLE 27
DNA Sequences of the Toxins Tested in Efficacy Test

| Toxin | DNA | |
|---|---|---|
| Name | Sequence ID | DNA Sequence (5′→3′) |
| sprA1 | BP_DNA_035 | ATGCTTATTTTCGTTCACATCATAGCACCAGT |
| | | CATCAGTGGCTGTGCCATTGCGTTTTTTTCTT |
| | | ATTGGCTAAGTAGACGCAATACAAAATAG |
| | | (SEQ ID NO: 25) |
| hokB | BP_DNA_067 | ATGAAGCACAACCCTCTGGTGGTGTGTCTGCT |
| | | CATTATCTGCATTACGATTCTGACATTCACAC |
| | | TCCTGACCCGACAAACGCTCTACGAACTGCGG |
| | | TTCCGGGACGGTGATAAGGAGGTTGCTGCGCT |
| | | CATGGCCTGCACGTCCAGGTAA (SEQ ID |
| | | NO: 35) |
| hokD | BP_DNA_068 | ATGAAGCAGCAAAAGGCGATGTTAATCGCCCT |
| | | GATCGTCATCTGTTTAACCGTCATAGTGACGG |
| | | CACTGGTAACGAGGAAAGACCTCTGCGAGGTA |
| | | CGAATCCGAACCGGCCAGACGGAGGTCGCTGT |
| | | CTTCACAGCTTACGAACCTGAGGAGTAA |
| | | (SEQ ID NO: 36) |
| mazF | BP_DNA_069 | ATGGTAAGCCGATACGTACCCGATATGGGCGA |
| | | TCTGATTTGGGTTGATTTTGACCCGACAAAAG |
| | | GTAGCGAGCAAGCTGGACATCGTCCAGCTGTT |
| | | GTCCTGAGTCCTTTCATGTACAACAACAAAAC |
| | | AGGTATGTGTCTGTGTGTTCCTTGTACAACGC |
| | | AATCAAAAGGATATCCGTTCGAAGTTGTTTTA |
| | | TCCGGTCAGGAACGTGATGGCGTAGCGTTAGC |
| | | TGATCAGGTAAAAAGTATCGCCTGGCGGGCAA |
| | | GAGGAGCAACGAAGAAAGGAACAGTTGCCCCA |
| | | GAGGAATTACAACTCATTAAAGCCAAAATTAA |
| | | CGTACTGATTGGGTAG (SEQ ID NO: 37) |
| yafQ | BP_DNA_070 | ATGATTCAAAGGGATATTGAATACTCGGGACA |
| | | ATATTCAAAGGATGTAAAACTTGCACAAAAGC |
| | | GTCATAAGGATATGAATAAATTGAAATATCTT |
| | | ATGACGCTTCTTATCAATAATACTTTACCGCT |
| | | TCCAGCTGTTTATAAAGACCACCCGCTGCAAG |
| | | GTTCATGGAAAGGTTATCGCGATGCTCATGTC |
| | | GAACCGGACTGGATCCTGATTTACAAACTTAC |
| | | CGATAAACTTTTACGATTTGAGAGAACTGGAA |
| | | CTCACGCGGCGCTCTTTGGGTAA (SEQ ID |
| | | NO: 38) |
| relE | BP_DNA_071 | ATGGCGTATTTTCTGGATTTTGACGAGCGGGC |
| | | ACTAAAGGAATGGCGAAAGCTGGGCTCGACGG |
| | | TACGTGAACAGTTGAAAAAGAAGCTGGTTGAA |
| | | GTACTTGAGTCACCCCGGATTGAAGCAAACAA |
| | | GCTCCGTGGTATGCCTGATTGTTACAAGATTA |
| | | AGCTCCGGTCTTCAGGCTATCGCCTTGTATAC |
| | | CAGGTTATAGACGAGAAAGTTGTCGTTTTCGT |
| | | GATTTCTGTTGGGAAAAGAGAACGCTCGGAAG |
| | | TATATAGCGAGGCGGTCAAACGCATTCTCTGA |
| | | (SEQ ID NO: 39) |

Table 28A shows one strand of the double stranded DNA sequences that were used as homology arms to target the location of the integrations described in this report. For sequence BP_DNA_075 (SEQ ID NO: 40), the underlined sequence is the P_{XYL/tet} promoter sequence and the bold portion is the sequence for the tetR gene. The bold portion in BP_DNA_076 (SEQ ID NO: 41) corresponds to the kanR gene.

TABLE 28A
DNA Sequences and Sequence IDs for ΔuidA Homology
Arms

| | DNA Se- | |
|---|---|---|
| DNA | quence | |
| Name | ID | DNA Sequence (5′→3′) |
| Up- | BP_DNA_{—} | GGAACCGATTGAAGGGATTCATTTCGTTGACTAT |
| stream | 016 | ATGGTCGAGTCCATTGTCTCTCTCACCCATGAAG |
| HA | | CCTTTGGACAACGGGCGCTGGTGGTTGAAATTAT |
| | | GGCGGAAGGGATGCGTAACCCACAGGTCGCCGCC |
| | | ATGCTTAAAAATAAGCATATGACGATCACGGAAT |
| | | TTGTTGCCCAGCGGATGCGTGATGCCCAGCAAAA |
| | | AGGCGAGATAAGCCCAGACATCAACACGGCAATG |
| | | ACTTCACGTTTACTGCTGGATCTGACCTACGGTG |
| | | TACTGGCCGATATCGAAGCGGAAGACCTGGCGCG |
| | | TGAAGCGTCGTTTGCTCAGGGATTACGCGCGATG |
| | | ATTGGCGGTATCTTAACCGCATCCTGATTCTCTC |
| | | TCTTTTCGGCGGGCTGGTGATAACTGTGCCCGCG |
| | | TTTCATATCGTAATTTCTCTGTGCAAAAATTATC |
| | | CTTCCCGGCTTCGGAGAATTCCCCCCAAAATATT |
| | | CACTGTAGCCATATGTCATGAGAGTTTATCGTTC |
| | | CCAATACGCTCGAACGAACGTTCGGTTGCTTATT |
| | | TTATGGCTTCTGTCAACGCTGTTTTAAAGATTAA |
| | | TGCGATCTATATCACGCTGTGGGTATTGCAGTTT |
| | | TTGGTTTTTTGATCGCGGTGTCAGTTCTTTTTAT |
| | | TTCCATTTCTCTTCCATGGGTTTCTCACAGATAA |
| | | CTGTGTGCAACACAG (SEQ ID NO: 13) |
| Down- | BP_DNA_{—} | GTTTCAGGGTTTGCAGACTGATATTCAATGACGG |
| stream | 017 | CTGCGCAACGATACGTACCACATTCTCACGCGTC |
| HA | | GATTTGAAGCAGATGAAGTAAAGCACCATTCCGG |
| | | CAATCGCCAGCACAATTGTCCAGAAATGGTATAC |
| | | CGACACCATCTCTTCCGGGCTGGAGTTCTTAATG |
| | | CTCGGTCCTATCAGAAATGCCAGGCAGACAAAGG |
| | | TCAATGAAGCGGCAATCCCACGAGCCGCGCCCAG |
| | | ACGGGCGCGGGATTGTGGTTGTTGGGTCATCGCG |
| | | GTAGCAAGTGAACCATAAGGAATATTCACCAGGC |
| | | TGTAGCAAAGCCCGAGGCCCATGTAGGTCAAATA |
| | | TGCATACACCACTTTGCTACCATGGCTCCAGTCG |
| | | GTCAGCACCCAGAATACCAGCACGCTGAAGATCA |
| | | TTAACGGCGCAGTACCGAAGAGTAAAAACGGGCG |
| | | GAATTTTCCCCAGCGGGTATTCACACTGTCCACC |
| | | ACTCGTCCGGCAAAGACGTCGGCGAAGGCATCGA |
| | | ATACCCGCACCAGTAACAGCATGGTGCCCGCCGC |
| | | AGCGGCACCGACGCCAGCGACGTCGGTGTAGTAA |
| | | CTCAACAGGAAGAGCGCCCCCATTGCGAAGGCGA |
| | | AGTTATTGGCGACGTCACCGAGGCTGTAGCCGAC |
| | | GATGGTGCGCCAGGAGAGTTGTTGAT (SEQ ID |
| | | NO: 14) |
| tetR_{—} | BP_DNA_{—} | GCATGTAACTGGGCAGTGTCTTAAAAAATCGACA |
| P_{WYL-tet} | 075 | CTGAATTTGCTCAAATTTTTGTTTGTAGAATTAG |
| | | AATATATTTATTTGGCTCATATTTGCTTTTTAAA |
| | | AGCTTGCATGCCTGCAGGTCGACGGTATCGATAA |
| | | CTCGACATCTTGGTTACCGTGAAGTTACCATCAC |
| | | GGAAAAAGGTTATGCTGCTTTTAAGACCCACTTT |
| | | CACATTTAAGTTGTTTTTCTAATCCGCATATGAT |
| | | CAATTCAAGGCCGAATAAGAAGGCTGGCTCTGCA |
| | | CCTTGGTGATCAAATAATTCGATAGCTTGTCGTA |
| | | ATAATGGCGGCATACTATCAGTAGTAGGTGTTTC |
| | | CCTTTCTTCTTTAGCGACTTGATGCTCTTGATCT |
| | | TCCAATACGCAACCTAAAGTAAAATGCCCCACAG |
| | | CGCTGAGTGCATATAATGCATTCTCTAGTGAAAA |
| | | ACCTTGTTGGCATAAAAAGGCTAATTGATTTTCG |
| | | AGAGTTTCATACTGTTTTTCTGTAGGCCGTGTAC |
| | | CTAAATGTACTTTTGCTCCATCGCGATGACTTAG |
| | | TAAAGCACATCTAAAACTTTTAGCGTTATTACGT |
| | | AAAAAATCTTGCCAGCTTTCCCCTTCTAAAGGGC |
| | | AAAAGTGAGTATGGTGCCTATCTAACATCTCAAT |
| | | GGCTAAGGCGTCGAGCAAAGCCCGCTTATTTTTT |
| | | ACATGCCAATACAATGTAGGCTGCTCTACACCTA |
| | | GCTTCTGGGCGAGTTTACGGGTTGTTAAACCTTC |
| | | GATTCCGACCTCATTAAGCAGCTCTAATGCGCTG |
| | | TTAATCACTTTACTTTTATCTAATCTAGACATCA |
| | | TTAATTCCTCCTTTTTGTTGACATTATATCATTG |
| | | ATAGAGTTATTTGTCAAACTAGTTTTTTATTTGG |
| | | ATCCCCTCGAGTTCATGAAAAACTAAAAAAAATA |
| | | TTGACACTCTATCATTGATAGAGTATAATTAAAA |
| | | TAAGCTCTCTATCATTGATAGAGTATGATGGTAC |
| | | CGTTAACAGATCTGAGCCGCAGAGAGGAGGTGTA |
| | | TAAGGTG (SEQ ID NO: 40) |
| kanR | BP_DNA_{—} | GTACCCAGGAAACAGCTATGACCATGTAATACGA |
| Frag- | 076 | CTCACTATACGGGGATATCGTCGGAATTGCCAGC |
| ment | | TGGGGCGCCCTCTGGTAAGGTTGGGAAGCCCTGC |
| | | AAAGTAAACTGGATGGCTTTCTTGCCGCCAAGGA |
| | | TCTGATGGCGCAGGGGATCAAGATCTGATCAAGA |
| | | GACAGGATGAGGATCGTTTCGCATGATTGAACAA |
| | | GATGGATTGCACGCAGGTTCTCCGGCCGCTTGGG |
| | | TGGAGAGGCTATTCGGCTATGACTGGGCACAACA |
| | | GACAATCGGCTGCTCTGATGCCGCCGTGTTCCGG |
| | | CTGTCAGCGCAGGGGCGCCCGGTTCTTTTTGTCA |
| | | AGACCGACCTGTCCGGTGCCCTGAATGAACTGCA |
| | | GGACGAGGCAGCGCGGCTATCGTGGCTGGCCACG |
| | | ACGGGCGTTCCTTGCGCAGCTGTGCTCGACGTTG |
| | | TCACTGAAGCGGGAAGGGACTGGCTGCTATTGGG |
| | | CGAAGTGCCGGGGCAGGATCTCCTGTCATCTCAC |
| | | CTTGCTCCTGCCGAGAAAGTATCCATCATGGCTG |
| | | ATGCAATGCGGCGGCTGCATACGCTTGATCCGGC |
| | | TACCTGCCCATTCGACCACCAAGCGAAACATCGC |
| | | ATCGAGCGAGCACGTACTCGGATGGAAGCCGGTC |
| | | TTGTCGATCAGGATGATCTGGACGAAGAGCATCA |
| | | GGGGCTCGCGCCAGCCGAACTGTTCGCCAGGCTC |
| | | AAGGCGCGCATGCCCGACGGCGAGGATCTCGTCG |
| | | TGACCCATGGCGATGCCTGCTTGCCGAATATCAT |
| | | GGTGGAAAATGGCCGCTTTTCTGGATTCATCGAC |
| | | TGTGGCCGGCTGGGTGTGGCGGACCGCTATCAGG |
| | | ACATAGCGTTGGCTACCCGTGATATTGCTGAAGA |
| | | GCTTGGCGGCGAATGGGCTGACCGCTTCCTCGTG |
| | | CTTTACGGTATCGCCGCTCCCGATTCGCAGCGCA |
| | | TCGCCTTCTATCGCCTTCTTGACGAGTTCTTCTG |
| | | AGCGGGACTCTGGGGTTCGAGAGCTCGCTTGGAC |
| | | TCCTGTTGATAGATCCAGTAATGACCTCAGAACT |
| | | CCATCTGGATTTGTTCAGAACGCTCGGTTG |
| | | (SEQ ID NO: 41) |

The DNA fragments were integrated into the genome of E. coli using the plasmid pKD46 which contains the RED genes to help facilitate recombination of the transformed DNA and the genome. The protocol for making edits using this method is as follows:
1) Make electrocompetent E. coli cells per the protocol outlined in Report_SOPO30 and use plasmid pKD46 to transform the fresh electrocompetent cells.
   a) Recover at 30° C. for 1 hour and plate the cells on LB agar plates with carbenicillin (100 μg/mL) and incubate at 30° C. for 36-48 hours.
2) When colonies are visible, using a sterile inoculation loop, pick a single colony and restreak for single colony isolation on a fresh LB agar plate with carbenicillin (100 μg/mL) and incubate the plates at 30° C. for 36-48 hours.
3) When single colonies have grown to sufficient size, prepare E. coli pKD46 electrocompetent cells again per the protocol outlined in Report_SOP030 with the following modifications:
   a) Add carbenicillin to all growth media prior to transformation to a working concentration of 100 μg/mL.
   b) Culture cells at 30° C. for overnight growth (Day 1 Step 1.4.).
   c) At Day 2 Step 7, after 2 hours of growth at 30° C., add 3.5 mL of 10% arabinose to the cell culture, transfer the flask to the 37° C. shaking incubator at 250 rpm, and incubate the culture for another 45 minutes to 1 h.
   d) Follow the remaining steps for preparing the cells for transformation while paying extra attention to keeping the cells cold but not frozen at all times.
   e) Use >400 ng of linear DNA to transform the E. coli cells and recover at 37° C. for 3 hours.
   f) Plate various volumes (25, 100, 250 μL) of recovered cells on LB agar plates with 50 μg/mL kanamycin added and incubate the plates overnight (16-24 hours) at 37° C.
4) The following day the cells were screened by colony PCR using a primer that binds outside the homology arms and one primer that binds to the putative toxin gene behind the P_{XYL/Tet} promoter.
   a) PCR products were run on a 1% agarose gel to check for colonies that are positive for the integration.
   b) Colonies that were positive for the integration had the DNA insertion and the surrounding region sequenced to confirm that there were no mutations in the inserted fragments.
   c) Once the sequence was confirmed it was struck out for single colony isolation and used in growth assays to observe the effects of inducing and overexpressing the putative toxin genes.

Results:

All of the toxins described above were successfully integrated into the genome an E. coli strain, along with the tetR and kanR genes described previously. Sequencing results showed no mutations in the DNA inserted into the genomes or the surrounding area (˜1000 bases upstream or downstream of the integration site. The synthetic strains are shown in Table 28B.

TABLE 28B
List of E. coli Synthetic Strains

| Strain Name | Genotype |
|---|---|
| BPEC_003 | (K12) ΔuidA::tetR_P_{XYL/tet}-mazF_kanR |
| BPEC_004 | (K12) ΔuidA::tetR_P_{XYL/tet}-relE_kanR |
| BPEC_005 | (K12) ΔuidA::tetR_P_{XYL/tet}-yafQ_kanR |
| BPEC_006 | (K12) ΔuidA::tetR_P_{XYL/tet}-sprA1_kanR |
| BPEC_007 | (K12) ΔuidA::tetR_P_{XYL/tet}-hokD_kanR |
| BPEC_008 | (K12) ΔuidA::tetR_P_{XYL/tet}-hokB_kanR |

Growth Assays for the newly constructed E. coli synthetic strains shown in Table 28B were performed as follows.
1. Start one 5 mL LB+kanamycin (50 μg/mL) culture for each toxin/strain to be tested from a single colony on fresh agar plates. Incubate overnight (12-18 h) in the shaking incubator at 37° C.
2. The next day measure OD600 of overnight cultures.
3. Calculate the volume (V) of overnight (O-N) culture needed to inoculate a fresh 5 mL of LB media to an OD600 of 0.05, V=(0.05/O-N OD600)×5000 μL.
4. Inoculate 2 tubes of LB+kanamycin (50 μg/mL) for each strain being tested using the calculated volume of inoculum from Step 3.
5. Immediately after inoculation and before putting the tubes in the 37° C. shaking incubator, briefly vortex to mix the culture and take the OD for the initial OD reading (t=0). Do not dilute because the OD will be very low (should be around 0.05).
6. Put culture tubes in the shaking incubator at 37° C. for 1 hour.
7. After 1 hour measure and record the OD600 readings, then add 4 μL of anhydrotetracycline (ATc) (1 mg/mL stock solution) to one set of the culture tubes (this is referred to as the spiked samples).
8. Place cultures back in the 37° C. shaking incubator and measure and record the OD600 values every hour for 4 more hours.
9. Enter recorded ODs in a table and plot the data on a graph to show the growth curves for all of the strains tested. The data below was collected from multiple days of experiments.

Results are shown in FIG. 23 to 26.

FIG. 23 shows a graph of the growth curves of (4) different E. coli (sprA1) strains grown in LB with an inducible sprA1 gene integrated in the genome. The dashed line represents the cultures that were induced with ATc and the solid line represents cultures that did not get induced with ATc. All 4 strains that got ATc spiked in the media at 1 h showed a significant decrease in the culture density throughout the entire assay compared to the cultures that did not get an ATc spike. Two different types of target E. coli strains were employed: strains 1, 2, and 15 are from E. coli K12-type target strain IM08B, and strain 16 is the bovine E. coli target strain obtained from Udder Health Systems. All induced strains showed significant decrease in growth over 2-5 hr time points.

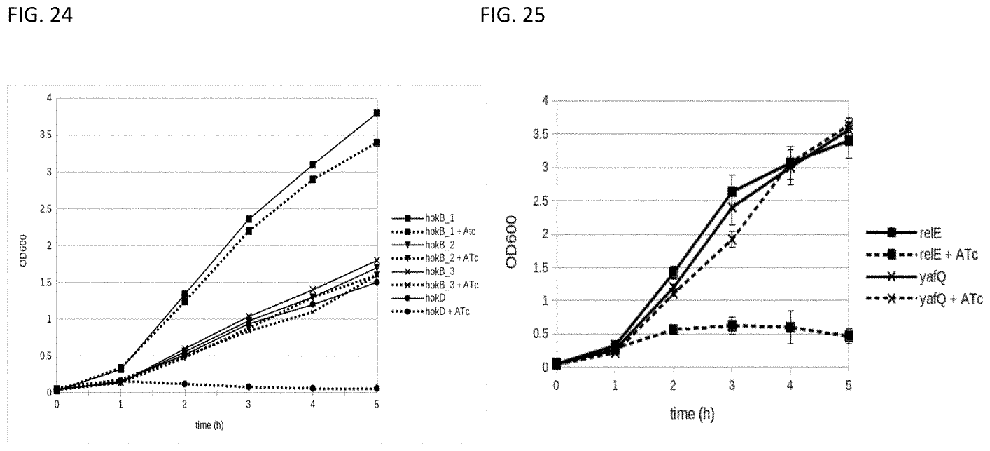

FIG. 24 shows a graph of the growth curves as OD600 values over 5 hrs with of (4) different synthetic E. coli isolates grown in LB with an inducible hokB or hokD gene integrated in the genome of K12-type E. coli target strain IM08B. Samples were induced by adding ATc to the culture 1 h post inoculation. The dashed line represents the cultures that were spiked with ATc to induce expression of the putative toxin genes and the solid line represents cultures that did not get induced by ATc. The hokD sample exhibited a diverging curve between the induced and uninduced samples. The hokB_1 is the bovine E. coli strain from Udder Health Systems and the spiked and unspiked samples grew much faster than the other 3 strains tested here

FIG. 25 shows a graph of the average (n=3) growth curves as OD600 values over 5 hrs of two synthetic E. coli strains with relE or yafQ gene integrated in the genome (n=3) grown in LB (+/−ATc). The dashed lines represent the cultures that were spiked with ATc to induce expression of the putative toxin genes and the solid lines represent cultures that did not get induced by ATc. The error bars represent one standard deviation for the averaged OD600 values for each strain. The relE gene showed diverging curves between the cultures that were induced and the uninduced cultures, where the induced cultures had significantly lower OD600 readings. The induced yafQ cultures showed a slightly slower growth between hours 2 and 4 than the uninduced cultures, but at 5 hours the two groups had nearly identical OD600 values.

Neither synthetic E. coli having genomically integrated mazF gene nor wild type bovine E. coli strain (Udder Health Systems) exhibited statistically significant growth curves over 5 hrs when grown in LB with and without the addition of ATc at t=1 hr to the culture (data not shown).

Synthetic E. coli having genomically integrated sprA1, hokD, and relE genes operably linked to inducible gene when overexpressed exhibited significantly reduced growth in liquid culture. Both sprA1 and hokD showed a fast kill switch activity on the density of the cultures, while relE seemed to have a toxic effect on the host cells 2 hours post induction of the gene.

Two different E. coli target strains were genomically modified under the control of the ATc-inducible P_{XYL/Tet} promoter to incorporate putative E. coli toxins hokB, hokD, relE, mazF, and yafQ, and known S. aureus toxin sprA1. Overexpression of hokD, sprA1, and relE genes resulted in a decrease in the optical density of the synthetic E. coli cell cultures indicating they function as toxins to the host cells. In contrast, overexpression of E. coli comprising hokB, mazF, and yafQ operably linked to the inducible promoter did not demonstrate a toxic effect towards the host cells under the conditions of this assay.

### Example 15. Kill Switch in Synovial Fluid

This example evaluated the phenotypic responses of two synthetic S. aureus BP_109 (kill switch) and BP_121 (control) in human synovial fluid (SF).

Synovial fluid is a viscous liquid found in articulating joints. The two principal functions of synovial fluid are to provide lubrication within articulating joint capsules, and to act as a nutrient transport medium for surrounding tissues. Nutrients are transported to synovial joints via the blood plasma, and likewise waste products are carried away from synovial fluid via the bloodstream. Like plasma, synovial fluid is a serum-derived fluid. Synovial fluid is essentially begins as ultra-filtered blood plasma. As such, many synovial fluid components are derived from blood plasma, and the proteome compositions of the two fluids have been shown to be highly comparable.

Septic arthritis is a condition caused by bacterial infection of joint tissue. Various microorganisms can cause septic arthritis and Staphylococcus aureus is a leading cause of the condition. Septic arthritis can originate from the spread of bacteria from another infection locus in the body via the bloodstream, or from direct inoculation of the joint via puncture wounds or surgery.

Based on the shared origin and compositional similarities among serum, plasma and synovial fluid, it was predicted that the synthetic microorganisms comprising a kill switch would be effective in synovial fluid and reduce cell viability. Two strains were selected for the assay, BP_109 and BP_121. BP_109 is a modified kill switch strain, while BP_121 is phenotypically wild type S. aureus that served as the control group. Control BP_121 (site 2:: code 1) has only a small integration in a non-coding region used for identification by PCR only. Table 29 shows genotypes and sequences of genomically inserted DNA fragments of synthetic S. aureus strains used in this assay.

TABLE 29
Synthetic S. aureus Strains Used synovial fluid assay

| | | DNA Sequence ID of |
|---|---|---|
| Strain | Genotype | Genomic Inserted Fragment |
| BP_121 | BP_001, site2::code 1 | BP_DNA_023 |
| BP_109 | BP_001, isdB::sprA1, | BP_DNA_003 |
| | PsbnA::sprA1, | BP_DNA_040 |
| | ΔsprA1 | BP_DNA_045 |

Media used in the synovial fluid assay are shown in Table 30.

TABLE 30
Media and Other Solutions ued in synovial fluid assay

| Name | Description | Manufacturer | Part Number |
|---|---|---|---|
| TSB | Tryptic Soy Broth (minus | Teknova | T1395 |
| | glucose) | | |
| SF | Human Synovial Fluid | BioChemed | BC51519HSF |
| | (Pooled, Mixed Gender) | | |
| PBS | Phosphate Buffered Saline | Teknova | P0200 |
| TSA | Tryptic Soy Agar Culture | Teknova | T0144 |
| Plates | Plates | | |

Table 31 shows DNA Sequences employed in synthetic strains. All DNA insertions and deletions are double stranded DNA. Only single stranded sequences are listed above.

TABLE 31
DNA Sequences used in BP_109 and BP_121

| Sequence | | Sequence of Insert or |
|---|---|---|
| ID | Genotype | Deletion |
| BP_DNA_023 | BP_001, | Cgatcttcgacatcggaccctagaaca |
| | site2::code | gaacta (SEQ ID NO: 19) |
| BP_DNA_003 | isdB::sprA1 | CGCAGAGAGGAGGTGTATAAGGTGATG |
| | | CTTATTTTCGTTCACATCATAGCACCA |
| | | GTCATCAGTGGCTGTGCCATTGCGTTT |
| | | TTTTCTTATTGGCTAAGTAGACGCAAT |
| | | ACAAAATAG (SEQ ID NO: 3) |
| BP_DNA_040 | PsbnA::sprA1 | CGCAGAGAGGAGGTGTATAAGGTGATG |
| | | CTTATTTTCGTTCACATCATAGCACCA |
| | | GTCATCAGTGGCTGTGCCATTGCGTTT |
| | | TTTTCTTATTGGCTAAGTAGACGCAAT |
| | | ACAAAATAG (SEQ ID NO: 26) |
| BP_DNA_045 | ΔsprA1 | ATATAATAGTAGAGTCGCCTATCTCTC |
| | (deletion | AGGCGTCAATTTAGACGCAGAGAGGAG |
| | of 5′ end) | GTGTATAAGGTGATGCTTATTTTCGTT |
| | | CACATCATAGCAC (SEQ ID NO: |
| | | 29) |

Synovial Fluid Assay protocol involves culture preparation, serial dilutions, plating and colony counting as shown below.
1. Culture Preparation
   1.1. Cultures were started by inoculating 5 mL TSB with single colonies of BP_109 and BP_121 in 14 mL sterile culture tubes, and placing them in the shaking incubator at 37° C. and 240 rpm to grow overnight. (3 tubes each for biological replicates)
   1.2. The following morning, the overnight cultures were cut back to 0.05 OD600 in 5.5 mL of fresh TSB.
      1.2.1. OD600 was measured in 1 cm cuvette on NanoDrop spectrophotometer.
      1.2.2. The resulting OD600 values were used to calculate the volume of overnight culture needed to inoculate fresh TSB to 0.05 OD600.
      1.2.3. Fresh 5.5 mL TSB cultures were inoculated with appropriate volumes of overnight culture and incubated for 2 hrs (37° C., 240 rpm) in order to get the cells growing in log phase again.
   1.2.4. After the 2 hour incubation the OD600 was measured for each culture.
   1.2.5. The cultures were then washed in sterile PBS.
      1.2.5.1. Cultures were centrifuged to pellet the cells using the swing out rotor (3500 rpm, 5 mins, RT), and washed with 5 mL PBS.
      1.2.5.2. Cultures were centrifuged to pellet the cells again, and resuspended in 1 mL sterile PBS.
   1.2.6. The OD600 values obtained after the 2 hour incubation were used to calculate the volume needed to inoculate 1.8 mL of Synovial Fluid or TSB to 0.05 OD600.
      1.2.6.1. (Measured OD600)(X mL)=(0.05 OD600)(1.8 mL)
   1.2.7. The following cultures were then inoculated in pre-warmed 37° C.:
      1.2.7.1. BP_109 in TSB (1 tube)
      1.2.7.2. BP_109 in Synovial Fluid (3 tubes)
      1.2.7.3. BP_121 in TSB (1 tube)
      1.2.7.4. BP_121 in Synovial Fluid (3 tubes)
   1.2.8. After addition of inoculum, cultures were mixed by pulse vortex and 100 uL samples were taken for determining cfu/mL by dilution plating (see below).
   1.2.9. The cultures were immediately placed in the 37° C. shaking incubator (240 rpm) and samples were taken after 2 hrs and again at 4 hrs to determine cfu/mL by dilution plating.
2. Serial Dilutions and Culture Plating
   2.1. Dilution plating was performed using the Opentrons OT-2 robot following the protocol described in Report_SOP017.
      2.1.1. Dilutions were carried out to a concentration where 30-300 colonies grew from plating 100 μL of diluted sample on TSA plates.
3. Incubation and Colony Counting
   3.1. TSA plates were incubated overnight for 12-16 hrs at 37° C.
   3.2. The following morning, plates were removed from the incubator and colony counting was performed to determine the concentration of viable cells at each time point (cfu/mL).
      3.2.1. Multiple dilutions were plated in duplicate for each condition at each time point, only plates with 30-300 colonies were used to calculate cfu/mL values.

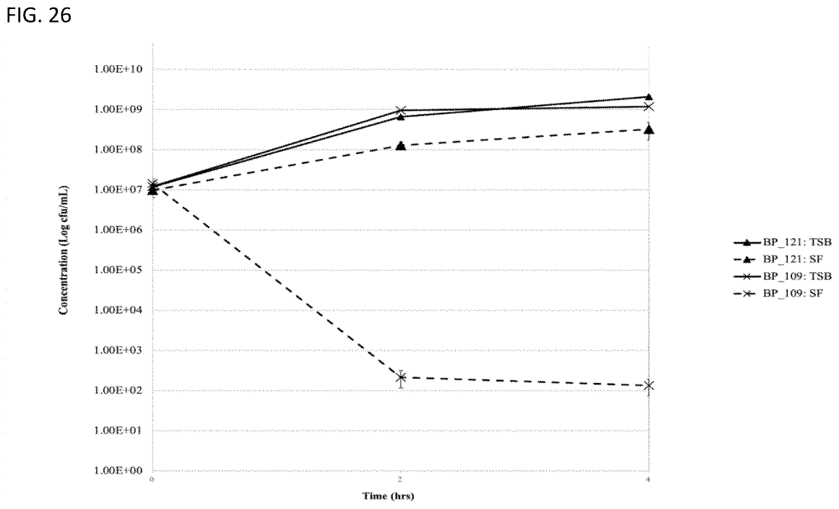

Results for the synovial fluid assay are shown in FIG. 26 showing a graph the concentrations of synthetic S. aureus BP_109 and BP_121 cells grown in in TSB and human synovial fluid over the course of a 4 hour growth assay. Both BP_121 (control) and BP_109 (kill switch) cultures grew in TSB. BP_109 showed a rapid decrease in viable cfu/mL in the synovial fluid condition.

The present study demonstrated that BP_109 behaves similarly in human synovial fluid as it does in human plasma and human serum. BP_109 in SF showed significant decreases in viable cfu/mL over the first two hours of the assay, and by the hour 4 only a few viable colonies remained. In contrast, BP_121 grew in synovial fluid at a rate similar to the BP_121 and BP_109 TSB control groups. The results of this assay support the conclusion that the genetically engineered kill switch strain BP_109 functions as designed. The kill switch appears to be activated in human synovial fluid which severely and suddenly reduces the concentration of viable cells in the fluid.

### Example 16. Kill Switch in Cerebrospinal Fluid

This experiment evaluated the phenotypic responses of synthetic S. aureus strains BP_109 (kill switch) and BP_121 (control) in rabbit cerebrospinal fluid (CSF) enriched with 2.5% human serum. BP_109 performed similarly in serum enriched CSF as it does in human plasma, human serum, and human synovial fluid. BP_109 in serum enriched CSF showed significant decreases in cfu/mL over the course of 6 hours.

Cerebrospinal fluid is a clear liquid that surrounds the central nervous system (CNS). CSF principally functions as a mechanical barrier to cushion the CNS, and is involved in the auto-regulation of cerebral blood flow. Additionally, CSF functions as a transport media, providing nutrients from the bloodstream to surrounding tissues and removing wastes, and as such has often been referred to as a “nourishing liquor.” Despite this characteristic as a nutrient transport media, CSF is a nutrient poor environment compared to blood plasma. Numerous species of bacteria, including S. aureus, have been reported to exhibit little to no growth in CSF in vitro. This phenomenon might be an evolutionary means to protect the central nervous system from bacterial invaders via nutrient sequestration. Additionally, CSF is protected from microbial invasion by the meninges, which are membranes that surround the brain and spinal cord. CSF occupies the subarachnoid space between the two innermost meninges, arachnoid mater and pia mater. Bacterial infection of these tissues produces inflammation, referred to as meningitis Aguilar et. al. “Staphylococcus aureus Meningitis Case Series and Literature Review.” Medicine, vol. 89, no. 2, pp. 117-125, 2010

There are two scenarios in which S. aureus meningitis may be likely to arise. The first is postoperative meningitis. This occurs when the structural integrity of the of the meningeal linings encompassing CSF become compromised during surgical procedures. In these circumstances infections can occur when bacteria are able to enter during surgery, spread from a nearby contagious infection, or enter through CSF shunts. The second pathogenic mechanism for S. aureus meningitis is known as hematogenous meningitis, which is a secondary infection caused by bacteremic spread from an infection outside of the CNS. In cases of methicillin resistant Staphylococcus aureus (MRSA) meningitis, the vast majority have been reported to be nosocomial in origin. Pinado et al. “Methicillin-Resistant Staphylococcus aureus Meningitis in Adults.” Medicine, vol. 91, no. 1, pp. 10-17, 2011.

Given the relative inability of S. aureus to grow in healthy spinal fluid in vitro, it was deemed appropriate to create conditions to mimic potentially susceptible states in vivo. The present study investigated the efficacy of a synthetic Staph aureus having a kill switch in CSF under mock conditions of a perturbed state, where the usually highly protected cerebrospinal fluid environment has become contaminated with nutrient rich serum, thus creating an environment susceptible to infection. Rabbit CSF was spiked with 2.5% human serum. It was hypothesized that the addition of this low level of serum would stimulate enough metabolic activity for kill switch activation in BP_109, resulting in dramatic reduction in viability. BP_121 (control), and synthetic strain BP_109 comprising a kill switch genomic modification, as described in example 15 were subjected to the CSF assay.

The protocol for the CSF assay was similar to that described in example 15, except synovial fluid was replaced with contaminated CSF which was rabbit CSF (New Zealand White RabbitRabbit Cerebrospinal Fluid, BioChemed) spiked with 2.5% human serum.

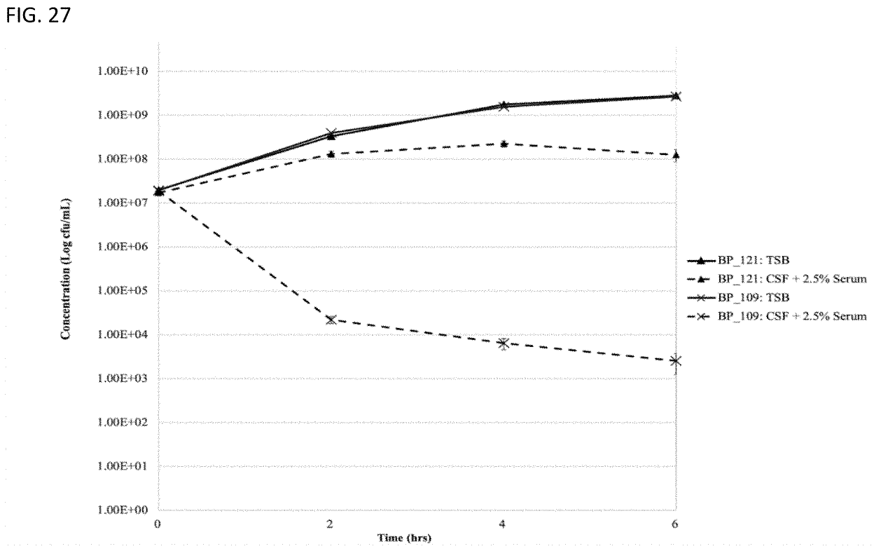

FIG. 27 shows a graph of the concentration of viable BP_109 and BP_121 cells in TSB and Serum Enriched CSF over the course of a 6 hour assay. Both BP_121 (control) and BP_109 (kill switch) cultures grew in TSB. BP_121 also grew in CSF enriched with 2.5% human serum; however, BP_109 showed a rapid decrease in cfu/mL in the CSF condition.

This experiment evaluated the phenotypic responses of BP_109 and BP_121 in cerebrospinal fluid. Both strains are genetically engineered versions of S. aureus 502a, however, BP_121 has only a small integration in a non-coding region, and is phenotypically wild type. BP_109 is a genetically engineered kill switch strain of 502a (BP_001) which has previously been shown to significantly decrease in cfu/mL after being introduced to human serum, plasma, and synovial fluid.

Despite the fact that S. aureus is capable of causing life-threatening meningitis, previous studies have shown that does not readily grow, or die, but rather remains stable in CSF in vitro. As such, human serum (2.5%) was added to CSF in order to provide basic nutrients necessary for growth. Under these serum enriched CSF conditions BP_109 decreased in viability by several orders of magnitude. The results of this assay support the conclusion that the genetically engineered kill switch strain BP_109 functions as designed in contaminated CSF. The kill switch appears to be activated in 2.5% serum enriched rabbit CSF and BP_109 dies.

### Example 17. Bacteremia Study in vivo Staphyloccocus aureus

An in vivo bacteremia mouse study to compare the clinical effects (bacteremia) in mice subjected to a tail vein injection of two Staph aureus microorganisms modified with kill switch (KS) technology with wild-type (WT) Staphylococcus aureus (SA).

In this study, all mice injected with 10\hat{ }7 CFU/mouse of synthetic Staph aureus (KS) survived the entire 8 day duration of the study and demonstrated health, lack of clinical symptoms, and maintained body weight. All positive controls (mice injected with 10\hat{ }7 CFU/mouse of WT SA) died or were determined moribund and euthanized by ethical standards.

Normal weight was defined as weight within 15% of the initial weight.

Synthetic strains of Staph aureus comprising kill switch genomic modifications exhibited good efficacy in human plasma, human serum, human synovial fluid, and contaminated rabbit cerebrospinal fluid assays in vitro as described herein. The present Bacteremia Study was designed to test the efficacy of two KS modified Staph strains, BP_109 and CX_013 (Table 32), in the prevention of bacteremia after tail vein injection. BP_001 and CX_001, are wild type organisms of the same lineage as BP_109 and CX_013, respectively, and were included in the study as positive controls.

Based on the kill switch activity of synthetic KS strains in vitro, it was hypothesized that the kill switch would also perform as designed in vivo and initiate artificially programmed cell death upon entering the bloodstream. It was predicted that mice in the kill switch groups would remain healthy and fail to develop bacteremic infections, and that wild type groups would develop severe bacteremia, or be diagnosed as moribund and euthanized. Results of the study met these expectations.

Materials

BioPlx engineered two organisms for use in the mouse bacteremia study. The two synthetic Staph aureus organisms are designated BP_109 and CX_013 and were generated through the genomic alteration of organisms BP_001 and CX_001, respectively as shown in Table 32.

TABLE 32
Strains Used in Mouse Bacteremia Study

| Strain | Genotype |
|---|---|
| BP_001 | wild type |
| BP_109 | BP_001, isdB::sprA1, PsbnA::sprA1, ΔsprA1 |
| CX_001 | wild type isolated from microbiome swab |
| CX_013 | isdB::sprA1 |

Table 33 shows the strains used and the targeted concentration of cells in CFU/mouse.

TABLE 33
Groups, Treatment and Dosing

| | Treatment | | |
|---|---|---|---|
| | (100 uL tail vein | Target Dose | |
| Group | injection) | (CFU/mouse) | Designation |
| 1 | Vehicle (Sterile PBS) | NA | Negative Control |
| 2 | Killed BP_001 | 10\hat{ }7 | Negative Control - |
| | | | Wild Type |
| 3 | BP_001 | 10\hat{ }7 | Positive Control - |
| | | | Wild Type |
| 4 | BP_109 | 10\hat{ }7 | Test Group - Kill |
| | | | Switch |
| 5 | CX_001 | 10\hat{ }7 | Positive Control - |
| | | | Wild Type |
| 6 | CX_013 | 10\hat{ }7 | Test Group - Kill |
| | | | Switch |

Methods

Test Article Preparation

The test articles were prepared as follows. Briefly, single colonies of each strain were picked and grown overnight in liquid tryptic soy broth (TSB). For each strain, 1 mL of the overnight culture was used to inoculate 100 mL of fresh TSB and then incubated for another 14 hours. After the 14 hour incubation period, the cells were washed three times with phosphate buffered saline (PBS), a sample was serially diluted and plated on tryptic soy agar (TSA) plates to determine the CFU/mL, and the cells were stored overnight at 4° C.

The next day the CFU plates were counted and the actual concentration was determined. Using the calculated CFU/mL cell concentrations of the PBS cell solutions, final test articles were prepared at the appropriate concentrations. An aliquot of BP_001 was made and treated with 70% isopropyl alcohol to kill the cells, then washed three times with PBS to remove any alcohol. While the alcohol treatment group was incubating, the remaining treatment groups were prepared from the PBS cell solutions. The test articles were then hand delivered to the facility where the dosing and observations occurred.

Non-GLP Mouse Study

A non-GLP exploratory study was performed. Five BALB/c male mice were assigned to each group for experimentation. Each animal was dosed once intravenously on study Day 0 by tail vein injection using sterile PBS as the vehicle. The treatment and dosing by group is shown in (Table 33).

BALB/c mice were selected as a suitable model for a bacteremia study as well as intravenous injection according to literature reports. Stortz et al. “Murine models of sepsis and trauma: can we bridge the gap?.” ILALR journal 58.1 (2017): 90-105. The bacteria levels (10\hat{ }7 CFU/mouse) were chosen based on similar peer-reviewed articles studying bacteremia effects in mice of the same species and of similar age. van den Berg et al. “Mild Staphylococcus aureus skin infection improves the course of subsequent Endogenous S. aureus bacteremia in mice.” PloS one 10.6 (2015): e0129150. Prior to injection, the animals were allowed 48 hours to acclimate to the new environment and body weights were obtained and recorded on study Day 0. Body weights were measured once each morning for the duration of the study. Mortality and morbundity checks were performed twice a day (once in the morning and once in the evening) for the duration of the study. Animals who experienced a 20% or greater loss in weight were deemed suitable for euthanasia.

All procedures conformed to USDA guidelines for animal care and handling. Study design and animal usage were approved by the USDA certified (84-R-0081) and OLAW assured facility (A4678-01) performing the study.

Results

The pre-dose body weights ranged from 21.9 to 30.7 g. Clinical observations and body weight measurements were all normal for Groups 1, 2, 4 and 6 (negative controls and kill switch test groups) with the exception of one observation of hypoactivity in one mouse from Group 4 on study Day 2.

Numerous abnormal clinical observations, including (but not limited to) significant weight loss, rough coat, milky eye excretions and death, were observed for all mice in Groups 3 and 5 (positive controls). All animals from Group 3 (BP_001 subjects) were deceased upon conclusion of the study. Three of the five animals from Group 5 (CX_001 subjects) were deceased upon conclusion of the study and the two survivors had beyond 20% weight loss declaring both fit for euthanasia.

Bacteremia results are depicted in FIG. 28. The graph values were generated by averaging and normalizing the body weight for each group of interest. Normalization was performed by dividing the group (average) weight at each time point by the initial group (average) weight. Each time point average was generated using only surviving mice. A graphic is shown at the bottom of the graph to represent adverse clinical observations and mortality.

A Bacteremia Study was performed in vivo in mice to compare the clinical effects (bacteremia) in a mouse model following tail vein injection of 10\hat{ }7 Staphylococcus aureus (SA) modified with kill switch (KS) technology or wild type (WT) target strains. The organisms modified with KS technology were designed to initiate artificially programmed cell death upon interacting with blood, serum, or plasma of the mammalian host.

All mice injected intravenously via tail vein injection with KS organisms as well as negative controls were healthy with no adverse clinical symptoms for the duration of the study, excluding one observation of hypoactivity which subsided by next observation. All mice injected with WT organisms experienced a wide variety of abnormal clinical observations, significant morbundity, and were either deceased or were fit for euthanasia by ethical standards. This study demonstrated the efficacy and safety of the kill switch KS technology with 100% survival and health of all test subjects over the 8 days of study. Synthetic Staph aureus strains comprising a kill switch may significantly de-risk protective organisms for use in methods for prevention and treatment of infectious disease.

### Example 18. SSTI Study In Vivo Staphylococcus aureus

An in vivo study was performed to compare the clinical effects (skin and soft tissue infection) in mice subjected to subcutaneous injections with wild-type (WT) Staphylococcus aureus (SA) vs two SA organisms modified with kill switch (KS) technology. Study duration was ten days.

In this study, all mice injected with 10\hat{ }7 synthetic Staphylococcus aureus KS strains demonstrated health in both clinical symptoms (i.e. no abscess formation) and maintained body weight for the duration of the study, while half of the positive controls (mice injected with WT SA strains) developed abscesses.

An in vivo mouse Skin and Soft Tissue (SSTI) Study was designed to test the efficacy of two KS-modified SA strains, BP_109 and CX_013 (Table 34), in the prevention of SSTI after subcutaneous injection. BP_001 and CX_001, are wild-type (WT) organisms of the same lineage as BP_109 and CX_013, respectively, and were included in the study as positive controls. Based on the kill switch efficacy achieved in vitro and in an in vivo Bacteremia Study it was hypothesized that the KS would also perform as designed in vivo after subcutaneous injection and initiate artificially-programmed cell death upon entering the body under the skin. It was predicted that mice in the KS groups would remain healthy throughout the study and fail to develop SSTI infections. The WT groups were expected to develop abscess formation (indicative of SSTI).

Materials

The SSTI study employed two synthetic Staph aureus KS strains designated BP_109 and CX_013 and two WT target microorganisms BP_001 and CX_001 as shown in Table 34.

TABLE 34
Staphylococcus aureus trains used in SSTI Study

| | | DNA Sequence ID of |
|---|---|---|
| Strain | Genotype | genomic inserted fragment |
| BP_001 | wild type | n/a |
| BP_109 | BP_001, isdB::sprA1, | BP_DNA_003 |
| | PsbnA::sprA1, | BP_DNA_003 |
| | ΔsprA1 | BP_DNA_045 |
| CX_001 | wild type | n/a |
| CX_013 | CX_001, isdB::sprA1 | BP_DNA_003 |

Table 35 shows treatment groups, target dose and strain types employed in the SSTI study.

TABLE 35
SSTI Treatment Groups, Treatment and Dosing

| | | | Actual Dose | |
|---|---|---|---|---|
| | Treatment | Target Dose | Administered | Strain |
| Group | (100 uL SC) | (CFU/mouse) | (CFU/mouse) | Type |
| 1 | Vehicle (Sterile | n/a | n/a | n/a |
| | PBS) | | | |
| 2 | Killed BP_001 | 10\hat{ }7 | 0 | WT (neg) |
| 3 | BP_001 | 10\hat{ }7 | 6.00E+06 | WT (pos) |
| 4 | BP_109 | 10\hat{ }7 | 1.61E+07 | KS (test) |
| 5 | CX_001 | 10\hat{ }7 | 1.21E+07 | WT (pos) |
| 6 | CX_013 | 10\hat{ }7 | 7.95E+06 | KS (test) |

SC—Subcutaneous Injection;
Neg—Negative;
Pos—Positive;
WT—Wild Type;
KS—Kill Switch

Test Article Preparation

The test articles were prepared according to a protocol described by Malachowa et al. 2013. Malachowa, Natalia., et at “Mouse model of Staphylococcus aureus skin infection.” Mouse Models of Innate Immunity. Humana Press, Totowa, N.J., 2013. 109-116.

Briefly, single colonies of each strain were picked and grown overnight in liquid tryptic soy broth (TSB). For each strain, 1 mL of the overnight culture was used to inoculate 100 mL of fresh TSB and then incubated for another 14 hours. After the 14-hour incubation period, the cells were washed three times with phosphate buffered saline (PBS), a sample was serially diluted and plated on tryptic soy agar (TSA) plates to determine the CFU/mL, and the cells were stored overnight at 4° C. The next day the CFU plates were counted and the actual concentration was determined. Using the calculated CFU/mL cell concentrations of the PBS cell solutions, final test articles were prepared at the appropriate concentrations. One aliquot of BP_001 was made and treated with 70% isopropyl alcohol to kill the cells, then washed three times with PBS to remove any alcohol. While the alcohol treatment group was incubating, the remaining treatment groups were prepared from the PBS cell solutions. The test articles were then hand-delivered to the facility where the dosing and observations occurred.

A non-GLP exploratory study was performed over 10 days. Five BALB/c male mice (Charles River) were assigned to each group for experimentation. Each animal was dosed once subcutaneously on study Day 0 using sterile PBS as the vehicle and observed for 10 days post injection. The treatment and dosing by group is shown in Table 35. The bacteria levels (10\hat{ }7 CFU/mouse) were chosen based on similar peer-reviewed articles studying SSTIs as well as systemic bacterial effects in mice of the same species and of similar age. Prior to injection, body hair was removed from the animals in the areas surrounding the injection site (dorsal surface). The animals were allowed adequate acclimation time, both before and after hair removal, to stabilize. Body weights were obtained and recorded on study Day 0. Pictures of the injection site/abscess were photographed once per day for all subjects in all groups. Abscesses present were measured once daily (length and width) using calipers. Body weights were measured once each morning for the duration of the study. Mortality and morbundity checks were performed twice a day (once in the morning and once in the evening) during business days and once on the weekends. Animals who experienced a 20% or greater loss in weight were deemed moribund suitable for euthanasia. All procedures abided by USDA guidelines for animal care and handling. Study design and animal usage were approved by the Institutional Animal Care and Use Committee (IACUC) in a USDA certified (84-R-0081) and OLAW assured facility (A4678-01).

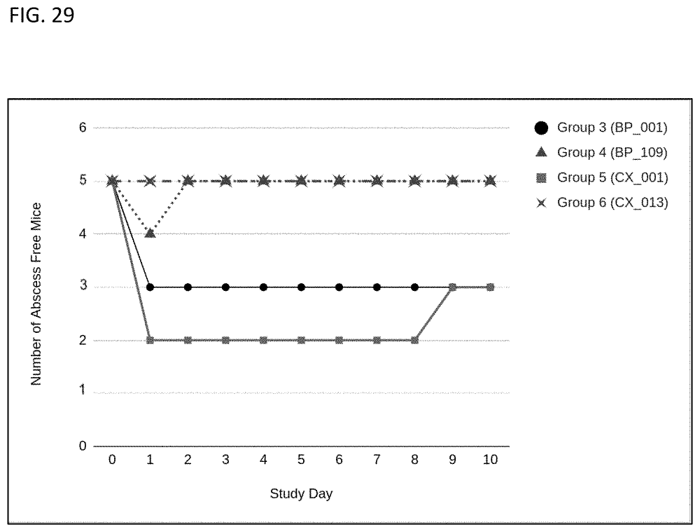

FIG. 29 shows a graph of animal health in the SSTI study as measured by abscess formation, or the lack thereof over the 10 day duration of the study. Mice in Groups 4 and 6, BP_109 and CX_013, respectively, maintained health over the course of this study, as compared to their wild type parent strains BP_001 and CX_013, respectively. Animals in the negative control Groups 1 (vehicle) and 2 (killed WT BP_001) all remained healthy throughout the study and are not shown.

On Study Day 1—the day following injection—clinical observations were normal for mice in the negative control Groups 1 and 2. Likewise, none of the mice in the KS groups—Groups 4 and 6—exhibited adverse clinical observations one day post injection, with the exception of one minor reaction. A small, light colored bump was observed on one mouse from Group 4, BP_109, on study Day 1. By study Day 2 the bump was no longer present on the Group 4 mouse, and all mice from the KS groups maintained good health with no adverse clinical observations for the remainder of the study. Images of the injection site were collected (FIGS. 1-2).

In contrast, half of the mice in the WT positive control groups began to exhibit signs of infection shortly after the onset of the study. Five of the ten mice from the WT positive control groups experienced abscess formation by study Day 1. This included two mice from Group 3, BP_001, and three mice from Group 5, CX_001. Signs of infection in the BP_001 group initially presented as yellow colored formations, which quickly progressed into large off-white colored abscesses surrounded by irritated red margins. Abscesses were present for the remainder of the study for both mice in Group 3.

The SSTIs in Group 5 presented as small red abscesses, and one mouse in Group 5 was observed to return to normal clinical observations by study Day 9. Abscesses were present for the duration of the study for the other two mice in Group 5.

The pre-dose mouse body weights ranged from 19.0 g to 24.1 g. All subjects maintained normal body weight for the duration of the study. Therefore, a hypothesis test for binomial distributions was used to compare the KS test subjects to the positive control subjects for significance. This was done by strain derivation; i.e. BP_109 was compared to BP_001 and CX_013 was compared to CX_001. Animals with abscess formation were assigned a value of 1 and those without abscess formation were assigned a value of 0, as shown in Table 36. As compared to WT SA subcutaneous injection, the BioPlx KS groups exhibited significantly fewer SSTIs (p<0.01).

Statistical Analysis

No weight deviation occurred for any of the groups involved in the study, so a dichotomous score was used to compare groups by an absolute measure. Any abscess formation throughout the study assigned a mouse a value of 1 and complete absence of abscess formation for the duration of the study assigned a mouse a value of 0. As such, the results were as follows:

TABLE 36
Dichotomous Score for Abscess Formation by Group per Mouse

| Group | | | | | | Group |
|---|---|---|---|---|---|---|
| Treatment | Mouse 1 | Mouse 2 | Mouse 3 | Mouse 4 | Mouse 5 | Score |
| BP_001 | 0 | 1 | 0 | 1 | 0 | 2/5 |
| BP_109 | 0 | 0 | 0 | 0 | 0 | 0/5 |
| CX_001 | 0 | 1 | 1 | 1 | 0 | 3/5 |
| CX_013 | 0 | 0 | 0 | 0 | 0 | 0/5 |
| Killed | 0 | 0 | 0 | 0 | 0 | 0/5 |
| BP_001 | | | | | | |

Abscess Formation = 1;
No Abscess Formation = 0

The hypothesis test for binomial distributions was used to compare groups by parent/daughter strains. In other words, the analysis was used to compare BP_001 to BP_109 and CX_001 to CX_013 as the latter were derived from the former. Probability was assigned by the WT groups' presence of abscess formation, and alpha was set to 99% confidence.

The hypothesis test for binomial distributions determined that five out of five mice in the test group must be abscess free for both strains to achieve a 99% confidence. As all five mice from both test groups, BP_109 and CX_013, were completely abscess free, we may report that both test groups are significantly different to the comparative WT groups with a p-value <0.01.

In this SSTI study, all mice injected subcutaneously with SA KS organisms as well as negative controls were healthy and normal for the duration of the study, excluding one minor reaction on a test subject on study Day 1, which was resolved by the morning of Day 2. Half of the mice injected with WT SA organisms had abscess formations present for most of the study.

### Example 19. Bacteremia and SSTI Study High Dose Staph aureus

The present in vivo Bacteremia and SSTI high dose study was designed to test the upper limits of four synthetic KS-modified Staph aureus strains in the ability to prevent bacteremia and skin and soft tissue infection (SSTI) in a mouse model.

The study objective was to compare the clinical effects (bacteremia and SSTI abscesses) in mice subjected to intravenous tail vein or subcutaneous injection, respectively, with different synthetic and wild-type strains of Staphylococcus aureus (Staph aureus).

In the high dose Bacteremia Study mice were injected intravenously with high concentrations (10\hat{ }9 CFU/mouse) of Staph aureus synthetic strains BP_123 and CX_013-both modified with kill switch technology. All mice survived the duration of the study in good health with clinical symptoms only appearing on study Day 0, the same day as injection.

In the high dose SSTI Study mice were injected subcutaneously with high concentrations (10\hat{ }9 CFU/mouse) of kill switched organisms BP_123 and CX_013 were also in good health with no abscess formation between the two groups, whereas the comparative wild-type groups all developed abscesses of a high severity.

An “abscess” was defined as an acute persistent inflammatory response resulting in a visible accumulation of purulent material in either an encapsulated or ruptured state. For injections of 10\hat{ }9 CFU of Staph aureus, the accumulated material must be present for more than 3 days to qualify as a persistent abscess.

Materials

Four organisms were designated for use in the present study. The four modified organisms are designated BP_092, BP_109, BP_123 and CX_013. The BP strains and CX_013 strain were generated through the alteration of wild type organisms BP_001 and CX_001, respectively. Stains used in the present study are shown in Table 37.

TABLE 37
Strains used in High Dose Bacteremia and SSTI Studies

| Strain | Genotype |
|---|---|
| BP_001 | wild type |
| BP_092 | BP_001, PsbnA::sprA1 |
| BP_109 | BP_001, isdB::sprA1, PsbnA::sprA1, ΔsprA1 |
| BP_123 | BP_001, isdB::sprA1, ΔsprA1 |
| CX_001 | wild type from microbiome swab |
| CX_013 | CX_001, isdB::sprA1 |

Table 38 shows the strains used for each group and the targeted concentration of cells in CFU/mouse. Negative controls were prepared at doses of 1.00E+09 CFU/mouse and were heat-killed prior to injection.

TABLE 38
High Dose Bacteremia and SSTI Study
Groups, Treatment and Dosing

| | | | Actual Dose | | |
|---|---|---|---|---|---|
| | | Target Dose | Administered | | Strain |
| Group | Treatment | (CFU/mouse) | (CFU/mouse) | Route | Type |
| 1 | BP_001 | 1.00E+09 | 9.60E+08 | IV | WT (pos) |
| 2 | CX_001 | 1.00E+09 | 9.10E+08 | IV | WT (pos) |
| 3 | BP_092 | 1.00E+09 | 9.15E+08 | IV | KS (test) |
| 4 | BP_109 | 1.00E+09 | 9.35E+08 | IV | KS (test) |
| 5 | BP_123 | 1.00E+09 | 9.05E+08 | IV | KS (test) |
| 6 | CX_013 | 1.00E+09 | 9.50E+08 | IV | KS (test) |
| 7 | Killed | 0 | 0 | IV | WT (neg) |
| | BP_001 | | | | |
| 8 | BP_001 | 1.00E+09 | 9.80E+08 | SC | WT (pos) |
| 9 | CX_001 | 1.00E+09 | 1.01E+09 | SC | WT (pos) |
| 10 | BP_092 | 1.00E+09 | 1.13E+09 | SC | KS (test) |
| 11 | BP_109 | 1.00E+09 | 9.00E+08 | SC | KS (test) |
| 12 | BP_123 | 1.00E+09 | 1.00E+09 | SC | KS (test) |
| 13 | CX_013 | 1.00E+09 | 1.33E+09 | SC | KS (test) |
| 14 | Killed | 0 | 0 | SC | WT (neg) |
| | BP_001 | | | | |

WT: Wild-type;
KS: Kill switch;
pos—positive control;
test—experimental strain;
neg—negative control;
IV: Intravenous;
SC: Subcutaneous

Methods

Test Article Preparation

The test articles used in this study were prepared as follows. Briefly, overnight cultures were grown in shake flasks. The following day, the cells were harvested and concentrated. Identical aliquots of each strain were prepared and frozen in cryovials at −80° C. Several frozen aliquots were later thawed, washed three times with phosphate buffered saline (PBS), resuspended in the original volume of PBS, and CFU counts were determined by dilution plating. The remaining frozen aliquots, of known concentration, were stored at −80° C. Directly prior to use in animal model studies, aliquots of known concentration were thawed, washed with PBS, and resuspended in appropriate volumes of PBS to reach the target concentrations. The concentration of each test article was determined by dilution plating, and the phenotype of each test article was confirmed by serum assay (data not shown).

Non-GLP Mouse Study

A non-GLP exploratory 7-day study was performed. Five BALB/c male mice were assigned to each group 1-14. Each animal was weighed immediately prior to injection for the initial weight measurement. Each animal was dosed once on study Day 0 using sterile PBS as the vehicle and observed for 7 days post injection. The treatment and dosing by group is shown in Table 38.

The high bacteria levels (10\hat{ }9 CFU/mouse) were chosen based on two prior animal studies described herein using 10\hat{ }7 CFU/mouse. This study was designed to test the upper limits of the protective organism, so a 100-fold dosage increase was chosen.

Animals in high dose bacteremia study belonging to Groups 1-7 received 50 μL intravenous tail vein injection of Staph aureus at 10\hat{ }9 CFU/mouse and were observed for clinical observations of bacteremic infection.

Animals belonging to high dose SSTI study Groups 8-14 received 100 μL subcutaneous injection of Staph aureus at 10\hat{ }9 CFU/mouse and were observed for clinical observations of SSTI, mainly abscess formation. Prior to injection, body hair was removed from areas surrounding injection site (dorsal surface) for animals receiving subcutaneous injection and being observed for abscess formation (Groups 8-14).

In order to better understand the cause of any abscess that may form in a mouse injected subcutaneously with kill switched Staph aureus, following euthanization of the mice, a sample of the fluid in the abscess was taken using a sterile inoculating loop and plated on non-selective agar media to culture any possible bacteria in the abscess. The colonies that grew on the plates were screened by PCR for the genomically integrated kill switches that pertain to the test article group (data not shown).

Results

The pre-dose body weights ranged from 17.3 g to 22.4 g. All animals (Groups 1-14) were allowed adequate acclimation time—both before and after hair removal for applicable groups—to stabilize. Table 39 shows clinical observations for Bacteremia Groups 1-7.

TABLE 39
Clinical Observations for High Dose Bacteremia Groups 1-7

| Group | Study Observations (7-Day Study) |
|---|---|
| 1. BP_001 WT | Day 0 - Three animals lethargic (1001, 1004, 1005) |
| Positive Control, | Day 1 - Three animals found dead (1001, 1003, 1004), and one animal |
| IV 10\hat{ }9 CFU/ | euthanized (1005). |
| mouse. | Day 2 - None. |
| n = 5 | Day 3 - None. |
| | Day 4 - None. |
| | Day 5 - One animal euthanized (1002). All animals deceased. |
| 2. CX_001 WT | Day 0 - All five animals lethargic, some with hunched posture and milky |
| Positive Control, | secretions from eyes (2001-2005). |
| IV 10\hat{ }9 CFU/ | Day 1 - Three animals euthanized (2001, 2004, 2005), and two animals are |
| mouse. | lethargic, hunched posture, and milky secretions from eyes (2002, 2003). |
| n = 5 | Day 2 - Two animals euthanized (2002, 2003). All animals deceased. |
| 3. BP_092 KS | Day 0 - Two animals lethargic (3002, 3003). |
| Strain, IV 10\hat{ }9 | Day 1 - Four animals found dead (3001, 3002, 3003, 3005). and one animal |
| CFU/mouse. | euthanized (3004). All animals deceased. |
| n = 5 | |
| 4. BP_109 KS | Day 0 - Two animals found dead within two hours of injection (4001, |
| Strain, IV 10\hat{ }9 | 4006). Two animals lethargic (4004, 4005). |
| CFU/mouse. | Day 1 - One animal has skin tenting and appears to be shivering (4004). |
| n = 6 | Day 2 - One animal euthanized (4004), and one has a prolapsed penis |
| (n = 4 for | (4005). |
| analysis) | Day 3 - One animal found dead (4005). |
| | Day 4 - None. |
| | Day 5 - None. |
| | Day 6 - None. |
| | Day 7 - None. The two remaining animals (4002, 4003) maintained good |
| | health and weight for the entire duration of the study. |
| 5. BP_123 KS | Day 0 - Four animals are lethargic with milky secretions from eyes (5001. |
| Strain, IV 10\hat{ }9 | 5002, 5003, 5005). |
| CFU/mouse. | Day 1 - None. |
| n = 5 | Day 2 - None. |
| | Day 3 - None. |
| | Day 4 - None. |
| | Day 5 - None. |
| | Day 6 - None. |
| | Day 7 - None. All five animals (5001-5005) maintained good health and |
| | weight for the entire duration of the study excluding some adverse |
| | reactions on Day 0. |
| 6. CX_013 KS | Day 0 - One animal lethargic with milky secretions from eyes (6001). |
| Strain, IV 10\hat{ }9 | Day 1 - None. |
| CFU/mouse. | Day 2 - None. |
| n = 5 | Day 3 - None. |
| | Day 4 - None. |
| | Day 5 - None. |
| | Day 6 - None. |
| | Day 7 - None. All five animals (6001-6005) maintained good health and |
| | weight for the entire duration of the study excluding one case of adverse |
| | reactions on Day 0. |
| 7. Killed BP_001 | None. All five animals (7001-7005) maintained good health and weight for |
| Negative | the entire duration of the study. |
| Control, IV 10\hat{ }9 | |
| CFU/mouse. | |
| n = 5 | |

WT—wild type;
KS—kill switch;
CFU—colony forming unit;
IV—intravenous injection

Table 39 shows all ten animals from both WT positive control groups—mice injected intravenously with BP_001 and CX_001—experienced severe adverse reactions to the injection and were all dead by Day 5 and Day 2, respectively. On the other hand, all ten animals from two KS test groups—mice injected intravenously with BP_123 and CX_013—maintained good health and weight for the entire duration of the study, excluding some reactions on Day 0, the same day as injection. Two of the animals from KS test group BP_109 maintained good health and weight for the entire duration of the 7-day study. Another two from this group became ill following injection and never recovered—both were deceased by Day 3. There were another two animals from this group that died within two hours following injection—one original and another one to replace the original. All five animals from KS test group BP_092 experienced similar reactions to the positive control groups and were dead by Day 1 of the study. All five animals from the negative control group Killed BP_001 maintained good health and weight for the entire duration of the study.

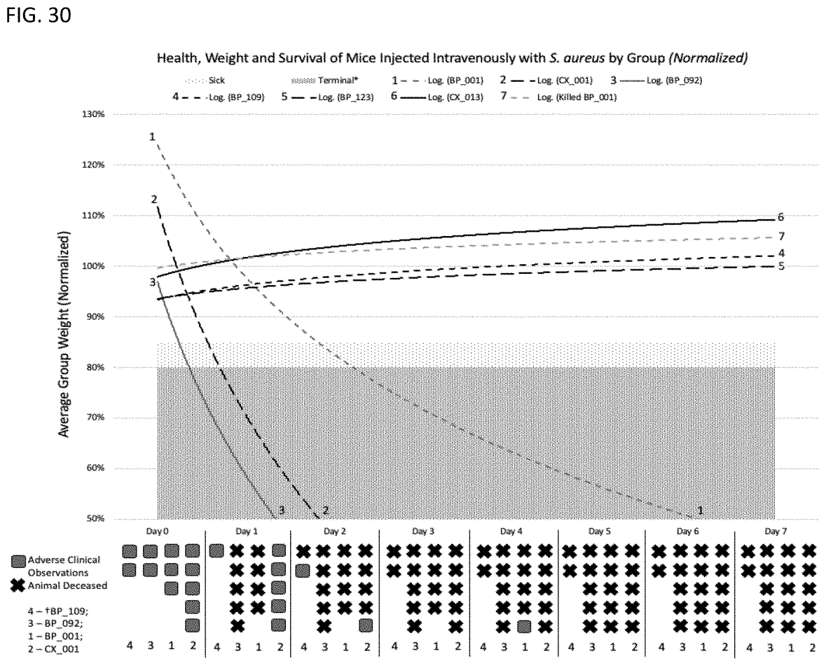

FIG. 30 shows health, weight and survival of mice in high dose bacteremia study after Staph aureus high dose 109 injection in Groups 1-7. The graph values were generated by averaging and normalizing the weight for each group of interest. Normalization was performed by dividing the group (average) weight at each time point by the initial group (average) weight and multiplying the value by 1000. Each time point body weight average was generated using only surviving mice. A graphic at the bottom of FIG. 30 represents adverse clinical observations and mortality. †The Group 4 BP_109 10\hat{ }9 CFU/mouse group only includes four animals as two mice (an original member of the BP_109 group and its replacement) died within two hours of being injected which is not the typical progression of bacteremia or similar to the groups injected with wild-type Staph aureus. They were therefore excluded from analysis. Table 40 shows clinical observations for high dose SSTI Groups 8-14.

TABLE 40
Clinical Observations for SSTI Groups 8-14

| Group | Study Observations (7-Day Study) |
|---|---|
| 8. BP_001 WT | Day 0 - None. |
| Positive Control, | Day 1 - Three animals have abscess formation (8001, 8004, 8005), and two |
| SC 10\hat{ }9 CFU/ | animals have swollen/red areas on back (8002, 8003). |
| mouse. | Day 2 - Four animals have an abscess (8001, 8003, 8004, 8005), and one |
| n = 5 | animal has red skin with discoloration (8002). |
| | Day 3 - Three animals have an abscess (8002-8004), and two animals |
| | have scabbing (8001, 8005). |
| | Day 4 - Three animals have an abscess (8002-8004), and two animals |
| | have scabbing (8001, 8005). |
| | Day 5 - Three animals have an abscess (8002-8004), and two animals |
| | have scabbing (8001, 8005). |
| | Day 6 - Three animals have an abscess (8002-8004), and two animals |
| | have scabbing (8001, 8005). |
| | Day 7 - Three animals have an abscess (8002-8004), and two animals |
| | have scabbing (8001, 8005). |
| | *5/5 animals developed an abscess during the study. |
| 9. CX_001 WT | Day 0 - None. |
| Positive Control, | Day 1 - Four animals have abscess formation (9001-9004), and one |
| SC 10\hat{ }9 CFU/ | animal has swollen eyes and red discoloration on back (9005). |
| mouse. | Day 2 - Four animals have an abscess (9001-9004), and one animal has |
| n = 5 | possible abscess formation and red discoloration on back (9005). |
| | Day 3 - Four animals have an abscess (9001-9004), and one animal has |
| | multiple abscess formation (9005). One animal is euthanized (9002). |
| | Day 4 - One animal has an abscess (9003), one animal has multiple |
| | abscesses (9005), and two animals have scabbed abscesses (9001, 9004). |
| | Day 5 - One animal has an abscess (9003), one animal has multiple |
| | abscesses (9005), and two animals have scabbed abscesses (9001, 9004). |
| | Day 6 - One animal has an abscess (9003), one animal has multiple |
| | abscesses (9005), and two animals have scabbed abscesses (9001, 9004). |
| | One animal is euthanized (9005). |
| | Day 7 - Two animals have an abscess (9003, 9004), and one animal has |
| | scabbing (9001). |
| | *5/5 animals developed an abscess during the study. |
| 10. BP_092 KS | Day 0 - None. |
| Strain, SC 10\hat{ }9 | Day 1 - Four animals have abscess formation (10001, 10003-10005), and |
| CFU/mouse. | one animal has hunched posture and the entire back is discolored (10004). |
| n = 5 | Day 2 - All animals have discolored skin (10001-10005). |
| | Day 3 - All animals euthanized (10001-10005). |
| | 5/5 animals had non-abscess complications during the study. |
| 11. BP_109 KS | Day 0 - None. |
| Strain, SC 10\hat{ }9 | Day 1 - Two animals have a fluid-filled bump (11001, 11002), and two |
| CFU/mouse. | animals have possible abscess formation (11004, 11005). |
| n = 5 | Day 2 - Two animals have a fluid-filled bump (11001, 11003), one animal |
| | has discoloration on back (11002), one animal has abscess formation |
| | (11004), and one animal has possible abscess formation (11005). |
| | Day 3 - Four animals have small abscess formation (11002-11005), and |
| | one animal has possible small abscess formation (11001). |
| | Day 4 - Four animals have an abscess (11002-11005). |
| | Day 5 - Four animals have an abscess (11002-11005). |
| | Day 6 - Two animals have an abscess (11004, 11005), and one animal has |
| | scabbing (11002). |
| | Day 7 - Three animals have an abscess (11003-11005), and one animal |
| | has scabbing (11002). |
| | *‡2/5 animals developed an abscess during the study. |
| 12. BP_123 KS | Day 0 - None. |
| Strain, SC 10\hat{ }9 | Day 1 - None. |
| CFU/mouse. | Day 2 - One animal has a small patch of raised skin (12004), and one |
| n = 5 | animal has a prolapsed penis (12005). |
| | Day 3 - Three animals have an elevated bump on their back (12001, 12004, |
| | 12005). |
| | Day 4 - Two animals have an elevated bump on their back (12001, 12005). |
| | and one animal has abscess formation (12004). |
| | Day 5 - Two animals have an elevated bump on their back (12001, 12005), |
| | and one animal has an abscess (12004). |
| | Day 6 - One animal has a small abscess (12004). |
| | Day 7 - None. |
| | *0/5 animals developed an abscess during the study. |
| 13. CX_013 KS | Day 0 - None. |
| Strain, SC 10\hat{ }9 | Day 1 - None. |
| CFU/mouse. | Day 2 - None. |
| n = 5 | Day 3 - One animal has very small abscess formation (13005). |
| | Day 4 - One animal has a small abscess (13005). |
| | Day 5 - Two animals have an abscess (13002, 13005). |
| | Day 6 - One animal has a small abscess (13002). |
| | Day 7 - One animal has a small abscess (13002). |
| | *†0/5 animals developed an abscess during the study. |
| 14. Killed | None. |
| BP_001 | *0/5 animals developed an abscess during the study. |
| Negative | |
| Control, SC 10\hat{ }9 | |
| CFU/mouse. | |
| n = 5 | |

WT—wild type;
KS—kill switch;
CFU—colony forming unit;
SC—subcutaneous injection.
*By the abscess definition for mice injected with a Staph aureus concentration of 10\hat{ }9 CFU/mouse, a visible accumulation of purulent material must persist for more than three days to be deemed an abscess.
†One mouse injected with CX_013 (13002) had abscess formation on Day 5 and still had presence of a small abscess on Day 7, the last day of the study. We are unable to definitively say whether the abscess would persist more than 3 days, but interpolated from two similar mice (13005 and 12004) that it was likely to be resolved within three days.
‡BP_109 has shown in other bacteremia groups to cause severe immune responses at 10\hat{ }9 CFU, similar to the condition in humans known as a Jarisch-Herxheimer reaction, which could be the cause for abscess formation in this group.

To summarize Table 40, all ten animals from both WT positive control groups—mice injected subcutaneously with BP_001 and CX_001—had an abscess develop during the study. Two mice from the CX_001 group (Group 9) experienced such severe adverse reactions that they were deemed moribund and euthanized. By comparison, none of the ten animals from two KS test groups—mice injected subcutaneously with BP_123 and CX_013—developed an abscess during the study. The single abscess formations reported from three of these mice (one from BP_123 and two from CX_013) were all reported as small or very small growths and were resolved within three days of first appearance. Two of the five mice from KS test group BP_109 had an abscess develop. By visual comparison to WT parent strain BP_001 and other BP-strain KS BP_123, the abscess is moderate in severity. All five animals from KS test group BP_092 had acute non-abscess forming symptoms unlike any other group, particularly when compared to WT parent strain, BP_001. The BP_092 animals experienced skin discoloration and necrotic tissue underneath the skin and they were all deemed moribund and euthanized by the morning of Day 3. All five animals from the negative control group Killed BP_001 were free of adverse clinical symptoms, including abscess formation, for the entire duration of the 7-day study.

Statistical Analysis:

As no weight decline occurred for any of the groups involved in the surviving mice in the study, a dichotomous score was used to compare groups by an absolute measure. The hypothesis test for binomial distributions was used to compare groups by parent/daughter strains. Any abscess formation throughout the study assigned a mouse a value of 1 and complete absence of abscess formation for the duration of the study assigned a mouse a value of 0, as shown in Table 41.

TABLE 41
Dichotomous Score for high dose SSTI Abscess Formation by Group per Mouse

| | | | | | | | Group |
|---|---|---|---|---|---|---|---|
| Group | Treatment | Mouse 1 | Mouse 2 | Mouse 3 | Mouse 4 | Mouse 5 | Score |
| 8 | BP_001 | 1 | 1 | 1 | 1 | 1 | 5/5 |
| 9 | CX_001 | 1 | 1 | 1 | 1 | 1 | 5/5 |
| 11 | BP_109 | 0 | 0 | 0 | 1 | 1 | 2/5 |
| 12 | BP_123 | 0 | 0 | 0 | 0 | 0 | 0/5 |
| 13 | CX_013 | 0 | 0 | 0 | 0 | 0 | 0/5 |
| 14 | Killed | 0 | 0 | 0 | 0 | 0 | 0/5 |
| | BP_001 | | | | | | |

Abscess Formation = 1;
No Abscess Formation = 0

The hypothesis test for binomial distributions was used to compare groups by parent/daughter strains. In other words, the analysis was used to compare BP_001 to BP_123 and CX_001 to CX_013 as the latter were derived from the former. Probability was assigned by the WT groups' presence of abscess formation, and alpha was set to 99% confidence.

The analysis determined that only one out of five mice in the test group must be abscess free for both BP- and CX-derived strains to achieve a 99% confidence. As only two of the five from test group BP_109 developed an abscess, and none of the mice from test groups BP_123 and CX_013 developed abscesses, we may report that BP_109, BP_123 and CX_013 test groups are significantly different to the comparative WT groups with a p-value <0.01.

Group 10 (BP_092) is excluded from Table 41 because all animals in the group were not observed to experience abscess formation according to the definition used in this study, and experienced significantly different reactions as compared to the WT parent strain BP_001 (Group 8) and all other groups in the study, both experimental groups and controls. Group 10 had white skin discoloration across the majority of the animal's back that lead to necrotic tissue formation, which was unlike any other symptom observed, and outside of the scope of the study. Further, all animals in Group 10 were euthanized due to their moribund state on Day 3, and following euthanization, swabs were taken using sterile inoculating needles between the skin and necrotic tissue, and then the loop was struck out on a TSB agar plate to culture the bacteria picked up by the loop. The most bacteria that were cultured this way from one mouse was 25 colonies on a plate.

All mice injected with high dose modified KS strains BP_123 and CX_013 did not develop bacteremia and only experienced minor adverse reactions on Day 0, the same day as injection. Both WT parent strains—BP_001 and CX_001—caused mortality in the bacteremia study, and severe illness and abscess formation in the SSTI study at 10\hat{ }9 CFU/mouse.

BP_109 is an engineered Staph aureus strain with two genomically integrated kill switches, and through in vitro testing has been shown to be the most potent kill switch combination to date. It may be possible the increased potency of the kill switches may have produced a sudden release of bacterial cell components into the bloodstream of the mice soon after intravenous injection, which could be responsible for the adverse reaction that 4 of the mice in the bacteremia group experienced within hours of the dosing, and the abscess formation in the SSTI group. A similar condition has been identified in humans, called a Jarisch-Herxheimer reaction, which occurs when a sudden release of endotoxin-like products occurs due to antibiotic treatments of an infection.

BP_092 has been demonstrated to be the least potent KS tested under in vitro conditions employing gene sbnA, which is induced at a much lower rate in blood and serum than isdB. All of the mice injected intravenously were dead or euthanized by the end of Day 1, indicating that 10\hat{ }9 CFU injected intravenously via tail vein is too high a cell concentration for strain BP_092 to protect the mice from death.

### Example 20. Tuning the sprA1 Kill Switch in Staph aureus

A series of experiments was designed to evaluate the effect of iron concentration on the viability of different synthetic S. aureus KS strains in different media and biological fluids, and the ability to “tune” the efficacy of the KS with additional copies of the antitoxin integrated into the genome. The addition of a second sprA1_{AS} expression cassette into the genome will result in increased copies of sprA1_{AS} sRNA transcripts in the cytoplasm. It was hypothesized that this increase in sprA1_{AS} sRNA could be exploited to inhibit PepA1 peptide toxin expression, and thus “tune” the KS to withstand lower levels of available iron than strains harboring only one copy of sprA1_{AS}.

Four different types of assays were performed to assess KS tunability in response to the integration of an additional copy of PsprA1_{AS}-sprA1_{AS}: 1) Serum Assay, 2) Iron Spiked Serum Assay, 3) RPMI 1640 Assay, and 4) Iron Spiked RPMI 1640 Assay.

Human serum and RPMI 1640 were used as “base media” to simulate iron deficient conditions in respective assays. FeCl₃ was then spiked from a stock solution into each of these media types at various levels to assess the effect of available iron on bacterial cell growth. The full protocol for iron spike assays is listed below and iron concentrations can be found in Table 10 in the appendix.

This set of experiments focused on modified KS strains BP_109 and BP_144. The KS strain BP_144 was generated by integrating an additional copy of the native sprA1_{AS} expression cassette in Site2 of the BP_109 genome. This additional sprA1_{AS} cassette in BP_144 should inhibit the translation of the sprA1 toxin gene in environments with partially limited iron to a higher degree than its parent strain, BP_109, which has only one copy of the sprA1_{AS} expression cassette. Because the levels of available iron could vary greatly in S. aureus's native niche on the skin, the intent is to create a KS strain that is more stable at living in these variable conditions. BP_001 and BP_121 are used as wild type controls in the assays discussed in this report. BP_001 is a wild type Staph aureus strain and the parent for all strains discussed in this report. BP_121 has a 33 bp integration into a non coding portion of the Staph aureus genome for use only as a genomic ID tag to more easily identify the strain by PCR. Previous testing done on this strain has shown it to be phenotypically similar to the wild type parent strain BP_001.

In addition, KS activation in rabbit cerebrospinal fluid (CSF) was investigated. CSF is a nutrient poor environment, and does not readily promote S. aureus growth. However, S. aureus can still become pathogenic if adequate nutrients for growth become available in CSF, such as following trauma. The viability of KS strain BP_109 in rabbit CSF and rabbit CSF spiked with different amounts of human serum was investigated. The results were compared to a phenotypically wild-type strain BP_121 grown in CSF.

Table 42 shows the strains used in the tunability study, along with the genotype of the strains, the DNA sequence IDs applicable to each strain, and the strain report describing how the strain was constructed and tested. BP_144 was constructed from parent strain BP_109. BP_121 contains a small insertion sequence used only for identification purposes.

TABLE 42
Bacterial Strains Used in KS Tunability Study

| Strain | Parent | Kill | | DNA Sequence |
|---|---|---|---|---|
| Name | Strain | Switch | Genotype | ID |
| BP_001 | n/a | No | Wild type | BP_001 |
| BP_109 | BP_001 | Yes | isdB::sprA1, | BP_DNA_003 |
| | | | PsbnA::sprA1, ΔsprA1 | BP_DNA_003 |
| | | | | BP_DNA_045 |
| BP_121 | BP_001 | No | Site2::code_1 | BP_DNA_023 |
| BP_144 | BP_109 | Yes | Site2::PsprA1_{AS}-sprA1_{AS} | BP_DNA_003 |
| | | | | BP_DNA_003 |
| | | | | BP_DNA_045 |
| | | | | BP_DNA_005 |

Table 43 shows one strand in the 5′ to 3′ direction of the double-stranded DNA sequences that are important to, or used in, the experiments described in this report. For BP_DNA_003, the bold sequence represents the sprA1 reading frame, and underlined sequence represents the 5′ untranslated region (control arm).

TABLE 43
DNA Sequences used in this Study

| Se | | Sequence of Insert or |
|---|---|---|
| quence | | |
| ID | Genotype | Deletion |
| BP_DNA_{—} | PsprA1_{AS}- | CAGTCATCAAGCACAGTTTGACTGGAA |
| 005 | sprA1_{AS} | AGAAGGCATTAACTTTAAAACGAAGGA |
| | Fragment | TAATCAAATGGTCCTTTAGAAGGGATA |
| | | AACAACAAAATAAAATTAATTAAACGT |
| | | ACATCTTTTGGTTAAGGAAGTTATAAT |
| | | CATTTGCGAAATCGAATATTATTATGT |
| | | TCAAAACTTTACGCTCCAAAAAGTAAA |
| | | AAGGAAGCTAAGCAATGTTTAGTTGCC |
| | | TAACTTCCGATATTGAACTCATCAGGC |
| | | CAATTTGGCATAGAGCCTTTTTTAGTT |
| | | CTTGATGTTTCTCTTTAAAACCTTGCA |
| | | TATTTTACAAAGAGAAAGATTAGCAGT |
| | | ATAATTGAGATAACGAAAATAAGTATT |
| | | TACTTATACACCAATCCCCTCACTATT |
| | | TGCGGTAGTGAGGGGATTTTTATTGGT |
| | | GCGGCTATATGTCACCTATTTTGTATT |
| | | GCGTCTACTTAGCC (SEQ ID NO: |
| | | 5) |
| BP_DNA_{—} | Inserted | CGCAGAGAGGAGGTGTATAAGGTGATG |
| 003 | sprA1 frag- | CTTATTTTCGTTCACATCATAGCACCA |
| | ment for kill | GTCATCAGTGGCTGTGCCATTGCGTTT |
| | switches | TTTTCTTATTGGCTAAGTAGACGCAAT |
| | | ACAAAATAG (SEQ ID NO: 3) |
| BP_DNA_{—} | ΔsprA1 (dele- | ATATAATAGTAGAGTCGCCTATCTCTC |
| 045 | tion of 5′ | AGGCGTCAATTTAGACGCAGAGAGGAG |
| | end) | GTGTATAAGGTGATGCTTATTTTCGTT |
| | | CACATCATAGCAC (SEQ ID NO: |
| | | 29) |
| BP_DNA_{—} | Site2::code_1 | CGATCTTCGACATCGGACCCTAGAACA |
| 023 | | GAACTA (SEQ ID NO: 19) |

Methods

The serum and RPMI assays in this experiment follow the protocol similar to the Serum Assay used herein, but employed two phosphate buffered saline (PBS) wash steps instead of one prior to resuspending the cells in the media with specific levels of iron for the growth assay. The additional PBS wash step was implemented during the preparation of each culture to ensure full removal of components from previous growth media prior to introduction into iron deficient media.

The serum spiked CSF assay was carried out according to the protocol described herein above and was assayed in CSF, CSF+1% serum, and CSF+2.5% serum, and BP_121 was also grown in CSF as the wild-type control.

Iron Spike Assay
1. Culture Preparation
   1.1. Cultures were started by inoculating 5 mL TSB with single colonies of selected strain (BP_109 and/or BP_144) in 14 mL sterile culture tubes, and placing them in the shaking incubator at 37° C. and 240 rpm to grow overnight.
   1.2. The following morning, the overnight cultures were cut back to 0.05 OD600 in 5.5 mL of fresh TSB.
      1.2.1. OD600 was measured in 1 cm cuvettes in a NanoDrop spectrophotometer.
      1.2.2. The resulting OD600 values were used to calculate the volume of overnight culture needed to inoculate fresh TSB to 0.05 OD600.
      1.2.3. Fresh 5.5 mL TSB cultures were inoculated with appropriate volumes of overnight culture and incubated for 2 hrs (37° C., 240 rpm) in order to get the cells growing in log phase again.
   1.2.4. After the 2-hour incubation, the OD600 was measured for each culture.
   1.2.5. The cultures were then washed twice in sterile PBS.
      1.2.5.1. Cultures were centrifuged to pellet the cells using the swing out rotor (3500 rpm, 5 mins, RT), and washed with 5 mL PBS.
      1.2.5.2. Repeat step 1.2.5.1 for a second PBS wash.
      1.2.5.3. Cultures were centrifuged to pellet the cells again, and resuspended in 1 mL sterile PBS.
   1.2.6. The OD600 values obtained after the 2-hour incubation were used to calculate the volume needed to inoculate 5 mL of human serum or RPMI 1640 to 0.05 OD600.
      1.2.6.1. (Measured OD600)(X mL)=(0.05 OD600)(5 mL)
   1.2.7. Before cultures were inoculated, the media was spiked with iron at desired concentration.
      1.2.7.1. Various iron concentrations were added as defined.
   1.2.8. After addition of iron, spiked media were mixed by pulse vortex.
   1.2.9. Cultures were then inoculated with volume calculated in step 1.2.6. Each series of concentrations was inoculated with the same sample.
   1.2.10. After addition of inoculum, cultures were mixed by pulse vortex and 100 μL samples were taken for determining CFU/mL by dilution plating (see Step 2).
   1.2.11. The cultures were immediately placed in the 37° C. shaking incubator (240 rpm) and samples were taken after 2 hours and again at 4 hours to determine CFU/mL by dilution plating.
2. Serial Dilutions and Culture Plating
   2.1. Dilution plating was performed using the Opentrons OT-2 robot.
      2.1.1. Dilutions were carried out to a concentration where 30-300 colonies grew from plating 100 μL of diluted sample on TSA plates.
      2.1.2. The OT-2 robot performed all serial dilutions and plated 100 μL of the diluted culture TSAbp_109 serum spike 0.6 bp_109 serum spike 0.6 bp_109 serum spike 0.6 bp_109 serum spike 0.6 plates, and sterile spreaders were used to spread the culture to maximize the surface area used on the plates.
      2.1.3. Dilutions were plated in duplicate and the average of the two plates was calculated and used for the single replicate data point.
3. Incubation and Colony Counting
   3.1. TSA plates were incubated overnight for 12-16 hours at 37° C.
   3.2. The following morning, plates were removed from the incubator and colony counting was performed to determine the concentration of viable cells at each time point (CFU/mL).
      3.2.1. Multiple dilutions were plated in duplicate for each condition at each time point, only plates with 30-300 colonies were used to calculate the CFU/mL values.

Results:

In order to fulfill the objective of specifically quantifying the concentration of available iron necessary to sustain growth of KS strains, a more defined and reliable growth media was required. Rather than dealing with the inconsistencies associated with biological fluids, serum was replaced with RPMI 1640 for the iron spike-in assays.

In order to quantify the concentration of iron required to sustain growth, FeCl₃ solutions were prepared and spikes of these solutions were added to RPMI1640 at varying molar Fe(III) concentrations.

RPMI 1640 is a defined media used in culturing mammalian cell lines, in which wild-type S. aureus can sustain growth. RPMI 1640 does not contain any proteins or other substances that may sequester iron spiked into the media creating inconsistencies during assays, and has no iron available in the media as is, allowing for precise measurements of iron in solution. BP_001 growth was tested in RPMI with 0 and 3 μM Fe(III) and no difference in growth was observed.

A and B shows the average CFU/mL (n≥3) during a 4-hour growth period in RPMI 1640 liquid media spiked with different levels of Fe(III) using strains (A) BP_109, and (B) BP_144 to determine the iron concentration levels where kill switch activation occurs. (A) For BP_109, as the levels of iron in the media increases from 0 to 3 μM Fe(III), at which the growth pattern between the wild-type BP_001 and BP_109 look very similar and have overlapping error bars. (B) For BP_144, as the levels of iron in the media increases from 0 to 1 M Fe(III) the number of viable cells/mL also increases. The growth curves at both 1 and 3 μM Fe(III) overlap with the wild type BP_001 for the BP_144 strain. The error bars represent one standard deviation for the averaged replicates (n=2-4).

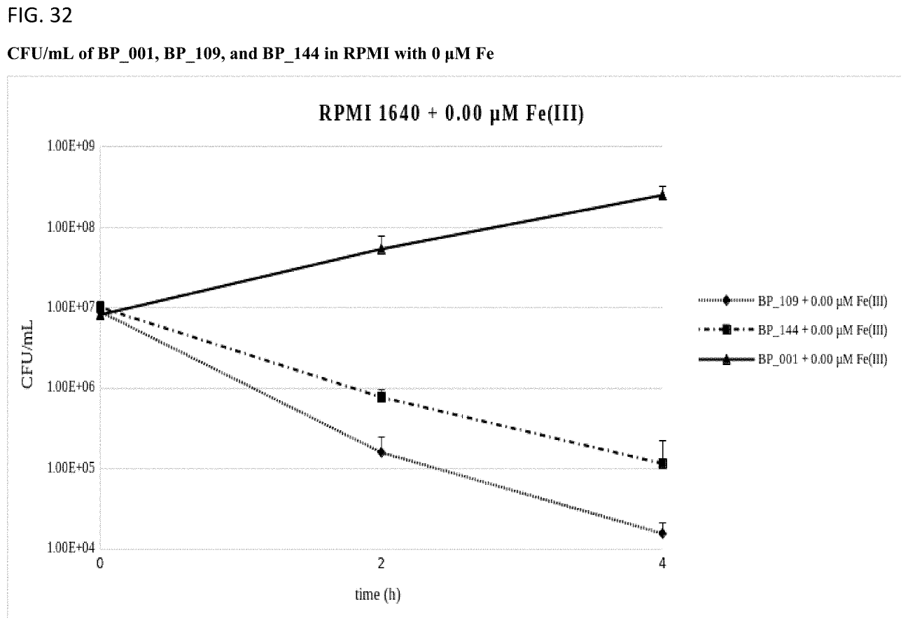

FIG. 32 shows the average (n≥3) CFU/mL from growth assays of Staph aureus BP_001 (WT), BP_109 (KS) and BP_144 (KS+AS) performed in RPMI with 0.00 μM Fe(III). The viable cell counts of BP_109 decreased over the four-hour period. The error bars represent one standard deviation from the averaged replicates. BP_144 had increased viable CFU/mL compared to its parent strain BP_109.

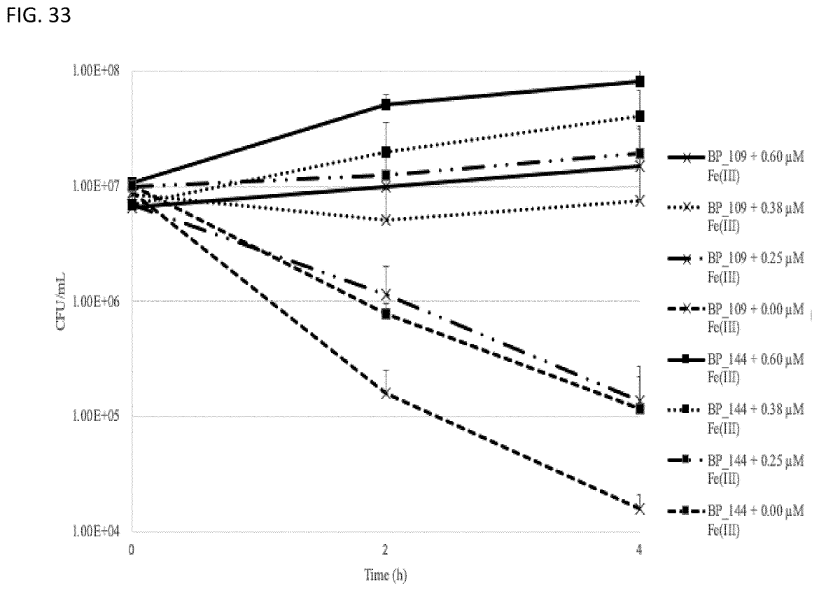

FIG. 33 shows a graph of viable cell growth as CFU/mL of strains BP_109 and BP_144 grown in RPMI 1640 spiked with different levels of Fe(III) (0, 0.25, 0.38, and 0.60 μM) over 4 hours. BP_144 had increased viable CFU/mL compared to its parent strain BP_109 in each level of iron tested during the 4-hour growth period.

A similar cell growth assay was performed replacing RPMI media with rabbit CSF, or CSF spiked with human serum, comparing BP_109 (KS) with a control synthetic Staph aureus strain BP_121 (no KS). In the CSF assay shown in FIG. 34, a trend can be seen where BP_109 loses viability as the concentration of human serum in the CSF increases. The wild-type control, BP_121, was not grown in the CSF+1.0% serum spiked condition, due to limited CSF availability; however, BP_121 readily grows in human serum and has been demonstrated to show increased viability when cultured in serum-enriched CSF conditions. The data shown here indicate that the level of KS activation in CSF may be linked to the nutrient levels in the environment and the corresponding levels of metabolic activity in the cell.

FIG. 34 shows a graph cell growth assays comparing Staph aureus strains BP_121 (no kill switch) and BP_109 (iron sensitive kill switch) in CSF and BP_109 in rabbit CSF spiked with 1.0% and 2.5% human serum. Strains were cultured in CSF or CSF+serum at a total volume of 500 μL (n=1). BP_121+2.5% human serum was analyzed in a separate assay (n=3). A trend can be seen where BP_109 loses viability as the concentration of human serum in the CSF increases. Conversely, BP_121 increases in viable cell counts upon introduction of serum to the CSF.

In engineered kill switch strains modified with an additional copy of the native sprA1_{AS} expression cassette, viable cell counts were higher at the termination of growth assays in iron deficient media, as compared to their parent strains. It was demonstrated that increasing the number of sprA1_{AS} expression cassettes in a genome can change the efficacy of the sprA1 kill switches when the cells are grown in iron-limiting media. As shown in FIGS. 31 to 34, a linear relationship was demonstrated for a specific range of available iron in the media to the number of viable CFU/mL in a culture.

### Example 21. Inducible Bacteriostasis Kill Switch using sprG Action Genes

Action genes sprG2 and sprG3 were tested for their ability to cause bacteriostasis in E. coli and S. aureus using the pRAB11 expression vector.

The sprG2 and sprG3 genes are native to many S. aureus species. The genes belong to a type I TA system and are both capable of causing bacteriostasis when overexpressed in Staph aureus. In this example, two plasmids using the pRAB11 backbone were prepared by adding the sprG2 and sprG3 genes (designated p305 and p306, respectively) behind the ATc-inducible promoter located on the plasmid. Overexpression of the sprG2 and sprG3 genes from the plasmid caused bacteriostasis in S. aureus and E. coli, however, sprG3 was only able to slow the growth of E. coli for a short period of time after induction.

Table 44 shows the oligo names and sequences used to generate plasmids p305 and p306. The assembly required a stitch PCR and gibson assembly. The single stranded DNA sequences are in the 5′ to 3′ direction.

TABLE 44
Oligos and Their Sequences

| Name | Plasmid | Sequence (5′ to 3′) |
|---|---|---|
| BP_542 | p305/6 | CATCACCTTATACACCTCCTCTCTGC (SEQ ID |
| | | NO: 240) |
| BP_717 | p305/6 | ACTCTTTGAAGTCATTCTTTACAGGAG (SEQ ID |
| | | NO: 241) |
| BP_718 | p305/6 | CTCCTGTAAAGAATGACTTCAAAGAGT (SEQ ID |
| | | NO: 242) |
| DR_215 | p305/6 | CCGACCTCATTAAGCAGCTCTAATGCGCTG (SEQ |
| | | ID NO: 243) |
| DR_216 | p305/6 | GGTGTGAAATACCGCACAGATGCGTAAGG (SEQ |
| | | ID NO: 244) |
| DR_725 | p305 | GCAATAAAAAATAAGTGACATATAGCCGCACCAAT |
| | | AAAAATTGATAATAGC (SEQ ID NO: 245) |
| DR_726 | p305 | GGTGCGGCTATATGTCACTTATTTTTTATTGCTTA |
| | | AATTTATTATTGCTACTACTATACC (SEQ ID |
| | | NO: 246) |
| DR_727 | p305 | CGCAGAGAGGAGGTGTATAAGGTGATGATATCTAT |
| | | TGCAAACGCATTAC (SEQ ID NO: 247) |
| DR_728 | p306 | TGGTGCGGCTATATGTCACTTATTTTTTATGGTCT |
| | | TGAGTACTAATCAATACTAAACC (SEQ ID NO: |
| | | 248) |
| DR_729 | p306 | CGCAGAGAGGAGGTGTATAAGGTGATGTCTGATTT |
| | | TGAAATGCTGATGGTTG (SEQ ID NO: 249) |
| DR_730 | p305/6 | GACCATAAAAAATAAGTGACATATAGCCGCACCAA |
| | | TAAAAATTGATAATAGCTG (SEQ ID NO: |
| | | 250) |
| DR_733 | p305 | GTGCGGCTATATGTCACTTATTTTTTATTGC |
| | | (SEQ ID NO: 251) |
| DR_734 | p305 | CGCAGAGAGGAGGTGTATAAG (SEQ ID NO: |
| | | 252) |

Two plasmids were generated using the high-copy expression vector, pRAB11. This plasmid may be used for anhydrotetracycline (ATc)-dependent expression of genes in E. coli or S. aureus. Plasmid pRAB11 was generated by adding another tetO operator to the TetR-regulated promoter, P_{xyl/tet}, in plasmid pRMC2 in order to provide tighter regulation of the gene downstream of the promoter. TetR is a transcriptional repressor protein that binds to DNA if the tetO sequence is present. The P_{XYL/tet} promoter in pRAB11 has two tetO sequences that flank the transcriptional start site which represses the transcription of any gene just downstream of the promoter. When ATc is added to the culture, it will bind to the repressor protein TetR and inhibit the protein's ability to bind to TetO within the promoter allowing promoter activation and gene overexpression. Helle, Leonie, et al. “Vectors for improved Tet repressor-dependent gradual gene induction or silencing in Staphylococcus aureus.” Microbiology 157.12 (2011): 3314-3323.

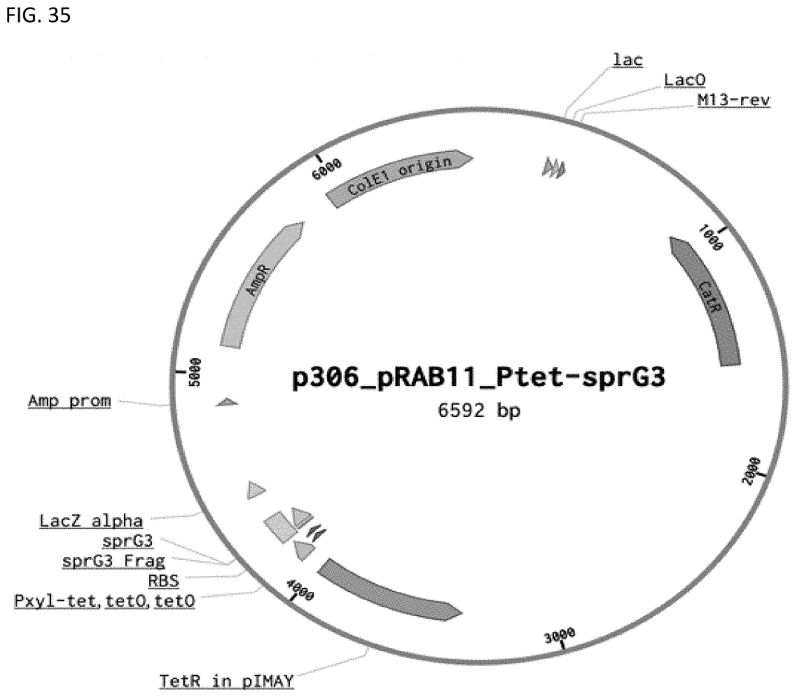

FIG. 35 shows a plasmid map for plasmid p306 comprising Ptet::sprG3 DNA on pRAB11 Vector. It is also representative of the plasmid map for p305 comprising Ptet::sprG2, as the only difference is the action gene sprG2 is present as opposed to sprG3.

Table 45 shows the plasmids transformed into S. aureus and E. coli. *The sprG2 gene within the p305 plasmid has an ATG start site and a single point mutation following the start codon making it slightly different from the native BP_001 sprG2 gene.

TABLE 45
Plasmid Names and Function

| | DNA on pRAB11 Vector |
|---|---|
| Name | to be Transformed |
| p305 | Ptet::sprG2* |
| p306 | Ptet::sprG3 |

Table 46 shows the strains used or created for this study. The sequences shown for the generated strains are the sprG3 and sprG2* genes only as the pRAB11 backbone is over 6 kb long.

TABLE 46
Strains Used in the Study

| Strain | Genotype | DNA Sequence of Insert |
|---|---|---|
| BP_001 | Wild type S. | n/a |
| | aureus | |
| IM08B | Wild type E. | n/a |
| | coli | |
| BP_164 | BP_001, | ATGTCTGATTTTGAAATGCTGATGGTTG |
| | pRAB11_{—} | TATTAACAATCATTGGTTTAGTATTGAT |
| | Ptet::sprG3 | TAGTACTCAAGACCATAAAAAATAA |
| | | (SEQ ID NO: 253) |
| BPEC_024 | IM08B, | ATGTCTGATTTTGAAATGCTGATGGTTG |
| | pRAB11_{—} | TATTAACAATCATTGGTTTAGTATTGAT |
| | Ptet::sprG3 | TAGTACTCAAGACCATAAAAAATAA |
| | | (SEQ ID NO: 253) |
| BP_165 | BP_001, | ATGcTATCTATTGCAAACGCATTACATT |
| | pRAB11_{—} | TAATGTTAAGTTTCGGTATGTTTATCGT |
| | Ptet::sprG2* | CACTTTCATTGGTATAGTAGTAGCAATA |
| | | ATAAATTTAAGCAATAAAAAATAA |
| | | (SEQ ID NO: 254) |
| BPEC_025 | IM08B, | ATGcTATCTATTGCAAACGCATTACATT |
| | pRAB11_{—} | TAATGTTAAGTTTCGGTATGTTTATCGT |
| | Ptet::sprG2* | CACTTTCATTGGTATAGTAGTAGCAATA |
| | | ATAAATTTAAGCAATAAAAAATAA |
| | | (SEQ ID NO: 254) |

*Two base pair substitutions occurred in sprG2 making it different from its native sequence in BP_001. The start codon was intentionally altered from a GTG to an ATG to increase the likelihood of expression, and the lowercase c was an unintentional point mutation that occurred during plasmid generation changing an A to a C. This base pair substitution causes the amino acid to change from an isoleucine to a leucine (sprG2.V1M, I2L).

PCR Fragment generation

The following PCR reactions were performed using Q5 High Fidelity Hot Start Master Mix (NEB) per the manufacturer's instructions.

BP_DNA_095 pRAB11 Linearized Plasmid Backbone (p151)=SEQ ID NO: 52.
Fragment of BP_DNA_095—p174 Backbone Fragment 1 (p305 & p306)
   BP_717/BP_542
Fragment of BP_DNA_095—p174 Backbone Fragment 2 (p305 & p306)
   BP_718/BP_725 (p305)
   BP_718/BP_730 (p306)
BP_DNA_125(SEQ ID NO: 71)—sprG2 (Inserted sequence on pRAB11 vector) (p305)
   DR_726/DR_727
BP_DNA_113 (SEQ ID NO: 62)—sprG3 (Inserted sequence on pRAB11 vector) (p306)
   DR_729/DR_728
1) The above PCR fragments were checked on a 1% or 2.5% agarose gel to confirm a clean band, and then purified using a Qiaquick PCR Purification Kit (Qiagen) per the manufacturer's instructions.
2) The p174 fragment was treated with DpnI (NEB) to remove the pRAB11 plasmid used as the template for the PCR, and purified again using the PCR Cleanup Kit (NEB) per the manufacturer's instructions.
3) The DNA fragments for each plasmid were stitched together using stitch PCR, then used in a Gibson Assembly reaction (NEB) to create circular plasmids per the manufacturer's instructions.
4) The assembled plasmids were then transformed into IM08B per the transformation protocol in Report_SOPO30, plated on LB (carb) agar plates, and incubated overnight at 37° C.
5) The following day, colonies were screened for fully assembled plasmids by colony PCR to check for the presence of the sprG2 or sprG3 on the pRAB11 plasmid.
6) Three positive colonies were picked, grown overnight at 37° C. in 5 mL of LB (plus carbenicillin, 100 ug/mL), and the plasmid was extracted using the ZymoPURE plasmid miniprep kit per the manufacturer's instructions. The plasmids were then sequenced to confirm the DNA sequence of the sprG2 or sprG3 gene and the promoter upstream of the inserted genes.
7) The sequencing was aligned in silico using the sequence alignment tool in Benchling.
   a) One of the colonies whose sequencing alignment showed a perfect alignment to the reference map's sequence was picked and stocked in the plasmid database per the protocol in Report_SOP028 Preparing Strain and Plasmid Stocks.
      Note: For sprG2, there was a single point mutation on each sequence plasmid (see Table 4). One was picked and tested despite the mutation.
8) The generated cultures from Step 6 were subjected to a 6-hour growth assay to test the effect that overexpression of the inserted gene has on the host cell.
   a) Start 5 mL cultures of the sequence verified clones to be tested, and add the appropriate antibiotic to ensure plasmid maintenance. Grow cultures overnight at 37° C. shaking at 250 rpm.
   b) The following day, measure the absorbance of the cultures by measuring the OD600.
   c) Calculate the volume of the overnight culture needed to inoculate a fresh 5 mL culture at an OD600 of 0.05.
   d) For each overnight culture started, inoculate two fresh cultures using the volume calculated in Step 8c.
   e) Mix the cultures and measure the OD to determine the density of the culture at the start of the assay. Place the culture tubes in the 37° C. incubator shaking at 250 rpm.
      i) Measure the OD of the cultures as described above every hour for 6 hours.
      ii) After the 2-hour OD measurement, induce the overexpression of the sprG2 sprG3 genes by adding ATc (10 ug/mL) to one set of the culture tubes, and place all tubes back in the incubator.
      iii) Continue to measure the OD every hour until the assay is complete.
   9) The sequence verified plasmids were also transformed into BP_001 per the transformation protocol in Report_SOP029, plated on BHI (chlor/x-gal), and incubated overnight at 37° C.
   10) Three positive colonies were picked, grown overnight in 5 mL of TSB (plus chloramphenicol, 10 ug/mL), and subjected to a 6-hour growth assay using the same protocol as in Step 8.

Strains and results of an ATc-Induced Toxin Assay Results Averaged for sprG2 and sprG3 in E. coli and S. aureus are shown in Table 47.

TABLE 47
ATc-Induced Toxin Assay Results Averaged for sprG2 and sprG3 in E. coli and S. aureus

Condition

| (n = 3, unless | OD600 ± Std Dev at Time (h) | | | | | | |
|---|---|---|---|---|---|---|---|
| denoted otherwise) | 0 h | 1 h | 2 h | 3 h | 4 h | 5 h | 6 h |
| sprG2* in E. coli | 0.09 ± 0.00 | 0.09 ± 0.00 | 0.29 ± 0.01 | 0.67 ± 0.05 | 1.16 ± 0.02 | 1.77 ± 0.06 | 2.03 ± 0.06 |
| (BPEC_025) | | | | | | | |
| sprG2* in E. coli | 0.09 ± 0.00 | 0.09 ± 0.01 | 0.29 ± 0.01 | 0.27 ± 0.03 | 0.25 ± 0.01 | 0.24 ± 0.02 | 0.25 ± 0.01 |
| (BPEC_025) + ATc | | | | | | | |
| sprG2* in S. aureus | 0.10 ± 0.01 | 0.13 ± 0.03 | 0.62 ± 0.08 | 1.49 ± 0.08 | 2.30 ± 0.10 | 3.07 ± 0.12 | 3.73 ± 0.23 |
| (BP_165) | | | | | | | |
| sprG2* in S. aureus | 0.10 ± 0.00 | 0.13 ± 0.03 | 0.67 ± 0.12 | 0.58 ± 0.11 | 0.53 ± 0.06 | 0.53 ± 0.08 | 0.49 ± 0.06 |
| (BP_165) + ATc | | | | | | | |
| sprG3 in E. coli | 0.08 | n/a | 0.32 | 0.68 | 1.50 | 1.90 | 1.90 |
| (BPEC_024) (n = 1) | | | | | | | |
| sprG3 in E. coli | 0.09 | n/a | 0.33 | 0.44 | 0.90 | 1.00 | 1.30 |
| (BPEC_024) + ATc | | | | | | | |
| (n = 1) | | | | | | | |
| sprG3 in S. aureus | 0.03 ± 0.01 | 0.09 ± 0.01 | 0.61 ± 0.04 | 1.33 ± 0.05 | 1.83 ± 0.35 | 2.50 ± 0.00 | 3.03 ± 0.06 |
| (BP_164) | | | | | | | |
| sprG3 in S. aureus | 0.04 ± 0.01 | 0.10 ± 0.02 | 0.66 ± 0.07 | 0.52 ± 0.03 | 0.38 ± 0.02 | 0.32 ± 0.02 | 0.32 ± 0.00 |
| (BP_164) + ATc | | | | | | | |

Table 47 shows the average and standard deviation of the triplicate OD600 taken at each time point, except for sprG3 in E. coli (n=1).

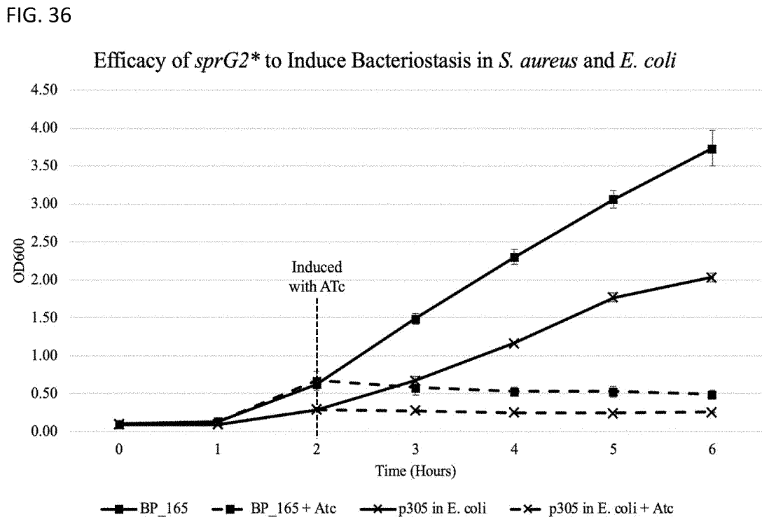

Action gene sprG2* (*V1M, I2L) was able to induce bacteriostasis in both E. coli (BPEC_025) and S. aureus (BP_165) upon the addition of ATc (added at t=2 h) leading to overexpression of the gene (FIG. 36).

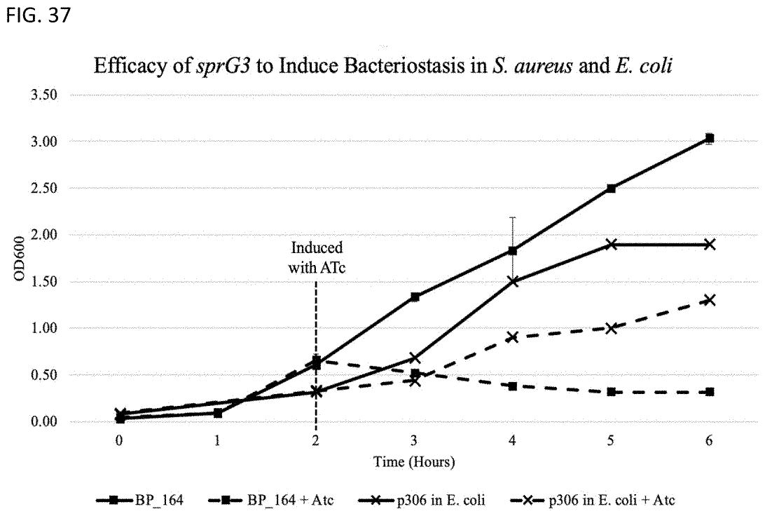

Action gene sprG3 was able to induce bacteriostasis in S. aureus (BP_164) upon the addition of ATc (added at t=2 h), but was only able to do so temporarily and less effectively in E. coli (BPEC_024) (FIG. 37).

In this example, two plasmids using the pRAB11 backbone were prepared by adding the sprG2 and sprG3 genes (designated p305 and p306, respectively) behind the ATc-inducible promoter located on the plasmid.

Overexpression of the sprG2 and sprG3 genes from the plasmids p305 and 306, respectively caused bacteriostasis in S. aureus and E. coli, however, sprG3 was only able to slow the growth of E. coli for a short period of time after induction.

This example demonstrates the ability to design synthetic microorganisms comprising effective bacteriostasis switches, for example, to prevent growth of S. aureus or E. co/i. The pRAB11 vector containing the tightly-regulated P_{XYL/tet} promoter allowed for easy induction and overexpression of the genes. In the future, these genes may be genomically inserted into S. aureus using a pIMAYz E. coli S. aureus shuttle vector for expression using alternative promoters sensitive to environmental changes.

### Example 22. Plasmid Construction for p174 & p229

In this example, the plasmids p229 and p174 were made successfully and used to transform into S. agalactiae. The sequencing results showed no mutations.

Since the pRAB11 plasmid is a high copy vector with tight regulation of the genes downstream of the P_{xyl/tet} promoter, the system produces an easily detectable response from the genes downstream of the promoter. In plasmid p174 the toxin gene sprA1 was added to the pRAB11 plasmid and operably linked to P_{xyl/tet} for ATc-dependent TetR induction. In plasmid p229, green fluorescent protein (GFPmut2) was added to the pRAB11 plasmid and operably linked to P_{xyl/tet} for ATc-dependent TetR induction.

The pRAB11 plasmid is a high-copy expression vector used for anhydrotetracycline (ATc)-dependent expression of genes in either E. coli or Staph aureus. Plasmid pRAB11 was generated by adding another tetO operator to the TetR-regulated promoter, P_{xyl/tet}, in plasmid pRMC2. Helle, Leonie, et al., Microbiology 157.12 (2011): 3314-3323.

TetR is a transcriptional repressor protein that binds to DNA if the tetO sequence is present. The P_{XYL/tet} promoter in pRAB11 has two tetO sequences that flank the transcriptional start site which represses the transcription of any gene just downstream of the promoter. When ATc is added to the culture, it will bind to the repressor protein TetR and inhibit its ability to bind to tetO within the promoter. With the TetR proteins deactivated, the constitutive promoter is derepressed and is uninhibited when recruiting RNA polymerase to transcribe the putative toxin at a high rate.

For the construction of p174, the toxin gene sprA1 was added to pRAB11 and operably linked to P_{xyl/tet} for ATc-dependent TetR induction. The sprA1 gene is native to Staph aureus and is part of a type I toxin antitoxin system. The sprA1 gene codes for a membrane porin protein called PepA1, which accumulates in the cell's membrane and induces apoptosis in dividing cells. The sprA1 gene used here was PCR amplified from the genome of a 502a-like strain named in BioPlx's databases as BP_001.

For the construction of p229, a green fluorescent protein (GFPmut2) was added to pRAB11 behind the P_{xyl/tet} promoter for ATc-dependent expression. The expression of both proteins should go from a state of being transcriptionally repressed by the TetR protein to induced and expressed upon the addition of ATc to the system.

Table 48 shows the single stranded DNA sequences for the primers used during the construction or sequencing of plasmid p174 and p229. All of the sequences are in the 5 prime to 3 prime direction

TABLE 48
Primers Used to Make Plasmids p174 and p229

| | Primer | |
|---|---|---|
| Plasmid | Name | Primer Sequence (5′→3′) |
| p174 | BP_672 | gagtatgatggtaccgttaacagatctgagcCGC |
| | | AGAGAGGAGGTGTATAAGGTG (SEQ ID NO: |
| | | 235) |
| | BP_677 | gttgtaaaacgacggccagtgCCCGGGCTCAGCT |
| | | ATTATCA (SEQ ID NO: 255) |
| | BP_670 | GCTCAGATCTGTTAACGGTACCATCATACTC |
| | | (SEQ ID NO: 256) |
| | BP_671 | CACTGGCCGTCGTTTTACAAC (SEQ ID NO: |
| | | 257) |
| p229 | BP_717 | ACTCTTTGAAGTCATTCTTTACAGGAG (SEQ |
| | | ID NO: 241) |
| | DR_244 | CATCACCTTATACACCTCCTCTCTGCGG (SEQ |
| | | ID NO: 229) |
| | DR_476 | CCGCAGAGAGGAGGTGTATAAGGTGATGAGTAAA |
| | | GGAGAAGAACTTTTCAC (SEQ ID NO: 258) |
| | DR_247 | CAATTTTTATTGGTGCGGCTATATGTCACTTATT |
| | | TGTATAGTTCATCCATGCCATGTG (SEQ ID |
| | | NO: 259) |
| | BP_718 | CTCCTGTAAAGAATGACTTCAAAGAGT (SEQ |
| | | ID NO: 242) |
| | DR_245 | GTGACATATAGCCGCACCAATAAAAATTGATAAT |
| | | AGCTGAGCC (SEQ ID NO: 260) |

Table 49 shows the DNA sequences used in the construction of p174 and p229. The sequences represent one strand of the double stranded DNA fragments.

TABLE 49
Sequences of PCR Fragments Inserted into Plasmid

| Plas- | | Seq. | |
|---|---|---|---|
| mid | Name | ID | Sequence |
| p174 | sprA1 | BP_{—} | CGCAGAGAGGAGGTGTATAAGGTGATGCTT |
| | | DNA_{—} | ATTTTCGTTCACATCATAGCACCAGTCATC |
| | | 150 | AGTGGCTGTGCCATTGCGTTTTTTTCTTAT |
| | | | TGGCTAAGTAGACGCAATACAAAATAGGTG |
| | | | ACATATAGCCGCACCAATAAAAAT (SEQ |
| | | | ID NO: 261) |
| p229 | GFPmut2 | BP_{—} | ATGAGTAAAGGAGAAGAACTTTTCACTGGA |
| | | DNA_{—} | GTTGTCCCAATTCTTGTTGAATTAGATGGT |
| | | 077 | GATGTTAATGGGCACAAATTTTCTGTCAGT |
| | | | GGAGAGGGTGAAGGTGATGCAACATACGGA |
| | | | AAACTTACCCTTAAATTTATTTGCACTACT |
| | | | GGAAAACTACCTGTTCCATGGCCAACACTT |
| | | | GTCACTACTTTCGCGTATGGTCTTCAATGC |
| | | | TTTGCGAGATACCCAGATCATATGAAACAG |
| | | | CATGACTTTTTCAAGAGTGCCATGCCCGAA |
| | | | GGTTATGTACAGGAAAGAACTATATTTTTC |
| | | | AAAGATGACGGGAACTACAAGACACGTGCT |
| | | | GAAGTCAAGTTTGAAGGTGATACCCTTGTT |
| | | | AATAGAATCGAGTTAAAAGGTATTGATTTT |
| | | | AAAGAAGATGGAAACATTCTTGGACACAAA |
| | | | TTGGAATACAACTATAACTCACACAATGTA |
| | | | TACATCATGGCAGACAAACAAAAGAATGGA |
| | | | ATCAAAGTTAACTTCAAAATTAGACACAAC |
| | | | ATTGAAGATGGAAGCGTTCAACTAGCAGAC |
| | | | CATTATCAACAAAATACTCCAATTGGCGAT |
| | | | GGCCCTGTCCTTTTACCAGACAACCATTAC |
| | | | CTGTCCACACAATCTGCCCTTTCGAAAGAT |
| | | | CCCAACGAAAAGAGAGACCACATGGTCCTT |
| | | | CTTGAGTTTGTAACAGCTGCTGGGATTACA |
| | | | CATGGCATGGATGAACTATACAAATAA |
| | | | (SEQ ID NO: 262) |

### PCR Fragment Generation

The following PCR reactions were performed using Q5 High Fidelity Hot Start Master Mix (NEB) per the manufacturer's instructions.
BP_DNA_095—p151 Backbone Fragment (p174)
   BP_670/BP_671
BP_DNA_095—p174 Backbone Fragment (p229)
   DR_244/DR_245
BP_DNA_—sprA1 (Inserted sequence) (p174)
   BP_672/BP_677
BP_DNA_077—GFPmut2 (Inserted sequence) (p229)
   DR_476/DR_247

The above PCR fragments were checked on a 1% agarose gel to confirm a clean band, and then purified using a Qiaquick PCR Purification Kit (Qigagen) per the manufacturer's instructions. The p174 fragment was treated with DpnI (NEB) to remove the pRAB11 plasmid used as the template for the PCR, and purified again using the PCR Cleanup Kit (NEB) per the manufacturer's instructions. The DNA fragments were used in a Gibson Assembly (NEB) to create a circular plasmid per the manufacturer's instructions. The assembled plasmid was then transformed into IM08B, plated on LB (carb), and incubated overnight at 37° C. The following day, colonies were screened for fully assembled plasmids by colony PCR to check for the presence of the GFP or sprA1 on the pRAB11 plasmid within the colony. Three positive colonies were picked, grown overnight in 5 mL of LB (plus carbenicillin, 100 ug/mL), and the plasmid was extracted using the ZymoPURE plasmid miniprep kit per the manufacturer's instructions. The plasmid was then sequenced to confirm the DNA sequence of the GFPmut2 or sprA1 gene. The sequencing was aligned in silico using the sequence alignment tool in Benchling. One of each of the colonies whose sequencing alignment that showed a perfect alignment to the reference map's sequence was picked and stocked in the plasmid database.

### Example 23. Transformation of Electrocompetent Streptococcus agalactiae Cells

Streptococcus agalactiae was transformed by a variation of procedures from Framson et al. and Duny et al. (Framson, et al., Appl. Environ. Microbiol. 1997, 63 (9), 3539-3547, Dunny et al., Appl. Environ. Microbiol. 1991, 57 (4), 1194-1201).

Briefly, the electrocompetent cell protocol starts by inoculating a single overnight culture of S. agalactiae A909 (BPST_002) in M9 Media with 1% Casamino Acids and 0.3% Yeast Extract (M9-YE) and incubating overnight at 37° C. The next day, that culture was used to inoculate a larger volume of the same media but with 1.2% glycine. The new culture was statically incubated at 37° C. for 12 to 15 h. Glycine disrupts the biosynthesis of the peptidoglycan cell wall by replacing the L-alanine in the peptide crosslinker. This causes pore formation in the electrocompetent cells and therefore increases the likelihood of DNA uptake during transformation. After the incubation period, the culture with glycine will be added into a larger volume of fresh M9-YE+1.2% glycine and incubated for 1 h at 37° C. After the growth period, the OD was checked and found to be in the target range of 0.1-0.25 OD. After the culture reached the target OD, the cells were pelleted by centrifuging the culture and the resulting supernatant was removed. The cell pellet was resuspended in an osmoprotectant solution (0.625 M Sucrose, pH 4), pelleted again through centrifugation and the supernatant removed. The cells were resuspended in a small volume of the osmoprotectant solution. After the final resuspension, the cells were either chilled on ice for 30 to 60 minutes and used for electroporation, or immediately stored in the −80° C. freezer.

The electroporation protocol followed the procedure by Duny et al. but used recovery media from the Framson et al. protocol.

Briefly, competent S. agalactiae cells were thawed on ice, transferred to a 2-mm electroporation cuvette where at least 300 ng of plasmid DNA was added directly to the competent cells, and the cells are electroporated at 2.0 kV with a 200Ω resistance. Afterwards, the cuvette was briefly placed on ice, 0.5 M sucrose in THB is added to the cells and the suspension is transferred to a culture tube. The transformation is statically recovered at 37° C. for 1 hr before being plated on THB agar plates with the appropriate antibiotic selection. The plates are incubated overnight at 37° C. and the presence of colonies indicates that plasmid has been taken up by S. agalactiae.

### Example 24. Toxin Efficacy Test in S. agalactiae Using Inducible Gene Expression

The putative Staphylococcus aureus toxin gene sprA1 under the control of the P_{XYL/Tet} promoter on the pRAB11 vector was transformed into Streptococcus agalactiae A909 (BPST_002) by the method of Example 23.

In the present example the ability of the sprA1 toxin gene from Staphylococcus aureus (S. aureus) to cause cell death or prevent cell growth when expressed from a pRAB11 plasmid transformed into Streptococcus agalactiae (S. agalactiae) was tested. A strong inducible and tightly controlled promoter system, P_{XYL/Tet} on pRAB11 was employed. The effect of sprA1 overexpression on the growth of S. agalactiae was observed by measuring the optical density (OD) of the culture over the growth period.

Overexpression of the sprA1 gene prevented growth of the BPST_002 cell cultures, indicating the production of PepA1 functions as a bacteriostatic toxin to the host cells. To verify the P_{XYL/Tet} promoter, a plasmid with a GFP operably linked to the P_{XYL/Tet} promoter was also transformed into S. agalactiae A909 (BPST_002). Induction of the GFP-containing plasmid showed a 10-fold increase in the amount of fluorescence between induced cultures and uninduced cultures.

pRAB11 plasmids p174 and p229 containing a toxin and green fluorescence protein (GFP), respectively, under the control of the P_{XYL/Tet} promoter system were transformed into BPST_002.

In plasmid p174, the sprA1 gene was added directly after the promoter system. The toxin is native to Staph aureus, and is part of a type I toxin antitoxin system. The sprA1 gene used here was PCR amplified from the genome of a Staphylococcus aureus 502a-like strain BP_001.

In plasmid p229, a GFPmut2 was added to pRAB11 behind the P_{xyl/tet} promoter. The expression of both proteins was expected to go from a state of being transcriptionally repressed by the TetR protein to induced and expressed upon the addition of ATc to the system.

This system was used to test the effect of overexpression of the sprA1 toxin, PepA1, on the growth of BPST_002 (S. agalactiae A909). The sprA1 gene codes for a membrane porin protein called PepA1, which accumulates in the cell's membrane and induces apoptosis in dividing cells. This effect was expected to cause cell death or failure of cells to grow in cultures induced with Atc, as measured by OD600. To confirm the effectiveness of the P_{XYL/tet} promoter, the fluorescence of induced and uninduced cultures was measured using a plate reader.

Table 50 shows the plasmid numbers and descriptions that were transformed into BPST_002.

TABLE 50
Plasmids Transformed into Streptococcus agalactiae BPST_002

| Number | Name | Description |
|---|---|---|
| p174 | pRAB11_Ptet- | sprA1 toxin gene (without antitoxin |
| | sprA1 | sequence) under control of tetracycline- |
| | | inducible promoter. The gene includes some |
| | | sequence upstream of the start codon. |
| p229 | pRAB11_P(xyl- | Green fluorescent protein gene under control |
| | tet)-GFPmut2 | of anhydrotetracycline-inducible promoter |

Transformation and PCR Screen

The plasmids were electroporated into BPST_002 electrocompetent cells and colonies were PCR screened for the presence of the plasmid using DR_216/DR_217. Plasmids p229 and p174 were transformed into the S. agalactiae BPST_002 electrocompetent cells using the protocol above. The transformation was recovered statically at 37° C. for 1 hr and plated on THB agar plates with 1 ug/mL of chloramphenicol. The plates were incubated for 16-24 hrs. When colonies were visible, a sterile inoculation loop was employed to pick single colonies from each transformation and restreak for single colony isolation on fresh THB agar plates with 1 g/mL of chloramphenicol. The plates were incubated at 37° C. for 12-16 hrs.

The following day, colonies were PCR screened on new streak plates for the presence of the plasmid using DR_215/DR_216. PCR products were run on a 1% agarose gel to check for colonies that are positive for the integration. If all colonies are positive for the presence of the plasmid, the streak plate was used to start cultures for growth assays.

Growth Assay with Stationary Phase Cultures
1. Start three 5 mL THB+chloramphenicol (1 μg/mL) culture for each plasmid to be tested from a single colony on fresh agar plates. Statically incubate for 8 hr at 37° C.
2. After the incubation period, measure the OD600 of the cultures.
3. Add 5 μL of anhydrotetracycline (ATc) (1 ug/mL) to two of the three samples. The unspiked sample is the control.
4. Statically incubate culture tubes at 37° C. for 1 hour.
5. After the incubation period, measure the OD600 of the cultures.
6. Enter recorded ODs in a table and plot the data on a graph to show the growth curves for all of the strains tested.

Growth Assay with Exponential Phase Cultures
1. Start three 5 mL THB+chloramphenicol (1 μg/mL) culture for each plasmid to be tested from a single colony on fresh agar plates. Statically incubate for 8 hr at 37° C.
2. After the incubation period, measure the OD600 of the cultures.
3. Add 500 μL of cultures to 4.5 mL of fresh THB+chloramphenicol (1 μg/mL), briefly vortex to mix the culture.
4. Remove 500 μL of each culture and measure the OD600.
5. Add 4.5 μL of anhydrotetracycline (ATc) (1 mg/mL) to two of the three samples. The unspiked sample is the control.
6. Immediately after the addition of the ATc and before putting the tubes in the 37° C. incubator, briefly vortex to mix the culture.
7. Statically incubate culture tubes at 37° C. for 1 hour.
8. After 1 hour measure and record the OD600 readings,
9. Place cultures back in the 37° C. incubator and measure and record the OD600 values every hour for a total of 3 hrs.

Fluorescence Sample Preparation and Measurements
10. After 3 hrs of incubation, spin down the p229 in BPST_002 cultures for 5 minutes at 3500 rpm.
11. Remove the supernatant and add 5 mL of PBS. Resuspended the cultures by briefly vortexing.
12. Centrifuge cultures again for 5 minutes at 2800×g.
13. Remove the supernatant and resuspend cell pellet in 1 mL of PBS.
14. Add 200 uL of each cell suspension to a 96-well plate (Greiner Bio, Part #655900) in triplicate. Include PBS in triplicate as a blank.
15. Read the plate with the following settings:
   a. Ex: 485/20
   b. Em: 530/25
   c. Sensitivity: 80
16. Subtract the blank reading from the experimental samples and record all values.

### Results:

Both plasmids p174 and p229 were successfully transformed into Streptococcus agalactiae BPST_002 and PCR confirmed with DR_215 and DR_216. Growth assays were performed on a single day with cultures started directly from a single colony. The assays were performed in the exact same manner each time according to the protocol described above.

Table 51 shows the OD₆₀₀ readings for p174 & p229 in BPST_002 grown in THB. The OD600 for induced cultures where ATc was added to induce the expression of the sprA1 toxin or GFP reporter gene, were compared to uninduced cultures (control, no ATc).

TABLE 51
OD Values of p174 & p229 in BPST_002 (+/−ATc) over 3 hours

Time (hours)

| Sample Name | 0 | 1 | 2 | 3 |
|---|---|---|---|---|
| p174 + ATc #1 | 0.22 | 0.23 | 0.23 | 0.24 |
| p174 + ATc #2 | 0.25 | 0.23 | 0.23 | 0.22 |
| p174 (control) | 0.26 | 0.7 | 2.4 | 2.4 |
| p229 + ATc #1 | 0.28 | 0.5 | 1.0 | 1.4 |
| p229 + ATc #2 | 0.29 | 0.5 | 1.2 | 1.6 |
| p229 (control) | 0.27 | 0.7 | 2.1 | 2.3 |

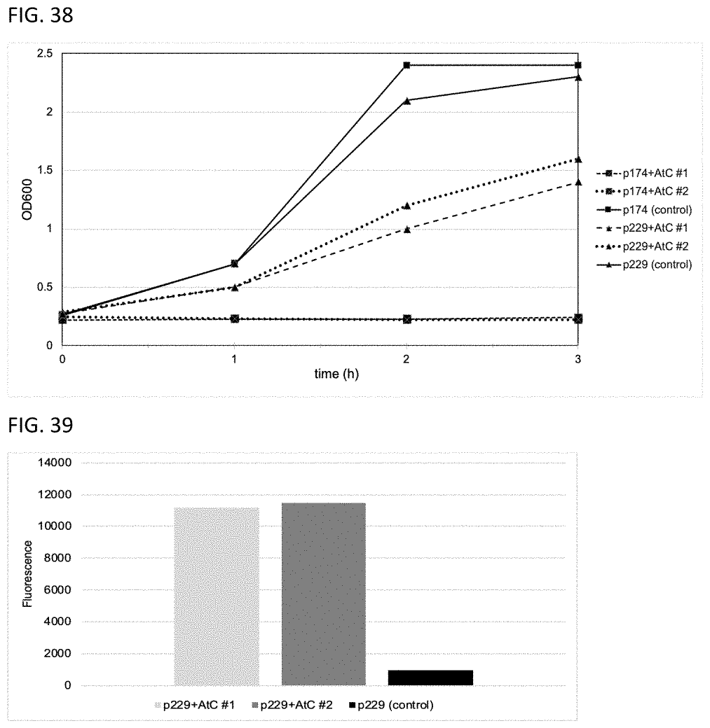

The data from Table 51 is plotted on a graph in FIG. 38. FIG. 38 shows a graph of OD600 growth curves over 3 hours for Streptocccus agalactiae (BPST_002) transformed with plasmids p174 (sprA1) or p229 (GFP). The starting cultures were inoculated at a 1:10 dilution from stationary phase cultures. The t=0 hr OD was taken before ATc induction. The dashed line represents the cultures that were induced with ATc and the solid line represents control cultures. overexpression of sprA1 toxin gene is able to inhibit S. agalactiae cell growth in exponential phase All data points represent single cultures.

The results show that overexpression of sprA1 toxin gene is able to inhibit S. agalactiae cell growth in exponential phase. The OD600 values of the ATc spiked samples did not increase after the addition of ATc, while the control samples continued to grow. This indicates that the sprA1 gene from S. aureus is capable of inhibiting growth and possibly killing S. agalactiae cells when overexpressed.

To show that ATc is not inherently toxic to the cells and therefore responsible for the inhibition of cell growth, cultures of wild-type BPST_002 were grown overnight. One culture was induced with ATc and the resulting OD was compared to the non-induced culture. The ATc culture had a 10% higher OD600 as compared to the control culture (data not shown). Therefore, the addition of ATc at a concentration of 1 ug/mL was not toxic to BPST_002 cell growth.

FIG. 39 shows a bar graph of fluorescence values at 3 hours after induction of Streptococcus agalactiae (BPST_002) transformed with plasmid p229 (GFP). The starting cultures were inoculated at a 1:10 dilution from stationary phase cultures. Cultures were grown in duplicate and fluorescence readings were performed in triplicate. Increased fluorescent values of induced p229 cultures indicate the ability of the P_{XYL/Tet} promoter system of pRAB11 to function as an ATc inducible promoter in S. agalactiae.

### Example 25. Stability of a Mixture of Staphylococcus aureus, Streptococcus agalactiae and Escherichia coli

The stability of a mixture of synthetic Staphylococcus aureus (BP_123), synthetic Escherichia coli (BPEC_006), and Streptococcus agalactiae (BPST_002, WT A909) in PBS was determined.

Cell suspensions of BP_123, BPST_002 and BPEC_006 in PBS were relatively stable after 24 h storage at 4° C. as assessed by CFU plating. After 24 h, BP_123 decreased by 25% in a mixture with BPST_002 and BPEC_009, but also decreased in a suspension that contained only BP_123. BPST_002 and BPEC_009 remained within +/−10% of the original t=0 samples in the cell suspension mixture with all 3 bacteria types. Colonies were visually differentiated by growth characteristics on TSB and supported by PCR strain screen data.

SSTI's such as mastitis can be caused by three main bacterial species; Staphylococcus aureus, Streptococcus agalactiae and Escherichia coli. These bacteria can live naturally within the microbiome or environment but can cause mastitis if an opportunistic infection occurs, e.g., in the udder.

Synthetic strains of all of these species can be prepared by genomically integrating a safety switch using kill switch technology in order to cause immediate bacterial cell death upon entering the bloodstream or tissue.

A live biotherapeutic composition containing a mixture of all three bacterial types must ensure that the viability of each of the bacteria remains stable when mixed together. This example assesses the stability of S. aureus (BP_123), S. agalactiae (BPST_002) and E. coli (BPEC_006) when suspended in phosphate buffered saline (PBS) together for future use as a biotherapeutic intervention for treatment of, e.g., an SSTI in a subject.

Briefly, BP_123, BPEC_006 and BPST_002 were grown in overnight overnight cultures. The following day the cells were harvested, washed three times in PBS and concentrated. The concentration of viable colony forming units (CFUs) was determined by performing a serial dilution of the cell suspension, plating several different dilutions on non-selective agar plates, and counting the colonies the following day to calculate the cell concentration. The washed cultures were then resuspended in an appropriate volume of PBS to reach the target concentration of 1×10⁷ CFU/mL. The stability suspensions were plated on TSB plates and the suspensions were stored at 4° C. After 24 hrs of storage the stability suspensions were plated again and the final CFU/mL compared to the t=0 CFU/mL.

Table 52 shows the strain numbers and description of strains that were used in the stability study.

TABLE 52
Strains in Stability Study

| Number | Bacteria | Strain Description |
|---|---|---|
| BPST_002 | S. agalactiae | Strain A909, wild-type |
| BPEC_006 | E. coli | E. coli isolated from |
| | | bovine sample (Udder Health Systems, |
| | | Inc.) Genetically modified: |
| | | DuidA::tetR_Pxyl/tet-sprA1_kanR |
| BP_123 | S. aureus | Strain 502a, Genetically modified: |
| | | ΔsprA1; isdB::sprA1 |

Table 53 shows stability suspension mixtures, the final target concentration and final volume of PBS.

TABLE 53
Stability Suspension Mixtures of S. agalactiae,
E. coli, and S. aureus

| | Stability | Target | Final Volume |
|---|---|---|---|
| | Samples | Concentration | (uL) |
| A | BP_123 | 1.00E+07 | 5000 |
| B | BPST_002 | 1.00E+07 | 5000 |
| C | BPEC_006 | 1.00E+07 | 5000 |
| D | BP_123 | 1.00E+07 | 5000 |
| | BPST_002 | 1.00E+07 | 5000 |
| | BPEC_006 | 1.00E+07 | 5000 |
| E | BP_123 | 1.00E+07 | 5000 |
| | BPST_002 | 1.00E+07 | 5000 |
| F | BP_123 | 1.00E+07 | 5000 |
| | BPEC_006 | 1.00E+07 | 5000 |
| G | BPST_002 | 1.00E+07 | 5000 |
| | BPEC_006 | 1.00E+07 | 5000 |

A 10⁻⁵ dilution of Stability Suspension D containing BP_123, BPST_002 and BPEC_006 was plated on TSB. Colonies were visibly different so BP_123 colonies could be differentiated from BPST_002 and BPEC_006 and vice versa.

Strain identities were confirmed using PCR. The PCRs products were run on a 100 agarose gel of the strain screen from lysed colonies from stability suspension D TSB plate. All colonies were screened from a single 10⁻⁵ dilution plate using the SA lysis procedure. Visibly like colonies were grouped together and the 3 PCRs were run on all of the lysates. Primers are shown in Table 54

TABLE 54
PCR Band Size and Primer Details for Strain Screen

| | | PCR | Primer Sequence | PCR band | |
|---|---|---|---|---|---|
| Number | Bacteria | Primers | (5′→3′) | size (bp) | Target Area |
| BP_123 | S. | DR_254 | ATGCTTATTTTCGTTCACAT | 1391 | sprA1 |
| | aureus | | CATAGCACCAGTCATCAGT | | integration |
| | | | G (SEQ ID NO: 206) | | site to DNA |
| | | DR_534 | CAGCTGTTGATAATGCCAT | | outside of |
| | | | TTTTGCACGAG (SEQ ID NO: | | integration |
| | | | 208) | | area |
| BPEC_006 | E. coli | DR_372 | GCCATCTGTAAATCTTGCG | 2114 | sprA1 |
| | | | CCATTAGTCC (SEQ ID NO: | | integration |
| | | | 197) | | site to DNA |
| | | DR_254 | ATGCTTATTTTCGTTCACAT | | outside of |
| | | | CATAGCACCAGTCATCAGT | | integration |
| | | | G (SEQ ID NO: 206) | | area |
| BPST_002 | S. aga- | BM_152 | AGGAATACCAGGCGATGAA | 952 | dltS gene |
| | lactiae | | CCGAT (SEQ ID NO: 263) | | |
| | | BM_153 | TGCTCTAATTCTCCCCTTAT | | |
| | | | GGC (SEQ ID NO: 264) | | |

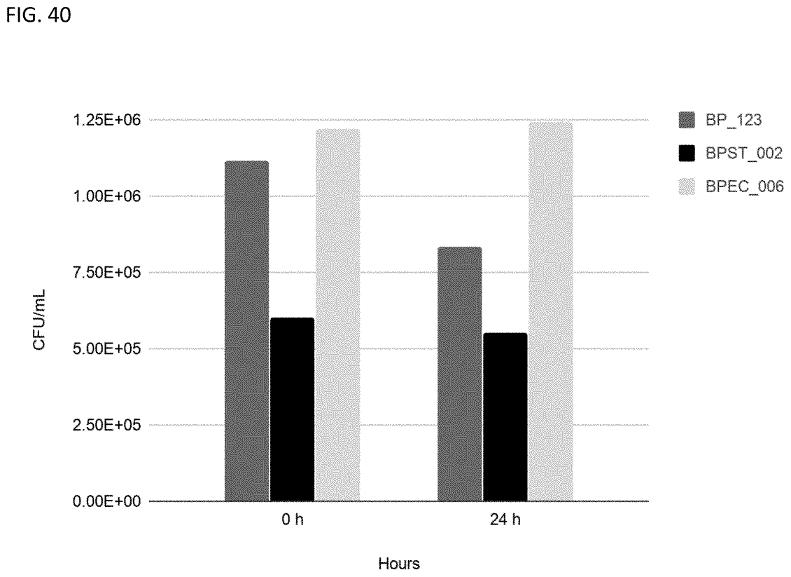

Stability results are shown in FIG. 40 showing a bar graph calculated from the CFU/mL data of Stability Suspension D containing BP_123, BPST_002, BPEC_006 at 0 and 24 hours. All dilutions were plated in duplicate on TSB plates. CFU/mL data was calculated from the 104 dilution.

The observed CFU/mL at t=0 and 24 h supports the stability of cell suspensions containing a mixture of S. aureus, S. agalactiae and E. coli. In stability suspension D, CFU/mL of BPST_002 and BPEC_006 remained stable after a period of 24 h but BP_123 viability decreased by roughly 25% as seen in FIG. 40. Cell suspension A, containing only BP_123, also decreased significantly from t=0 h. Based on this data, BP_123 decreased independently of being mixed with BPEC_006 or BPST_002. The CFU/mL of BPST_002 and BPEC_006 in stability suspension D were comparable to stability suspensions B and C which contained only one type of bacteria. This also leads to the conclusion that a mixed cell population does not influence the CFU/mL of different bacterial types which is important for the development of a biotherapeutic composition, e.g., for treatment of an SSTI in a subject.

1. A method of preparing a synthetic microorganism comprising:
transforming a target microorganism in the presence of a plasmid comprising
a synthetic nucleic acid sequence comprising an action gene flanked by an upstream homology arm and a downstream homology arm, wherein the upstream and downstream homology arms comprise a first and a second complementary nucleic sequence, respectively, for targeting insertion of the action gene behind a native inducible promoter gene in the genome of the target microorganism.

2. The method of claim 1, further comprising
selecting a native inducible promoter gene in the target strain for targeted insertion of the synthetic nucleic acid sequence comprising the action gene, comprising
comparing the relative RNA transcription levels of a native inducible gene in the target microorganism when grown in a first environmental condition compared to a second environmental condition, wherein the target microorganism exhibits at least a 10-fold increase in RNA transcription level when grown in the second environmental condition compared to the first for a comparable period of time.

3. The method of claim 2, wherein the period of time is selected from the group consisting of at least about 15 min, 20 min, 30 min, 40 min, 45 min, 50 min, 60 min, 75 min, 90 min, 120 min, 180 min, 210 min, 240 min, 270 min, 300 min, 330 min, and 360 min, or any time point in between, and optionally wherein the RNA transcription levels in the target microorganism are assessed using an RNA-seq assay.

4. The method of claim 1, wherein the target microorganism is a bacterial species capable of colonizing a first environmental niche and is a member of a genus selected from the group consisting of Staphylococcus, Streptococcus, Escherichia, Bacillus, Acinetobacter, Mycobacterium, Mycoplasma, Enterococcus, Corynebacterium, Klebsiella, Enterobacter, Trueperella, and Pseudomonas.

5. The method of claim 4, wherein the first environmental condition is a complete media or a dermal, gastrointestinal, genitourinary, or mucosal niche in a subject.

6. The method of claim 5, wherein the second environmental condition comprises exposure to or an increase in concentration of blood, plasma, serum, interstitial fluid, synovial fluid, contaminated cerebral spinal fluid, lactose, glucose, or phenylalanine.

7. The method of claim 1, wherein the synthetic microorganism comprises
a first molecular modification inserted to the genome of the target microorganism, the molecular modification comprising a first recombinant nucleotide comprising the action gene,
wherein the first recombinant nucleotide is operatively associated with an endogenous first regulatory region comprising a native inducible first promoter gene, and
wherein the native inducible first promoter imparts conditionally high level gene transcription of the first recombinant nucleotide in response to exposure to the second environmental condition of at least 10-fold increase compared to the first environmental condition.

8. The method of claim 7, wherein the action gene is selected from the group consisting of a cell death action gene, virulence block action gene, metabolic modification action gene, nanofactory action gene, transcriptional regulator TetR-family gene, lacZ gene which codes for β-galactosidase (lactase or β-gal), or a gene which encodes an enzyme or hormone, optionally selected from the group consisting of sortase A (srt A), aerobic glycerol-3-phosphate dehydrogenase gene (glpD), thymidine kinase (tdk), glutenase, endopeptidase, prolyl endopeptidase (PEP), endopeptidase 40, insulin, and insulin precursor.

9. The method of claim 8, wherein the action gene is a cell death gene.

10. The method of claim 9, wherein the plasmid is derived from a shuttle vector suitable for use in both a pass through microorganism and the target microorganism.

11. The method of claim 10, wherein the pass through microorganism is a synthetic pass through strain comprising
(a) a first genomic modification comprising a first synthetic nucleic acid sequence encoding a DNA methylation enzyme and/or acetylation enzyme derived from the target microorganism; and
(b) a second genomic modification comprising a second synthetic nucleic acid sequence comprising an antitoxin gene encoding an antisense RNA sequence capable of hybridizing with at least a portion of the cell death gene.

12. The method of claim 11, wherein the presence of the antisense genomic modification in the pass through strain allows the pass through strain to propagate the plasmid comprising the cell death gene, and allows the pass through strain to survive leaky expression of the toxin gene in the plasmid.

13. The method of claim 12, wherein the presence of the genomic modification encoding the methylation enzyme and/or acetylation enzyme in the pass through strain allows the pass through strain to impart
a methylation pattern and/or acetylation pattern on the plasmid DNA similar enough to the methylation pattern and/or acetylation pattern of the target microorganism, to enable or enhance efficiency of transformation of the target strain with the plasmid propagated in the pass through strain.

14. The method of claim 11, wherein the pass through strain is an Escherichia coli strain or a yeast strain.

15. The method of claim 14, wherein the target microorganism has the same genus and species as an undesirable microorganism capable of causing bacteremia or SSTI in the subject.

16. The method of claim 15, wherein undesirable microorganism is capable of causing bacteremia or SSTI in the subject.

17. The method of claim 9, wherein measurable average cell death of the synthetic microorganism occurs within at least a preset period of time following exposure to the second environmental condition.

18. The method of claim 17, wherein the measurable average cell death occurs within the preset period of time selected from the group consisting of within at least about 15, 30, 60, 90, 120, 180, 240, 300, or 360 min minutes following exposure to the second environmental condition.

19. The method of claim 18, wherein the first environmental condition is a complete media or a dermal, or mucosal niche in a subject.

20. The method of claim 19, wherein the second environmental condition comprises exposure to or an increase in concentration of blood, plasma, serum, interstitial fluid, synovial fluid, or contaminated cerebral spinal fluid.

21. The method of claim 20, wherein the measurable average cell death is a cfu count reduction of at least 50%, at least 70%, at least 80%, at least 90%, at least 95%, at least 99%, at least 99.5%, at least 99.8%, or at least 99.9% cfu count reduction following the preset period of time.

22. The method of claim 21, wherein the synthetic microorganism is incapable of causing bacteremia or SSTI in a subject.

23. The method of claim 1, wherein target microorganism is derived from a Staphylococcus aureus strain.

24. The method of claim 23, wherein the action gene is a cell death gene selected from or derived from the group consisting of sprA1, sprA2, sprG, mazF, relE, relF, hokB, hokD, yafQ, rsaE, yoeB, yefM, kpn1, sma1, or lysostaphin toxin gene.

25. The method of claim 24, wherein the action gene comprises a nucleotide sequence selected from the group consisting of SEQ ID NOs: BP_DNA_003(SEQ ID NO: 3), BP_DNA_008 (SEQ ID NO: 8), BP_DNA 0032, BP_DNA_035 (SEQ ID NO:25), BP_DNA_045 (SEQ ID NO: 29), BP_DNA_065 (SEQ ID NO: 34), BP_DNA 067 (SEQ ID NO: 35), BP_DNA_068 (SEQ ID NO: 36), BP_DNA 069 (SEQ ID NO: 37), BP_DNA 070 (SEQ ID NO: 38), BP_DNA 071 (SEQ ID NO: 39), or a substantially identical nucleotide sequence.

26. The method of claim 23, wherein the target microorganism is a S. aureus strain, and the inducible first promoter gene is selected from the group consisting of isdA (iron-regulated surface determinant protein A), isdB (iron-regulated surface determinant protein B), isdG (heme-degrading monooxygenase), hlgA (gamma-hemolysin component A), hlgA1 (gamma-hemolysin), hlgA2 (gamma-hemolysin), hlgB (gamma-hemolysin component B), hrtAB (heme-regulated transporter), sbnC (luc C family siderophore biosyntheis protein), sbnD, sbnI, sbnE (lucA/lucC family siderophore biosynthesis protein), isdI, lrgA (murein hydrolase regulator A), lrgB (murein hydrolase regulator B), ear (Ear protein), fhuA (ferrichrome transport ATP-binding protein fhuA), fhuB (ferrichrome transport permease), hlb (phospholipase C), heme ABC transporter 2 gene, heme ABC transporter gene, isd ORF3, sbnF, alanine dehydrogenase gene, diaminopimelate decarboxylase gene, iron ABC transporter gene, threonine dehydratase gene, siderophore ABC transporter gene, SAM dep Metrans gene, HarA, splF (serine protease SplF), splD (serine protease SplD), dps (general stress protein 20U), SAUSA300_2617 (putative cobalt ABC transporter, ATP-binding protein), SAUSA300_2268 (sodium/bile acid symporter family protein), SAUSA300_2616 (cobalt family transport protein), srtB (Sortase B), sbnA (probable siderophore biosynthesis protein sbnA), sbnB, sbnG, leuA (2-isopropylmalate synthase amino acid biosynthetic enzyme), sstA (iron transport membrane protein), sirA (iron ABC transporter substrate-binding protein), isdA (heme transporter), and spa (Staphyloccocal protein A).

27. The method of claim 26, wherein the inducible first promoter gene comprises a nucleotide sequence complementary to an upstream and or downstream homology arm having a nucleic acid sequence selected from the group consisting of BP_DNA_001 (SEQ ID NO: 1), BP_DNA_002 (SEQ ID NO: 2), BP_DNA_004 (SEQ ID NO: 4), BP_DNA_006 (SEQ ID NO: 6), BP_DNA_007 (SEQ ID NO: 7), BP_DNA_010 (SEQ ID NO: 9), BP_DNA_BP_DNA_012 (SEQ ID NO: 10), BP_DNA_013 (SEQ ID NO: 11), BP_DNA_014 (SEQ ID NO: 12), BP_DNA_016 (SEQ ID NO: 13), BP_DNA_017 (SEQ ID NO: 14), BP_DNA_029 (SEQ ID NO: 20), BP_DNA_031 (SEQ ID NO: 22), BP_DNA_033 (SEQ ID NO: 24), BP_DNA_041 (SEQ ID NO: 27), and BP_DNA_057 (SEQ ID NO: 31), or a substantially identical nucleotide sequence thereof.

28. The method of claim 1, wherein the method further comprises
inserting at least a second molecular modification (expression clamp) into the genome of the target microorganism, the second molecular modification comprising
a (anti-action) regulator gene encoding a small noncoding RNA (sRNA) specific for the control arm or action gene, wherein the regulator gene is operably associated with
an second regulatory region comprising a second promoter gene which is transcriptionally active (constitutive) when the synthetic microorganism is grown in the first environmental condition, but is not induced, induced less than 1.5-fold, or is repressed after exposure to the second environmental condition for a period of time of at least 120 minutes.

29. The method of claim 28, wherein regulator gene. encodes an sRNA sequence capable of hybridizing with at least a portion of the action gene.

30. The method of claim 28, wherein the second molecular modification comprises or is derived from the group consisting of a sprA1 antitoxin gene, sprA2 antitoxin gene, sprG antitoxin gene or sprF, holin antitoxin gene, 187-lysK antitoxin gene, yefM antitoxin gene, lysostaphin antitoxin gene, or mazE antitoxin gene, kpn1 antitoxin gene, sma1 antitoxin gene, relF antitoxin gene, rsaE antitoxin gene, or yoeB antitoxin gene, respectively.

31. The method of claim 30, wherein the second molecular modification comprises a nucleotide sequence comprising BP_DNA_005 (SEQ ID NO: 5), or a substantially identical nucleotide sequence.

32. The method of claim 28, wherein the second promoter comprises or is derived from a gene selected from the group consisting of PsprA1as (sprA1as native promoter), clfB (Clumping factor B), sceD (autolysin, exoprotein D), walKR (virulence regulator), atlA (Major autolysin), oatA (O-acetyltransferase A); phosphoribosylglycinamide formyltransferase gene, phosphoribosylaminoimidazole synthetase gene, amidophosphoribosyltransferase gene, phosphoribosylformylglycinamidine synthase gene, phosphoribosylformylglycinamidine synthase gene, phosphoribosylaminoimidazole-succinocarboxamide gene, trehalose permease IIC gen, DeoR faimly transcriptional regulator gene, phosphofructokinase gene, PTS fructose transporter subunit IIC gene, galactose-6-phosphate isomerase gene, NarZ, NarH, NarT, alkylhydroperoxidase gene, hypothetical protein gene, DeoR trans factor gene, lysophospholipase gene, protein disaggregation chaperon gene, alkylhydroperoxidase gene, phosphofructokinase gene, gyrB, sigB, and rho.

33. The method of claim 8, wherein the action gene encodes a β-galactosidase (lactase or β-gal) enzyme.

34. The method of claim 33, wherein the β-galactosidase enzyme is a prokaryotic β-galactosidase enzyme.

35. The method of claim 33, wherein the β-galactosidase enzyme comprises an amino acid sequence selected from the group consisting of SEQ ID NO: 94, 268, and 270.

36. The method of claim 8, wherein the action gene encodes a glutenase.

37. The method of claim 36, wherein the glutenase is a prolyl endopeptidase.

38. The method of claim 37, wherein the prolyl endopeptidase comprises an amino acid sequence selected from the group consisting of SEQ ID NOs: 92 and 93.

39. The method of claim 8, wherein the action gene encodes an insulin or insulin precursor.

40. The method of claim 39, wherein the insulin or insulin precursor comprises an amino sequence of SEQ ID NO: 105.

41. A synthetic microorganism comprising
a first molecular modification inserted to the genome of a target microorganism, the molecular modification comprising a first recombinant nucleotide comprising an action gene,
wherein the first recombinant nucleotide is operatively associated with an endogenous first regulatory region comprising a native inducible first promoter gene, and
wherein the native inducible first promoter imparts conditionally high level gene transcription of the first recombinant nucleotide in response to exposure to a change in state of at least three fold increase compared to basal productivity.

42. A synthetic microorganism comprising
a first molecular modification inserted to the genome of a target microorganism, the molecular modification comprising a recombinant nucleotide comprising a first regulatory region comprising an inducible first promoter gene,
wherein the inducible first promoter gene is operably associated with an endogenous action gene, and
wherein the inducible first promoter imparts conditionally high level gene transcription of the endogenous action gene in response to a change in state of at least three fold increase of basal productivity.

43.-94. (canceled)

95. A method of preparing a synthetic microorganism comprising a genomically stable, genomically incorporated kill switch (KS) molecular modification, comprising
identifying a target microorganism;
selecting a fluid or environment of interest for KS activation in target microorganism;
mapping at least a part of the target microorganism genome for KS integration;
finding an upregulated gene or promoter region in the target microorganism genome by exposing the target microorganism to the fluid or environment of interest;
identifying a candidate toxin gene that is native or non-native to the target microorganism;
creating a plasmid containing the candidate toxin gene underneath the control of an inducible promoter;
transforming the plasmid into the target microorganism, inducing the inducible promoter, and screening for cell death;
selecting a lethal candidate toxin gene for genomic integration in the target microorganism under the regulation of the upregulated gene or promoter region in the fluid or environment of interest;
inserting the candidate toxin gene near the gene or promoter region in the target microorganism genome that is upregulated in fluid or environment of interest to create the synthetic microorganism comprising a genomically stable, genomically incorporated kill switch (KS) molecular modification.

96.-106. (canceled)


SEQUENCE LISTING (280 sequences)

SEQ ID NO: 1 - DNA, 980 bp, Artificial Sequence
  BP_DNA_001 isdB Upstream Homology Arm w/ Frameshift
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaataataaatc

SEQ ID NO: 2 - DNA, 950 bp, Artificial Sequence
  BP_DNA_002 isdB Downstream Homology Arm
gtctttatatttaattattaaattaacaaattttaattggcggatgaggtatccagttacctcgttcgccaattatttttcgcaatataaaaagtcccacttaaaacaatcattttaagcgggactttttatattgagtaactaaaattatttagctgctacttcttcgccattgtaagaaccacagtttttacatacacggtgtgataatttgtattcgccacagtttgggcattcagtcatacctggtactgaaattttgaaatgcgtacgacgtttgttttttctagttttagaagttcttctttttggtactgccatgatatatcctccttagattataaacgaaaaatactaaatgttagtttaattaacaacattatatcattaattaaactacttattgctctttatcatataattgttgtaatttttgaagccttggatcaacttgtcgtgattctgaatcatcttgttcttgctgtttagcaagctcatctaattgatcctcatcgattacttcccaaccattacctactgtcaacatttggtcactttgctctgaataagctctcattggtttctcaataataactatatcctcgacaatatcctgaagattaaccataccatctttaataatgtgatagtgttcatctacatcatcttgatcatcgttatactgattgtacccttctaaatcaaatacttctgtagtagttacatctagtgggacttttactggtacaagagtacgtgcacaaggcattgtatacgttccagtaatgtgaatatccgcaacgacttctgttgacttaatggttaactgaccttggattgtaattggagataaatcaattaaatctaatgattcttttaaattgtcaaaactcaccgtttgatcaaattcaaatggcttaccttgatatttccttaattgcgtaattgac

SEQ ID NO: 3 - DNA, 117 bp, Artificial Sequence
  BP_DNA_003 Fragment containing sprA1
cgcagagaggaggtgtataaggtgatgcttattttcgttcacatcatagcaccagtcatcagtggctgtgccattgcgtttttttcttattggctaagtagacgcaatacaaaatag

SEQ ID NO: 4 - DNA, 1000 bp, Artificial Sequence
  BP_DNA_004 Site_2 Upstream Homology Arm
agatattgccaatttatctattcatcttcgtacacgtaaagaaatgagtaaagtagatgcacattgggaattaatcgaagctattaaaaatttacgtgacgaaattgcaccaaatacattgttaacaattaacggtgatattcccgatagaaaaacaggacttgaactggcagaaaaatatggcatcgatggtgtcatgattggtagaggcattttccataatccattcgcttttgaaaaagaaccacgcgaacacacaagcaaggaactattagatctattgagattgcatttatcattgtttaacaaatatgaaaaagatgaaatacgacaattcaagagcttgcgtagattctttaaaatctatgtgcgtggcataagaggcgctagcgaacttcgccatcaattgatgaacacacaatcaattgcagaagcacgagcacttctagatgaatttgaagcccaaatggacgaagacgttaaaatagaattatagtatgagtaataaagtttatggatgatatttcccaatttaacacggattgaacactttctacttaggtattatcttggttttcctgataggataactcccgatgatgcctttttacttatgtcacttatccttttggaaatggtgcaaaattaaaaatacatcaggatatcgtttaactcagaaaaaaactccaatcaaaaaaaatgtttacaacacattatgaaattaaaattgctcttagtgttcaacagacaaagttattgaaattactattggaggtacataccaagctatcattttgagtacacgtggtttgaatatctggcaagaaatggaaattggtaatgtcagtcataaagtcgctaaacgtgctcaaataccattaattatttttaaataatttagaggatgaggtataaacctttaaaaaacagcagtgagatgattttcaattagaaaatatcttactgctgtttctattttcataatcattcttattgaatg

SEQ ID NO: 5 - DNA, 446 bp, Artificial Sequence
  BP_DNA_005 sprA1(AS) insertion in SA Site_2
cagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattgagataacgaaaataagtatttacttatacaccaatcccctcactatttgcggtagtgaggggatttttattggtgcggctatatgtcacctattttgtattgcgtctacttagcc

SEQ ID NO: 6 - DNA, 950 bp, Artificial Sequence
  BP_DNA_006 Site_2 Downstream Homology Arm
acttcatcgtgatcatttaaaacagtaacaactttttcaatcttatactatttagagctaaaaattcacgacatataaacacaacactgacacgtcatttaaatacttaatcttttatagtaaaagatttttcattaaaaatttattgttttctgtactttattatattaatccttttagcatgccgtaccaatacattttaggtaataaatctttcttaaatatatacatacttctacgttctttggcctgattaaacggcattgtttctttagtatttttattataatcaaactctgcaagtattaacctattatatccagtaacaatagggcatgaagtataaccatcataatgatgcgttaacatttgattattcatcacttgcaataaattattagcgacgataggtgcttgcttacgaatagctgcgcctgtttttgaagtaggtacatttgaagcatcaccaagtgcaaatacattagagtagcttttatgctgtaatgtggttgggttaacatctacccaaccctcactatctgaaagtgtactttcttttactacatctaagggacccataggtggtgttacatgtaacatatcataacttattgtttttctatcgtatgctttgatatgttcgaatgtagccacttttttgtcaccgtcgatttcaacaaggttataattataattgactgttatatttctttcttcaacaatcctttctaattctttattatattttcctacgtcaaataaagcatcttttggcgttgcatatatcacattagcgttagaacggattttatgtttcctaaaataatcttcagctaaatacataattttcataggcgcacctccacactttataggagtgtttggatgcgtaaaaatggcatttccttgtttaaaattagaaatttggttccaagtttcgttaacatagt

SEQ ID NO: 7 - DNA, 909 bp, Artificial Sequence
  BP_DNA_007 PsbnA Downstream Homology Arm
acgatcgcctcgatctggtaaaatcgtgacaattgttgcaccttcttcaattgacgttatcaactgctcaatcgctgcaataatcgagcctgttgaacctccggcaaatatgccttcataatcaatcagttttcgacagcccaaagcagattgataatcatctacatggatcacttgattaatttctgatctattcaatatttcgggtacacgactagcaccgataccaggtaattctctattaataggtttgtcaccaaaaatgactgaccctttcgcatcaacagcaacaatttgtgcgtttggatgcacttcttttatttttctactcatacccataatgctacctgtcgtgctgactggcgcgacaaaataatctataggttgcttaattgtttcaacaatctctgtgcctgcaccatgataatgggattgccaatttaactcattcgcatattgattaatccaatatgcatcgtcaatagtggctaacagttcttgcacctttgcaatacgagtcattaaataacccccatgtgcatcaggttcttcaaccatttctacattggcaccataacttttaataattttcaaatttgttggtgatattttaggatcaacaacacacgtgagttttaatcccttgattttagctatcattgccaacgcaatgcctaaattaccagaagtactttcaattaaatgtgtattctcagtgattaaaccatgtttaataccatgttcaatgatgtacttggcaggtcgatctttcatgctgcctccaggattcatatactctaactttgcaaacacttcatgtttcggaaatagttgatgaagttgaaccataggtgtttgccctacagaatctaacaatgaatcgtgacatgcttgacttttttcaatcaa

SEQ ID NO: 8 - DNA, 131 bp, Artificial Sequence
  Staphylococcus aureus Fragment containing sprA2
cgcagagaggaggtgtatgatggatgttcaatttattaattaacatcatgacttcagctttaagcggctgtcttgttgcgttttttgcacattggttacgaacgcgcaacaataaaaaaggtgacaaataa

SEQ ID NO: 9 - DNA, 976 bp, Artificial Sequence
  BP_DNA_010 isdB Upstream Homology Arm for Triple stop insertion (p260)
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaat

SEQ ID NO: 10 - DNA, 1063 bp, Artificial Sequence
  BP_DNA_012 Upstream HA (Delta PsprA1)
gccactggcgtcaaatactgtaatgaagaatgtggtctaaaattgttgtaccaatttacataatcaaataattctgtttctaactgttctaagttttcaaattgcatttgttttacaaattctgttttcattgctttcatcgttgcttccgcaactgcgttatcataaggacaacctttggtacttaatgaacgttttattttaaatgtttctaggacttcatctatcaaatgattatcaaattctttgtctctatcagtgtggaatagtttgatttgttcaagattatgatttattctgctgattgcttttgatactaaattggcgtccttatttttacctgcactgtaaccaacaatttctctattaaatagatctataaataaacatatgtaatgccatgttcctccgacttttacatatgtcaaatcacttactaatgtctccattggttgttctctattaaaagcgcgattcaaatgatttttaattcgtttttcattagtttctatttgatgatttttgtatttagctttcatataaacagaaactagattttttcataatcgacctatcttttgtccagatacagtgagacccttgtcatttaaatgatttttaattcgtcttgtactaaagacttttctattagaattaaaaatatttatggcggcacgttctacgtttgaatcatctttagtgattttattatcttttctttttatagaatcataataggtacttcttggtatttttaggactttacacattgctggtactgaatattgatgtgcattcttttgaatgacttctatttttgccccataatcagctctttttcttcatctgttaagttatcttggtgattgaatgtacccttgttttgatgttactttatccattttcctaacgtcaaaggtgttaaatcatactcgcgtataatttcatttcttggcttaccattttcatataatctaaccatttataacttaaactctgaactaaatgttcttctttcttaaattaacaaaatctactctcatagaatttgtccaattagatg

SEQ ID NO: 11 - DNA, 1063 bp, Artificial Sequence; identical to SEQ ID NO: 10
  BP_DNA_013 Upstream Homology Arm

SEQ ID NO: 12 - DNA, 1195 bp, Artificial Sequence
  BP_DNA_014 Downstream Homology Arm
cagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttatctcaattatactgctaatctttctctttgtaaaatatgcaaggttttaaagagaaacatcaagaactaaaaaaggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 13 - DNA, 695 bp, Artificial Sequence
  BP_DNA_016 uidA Upstream Homology Arm
ggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctacggtgtactggccgatatcgaagcggaagacctggcgcgtgaagcgtcgtttgctcagggattacgcgcgatgattggcggtatcttaaccgcatcctgattctctctcttttcggcgggctggtgataactgtgcccgcgtttcatatcgtaatttctctgtgcaaaaattatccttcccggcttcggagaattccccccaaaatattcactgtagccatatgtcatgagagtttatcgttcccaatacgctcgaacgaacgttcggttgcttattttatggcttctgtcaacgctgttttaaagattaatgcgatctatatcacgctgtgggtattgcagtttttggttttttgatcgcggtgtcagttctttttatttccatttctcttccatgggtttctcacagataactgtgtgcaacacag

SEQ ID NO: 14 - DNA, 672 bp, Artificial Sequence
  BP_DNA_017 uidA Downstream Homology Arm
atcaacaactctcctggcgcaccatcgtcggctacagcctcggtgacgtcgccaataacttcgccttcgcaatgggggcgctcttcctgttgagttactacaccgacgtcgctggcgtcggtgccgctgcggcgggcaccatgctgttactggtgcgggtattcgatgccttcgccgacgtctttgccggacgagtggtggacagtgtgaatacccgctggggaaaattccgcccgtttttactcttcggtactgcgccgttaatgatcttcagcgtgctggtattctgggtgctgaccgactggagccatggtagcaaagtggtgtatgcatatttgacctacatgggcctcgggctttgctacagcctggtgaatattccttatggttcacttgctaccgcgatgacccaacaaccacaatcccgcgcccgtctgggcgcggctcgtgggattgccgcttcattgacctttgtctgcctggcatttctgataggaccgagcattaagaactccagcccggaagagatggtgtcggtataccatttctggacaattgtgctggcgattgccggaatggtgctttacttcatctgcttcaaatcgacgcgtgagaatgtggtacgtatcgttgcgcagccgtcattgaatatcagtctgcaaaccctgaaac

SEQ ID NO: 15 - DNA, 329 bp, Artificial Sequence
  BP_DNA_019 PgyrB
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatat

SEQ ID NO: 16 - DNA, 83 bp, Artificial Sequence
  BP_DNA_020 sprA1(AS)(long)
ataacgaaaataagtatttacttatacaccaatcccctcactatttgcggtagtgaggggatttttattggtgcggctatatg

SEQ ID NO: 17 - DNA, 225 bp, Artificial Sequence
  BP_DNA_021 Fragment containing sprG1
aggagacgcctaatgattacaattagtaccatgttgcagtttggtttattccttattgcattgataggtctagtaatcaagcttattgaattaagcaataaaaaataaccatcgctaactttggctggtttcgatggttaaatggttattaatttaatctttaatctaaaatagccaccgtctttttaacgggctcattagggtaacatgtttgcgcatgttgcc

SEQ ID NO: 18 - DNA, 267 bp, Artificial Sequence
  Staphylococcus aureus yoeB
atgagcaattacacggttaagattaaaaattcagcgaaatcagatttaaggaaaataaaacattcttatttaaagaagtcatttttagaaattgttgagactttaaaaaatgatccgtataaaataacacaatcttttgaaaaattagagcctaaatatttagagcgatattcaagaagaattaaccatcagcacagggtcgtctataccgtagatgatcgaaataaagaagtattaatactatcggcatggtcacattatgattaa

SEQ ID NO: 19 - DNA, 33 bp, Artificial Sequence
  BP_DNA_023 code_1
cgatcttcgacatcggaccctagaacagaacta

SEQ ID NO: 20 - DNA, 981 bp, Artificial Sequence
  BP_DNA_029 isdB Upstream HA
gatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaataaatc

SEQ ID NO: 21 - DNA, 412 bp, Artificial Sequence
  BP_DNA_030 PgyrB-sprA1(AS)(long)
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatatataacgaaaataagtatttacttatacaccaatcccctcactatttgcggtagtgaggggatttttattggtgcggctatatg

SEQ ID NO: 22 - DNA, 990 bp, Artificial Sequence
  BP_DNA_031 harA Upstream Homology Arm
tcagttatggacggctttgttgaacatccattctatacagcaactttaaatggtcaaaaatatgtagtgatgaaaacaaaggatgacagttactggaaagatttaattgtagaaggtaaacgtgtcactactgtttctaaagatcctaaaaataattctagaacgttgattttcccatatatacctgacaaagcagtttacaatgcgattgttaaagtcgttgtggcaaacattggttatgaaggtcaatatcatgtcagaattataaatcaggatatcaatacaaaagatgatgatacatcacaaaataacacgagtgaaccgctaaatgtacaaacaggacaagaaggtaaggttgctgatacagatgtagctgaaaatagcagcactgcaacaaatcctaaagatgcgtctgataaagcagatgtgatagaaccagagtctgacgtggttaaagatgctgataataatattgataaagatgtgcaacatgatgttgatcatttatccgatatgtcggataataatcacttcgataagtatgatttaaaagaaatggatactcaaattgccaaagatactgatagaaatgtggataatagcgttggtatgtcatcgaatgtcgatactgataaagactctaataaaaataaagacaaagtcatacagcttgctcatattgccgataaaaataatcatactggaaaagcagcaaagcttgacgtagtgaaacaaaattataataatacagacaaagttactgacaaaaaaacaactgaacatctgccgagtgatattcataaaactgtagataaaacagtgaaaacaaaagaaaaagccggcacaccatcgaaagaaaacaaacttagtcaatctaaaatgctaccaaaaactggagaaacaacttcaagccaatcatggtggggcttatatgcgttattaggtatgttagctttattcattcctaaattcagaaaagaatctaaataa

SEQ ID NO: 23 - DNA, 116 bp, Artificial Sequence
  BP_DNA_032 sprA1.55delT.S21LfsX4 plus control arm
cgcagagaggaggtgtataaggtgatgcttattttcgttcacatcatagcaccagtcatcagtggctgtgccattgcgttttttcttattggctaagtagacgcaatacaaaatag

SEQ ID NO: 24 - DNA, 993 bp, Artificial Sequence
  BP_DNA_033 harA Downstream Homology Arm
ttaactaaatatagcatatgtatggttaactttgtaaacaatgtgaaagcaattaatttataaactattgattggtttaatggctttcctttagagtaaataaaaagaacagcagtgagaaattttctaattgaaaataatcttactgctgtttttaatatttggatgcattgttgtggttactttaaaaagtgagcatcaattaacgcttttttcgatttaacaaatgtgatttaatatcatattttaatgcgtcgttgtattctttttcagtgatttgatcttcgattaacatacgctttaatacataatgttgtctttgaatactatatttcaaatctttatccgattttaacgttccatctttttcgtagggtgtatagccataagggctttgcaacaaaccgataaggtatgcagattgtgcaattgataaatcttttggtggaataccaaacaaactatatgaagcggatgcaattccggaaatattagcgccattataatctctaccgaagggaactatatttaaatatgtatatataatttcatcttttgagagtaggtgttctaatctaattgctaggcgaagttcatttgcttttctactatatgttttttcgttggtaagaacttgatttttaacaagttgttgtgtaattgtgctaccacctgaactttgatcagtattaaaaatatcttgtatcattgctcttaaaatcgcctttggtaagatgccatcatgtttataaaataaagtgtcttcagatgatgttaatgctttaatgacatttggacttgatgttttagggcctataatgagtgagttttgagaatggttatactcatataataaatttttgttattatgatctaataattcatcgccaggtatttgtcgaactttttttattaaagcatcatctgataatgagtcggacgttttagttaaatgatgaaaatataaagacatcgcaatcacagcgatagcaa

SEQ ID NO: 25 - DNA, 93 bp, Artificial Sequence
  Staphylococcus aureus sprA1 toxin
atgcttattttcgttcacatcatagcaccagtcatcagtggctgtgccattgcgtttttttcttattggctaagtagacgcaatacaaaatag

SEQ ID NO: 26 - DNA, 398 bp, Artificial Sequence
  BP_DNA_040 PgyrB-sprA1(AS)(short)
taattaaaacgtcatcctttattttttggcaaaaataattctagatgcgtatgtaaaataaatttgacagcattttaaacagcaaataaaagacgccaattaaatttatgacaaatgtatccaaaatttaataagtgtgcttatatgccctttaaatttaaaattttaatagtcaataacaagttgaatattaaagttaaacgccgttaaatagcgttaaaaaattgaaaatgacagtattgccaaaaaataagaattaattatttatatgtaaacggtttctacctctattttaaatgaaatttgtgacaaaaaaaggtataatatatataacgaaaataagtatttacttatacaccaatcccctcactatttgcggtagtgaggggatttttatt

SEQ ID NO: 27 - DNA, 991 bp, Artificial Sequence
  BP_DNA_041 PsbnA Upstream Homology Arm
aagcgcttcctcctcaaatttaaaattctataatattgtgtgttacctaattgataatgattctcactatcaagtaattaggattatattttttatgcatttatatgtcaaataattataagttgcatgtaaatcataaatattttactgacttaggaaaaaatttaattcatactaaatcgtgataatgattctcattgtcatacatcacgaaggaggctaattagtcaatgaataaagtaattaaaatgcttgttgttacgcttgctttcctacttgttttagcaggatgtagtgggaattcaaataaacaatcatctgataacaaagataaggaaacaacttcaattaaacatgcaatgggtacaactgaaattaaagggaaaccaaagcgtgttgttacgctatatcaaggtgccactgacgtcgctgtatctttaggtgttaaacctgtaggtgctgtagaatcatggacacaaaaaccgaaattcgaatacataaaaaatgatttaaaagatactaagattgtaggtcaagaacctgcacctaacttagaggaaatctctaaattaaaaccggacttaattgtcgcgtcaaaagttagaaatgaaaaagtttacgatcaattatctaaaatcgcaccaacagtttctactgatacagttttcaaattcaaagatacaactaagttaatggggaaagctttagggaaagaaaaagaagctgaagatttacttaaaaagtacgatgataaagtagctgcattccaaaaagatgcaaaagcaaagtataaagatgcatggccattgaaagcttcagttgttaacttccgtgctgatcatacaagaatttatgctggtggatatgctggtgaaatcttaaatgatttaggattcaaacgtaataaagacttacaaaaacaagttgataatggtaaagatattatccaacttacatctaaagaaagcattccattaatgaacgctgatca

SEQ ID NO: 28 - DNA, 69 bp, Artificial Sequence
  BP_DNA_043 sprA1(AS)(short)
ataacgaaaataagtatttacttatacaccaatcccctcactatttgcggtagtgaggggatttttatt

SEQ ID NO: 29 - DNA, 94 bp, Artificial Sequence
  BP_DNA_045 delta sprA1 (p253)
atataatagtagagtcgcctatctctcaggcgtcaatttagacgcagagaggaggtgtataaggtgatgcttattttcgttcacatcatagcac

SEQ ID NO: 30 - DNA, 300 bp, Artificial Sequence
  BP_DNA_056 PsbnA (insert in p242)
tcccactacatcctgctaaaacaagtaggaaagcaagcgtaacaacaagcattttaattactttattcattgactaattagcctccttcgtgatgtatgacaatgagaatcattatcacgatttagtatgaattaaattttttcctaagtcaataaaatatttatgatttacatgcaacttataattatttgacatataaatgcataaaaaatataatcctaattacttgatagtgagaatcattatcaattaggtaacacacaatattatagaattttaaatttgaggaggaagcgctt

SEQ ID NO: 31 - DNA, 1247 bp, Artificial Sequence
  BP_DNA_057 Downstream HA (delta PsprA1)
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaatcccctcactaccgcaaatagtgaggggattggtgtataagtaaatacttattttcgttatctcaattatactgctaatctttctctttgtaaaatatgcaaggttttaaagagaaacatcaagaactaaaaaaggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 32 - DNA, 333 bp, Artificial Sequence
  BP_DNA_060 PsprA1(AS)
cagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattgag

SEQ ID NO: 33 - DNA, 8793 bp, Artificial Sequence
  BP_DNA_062 pIMAYz Vector
caagcttatcgataccgtcgacctcgagggggggcccggtacccagcttttgttccctttagtgagggttaattatcccattatgctttggcagtttattcttgacatgtagtgagggggctggtataatcacatacggccgataaagcaagcatataatattgcgtttcatctttagaagcgaatttcgccaatattataattatcaaagagaggggtggcaaacggtatttggcattattaggttaaaaaatgtagaaggagagtgaaacccatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataaaggaggatatccatggaagttactgacgtaagattacgggtcgaccgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataatatgagataatgccgactgtactttttacagtcggttttctaatgtcactaacctgccccgttagttgacatatgatcatcataattctgtctcattatataacatcctccataccttctattatagaatgtattgctattaatcgcaacatcaaaccaaaataaaaacccccttcgactttcgtcagggggcttttatttattcaataatccctcctctcaataaatctattgttgtacttaattcaacttccatttctctgtatctttcaatacgctcttttaagtccttaatttctttttttaattcctcattttcagcaaataactctttttctttgtttgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaaaagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatatgaacctttgatataatcaagtatctcaacatgagcaactgaactattccccaattttcgcttaatcttgttcctaacgctttctattgttacaggatttcgtgcaatatatataacgtgatagtgtggttttttatagtgctttccattttgtataatattactattattccatgtatctttatcttttttttcgtccatatcgtgtaaaggactgacagccatagatacgcccaaactctctaatttttctttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgcatgctgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacactctatcattgatagagttatttgtcaaactagttttttatttggatcgcgtcgagttcatgaaaaactaaaaaaaatattgacactctatcattgatagagtataattaaaataagcttgatgggaatatgataatagaaataccattaccaacaccgaactgagtgatttgatcaccaagccatattaagaaagcagttcctgctgtcaaaactagtgctattaataaataactcataattgactgattgataatcagcgcacctttgagataattattaaattggaatgccatacctatagattggataaatgctaaagaaattgctaaataacgagtaacgttatttaactttcttctacctacttcaccttgttttgcccattctgagaatttagggacaatatccatttgtaataattgcattacgattgatgcagtgatgtagggtacaatacccattgcaaaaatagaaaatcgtttcaaggctccgccaccaaaagtatttaataactcagtggcaccttgagaaccttggggattatcaaaagctgcaggatttactcctggagctggtatataagtccctattttaaaaattactaacattgctagtgtgaagaaaatcttgttacgaacctcttttgttctaaagaagttcacaagggtttgaatcattagatcaagatctcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggacgtcgtaatacgactcactatagggcgaattggagctccaccgcggtggcggccgctctagaactagtggatccc

SEQ ID NO: 34 - DNA, 124 bp, Artificial Sequence
  BP_DNA_065 BP_115 insertion sequence
tgaatagcgcagagaggaggtgtataaggtgatgcttattttcgttcacatcatagcaccagtcatcagtggctgtgccattgcgtttttttcttattggctaagtagacgcaatacaaaatag

SEQ ID NO: 35 - DNA, 150 bp, Artificial Sequence
  BP_DNA_067 hokB gene from E. coli (K12)
atgaagcacaaccctctggtggtgtgtctgctcattatctgcattacgattctgacattcacactcctgacccgacaaacgctctacgaactgcggttccgggacggtgataaggaggttgctgcgctcatggcctgcacgtccaggtaa

SEQ ID NO: 36 - DNA, 156 bp, Artificial Sequence
  BP_DNA_068 hokD gene from E. coli (K12)
atgaagcagcaaaaggcgatgttaatcgccctgatcgtcatctgtttaaccgtcatagtgacggcactggtaacgaggaaagacctctgcgaggtacgaatccgaaccggccagacggaggtcgctgtcttcacagcttacgaacctgaggagtaa

SEQ ID NO: 37 - DNA, 336 bp, Artificial Sequence
  BP_DNA_069 mazF gene from E. coli (K12)
atggtaagccgatacgtacccgatatgggcgatctgatttgggttgattttgacccgacaaaaggtagcgagcaagctggacatcgtccagctgttgtcctgagtcctttcatgtacaacaacaaaacaggtatgtgtctgtgtgttccttgtacaacgcaatcaaaaggatatccgttcgaagttgttttatccggtcaggaacgtgatggcgtagcgttagctgatcaggtaaaaagtatcgcctggcgggcaagaggagcaacgaagaaaggaacagttgccccagaggaattacaactcattaaagccaaaattaacgtactgattgggtag

SEQ ID NO: 38 - DNA, 279 bp, Artificial Sequence
  BP_DNA_070 yafQ gene from E. coli (K12)
atgattcaaagggatattgaatactcgggacaatattcaaaggatgtaaaacttgcacaaaagcgtcataaggatatgaataaattgaaatatcttatgacgcttcttatcaataatactttaccgcttccagctgtttataaagaccacccgctgcaaggttcatggaaaggttatcgcgatgctcatgtcgaaccggactggatcctgatttacaaacttaccgataaacttttacgatttgagagaactggaactcacgcggcgctctttgggtaa

SEQ ID NO: 39 - DNA, 288 bp, Artificial Sequence
  BP_DNA_071 relE gene from E. coli (K12)
atggcgtattttctggattttgacgagcgggcactaaaggaatggcgaaagctgggctcgacggtacgtgaacagttgaaaaagaagctggttgaagtacttgagtcaccccggattgaagcaaacaagctccgtggtatgcctgattgttacaagattaagctccggtcttcaggctatcgccttgtataccaggttatagacgagaaagttgtcgttttcgtgatttctgttgggaaaagagaacgctcggaagtatatagcgaggcggtcaaacgcattctctga

SEQ ID NO: 40 - DNA, 1027 bp, Artificial Sequence
  BP_DNA_075 tetR_Pxyl-tet
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtg

SEQ ID NO: 41 - DNA, 1084 bp, Artificial Sequence
  BP_DNA_076 kanR PCR fragment
gtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttg

SEQ ID NO: 42 - DNA, 717 bp, Artificial Sequence
  BP_DNA_077 GFPmut2
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 43 - DNA, 6183 bp, Artificial Sequence
  BP_DNA_086 linearized pMBsacB
ggagaggcggtttgcgtattggtcgggtaccgagctcgaattgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaataatagtaatattatacaaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattagcttgttgtaactgaaaaaggaaaattattgtgccaggcagttgaaagtcagcaccttttaacgagtgctgaaatgacggctaaatgggaaacgtatttaaaaaaaatcggtaaaagagaaggcaatcaagagaactttattacgaatatcaaaaaattcattgttcatttactggaagctgtacctaacgatatagaaaaactaaatttttctgattaccaggaacagaaagaaaaagaagcagaaaaaagtattgtaggaaaatgtcctaagtgtggcaacaatattgtattaaaaaaatcgttttatggttgttcaaattatcctgaatgtaagtttactttagctgaacattttagaaagaaaaaactcaccaaaacaaatgtaaaagaattactagagggaaaagaaaccctggtaaaaggaatcaaaacgaaagatagaaagtcctacaatgccgttgtaaaaatcggagaaaagggatatattgattttatatctttctcaaaataaacataaaagccctttaaagagggcttttatatattaatcacaaatcacttatcacaaatcacaagtgatttgtgattgttgatgataaaataagaataagaagaaatagaaagaagtgagtgattgtgggaaatttaggcgcacaaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccttaactaaaagtagtgaatttttgatttttggtgtgtgtgtcttgttgttagtatttgctagtcaagtgattaaatagaattcgaaaagccctgacaacccttgttcctaaaaaggaataagcgttcggtcagtaaataatagaaataaaaaatcagacctaagactgatgacaaaaagagaaaattttgataaaatagtcttagaattaaattaaaaagggaggccaaatataatgaaaaatatgaatgacaatgatgttgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataacgcatgcctagcgcctacggggaatttgtatcgataaggggtacaaattcccactaagcgctcggtggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatg

SEQ ID NO: 44 - DNA, 699 bp, Artificial Sequence
  BP_DNA_087 mKATE2 (Staph aureus codon optimizaed)
atggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatga

SEQ ID NO: 45 - DNA, 1938 bp, Artificial Sequence
  Staphylococcus aureus isdB
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 46 - DNA, 1968 bp, Artificial Sequence
  Staphylococcus aureus isdB.1943delC.STOP645NfsX11
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaataataaatccgcagagaggaggtgtataaggtga

SEQ ID NO: 47 - DNA, 72 bp, Artificial Sequence
  Staphylococcus aureus sprA1.55delT.S21LfsX4
atgcttattttcgttcacatcatagcaccagtcatcagtggctgtgccattgcgttttttcttattggctaa

SEQ ID NO: 48 - DNA, 738 bp, Artificial Sequence
  BP_DNA_091 lysostaphin
atgacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 49 - DNA, 991 bp, Artificial Sequence
  BP_DNA_092 PsbnA Upstream Homology Arm
aagcgcttcctcctcaaatttaaaattctataatattgtgtgttacctaattgataatgattctcactatcaagtaattaggattatattttttatgcatttatatgtcaaataattataagttgcatgtaaatcataaatattttattgacttaggaaaaaatttaattcatactaaatcgtgataatgattctcattgtcatacatcacgaaggaggctaattagtcaatgaataaagtaattaaaatgcttgttgttacgcttgctttcctacttgttttagcaggatgtagtgggaattcaaataaacaatcatctgataacaaagataaggaaacaacttcaattaaacatgcaatgggtacaactgaaattaaagggaaaccaaagcgtgttgttacgctatatcaaggtgccactgacgtcgctgtatctttaggtgttaaacctgtaggtgctgtagaatcatggacacaaaaaccgaaattcgaatacataaaaaatgatttaaaagatactaagattgtaggtcaagaacctgcacctaacttagaggaaatctctaaattaaaaccggacttaattgtcgcgtcaaaagttagaaatgaaaaagtttacgatcaattatctaaaatcgcaccaacagtttctactgatacagttttcaaattcaaagatacaactaagttaatggggaaagctttagggaaagaaaaagaagctgaagatttacttaaaaagtacgatgataaagtagctgcattccaaaaagatgcaaaagcaaagtataaagatgcatggccattgaaagcttcagttgttaacttccgtgctgatcatacaagaatttatgctggtggatatgctggtgaaatcttaaatgatttaggattcaaacgtaataaagacttacaaaaacaagttgataatggtaaagatattatccaacttacatctaaagaaagcattccattaatgaacgctgatca

SEQ ID NO: 50 - DNA, 1867 bp, Artificial Sequence
  BP_DNA_093 tetR_Ptet-GFPmut2 Fragment
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagtgacatatagccgcaccaataaaaattgataatagctgagcccgggcactggccgtcgttttacaacgtcgtgactgggaaaaccctggcgttacccaacttaatcgccttgcagcacatcc

SEQ ID NO: 51 - DNA, 723 bp, Artificial Sequence
  BP_DNA_094 mKATE2 with control arm
cgcagagaggaggtgtataaggtgatggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatga

SEQ ID NO: 52 - DNA, 6511 bp, Artificial Sequence
  BP_DNA_095 pRAB11 Linearized Plasmid Backbone (p151)
gtgacatatagccgcaccaataaaaattgataatagctgagcccgggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcggacagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggtttttaggcataaaactatatgatttacccctaaatctttaaaatgccccttaaaattcaaaataaaggcatttaaaatttaaatatttcttgtgataaagtttgttaaaaaggagtggttttatgactgttatgtggttatcgattataggtatgtggttttgtattggaatggcattttttgctatcaaggttattaaaaataaaaattagaccacgcatttatgccgagaaaatttattgtgcgttgagaagaacccttaactaaacttgcagacgaatgtcggcatagcgtgagctattaagccgaccattcgacaagttttgggattgttaagggttccgaggctcaacgtcaataaagcaattggaataaagaagcgaaaaaggagaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccggcgaggctagttacccttaagttattggtatgactggttttaagcgcaaaaaaagttgctttttcgtacctattaatgtatcgttttaaatgaatagtaaaaaacatacatagaaaggggaaaaagcaactttttttattgtcatagtttgtgaaaactaagttgtttttatgtgttataacatggaaaagtatactgagaaaaaacaaagaaatcaagtatttcagaaatttattaaacgtcatattggagagaatcaaatggatttagttgaagattgcaatacatttctgtcttttgtagctgataaaactttagaaaaacagaaattatataaagctaattcttgtaaaaatcgattttgtcctgtctgtgcttggagaaaagctagaaaagatgcattgggtttatctttgatgatgcaatatattaagcagcaagagaaaaaggagtttatctttttaactttgactacacctaatgtaatgagtgatgaattagaaaatgaaataaaacgttataataattcttttagaaaacttataaagagaaaaaaagtaggtagtgttataaagggatatgttcgtaagttagagattacatataataaaaaaagagatgattataatcctcattttcatgtgttaattgcagtaaataaatcgtatttcacagataaaagatattatattagccaacaagaatggttagatttatggcgtgatgtaacgggcatttcagaaataacacaagttcaagttcaaaaaataagacaaaataataataaagaattatatgaaatggctaagtattctggtaaagatagtgattatttaataaatcaaaaagtctttgatgcattttataaatcacttaaaggtaaacaggtattagtttattcaggattatttaaagaggctaaaaagaaattaaaaaatggggatttagattacttaaaagaaattgatccaaccgaatatatctatcaaattttttatatttggaaacaaaaagagtatttagctagtgaactttatgacttaacagaacaagaaaaaagagaaattaatcacaaaatgatagacgaaatcgaggaagaacaataacaaaatataagtgctaacagctgacctcccgataacaccatgtagttattgggaggtcagctgttgaattatgcacgagtattttaaaagttattgtgatgacgacgataaacgattatcaaaagtataatgttaaaatgctttattatactaacgttatataaacattatactttcgttatacaaattttaaccctgttaggaactataaaaaatcatgaaaattttaatttgcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtg

SEQ ID NO: 53 - DNA, 60 bp, Artificial Sequence
  Staphylococcus aureus sprA1(AS)
ataacgaaaataagtatttacttatacaccaatcccctcactatttgcggtagtgagggg

SEQ ID NO: 54 - DNA, 58 bp, Artificial Sequence
  Staphylococcus aureus sprA2(AS)
cataataaattgaacatctaaatacaccaaatcccctcactactgccatagtgagggg

SEQ ID NO: 55 - DNA, 143 bp, Artificial Sequence
  Staphylococcus aureus sprF1
tatatagaaaaagggcaacatgcgcaaacatgttaccctaatgagcccgttaaaaagacggtggctattttagattaaagattaaattaataaccatttaaccatcgaaaccagccaaagttagcgatggttattttttattg

SEQ ID NO: 56 - DNA, 252 bp, Artificial Sequence
  BP_DNA_107  yefm
atgcgtacaattagctacagcgaagcgcgtcagaatttgtcggcaacaatgatgaaagccgttgaagatcatgccccgatccttattactcgtcagaatggagaggcttgtgttctgatgtcactcgaagaatacaactcgctggaagagacggcttatctactgcgctcccccgctaacgcccggagattgatggactcaatcgatagcctgaaatcaggcaaaggaacggaaaaggacatcattgagtga

SEQ ID NO: 57 - DNA, 744 bp, Artificial Sequence
  Serratia marcescens smaI
atgagcagggatgaccaactctttacactttggggaaagcttaacgatcgtcagaaggataattttctaaaatggatgaaagcttttgatgtagagaaaacttaccaaaaaacaagtggggatattttcaatgatgattttttcgatatatttggtgatagattaattactcatcatttcagtagcacgcaagctttaacaaaaactttattcgaacatgcttttaatgactccttaaatgaatctggagttatatcctctcttgcggaaagtagaacaaaccctgggcatgacataacaatcgatagcataaaggttgctttaaaaacagaagcagctaaaaatattagcaaatcatatattcatgtaagtaagtggatggagttaggcaagggggagtggattctagaattattattagaacggtttttagagcatctagagaattatgaacgtattttcacactcagatattttaaaatatccgagtataaatttagctaccagcttgtagaaatacccaagagtcttttgttggaagcaaaaaatgcgaaattagaaataatgtcgggaagcaaacaaagccctaagcccggctatggatatgtgttagatgaaaatgaaaataagaagttttctctatactttgatggtggtgccgagagaaaacttcaaataaaacatttaaatttagaacattgcattgttcatggagtttgggattttattctaccgccgccttaa

SEQ ID NO: 58 - DNA, 177 bp, Artificial Sequence
  BP_DNA_109 delta sprA1-sprA1(AS)
agataacgaaaataagtatttacttatacaccaatcccctcactatttgcggtagtgaggggatttttattggtgcggctatatgtcacctattttgtattgcgtctacttagccaataagaaaaaaacgcaatggcacagccactgatgactggtgctatgatgtgaacgaaaata

SEQ ID NO: 59 - DNA, 555 bp, Artificial Sequence
  BP_DNA_110 Upstream HA for delta sprA1-sprA1 (AS)
agcatcaccttatacacctcctctctgcgtctaaattgacgcctgagagataggcgactctactattatatcatctaattggacaaattctatgagagtagattttgttaatttaagaaagaagaacatttagttcagagtttaagttataaatggttagattatatgaaaatggtaagccaagaaatgaaattatacgcgagtatgatttaacacctttgacgttaggaaaatggataaagtaacatcaaaacaagggtacattcaatcaccaagataacttaacagatgaagaaaaagagctgattatggggcaaaaatagaagtcattcaaaagaatgcacatcaatattcagtaccagcaatgtgtaaagtcctaaaaataccaagaagtacctattatgattctataaaaagaaaagataataaaatcactaaagatgattcaaacgtagaacgtgccgccataaatatttttaattctaatagaaaagtctttagtacaagacgaattaaaaatcatttaaatgacaagggtctcactgtatctggaca

SEQ ID NO: 60 - DNA, 108 bp, Artificial Sequence
  Staphylococcus aureus sprG2
gtgatatctattgcaaacgcattacatttaatgttaagtttcggtatgtttatcgtcactttcattggtatagtagtagcaataataaatttaagcaataaaaaataa

SEQ ID NO: 61 - DNA, 116 bp, Artificial Sequence
  Staphylococcus aureus sprF2
gtaaaaagacgacatgcaggaacatgtcgcctaatgagcccgttaaaaagacggtgactaaatgagattttctttaaccatcattctatgtcaaagttttgaaatgatggttattt

SEQ ID NO: 62 - DNA, 81 bp, Artificial Sequence
  Staphylococcus aureus sprG3
atgtctgattttgaaatgctgatggttgtattaacaatcattggtttagtattgattagtactcaagaccataaaaaataa

SEQ ID NO: 63 - DNA, 164 bp, Artificial Sequence
  Staphylococcus aureus sprF3
gtagtaagtagaagcaaaagatgaaaatctttaactcttgaaacacaaaaagggcaacactcggaaacatgttaccctaatgagcccgttaaaaagacggtgaccttatattttatttaaaaatagccttcaaaatgccggtcaaagcgaatagaaggttattt

SEQ ID NO: 64 - DNA, 531 bp, Artificial Sequence
  BP_DNA_115 Downstream HA for delta sprA1-sprA1 (AS)
aaattactttcagactctagtgaacaaaagttctactataagcttattctttggcaatatttgaaagataaacatggattaacctgtgcatattctacatttagagcttatatccttaaacatgatgaatttaatcgttattttatgaaaggttatcaacggttgtcgcctaaaggcaaaacgagattcgagacaaaagccagtcatcaagcacagtttgactggaaagaaggcattaactttaaaacgaaggataatcaaatggtcctttagaagggataaacaacaaaataaaattaattaaacgtacatcttttggttaaggaagttataatcatttgcgaaatcgaatattattatgttcaaaactttacgctccaaaaagtaaaaaggaagctaagcaatgtttagttgcctaacttccgatattgaactcatcaggccaatttggcatagagccttttttagttcttgatgtttctctttaaaaccttgcatattttacaaagagaaagattagcagtataattg

SEQ ID NO: 65 - DNA, 1849 bp, Artificial Sequence
  BP_DNA_116 tetR_Ptet-mKATE2 fragment
gcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagccgcagagaggaggtgtataaggtgatggtgtctgagttgattaaggagaatatgcacatgaagttatatatggagggtacggtgaacaatcatcactttaaatgcacgtctgaaggcgagggtaagccgtacgaaggaacgcagactatgagaatcaaggctgtagagggcggtccattaccatttgcgtttgatatcttagctacttctttcatgtatggttctaaaacttttattaatcatacgcaaggtatccctgatttcttcaagcaatcttttccagaaggttttacttgggaaagagtaactacttacgaggatggcggcgttttaacagcaacgcaggatacaagtttacaggacggttgcttaatatataatgttaaaatccgtggagtcaacttcccatcaaatggcccagtcatgcaaaagaagacgttgggctgggaggcgagtacagaaacgttatacccagcagacggtggtttagagggtagagctgacatggcgttaaagttggtaggtggaggacacttgatatgcaacttaaaaacgacttacagatctaaaaaaccagcaaagaatttgaaaatgcctggtgtgtattatgtagaccgtcgattggaacgaattaaagaagctgataaagaaacatacgtggagcaacacgaggtggcagtagcacgttattgcgatttaccgtcaaaattgggacaccgatgagtgacatatagccgcaccaataaaaattgataatagctgagcccgggcactggccgtcgttttacaacgtcgtgactgggaaaaccctggcgttacccaacttaatcgccttgcagcacatcc

SEQ ID NO: 66 - DNA, 741 bp, Artificial Sequence
  BP_DNA_117 GFPmut2 with control arm
cgcagagaggaggtgtataaggtgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 67 - DNA, 1063 bp, Artificial Sequence
  BP_DNA_118 Upstream HA (?sprA1-sprA1(AS) in p197)
catctaattggacaaattctatgagagtagattttgttaatttaagaaagaagaacatttagttcagagtttaagttataaatggttagattatatgaaaatggtaagccaagaaatgaaattatacgcgagtatgatttaacacctttgacgttaggaaaatggataaagtaacatcaaaacaagggtacattcaatcaccaagataacttaacagatgaagaaaaagagctgattatggggcaaaaatagaagtcattcaaaagaatgcacatcaatattcagtaccagcaatgtgtaaagtcctaaaaataccaagaagtacctattatgattctataaaaagaaaagataataaaatcactaaagatgattcaaacgtagaacgtgccgccataaatatttttaattctaatagaaaagtctttagtacaagacgaattaaaaatcatttaaatgacaagggtctcactgtatctggacaaaagataggtcgattatgaaaaaatctagtttctgtttatatgaaagctaaatacaaaaatcatcaaatagaaactaatgaaaaacgaattaaaaatcatttgaatcgcgcttttaatagagaacaaccaatggagacattagtaagtgatttgacatatgtaaaagtcggaggaacatggcattacatatgtttatttatagatctatttaatagagaaattgttggttacagtgcaggtaaaaataaggacgccaatttagtatcaaaagcaatcagcagaataaatcataatcttgaacaaatcaaactattccacactgatagagacaaagaatttgataatcatttgatagatgaagtcctagaaacatttaaaataaaacgttcattaagtaccaaaggttgtccttatgataacgcagttgcggaagcaacgatgaaagcaatgaaaacagaatttgtaaaacaaatgcaatttgaaaacttagaacagttagaaacagaattatttgattatgtaaattggtacaacaattttagaccacattcttcattacagtatttgacgccagtggc

SEQ ID NO: 68 - DNA, 970 bp, Artificial Sequence
  BP_DNA_119 Downstream HA (delta sprA1-sprA1(AS) in p197)
ggctctatgccaaattggcctgatgagttcaatatcggaagttaggcaactaaacattgcttagcttcctttttactttttggagcgtaaagttttgaacataataatattcgatttcgcaaatgattataacttccttaaccaaaagatgtacgtttaattaattttattttgttgtttatcccttctaaaggaccatttgattatccttcgttttaaagttaatgccttctttccagtcaaactgtgcttgatgactggcttttgtctcgaatctcgttttgcctttaggcgacaaccgttgataacctttcataaaataacgattaaattcatcatgtttaaggatataagctctaaatgtagaatatgcacaggttaatccatgtttatctttcaaatattgccaaagaataagcttatagtagaacttttgttcactagagtctgaaagtaatttttcgatgataggataatacttgtcaataatagactgacgatttctctttttggttggctcaaagccatttaaatatttatcaactgttcttctataaacacccatgtgtctcgctatttcacttttgtttattttcatgtttaagttctccatgacaatttttaattttggtaaatctgtaagagtagtaacttcaaaatcagtatttatgtctaaagataatttcattgttgttcatctcaataaaattatctataggtttttaaaaattgtacatgtttaaacaatcaaaagtgcacattattaaattatcatttccagttaaactgtcttgatgattgaatgactcagtattttggttttgttttgtctaatttgagagagttaatgatgttagattatattctcgtataatttcgtttctaggcttaccattttcataaagtttaattattttaatttaaattatttactaaaagctcttcagtctcttgtcacaataaaatcgcc

SEQ ID NO: 69 - DNA, 2040 bp, Artificial Sequence
  BP_DNA_120 D7VX69 prolyl endopeptidase (Chryseobacterium teaenense)
atgatgtatcccaaagcattaaaaggaaagcaaaccgataattattttggaactgctgttacagatccgttcagagatcttgaaaatgattctgaagccaccaaaaagtgggtcgacgaagaagtgaaacacagtcaggattatcttgcaaaaatccctttcagagaagaaatcaggaaacagctcaccgatatctggaactacgaaaaaatttcggctccttttaaagaaggtgattttacttattattataaaaacaacgggctacaggcacaatctgtactttacagaaccaacaataaaacgaaagaaacagaagtatttttagatccgaataaattttctgaaaaaggcaccacttcactttctcaattgtcttttaacaaaaaaggaaatctcgccgcttattctatttcagaaggaggaagtgactggaacaagatcattatcatagacgctttatctaaaaaacagatcgatgaaacgctggtagatgtaaagttcagcggaatttcctggcagggtgatgaaggtttctattattcaagctacgacaaaccgaaagagggaaccgtgctctccggcatgaccgataagcataaagtctattttcataagttaggaacaaagcagtccgaagatcaattgatttttggaggagataaaacaccgagaagatatttgggagcaggagtgtctgaagatcagagatatcttattatttctgctgcgaatgccaccaacggaaatgaattatacataaaagaccttaaaaacggaggagattttgtgcagattaataaaggttttgatatcaatgccgatatagtcgatacacaaggagacgatctgtatatctttaccgataaggatgccccgaatatgcgtctcgtaaaaacaaccattaaaaatcctgctccagagacttggaaagatgtaattccggaaacggaaaatgttttgggaatcacaacaggcggaggatatttctttgctacctatatggtggatgcgattgatcaggtaaaacaatacgacagagcaggaaaaatgatccgtgaaattacgcttcccggaaaaggaaatgtttctggttttggaggaaaggaaaacgagaaagaattgtatttttcattcaccaattatattacaccgggaacaacgtataaattcaatgcagattccggaaaatctgaggtttaccagaagccgaaggtgaaatttaatcctgaagattatgtctctgaacaagtattttacacctcaaaagacggtacaaaagttccgatgatgattaactataaaaaaggaactaagctcgacggtaaaaatcctacgattttatattcttacggaggttttaatatcagtttgcagccggccttctctgtagtcaatgccatctggatggaaaacggtggtatttatgccgttccgaacatccgtggaggtggtgaatatggaaaaaaatggcatgatgccggaacaaaaatgaataagaaaaacgtattcaacgatttcatcgctgccggagaatatttgcagcagaaaggctacacttccaaacagtttatggctctttcgggaagatcaaatggaggactgctggtgggtgcaacaatgacgatgcgtcccgatctggcaagagtcgctttcccgggagttggcgtgttggatatgctgaggtataataaattcacagccggtgcaggttggtcgtacgattacggaacctctgaagatagcaaagaaatgtttgaatatttaaaatcatattccccggttcataatgtaaaagccggaacatgctatccttccacaatgattatcaccagcgatcatgatgacagggtggttcctgcgcattctttcaaattcggagctgagcttcaggaaaagcaggcatgcgatcatccgattcttttaaggattgaaaaaaatgcaggtcacggagcaggcagatctacagatcaggtgatcggagaaaatgcagacttgatttctttcgctttatttgaaatggggattaaaaatttaaag

SEQ ID NO: 70 - DNA, 1197 bp, Artificial Sequence
  BP_DNA_121 Actinoallomurus sp. strain DSM 24988 ENDOPEP-40 gene, complete cds
atgtcacgacgcgtgaccgggaccatactgggcgggttgatcctcgccatggtccccttcctttccaccgcggccaacgccgcaccccaggccgcgccggcttccgtctcccacccgttccaccactcctgcgccacggtgaagccgggtcgggcgagctgcaatgccctcgtacgcagcgacatcgcccagagcgcggcgaccctcgcgcaccaagcggccgccccatccgggctctcgccggccaacctgcagagcgcctacaagctgccgtcctccacggccggatccggccagaccgtcgcgatcgtcgacgcctatgacgccccgaccgccgaagcggacttgaacgtgtaccgaagccagttcggactcggcgcgtgcacgaccgccaacggctgtttcaagaaggtcgaccagaacggcggcacgtcctatccgaggaaggacggcggctgggcgcaggagatctccctggacctcgacatggtctccgcggtctgccccaactgcaagatcgttctcgtcgaggcgaagaccaactcgttcgccaacctgggtaccgccgagaacaccgcggcgagtctcgcgaacgtcatcagcaacagctacggcggctcggacgcctctgacgcgagctatggctcgtactacaaccacccgggcaaggccatcacggtcagctccggcgacgccggctacggcgtggagtacccggcctcgtcccactacgtgaccgccgtcggcggcacctcgctgcgcaccgcgagcaccagccgcggctggagcgagaccgcgtggagcggcgcgggcagtggctgctcggcctacaacaccgcgctgtccggccagtccggcctcaccggctgctcccggcgcgccgtcgccgacgtctccgccgtggccgacccggccaccggcgtcgccgtctacgacagcacggcctaccagggccagagcggctggatggtcttcggcggcaccagcgtcgccgcaccgatcatcggtggcgtgtacggcctcgccgccaacgccgcgagcatcgacaacaactacccctacgcccacaccagctcgctcttcgacgtcacgtcgggcagcaacggcacctgcaccaccaccaagtggtgcaccgccggcaccggctgggacggccccaccggcctcggaacgccgaacggcaccggagccttctga

SEQ ID NO: 71 - protein, 655 aa, Artificial Sequence
  Staphylococcus aureus isdB
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNNKSAERRCIR

SEQ ID NO: 72 - protein, 30 aa, Artificial Sequence
  Staphylococcus aureus sprA1
MLIFVHIIAPVISGCAIAFFSYWLSRRNTK

SEQ ID NO: 73 - protein, 35 aa, Artificial Sequence
  Staphylococcus aureus sprA2
MFNLLINIMTSALSGCLVAFFAHWLRTRNNKKGDK

SEQ ID NO: 74 - protein, 645 aa, Artificial Sequence
  Staphylococcus aureus isdB.1943delC.STOP645NfsX11
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 75 - protein, 49 aa, Artificial Sequence
  BP_AA_005 hokB (E. coli, K12)
MKHNPLVVCLLIICITILTFTLLTRQTLYELRFRDGDKEVAALMACTSR

SEQ ID NO: 76 - protein, 51 aa, Artificial Sequence
  BP_AA_006 hokD (relF) (E. coli, K12)
MKQQKAMLIALIVICLTVIVTALVTRKDLCEVRIRTGQTEVAVFTAYEPEE

SEQ ID NO: 77 - protein, 111 aa, Artificial Sequence
  BP_AA_007 mazF (E. coli, K12)
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 78 - protein, 92 aa, Artificial Sequence
  BP_AA_008 yafQ (E. coli, K12)
MIQRDIEYSGQYSKDVKLAQKRHKDMNKLKYLMTLLINNTLPLPAVYKDHPLQGSWKGYRDAHVEPDWILIYKLTDKLLRFERTGTHAALFG

SEQ ID NO: 79 - protein, 95 aa, Artificial Sequence
  BP_AA_009 relE (E. coli, K12)
MAYFLDFDERALKEWRKLGSTVREQLKKKLVEVLESPRIEANKLRGMPDCYKIKLRSSGYRLVYQVIDEKVVVFVISVGKRERSEVYSEAVKRIL

SEQ ID NO: 80 - protein, 207 aa, Artificial Sequence
  BP_AA_010 tetR (artificial sequence, synthetic construct)
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 81 - protein, 264 aa, Artificial Sequence [sequence omitted]

SEQ ID NO: 82 - protein, 238 aa, Artificial Sequence
  BP_AA_012 GFP (green fluorescent protein)
MSKGEELFTGVVPILVELDGDVNGHKFSVSGEGEGDATYGKLTLKFICTTGKLPVPWPTLVTTFAYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 83 - protein, 232 aa, Artificial Sequence
  BP_AA_013 mKATE2 (RFP, red fluorescent protein)
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 84 - protein, 23 aa, Artificial Sequence
  BP_AA_014 sprA1.55delT.S21LfsX4 (truncated version of sprA1 in Staph aureus BP_001)
MLIFVHIIAPVISGCAIAFFLIG

SEQ ID NO: 85 - protein, 88 aa, Artificial Sequence
  Staphylococcus aureus yoeb
MSNYTVKIKNSAKSDLRKIKHSYLKKSFLEIVETLKNDPYKITQSFEKLEPKYLERYSRRINHQHRVVYTVDDRNKEVLILSAWSHYD

SEQ ID NO: 86 - protein, 245 aa, Artificial Sequence
  BP_AA_016 lysostaphin (artificial sequence)
MTHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 87 - protein, 83 aa, Artificial Sequence
  BP_AA_017 yefM (E.coli)
MRTISYSEARQNLSATMMKAVEDHAPILITRQNGEACVLMSLEEYNSLEETAYLLRSPANARRLMDSIDSLKSGKGTEKDIIE

SEQ ID NO: 88 - protein, 247 aa, Artificial Sequence
  Serratia marcescens smaI
MSRDDQLFTLWGKLNDRQKDNFLKWMKAFDVEKTYQKTSGDIFNDDFFDIFGDRLITHHFSSTQALTKTLFEHAFNDSLNESGVISSLAESRTNPGHDITIDSIKVALKTEAAKNISKSYIHVSKWMELGKGEWILELLLERFLEHLENYERIFTLRYFKISEYKFSYQLVEIPKSLLLEAKNAKLEIMSGSKQSPKPGYGYVLDENENKKFSLYFDGGAERKLQIKHLNLEHCIVHGVWDFILPPP

SEQ ID NO: 89 - protein, 35 aa, Artificial Sequence
  Staphylococcus aureus sprG2
VISIANALHLMLSFGMFIVTFIGIVVAIINLSNKK

SEQ ID NO: 90 - protein, 26 aa, Artificial Sequence
  Staphylococcus aureus sprG3
MSDFEMLMVVLTIIGLVLISTQDHKK

SEQ ID NO: 91 - protein, 31 aa, Artificial Sequence
  Staphylococcus aureus sprG1(short)
MITISTMLQFGLFLIALIGLVIKLIELSNKK

SEQ ID NO: 92 - protein, 680 aa, Artificial Sequence
  BP_AA_022 D7VX69 prolyl endopeptidase (Chryseobacterium taeanense)
MMYPKALKGKQTDNYFGTAVTDPFRDLENDSEATKKWVDEEVKHSQDYLAKIPFREEIRKQLTDIWNYEKISAPFKEGDFTYYYKNNGLQAQSVLYRTNNKTKETEVFLDPNKFSEKGTTSLSQLSFNKKGNLAAYSISEGGSDWNKIIIIDALSKKQIDETLVDVKFSGISWQGDEGFYYSSYDKPKEGTVLSGMTDKHKVYFHKLGTKQSEDQLIFGGDKTPRRYLGAGVSEDQRYLIISAANATNGNELYIKDLKNGGDFVQINKGFDINADIVDTQGDDLYIFTDKDAPNMRLVKTTIKNPAPETWKDVIPETENVLGITTGGGYFFATYMVDAIDQVKQYDRAGKMIREITLPGKGNVSGFGGKENEKELYFSFTNYITPGTTYKFNADSGKSEVYQKPKVKFNPEDYVSEQVFYTSKDGTKVPMMINYKKGTKLDGKNPTILYSYGGFNISLQPAFSVVNAIWMENGGIYAVPNIRGGGEYGKKWHDAGTKMNKKNVFNDFIAAGEYLQQKGYTSKQFMALSGRSNGGLLVGATMTMRPDLARVAFPGVGVLDMLRYNKFTAGAGWSYDYGTSEDSKEMFEYLKSYSPVHNVKAGTCYPSTMIITSDHDDRVVPAHSFKFGAELQEKQACDHPILLRIEKNAGHGAGRSTDQVIGENADLISFALFEMGIKNLK

SEQ ID NO: 93 - protein, 398 aa, Artificial Sequence
  BP_AA_023 Actinoallomurus sp. strain DSM 24988 ENDOPEP-40 gene, complete cds
MSRRVTGTILGGLILAMVPFLSTAANAAPQAAPASVSHPFHHSCATVKPGRASCNALVRSDIAQSAATLAHQAAAPSGLSPANLQSAYKLPSSTAGSGQTVAIVDAYDAPTAEADLNVYRSQFGLGACTTANGCFKKVDQNGGTSYPRKDGGWAQEISLDLDMVSAVCPNCKIVLVEAKTNSFANLGTAENTAASLANVISNSYGGSDASDASYGSYYNHPGKAITVSSGDAGYGVEYPASSHYVTAVGGTSLRTASTSRGWSETAWSGAGSGCSAYNTALSGQSGLTGCSRRAVADVSAVADPATGVAVYDSTAYQGQSGWMVFGGTSVAAPIIGGVYGLAANAASIDNNYPYAHTSSLFDVTSGSNGTCTTTKWCTAGTGWDGPTGLGTPNGTGAF

SEQ ID NO: 94 - protein, 1029 aa, Artificial Sequence
  BP_AA_024 E. coli lacZ gene EC3.2.1.23
MTMITPSFPGNSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 95 - DNA, 108 bp, Artificial Sequence
  BP_DNA_125 sprG2 (gDNA.1G>A, 4A>C)
atgctatctattgcaaacgcattacatttaatgttaagtttcggtatgtttatcgtcactttcattggtatagtagtagcaataataaatttaagcaataaaaaataa

SEQ ID NO: 96 - protein, 35 aa, Artificial Sequence
  BP_AA_025 sprG2 (protein.V1M, I2L)
MLSIANALHLMLSFGMFIVTFIGIVVAIINLSNKK

SEQ ID NO: 97 - DNA, 109 bp, Artificial Sequence
  Synthetic construct
ttaagtagcatcgttgcattcgtattacctagaaaacgtaaaaactaataaatccgcagagaggaggtgtataaggtgatgcttattttcgttcacatcatagcaccag

SEQ ID NO: 98 - protein, 15 aa, Artificial Sequence
  Synthetic construct
LSSIVAFVLPRKRKN

SEQ ID NO: 99 - protein, 10 aa, Artificial Sequence
  Synthetic construct
MLIFVHIIAP

SEQ ID NO: 100 - DNA, 108 bp, Artificial Sequence
  Synthetic construct
ttaagtagcatcgttgcattcgtattacctagaaaacgtaaaaataataaatccgcagagaggaggtgtataaggtgatgcttattttcgttcacatcatagcaccag

SEQ ID NO: 101 - protein, 25 aa, Artificial Sequence
  Synthetic construct
LSSIVAFVLPRKRKNNKSAERRCIR

SEQ ID NO: 102 - DNA, 110 bp, Artificial Sequence
  Synthetic construct
ttaagtagcatcgttgcattcgtattacctagaaaacgtaaaaactaattgatagcgcagagaggaggtgtataaggtgatgcttattttcgttcacatcatagcaccag

SEQ ID NO: 103 - DNA, 149 bp, Artificial Sequence
  Synthetic construct
cgcagagaggaggtgtataaggtgatgcttattttcgttcacatcatagcaccagtcatcagtggctgtgccattgcgtttttttcttattggctaagtagacgcaatacaaaataggtctttatatttaattaaattaacaaatttta

SEQ ID NO: 104 - protein, 7 aa, Artificial Sequence
  Synthetic construct
AERRCIR

SEQ ID NO: 105 - protein, 51 aa, Artificial Sequence
  Homo sapiens insulin, B-chain
GIVEQCCTSICSLYQLENYCNFVNQHLCGSHLVEALYLVCGERGFFYTPKT

SEQ ID NO: 106 - DNA, 22 bp, Artificial Sequence
  Synthetic construct; DR_606
gaacaacgtaacggcttcatcc

SEQ ID NO: 107 - DNA, 29 bp, Artificial Sequence
  Synthetic construct; DR_607
gttgctcgtgcatttagatgattcttatc

SEQ ID NO: 108 - DNA, 56 bp, Artificial Sequence
  Synthetic construct;  BP_948
ccctcgaggtcgacggtatcgataagcttggatgagcaagtgaaatcagctattac

SEQ ID NO: 109 - DNA, 48 bp, Artificial Sequence
  Synthetic construct; BP_949
cacctcctctctgcggatttattagtttttacgttttctaggtaatac

SEQ ID NO: 110 - DNA, 42 bp, Artificial Sequence
  Synthetic construct; BP_950
aaaaactaataaatccgcagagaggaggtgtataaggtgatg

SEQ ID NO: 111 - DNA, 55 bp, Artificial Sequence
  Synthetic construct;  BP_951
attaaatataaagacctattttgtattgcgtctacttagccaataagaaaaaaac

SEQ ID NO: 112 - DNA, 54 bp, Artificial Sequence
  Synthetic construct;  BP_952
cgcaatacaaaataggtctttatatttaattattaaattaacaaattttaattg

SEQ ID NO: 113 - DNA, 56 bp, Artificial Sequence
  Synthetic construct;  BP_953
gtggcggccgctctagaactagtggatcccgtcaattacgcaattaaggaaatatc

SEQ ID NO: 114 - DNA, 56 bp, Artificial Sequence
  Synthetic construct;  DR_511
cacctcctctctgcgctattcaattagtttttacgttttctaggtaatacgaatgc

SEQ ID NO: 115 - DNA, 40 bp, Artificial Sequence
  Synthetic construct; DR_512
ctaattgaatagcgcagagaggaggtgtataaggtgatgc

SEQ ID NO: 116 - DNA, 127 bp, Artificial Sequence
  frameshift sprA1 toxin; BP_DNA_063
ataataaatccgcagagaggaggtgtataaggtgatgcttattttcgttcacatcatagcaccagtcatcagtggctgtgccattgcgtttttttcttattggctaagtagacgcaatacaaaatag

SEQ ID NO: 117 - DNA, 25 bp, Artificial Sequence
  Primer; DR_022
caagcttatcgataccgtcgacctc

SEQ ID NO: 118 - DNA, 23 bp, Artificial Sequence
  Primer; DR_023
gggatccactagttctagagcgg

SEQ ID NO: 119 - DNA, 28 bp, Artificial Sequence
  Primer; DR_116
gggacgtcgtaatacgactcactatagg

SEQ ID NO: 120 - DNA, 40 bp, Artificial Sequence
  Primer; DR_117
ccaaagcataatgggataattaaccctcactaaagggaac

SEQ ID NO: 121 - DNA, 40 bp, Artificial Sequence
  Primer; DR_254
atgcttattttcgttcacatcatagcaccagtcatcagtg

SEQ ID NO: 122 - DNA, 60 bp, Artificial Sequence
  Primer; DR_518
gtggcggccgctctagaactagtggatcccgtcaattacgcaattaaggaaatatcaagg

SEQ ID NO: 123 - DNA, 56 bp, Artificial Sequence; identical to SEQ ID NO: 108
  Primer; BP_948

SEQ ID NO: 124 - DNA, 48 bp, Artificial Sequence; identical to SEQ ID NO: 109
  Primer; BP_949

SEQ ID NO: 125 - DNA, 42 bp, Artificial Sequence; identical to SEQ ID NO: 110
  Primer; BP_950

SEQ ID NO: 126 - DNA, 55 bp, Artificial Sequence; identical to SEQ ID NO: 111
  Primer; BP_951

SEQ ID NO: 127 - DNA, 54 bp, Artificial Sequence; identical to SEQ ID NO: 112
  Primer; BP_952

SEQ ID NO: 128 - DNA, 22 bp, Artificial Sequence
  Primer; BP_964
tcaaacttcagcaggttctagc

SEQ ID NO: 129 - DNA, 22 bp, Artificial Sequence
  Primer; BP_965
gtaccaggtatgactgaatgcc

SEQ ID NO: 130 - DNA, 20 bp, Artificial Sequence
  Primer; gyrB; BPC802
ttggtacaggaatcggtggc

SEQ ID NO: 131 - DNA, 20 bp, Artificial Sequence
  Primer; BPC803
tccatccacatcggcatcag

SEQ ID NO: 132 - DNA, 20 bp, Artificial Sequence
  Primer; isdA; BPC114
gcaacagaagctacgaacgc

SEQ ID NO: 133 - DNA, 22 bp, Artificial Sequence
  Primer; BPC115
agagccatctttttgcacttgg

SEQ ID NO: 134 - DNA, 25 bp, Artificial Sequence
  Primer; isdB; BPC116
gcaacaattttatcattatgccagc

SEQ ID NO: 135 - DNA, 22 bp, Artificial Sequence
  Primer; BPC117
tggcaactttttgtcaccttca

SEQ ID NO: 136 - DNA, 21 bp, Artificial Sequence
  Primer; isdI; BPC764
accgaggatacagacgaagtt

SEQ ID NO: 137 - DNA, 21 bp, Artificial Sequence
  Primer; BPC765
tgctgtccatcgtcatcactt

SEQ ID NO: 138 - DNA, 21 bp, Artificial Sequence
  Primer; isdG; BPC120
aaccaatccgtaaaagcttgc

SEQ ID NO: 139 - DNA, 20 bp, Artificial Sequence
  Primer; BPC121
aggctttgatggcatgtttg

SEQ ID NO: 140 - DNA, 21 bp, Artificial Sequence
  Primer; sbnC; BPC768
agggaagggtgtctaagcaac

SEQ ID NO: 141 - DNA, 20 bp, Artificial Sequence
  Primer; BPC769
tcagtccttcttcaacgcga

SEQ ID NO: 142 - DNA, 20 bp, Artificial Sequence
  Primer; sbnE; BPC124
attcgctttagccgcaatgg

SEQ ID NO: 143 - DNA, 20 bp, Artificial Sequence
  Primer; BPC125
gcaacttgtagcgcatcgtc

SEQ ID NO: 144 - DNA, 21 bp, Artificial Sequence
  Primer; lrgA; BPC126
gataccggctggtacgaagag

SEQ ID NO: 145 - DNA, 21 bp, Artificial Sequence
  Primer; BPC127
tggtgctgttaagttaggcga

SEQ ID NO: 146 - DNA, 20 bp, Artificial Sequence
  Primer; lrgB; BPC128
acaaagacaggcacaactgc

SEQ ID NO: 147 - DNA, 20 bp, Artificial Sequence
  Primer; BPC129
ggtgtagcaccagccaaaga

SEQ ID NO: 148 - DNA, 21 bp, Artificial Sequence
  Primer; hlgB; BPC760
tggttggggaccttatggaag

SEQ ID NO: 149 - DNA, 20 bp, Artificial Sequence
  Primer; BPC761
ggcatttggtgttgcgctat

SEQ ID NO: 150 - DNA, 21 bp, Artificial Sequence
  Primer; fhuA; BPC132
cacgttgtctttgaccaccac

SEQ ID NO: 151 - DNA, 21 bp, Artificial Sequence
  Primer; BPC133
tgggcaatggaagttacagga

SEQ ID NO: 152 - DNA, 20 bp, Artificial Sequence
  Primer; fhuB; BPC134
caatacctgctggaacccca

SEQ ID NO: 153 - DNA, 20 bp, Artificial Sequence
  Primer; BPC135
gggtccgcatattgccaaac

SEQ ID NO: 154 - DNA, 21 bp, Artificial Sequence
  Primer; ear; BPC136
ccacttgtcagatctgctcct

SEQ ID NO: 155 - DNA, 24 bp, Artificial Sequence
  Primer; BPC137
ggtttggttacagatggacaaaca

SEQ ID NO: 156 - DNA, 19 bp, Artificial Sequence
  Primer; fnb; BPC772
cgcagtgagcgaccataca

SEQ ID NO: 157 - DNA, 20 bp, Artificial Sequence
  Primer; BPC773
ttggtccttgtgcttgacca

SEQ ID NO: 158 - DNA, 20 bp, Artificial Sequence
  Primer; hlb; BPC140
ctacgccaccatcttcagca

SEQ ID NO: 159 - DNA, 20 bp, Artificial Sequence
  Primer; BPC141
acacctgtactcggtcgttc

SEQ ID NO: 160 - DNA, 22 bp, Artificial Sequence
  Primer; splF; BPC142
tgcaattattcagcctggtagc

SEQ ID NO: 161 - DNA, 22 bp, Artificial Sequence
  Primer; BPC143
cctgatggcttattaccggcat

SEQ ID NO: 162 - DNA, 20 bp, Artificial Sequence
  Primer; splD; BPC144
agtgacatctgatgcggttg

SEQ ID NO: 163 - DNA, 21 bp, Artificial Sequence
  Primer; BPC145
aacaccaattgcttctcgctt

SEQ ID NO: 164 - DNA, 20 bp, Artificial Sequence
  Primer; dps; BPC146
agcggtaggaggaaaccctg

SEQ ID NO: 165 - DNA, 22 bp, Artificial Sequence
  Primer; BPC147
gttctgcagagtaacctttcgc

SEQ ID NO: 166 - DNA, 21 bp, Artificial Sequence
  Primer; srtB; BPC846
tgagcgagaacatcgacgtaa

SEQ ID NO: 167 - DNA, 20 bp, Artificial Sequence
  Primer; BPC847
ccgacatggtgcccgtataa

SEQ ID NO: 168 - DNA, 21 bp, Artificial Sequence
  Primer; emp; BPC854
tcgcgtgaatgtagcaacaaa

SEQ ID NO: 169 - DNA, 21 bp, Artificial Sequence
  Primer; BPC855
acttctgggcctttagcaaca

SEQ ID NO: 170 - DNA, 20 bp, Artificial Sequence
  Primer; sbnA; BPC858
cctggaggcagcatgaaaga

SEQ ID NO: 171 - DNA, 20 bp, Artificial Sequence
  Primer; BPC859
cattgccaacgcaatgccta

SEQ ID NO: 172 - DNA, 20 bp, Artificial Sequence
  Primer; CH52_360; BPC834
ttcaactcgaacgctgacga

SEQ ID NO: 173 - DNA, 20 bp, Artificial Sequence
  Primer; BPC835
ttgcacccattgttgcacct

SEQ ID NO: 174 - DNA, 20 bp, Artificial Sequence
  Primer; CH52_305; BPC838
ttcctggagcagtaccacca

SEQ ID NO: 175 - DNA, 22 bp, Artificial Sequence
  Primer; BPC839
cagcgcaatcgctgttaaacta

SEQ ID NO: 176 - DNA, 20 bp, Artificial Sequence
  Primer; CH521670; BPC842
gcgattatgggaccaaacgg

SEQ ID NO: 177 - DNA, 21 bp, Artificial Sequence
  Primer; BPC843
acttcatagcttgggtgtccc

SEQ ID NO: 178 - DNA, 20 bp, Artificial Sequence
  Primer; clfA; BPC850
tccagcacaacaggaaacga

SEQ ID NO: 179 - DNA, 20 bp, Artificial Sequence
  Primer; BPC851
tagcttcaccagttaccggc

SEQ ID NO: 180 - DNA, 20 bp, Artificial Sequence
  Primer; SAUSA300_2268; BPC778
gcttctacagctttgccgat

SEQ ID NO: 181 - DNA, 21 bp, Artificial Sequence
  Primer; BPC779
gatttggtgcttactgccacc

SEQ ID NO: 182 - DNA, 20 bp, Artificial Sequence
  Primer; SAUSA300_2616; BPC774
acaagcgcaacaagcaagag

SEQ ID NO: 183 - DNA, 23 bp, Artificial Sequence
  Primer; BPC775
tgcgtttgatacctttaacacgg

SEQ ID NO: 184 - DNA, 21 bp, Artificial Sequence
  Primer; SAUSA300_2617; BPC152
gggctgaaaaagttggcatga

SEQ ID NO: 185 - DNA, 20 bp, Artificial Sequence
  Primer; BPC153
acgcgttgtttttgacctcc

SEQ ID NO: 186 - DNA, 21 bp, Artificial Sequence
  Primer; hlgA2; BPC179
tgatttctgcaccttgaccga

SEQ ID NO: 187 - DNA, 20 bp, Artificial Sequence
  Primer; BPC180
agcccctttagccaatccat

SEQ ID NO: 188 - DNA, 22 bp, Artificial Sequence
  Primer; hrtAB; BPC713
acacaacaacaacgtgatgagc

SEQ ID NO: 189 - DNA, 20 bp, Artificial Sequence
  Primer; BPC714-
taacggtgcttgctctgctt

SEQ ID NO: 190 - DNA, 40 bp, Artificial Sequence
  Primer; DR_357
gagttgttgatggctaagtagacgcaatacaaaataggtg

SEQ ID NO: 191 - DNA, 38 bp, Artificial Sequence
  Primer; DR_410
cctgggtaccagtcatcaagcacagtttgactggaaag

SEQ ID NO: 192 - DNA, 29 bp, Artificial Sequence
  Primer; DR_359
ggaaccgattgaagggattcatttcgttg

SEQ ID NO: 193 - DNA, 34 bp, Artificial Sequence
  Primer; DR_409
ctcggttgctgtgttgcacacagttatctgtgag

SEQ ID NO: 194 - DNA, 42 bp, Artificial Sequence
  Primer; DR_361
tgcgtctacttagccatcaacaactctcctggcgcaccatcg

SEQ ID NO: 195 - DNA, 33 bp, Artificial Sequence
  Primer; DR_362
gtttcagggtttgcagactgatattcaatgacg

SEQ ID NO: 196 - DNA, 25 bp, Artificial Sequence
  Primer; DR_371
acatagcgcacgtagaacaacgacg

SEQ ID NO: 197 - DNA, 29 bp, Artificial Sequence
  Primer; DR_372
gccatctgtaaatcttgcgccattagtcc

SEQ ID NO: 198 - DNA, 39 bp, Artificial Sequence
  Primer; DR_407
gtgtgcaacacagcaaccgagcgttctgaacaaatccag

SEQ ID NO: 199 - DNA, 40 bp, Artificial Sequence
  Primer; DR_408
gtgcttgatgactggtacccaggaaacagctatgaccatg

SEQ ID NO: 200 - DNA, 40 bp, Artificial Sequence; identical to SEQ ID NO: 120
  Primer; DR_117

SEQ ID NO: 201 - DNA, 32 bp, Artificial Sequence
  Primer; DR_228
ctattttgtattgcgtctacttagccaataag

SEQ ID NO: 202 - DNA, 26 bp, Artificial Sequence
  Primer; DR_116
ccctgttgataccgggaagccctggg

SEQ ID NO: 203 - DNA, 1084 bp, Artificial Sequence; identical to SEQ ID NO: 41
  Escherichia coli kanR fragment; BP_DNA_015

SEQ ID NO: 204 - DNA, 446 bp, Artificial Sequence; identical to SEQ ID NO: 5
  PsprA1(as)-sprA1(as); BP_DNA_018

SEQ ID NO: 205 - DNA, 40 bp, Artificial Sequence; identical to SEQ ID NO: 120
  Primer; DR_117

SEQ ID NO: 206 - DNA, 40 bp, Artificial Sequence; identical to SEQ ID NO: 121
  Primer; DR_254

SEQ ID NO: 207 - DNA, 29 bp, Artificial Sequence
  Primer; DR_533
gattacgcttacattcgcttctctgtttc

SEQ ID NO: 208 - DNA, 30 bp, Artificial Sequence
  Primer; DR_534
cagctgttgataatgccatttttgcacgag

SEQ ID NO: 209 - DNA, 22 bp, Artificial Sequence
  Primer; BP_964
tcaaacttcagcaggttctagc

SEQ ID NO: 210 - DNA, 22 bp, Artificial Sequence
  Primer; BP_965
gtaccaggtatgactgaatgcc

SEQ ID NO: 211 - DNA, 48 bp, Artificial Sequence; identical to SEQ ID NO: 109
  Primer; BP_949

SEQ ID NO: 212 - DNA, 32 bp, Artificial Sequence; identical to SEQ ID NO: 201
  Primer; DR_228

SEQ ID NO: 213 - DNA, 42 bp, Artificial Sequence; identical to SEQ ID NO: 110
  Primer; BP_950

SEQ ID NO: 214 - DNA, 32 bp, Artificial Sequence
  Primer; DR_318
cgattacttcccaaccattacctactgtcaac

SEQ ID NO: 215 - DNA, 55 bp, Artificial Sequence
  Primer; BM_049
cgtactgattgggtaggtgacatatagccgcaccaataaaaattgataatagctg

SEQ ID NO: 216 - DNA, 40 bp, Artificial Sequence
  Primer; BM_015
ggctatatgtcacctacccaatcagtacgttaattttggc

SEQ ID NO: 217 - DNA, 40 bp, Artificial Sequence
  Primer; BM_014
ggtgtataaggtgatggtaagccgatacgtacccgatatg

SEQ ID NO: 218 - DNA, 37 bp, Artificial Sequence
  Primer; BM_013
tcggcttaccatcaccttatacacctcctctctgcgg

SEQ ID NO: 219 - DNA, 49 bp, Artificial Sequence
  Primer; DR_634
caggagagttgttgatgcatgtaactgggcagtgtcttaaaaaatcgac

SEQ ID NO: 220 - DNA, 37 bp, Artificial Sequence
  Primer; DR_636
cagttacatgcatcaacaactctcctggcgcaccatc

SEQ ID NO: 221 - DNA, 44 bp, Artificial Sequence
  Primer; BM_052
ggtgtataaggtgatgattcaaagggatattgaatactcgggac

SEQ ID NO: 222 - DNA, 31 bp, Artificial Sequence
  Primer; BM_027
gctatatgtcacttacccaaagagcgccgcg

SEQ ID NO: 223 - DNA, 35 bp, Artificial Sequence
  Primer; BM_025
ccctttgaatcatcaccttatacacctcctctctg

SEQ ID NO: 224 - DNA, 42 bp, Artificial Sequence
  Primer; BM_024
gctctttgggtaagtgacatatagccgcaccaataaaaattg

SEQ ID NO: 225 - DNA, 41 bp, Artificial Sequence
  Primer; BM_018
ggtgtataaggtgatggcgtattttctggattttgacgagc

SEQ ID NO: 226 - DNA, 38 bp, Artificial Sequence
  Primer; BM_019
ggctatatgtcactcagagaatgcgtttgaccgcctcg

SEQ ID NO: 227 - DNA, 37 bp, Artificial Sequence
  Primer; BM_017
aaaatacgccatcaccttatacacctcctctctgcgg

SEQ ID NO: 228 - DNA, 41 bp, Artificial Sequence
  Primer; BM_016
cgcattctctgagtgacatatagccgcaccaataaaaattg

SEQ ID NO: 229 - DNA, 28 bp, Artificial Sequence
  DR_244
catcaccttatacacctcctctctgcgg

SEQ ID NO: 230 - DNA, 50 bp, Artificial Sequence
  Primer; DR_661
ctgaggagtaagtgacatatagccgcaccaataaaaattgataatagctg

SEQ ID NO: 231 - DNA, 52 bp, Artificial Sequence
  Primer; DR_659
cgcagagaggaggtgtataaggtgatgaagcagcaaaaggcgatgttaatcg

SEQ ID NO: 232 - DNA, 47 bp, Artificial Sequence
  Primer; DR_660
gtgcggctatatgtcacttactcctcaggttcgtaagctgtgaagac

SEQ ID NO: 233 - DNA, 36 bp, Artificial Sequence
  Primer; DR_674
gtccaggtaagtacccaggaaacagctatgaccatg

SEQ ID NO: 234 - DNA, 39 bp, Artificial Sequence
  Primer; DR_673
agctgtttcctgggtacttacctggacgtgcaggccatg

SEQ ID NO: 235 - DNA, 40 bp, Artificial Sequence
  Primer; DR_672
ggaggtgtataaggtgatgaagcacaaccctctggtggtg

SEQ ID NO: 236 - DNA, 38 bp, Artificial Sequence
  Primer; DR_675
ggttgtgcttcatcaccttatacacctcctctctgcgg

SEQ ID NO: 237 - DNA, 38 bp, Artificial Sequence
  Primer; DR_280
gtagacgcaatacaaaataggtgacatatagccgcacc

SEQ ID NO: 238 - DNA, 45 bp, Artificial Sequence
  Primer; DR_278
cgcagagaggaggtgtataaggtgatgcttattttcgttcacatc

SEQ ID NO: 239 - DNA, 32 bp, Artificial Sequence; identical to SEQ ID NO: 201
  Primer; DR_228

SEQ ID NO: 240 - DNA, 26 bp, Artificial Sequence
  Synthetic construct; BP_542
catcaccttatacacctcctctctgc

SEQ ID NO: 241 - DNA, 27 bp, Artificial Sequence
  Synthetic construct; BP_717
actctttgaagtcattctttacaggag

SEQ ID NO: 242 - DNA, 27 bp, Artificial Sequence
  Synthetic construct; BP_718
ctcctgtaaagaatgacttcaaagagt

SEQ ID NO: 243 - DNA, 30 bp, Artificial Sequence
  Synthetic construct; DR_215
ccgacctcattaagcagctctaatgcgctg

SEQ ID NO: 244 - DNA, 29 bp, Artificial Sequence
  Synthetic construct; DR_216
ggtgtgaaataccgcacagatgcgtaagg

SEQ ID NO: 245 - DNA, 51 bp, Artificial Sequence
  Synthetic construct; DR_725
gcaataaaaaataagtgacatatagccgcaccaataaaaattgataatagc

SEQ ID NO: 246 - DNA, 60 bp, Artificial Sequence
  Synthetic construct; DR_726
ggtgcggctatatgtcacttattttttattgcttaaatttattattgctactactatacc

SEQ ID NO: 247 - DNA, 49 bp, Artificial Sequence
  Synthetic construct; DR_727
cgcagagaggaggtgtataaggtgatgatatctattgcaaacgcattac

SEQ ID NO: 248 - DNA, 58 bp, Artificial Sequence
  Synthetic construct; DR_728
tggtgcggctatatgtcacttattttttatggtcttgagtactaatcaatactaaacc

SEQ ID NO: 249 - DNA, 52 bp, Artificial Sequence
  Synthetic construct; DR_729
cgcagagaggaggtgtataaggtgatgtctgattttgaaatgctgatggttg

SEQ ID NO: 250 - DNA, 54 bp, Artificial Sequence
  Synthetic construct; DR_730
gaccataaaaaataagtgacatatagccgcaccaataaaaattgataatagctg

SEQ ID NO: 251 - DNA, 31 bp, Artificial Sequence
  Synthetic construct; DR_733
gtgcggctatatgtcacttattttttattgc

SEQ ID NO: 252 - DNA, 21 bp, Artificial Sequence
  Synthetic construct; DR_734
cgcagagaggaggtgtataag

SEQ ID NO: 253 - DNA, 81 bp, Artificial Sequence; identical to SEQ ID NO: 62
  Synthetic construct; sprG3 BP_164

SEQ ID NO: 254 - DNA, 108 bp, Artificial Sequence; identical to SEQ ID NO: 95
  Synthetic construct; sprG2 BP_165

SEQ ID NO: 255 - DNA, 41 bp, Artificial Sequence
  Synthetic construct Primer BP_677
gttgtaaaacgacggccagtgcccgggctcagctattatca

SEQ ID NO: 256 - DNA, 31 bp, Artificial Sequence
  Synthetic construct Primer BP_670
gctcagatctgttaacggtaccatcatactc

SEQ ID NO: 257 - DNA, 21 bp, Artificial Sequence
  Synthetic construct Primer BP_671
cactggccgtcgttttacaac

SEQ ID NO: 258 - DNA, 51 bp, Artificial Sequence
  Synthetic construct Primer DR_476
ccgcagagaggaggtgtataaggtgatgagtaaaggagaagaacttttcac

SEQ ID NO: 259 - DNA, 58 bp, Artificial Sequence
  Synthetic construct Primer DR_247
caatttttattggtgcggctatatgtcacttatttgtatagttcatccatgccatgtg

SEQ ID NO: 260 - DNA, 43 bp, Artificial Sequence
  Synthetic construct Primer DR_245
gtgacatatagccgcaccaataaaaattgataatagctgagcc

SEQ ID NO: 261 - DNA, 144 bp, Artificial Sequence
  Synthetic construct SprA1 insert BP_DNA_150
cgcagagaggaggtgtataaggtgatgcttattttcgttcacatcatagcaccagtcatcagtggctgtgccattgcgtttttttcttattggctaagtagacgcaatacaaaataggtgacatatagccgcaccaataaaaat

SEQ ID NO: 262 - DNA, 717 bp, Artificial Sequence; identical to SEQ ID NO: 42
  Synthetic construct GFPmut2 insert  BP_DNA_077

SEQ ID NO: 263 - DNA, 24 bp, Artificial Sequence
  Synthetic construct Primer BM_152
aggaataccaggcgatgaaccgat

SEQ ID NO: 264 - DNA, 23 bp, Artificial Sequence
  Synthetic construct Primer BM_153
tgctctaattctccccttatggc

SEQ ID NO: 265 - DNA, 6487 bp, Artificial Sequence
  Synthetic construct Plasmid BP_DNA_151 p151 linearized; p174 backbone
gtgacatatagccgcaccaataaaaattgataatagctgagcccgggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcggacagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttctgtaggtttttaggcataaaactatatgatttacccctaaatctttaaaatgccccttaaaattcaaaataaaggcatttaaaatttaaatatttcttgtgataaagtttgttaaaaaggagtggttttatgactgttatgtggttatcgattataggtatgtggttttgtattggaatggcattttttgctatcaaggttattaaaaataaaaattagaccacgcatttatgccgagaaaatttattgtgcgttgagaagaacccttaactaaacttgcagacgaatgtcggcatagcgtgagctattaagccgaccattcgacaagttttgggattgttaagggttccgaggctcaacgtcaataaagcaattggaataaagaagcgaaaaaggagaagtcggttcagaaaaagaaggatatggatctggagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgaaaaatccttttctttcttatcttgataataagggtaactattgccggcgaggctagttacccttaagttattggtatgactggttttaagcgcaaaaaaagttgctttttcgtacctattaatgtatcgttttaaatgaatagtaaaaaacatacatagaaaggggaaaaagcaactttttttattgtcatagtttgtgaaaactaagttgtttttatgtgttataacatggaaaagtatactgagaaaaaacaaagaaatcaagtatttcagaaatttattaaacgtcatattggagagaatcaaatggatttagttgaagattgcaatacatttctgtcttttgtagctgataaaactttagaaaaacagaaattatataaagctaattcttgtaaaaatcgattttgtcctgtctgtgcttggagaaaagctagaaaagatgcattgggtttatctttgatgatgcaatatattaagcagcaagagaaaaaggagtttatctttttaactttgactacacctaatgtaatgagtgatgaattagaaaatgaaataaaacgttataataattcttttagaaaacttataaagagaaaaaaagtaggtagtgttataaagggatatgttcgtaagttagagattacatataataaaaaaagagatgattataatcctcattttcatgtgttaattgcagtaaataaatcgtatttcacagataaaagatattatattagccaacaagaatggttagatttatggcgtgatgtaacgggcatttcagaaataacacaagttcaagttcaaaaaataagacaaaataataataaagaattatatgaaatggctaagtattctggtaaagatagtgattatttaataaatcaaaaagtctttgatgcattttataaatcacttaaaggtaaacaggtattagtttattcaggattatttaaagaggctaaaaagaaattaaaaaatggggatttagattacttaaaagaaattgatccaaccgaatatatctatcaaattttttatatttggaaacaaaaagagtatttagctagtgaactttatgacttaacagaacaagaaaaaagagaaattaatcacaaaatgatagacgaaatcgaggaagaacaataacaaaatataagtgctaacagctgacctcccgataacaccatgtagttattgggaggtcagctgttgaattatgcacgagtattttaaaagttattgtgatgacgacgataaacgattatcaaaagtataatgttaaaatgctttattatactaacgttatataaacattatactttcgttatacaaattttaaccctgttaggaactataaaaaatcatgaaaattttaatttgcatgtaactgggcagtgtcttaaaaaatcgacactgaatttgctcaaatttttgtttgtagaattagaatatatttatttggctcatatttgctttttaaaagcttgcatgcctgcaggtcgacggtatcgataactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctcctttttgttgacattatatcattgatagagttatttgtcaaactagttttttatttggatcccctcgagttcatgaaaaactaaaaaaaatattgacactctatcattgatagagtataattaaaataagctctctatcattgatagagtatgatggtaccgttaacagatctgagc

SEQ ID NO: 266 - DNA, 3018 bp, Artificial Sequence
  Synthetic construct Strep. thermophilus Beta-galactosidase (IDT E coli (K12) codon optimized) BP_DNA_152
atgaacacaacagaaaagatccagacatacttaaacgatccaaaaatcgtatcggttaacacagtagacgcccatagcgatcataagtattttgaatctttggctgaattctctgaaggcgagatgaaattgcgccaaagtcttaatggtaagtggaaaatccactacgcacaaaatacgaaccaggtattaaaggacttttataagacggaatttgacgaaacggaccttaactttatcaacgttcctggccaccttgaattccaaggtttcggctccccgcaatacgttaatacccagtatccgtgggacggcaaggaattcttacgccccccacaggtgccccaagagtctaatgcagtagcctcttacgtcaaacatttcactcttaacgacgcgcttaaagataaaaaggtattcatcagctttcagggagtggctacatcgatcttcgtctgggttaacggaaacttcgtgggctactctgaagactcattcactccttctgagttcgaaatctccgattaccttgttgaaggagataacaaattggccgttgctgtctaccgttactcaacagcttcgtggttggaggatcaagacttttggcgtctgtatgggatttttcgtgacgtgtacttatacgccattcccaaagtgcatgtacaagacttgtttgttaaaggagattatgattatcagaccaagaccggacagcttgatatcgatcttaaaacagtaggtgattacgaggacaagaaaatcaagtacgtgctgtccgatcacgagggtatcgtcacagaaggtgacgcctctgttaatggagatggagagctttctgttagccttgagaacttaaagattaagccttggtccgccgagtctcccaagctttataacctgactctgcatgttcttgatgatgatcaggtagtagaagttgttcccgtaaaggtaggcttccgtcacttcgagatcaaagacaagcttatgttgttaaatggaaagcgcatcgtttttaagggagtaaaccgccatgagtttaacgctcgtacaggccgctgtattacagaagaggacatgttgtgggacatcaaagtcatgaagcagcacaacatcaacgccgttcgtaccagtcattacccgaaccaaacacgttggtatgagctgtgcgacgagtatggactgtatgtgatcgatgaggctaaccttgagactcatggaacatggcaaaagttgggactgagtgaaccttcctggaatatcccagcctcggaaccagaatggctgcccgcatgcttagatcgtgctaataatatgtttcagcgcgacaaaaaccatgcttcggtgatcatctggtcttgtggcaacgagagttacgcgggcaaggacattgcggatatggcggattacttccgttccgtcgacaacacacgtcccgtccactacgagggtgtgacttggtgtcgcgagttcgactatatcaccgatatcgagtcacgcatgtacgcgaaacctgccgatattgaagagtatcttacaaacaacccgcagaaaccgtatatcagttgtgagtacatgcatacaatgggcaattcaggaggaggtctgaagttatatacagatttggagaagtatccggaataccaaggcggttttatttgggatttcatcgatcaagcaatctataagccccttcccaacggctccgaatttttgtcgtatggcggcgactggcatgaccgtccatcggattacgagttctgtggcaatggcattgtcttcgcggaccgcacattgacgccaaaacttcagacagtcaaacatctttattctaacattaaaattgcggtggatgagaaatccgtaacaattaacaatgacaatatttttgaagatctttccgcttatacatttttagcccgtgtctacgaggacggacgcaaggtttctgagtccgagtatcacttcgatgtcaaaccgggggaagaagctacgttcccagttgaattcgccgtaggagcatccaacgcggaacgtatctatgaagtggtttgcgtgcagaaggaagctacggaatgggcgccaaaaggtcacgagattgtacgtggacaatatgtagcggaaaaaatttctaccgaaacgcccgtcaaagctccactgaatgtggtggaaggtgatttcaatattgggatccagggacagaatttttctattcttttaagccgtgcacaaaacacgctggtctcagctaaatataacggtgttgagttcattgaaaaaggcccaaaattaaattttacgcgtgcttatacagacaacgatcgtggggccggctacccgtttgaaatggcggggtggaaagtggcgggaaattattccaaggttacggatacccagatccaaatcgaggacgattcggttaaggtcacttatgtccatgaacttccaggtctgagtgatgtggaagtcaaagtaacgtaccaggtggactataaagggcgtatttttgtcaccgcgaattacgatggaaaagcggggcttccaaatttccccgaattcgggcttgaatttgccattggatcacagttcacaaacctgtcctactatggatatggtgcggaggagtcttacctggataaattgcctggtgcatacctgggacgttatgagacgtcggtggaaaaaacttttgcaccgtatcttatgcctcaggaaagtggcaatcattatggaacacgcgagtttaccgtatcggatgataatcacaatggcctgaagtttactgctcttaacaaagcttttgaattttccgctctgcgtaactccactgagcagatcgaaaacgcccgtcaccagtatgaattgcagcaatcggatgcaacttggatcaaagtattagctgcgcaaatgggtgtaggcggtgacgactcgtggggcgcacccgtacatgacgaatttcttttgagtagcgcggactcttaccagttgggctttatgatcgagcccttatcataa

SEQ ID NO: 267 - DNA, 3090 bp, Artificial Sequence
  Synthetic construct E. coli Beta-galactosidase (IDT E coli (K12) codon optimized) BP_DNA_153
atgactatgatcacgccttctttcccgggtaattcattagcggttgtgttgcaacgtcgcgactgggagaatccgggggtgacgcagcttaatcgcttagcagctcatccccctttcgcatcatggcgtaattcagaggaagcccgcacggaccgtccctcgcagcaactgcgttctttaaacggtgaatggcgcttcgcttggtttccggcccctgaggccgtacctgagtcttggttggagtgtgatttgcctgaagcggataccgtagtggtgccttcgaactggcaaatgcatgggtacgatgctccgatttatactaatgttacataccctattacggtcaaccccccattcgttcctacagagaatcccaccgggtgttactctttgactttcaatgtagacgagtcgtggctgcaggagggacagacacgtatcattttcgatggcgtgaactccgccttccacttatggtgtaacggccgttgggttggttacgggcaggacagtcgtctgccatccgagtttgatttgagcgccttcctgcgtgcaggtgaaaatcgcctggctgttatggtccttcgctggagcgatggatcgtatttggaggatcaagatatgtggcgtatgagtgggattttccgtgacgtaagccttctgcataagcctacaacccaaattagtgatttccatgtggcgacacgcttcaacgatgatttttcgcgcgcagtcttagaggcggaagttcagatgtgtggcgaactgcgtgattacctgcgcgtcaccgtctctctgtggcaaggtgaaactcaagttgcttcagggactgcgcccttcggaggagaaattattgacgaacgcggagggtacgcagatcgcgtgactctgcgtcttaacgtggagaatccaaagttgtggtccgcagaaattcctaatctgtaccgtgccgtagtcgaacttcataccgcagacggcacgctgatcgaggctgaagcgtgcgatgtagggttccgtgaagtacgcattgaaaatggcctgttattgttgaacggcaaacccttacttatccgcggggtcaaccgtcatgagcaccatcctctgcacggacaagtaatggatgaacaaacaatggtacaagacattttgttaatgaagcagaacaatttcaacgcagtacgctgttcacattatccaaaccaccccttgtggtacacgctgtgtgaccgttatggactgtacgtcgttgacgaggcaaatattgagacccatggtatggtgcccatgaatcgcttaacggacgatccccgctggttgcccgcaatgagtgaacgtgtaacgcgtatggtccaacgtgaccgtaaccacccctcagtaatcatttggtcgctgggaaatgaatcagggcacggggccaaccacgacgctttatatcgttggatcaaatcagtagatccaagtcgcccagtgcagtatgaggggggtggagcggatactaccgcaacagacatcatctgcccaatgtacgcgcgtgtcgacgaagatcagccatttccggctgtaccaaagtggtcaatcaagaaatggttatctcttccaggtgagactcgccctctgatcctgtgcgagtatgcacacgcgatgggaaactccttgggtggatttgcgaaatattggcaagcatttcgccaatatccacgtttacaaggaggctttgtttgggactgggtggatcagtctcttattaagtacgatgaaaatgggaacccatggtccgcgtatggcggagattttggggacactccaaatgatcgtcaattttgtatgaatgggttggtgttcgctgaccgtaccccgcatccggcgctgacagaggccaaacaccagcagcagttttttcagtttcgcctgtcagggcaaactatcgaagttacatctgagtatttatttcgtcattccgacaatgagttgctgcattggatggttgctttagacgggaaacccttagctagtggagaagttccattagacgtcgcgccgcaagggaaacaacttattgaacttcctgagttgccgcagcctgaatcggctggacaactgtggcttacagtccgtgtagtacagcccaacgctacagcttggagcgaggcgggccacattagtgcatggcaacagtggcgccttgcggagaatctgtctgtgactcttccggctgcatcccatgctatcccccacttgaccacctcagagatggacttctgcattgagctgggcaacaagcgctggcagtttaatcgtcagtccggttttttgagccaaatgtggatcggagacaaaaagcagttattgacgcctctgcgcgatcaattcactcgcgctccgttagataacgacattggcgtctcggaggcgacccgcattgatcctaatgcctgggtagagcgctggaaagcggcgggacattaccaggcagaggcagcgttattacaatgcaccgccgacacacttgccgatgcggtgctgattacaacagcccacgcgtggcaacatcagggtaaaactcttttcatctcacgcaagacataccgtatcgatggctcaggccaaatggcaattacagtagacgtggaggttgcgtctgatacaccacatcctgcgcgtatcggccttaactgccaattagcccaggtcgcggagcgcgtcaactggttaggactgggtccacaggagaactacccggaccgccttacagccgcctgcttcgaccgctgggatttgccgttaagtgatatgtacactccctatgtttttcctagtgaaaatggcttacgctgtggtacacgcgaattaaactatggaccacaccaatggcgcggggatttccagtttaatattagtcgttatagtcaacaacaattgatggaaacttcacaccgtcacttgctgcatgcggaggaaggcacatggttgaatattgatggatttcacatgggaattggcggagacgactcatggtccccatctgtgagcgccgaatttcagttatccgctgggcgctaccattaccagctggtttggtgccagaaataa

SEQ ID NO: 268 - protein, 1029 aa, Artificial Sequence; identical to SEQ ID NO: 94
  Synthetic construct E. coli lacZ gene product EC3.2.1.23; bacterial beta-galactosidase BP_AA_024

SEQ ID NO: 269 - protein, 35 aa, Artificial Sequence; identical to SEQ ID NO: 96
  Synthetic construct sprG2 (protein.V1M, I2L) BP_AA_025

SEQ ID NO: 270 - protein, 1005 aa, Artificial Sequence
  Synthetic construct Strep therm Beta-galactosidase BP_AA_026
MNTTEKIQTYLNDPKIVSVNTVDAHSDHKYFESLAEFSEGEMKLRQSLNGKWKIHYAQNTNQVLKDFYKTEFDETDLNFINVPGHLEFQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDHEGIVTEGDASVNGDGELSVSLENLKIKPWSAESPKLYNLTLHVLDDDQVVEVVPVKVGFRHFEIKDKLMLLNGKRIVFKGVNRHEFNARTGRCITEEDMLWDIKVMKQHNINAVRTSHYPNQTRWYELCDEYGLYVIDEANLETHGTWQKLGLSEPSWNIPASEPEWLPACLDRANNMFQRDKNHASVIIWSCGNESYAGKDIADMADYFRSVDNTRPVHYEGVTWCREFDYITDIESRMYAKPADIEEYLTNNPQKPYISCEYMHTMGNSGGGLKLYTDLEKYPEYQGGFIWDFIDQAIYKPLPNGSEFLSYGGDWHDRPSDYEFCGNGIVFADRTLTPKLQTVKHLYSNIKIAVDEKSVTINNDNIFEDLSAYTFLARVYEDGRKVSESEYHFDVKPGEEATFPVEFAVGASNAERIYEVVCVQKEATEWAPKGHEIVRGQYVAEKISTETPVKAPLNVVEGDFNIGIQGQNFSILLSRAQNTLVSAKYNGVEFIEKGPKLNFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYLDKLPGAYLGRYETSVEKTFAPYLMPQESGNHYGTREFTVSDDNHNGLKFTALNKAFEFSALRNSTEQIENARHQYELQQSDATWIKVLAAQMGVGGDDSWGAPVHDEFLLSSADSYQLGFMIEPLS

SEQ ID NO: 271 - DNA, 437 bp, Artificial Sequence
  Synthetic construct Homology arm  for making version 2 of lacZ::(gene-X)_kanR for  piggyback in lactose pathway BP_DNA_154
gtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcg

SEQ ID NO: 272 - DNA, 416 bp, Artificial Sequence
  Synthetic construct Homology arm  for making version 2 of lacZ::(gene-X)_kanR for  piggyback in lactose pathway BP_DNA_155
cgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagcc

SEQ ID NO: 273 - DNA, 430 bp, Artificial Sequence
  Synthetic construct Homology arm  for making lacY::(gene-X)_kanR for  piggyback in lactose pathway  BP_DNA_156
gctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgcggcgcgagcgccttatccgaccaacatatcataacg

SEQ ID NO: 274 - DNA, 612 bp, Artificial Sequence
  Synthetic construct Homology arm  for making lacY::(gene-X)_kanR BP_DNA_157
ttgaacatgccaatgaccgaaagaataagagcaggcaagctatttaccgatatgtgcgaaggcttaccggaaaaaagacttcgtgggaaaacgttaatgtatgagtttaatcactcgcatccatcagaagttgaaaaaagagaaagcctgattaaagaaatgtttgccacggtaggggaaaacgcctgggtagaaccgcctgtctatttctcttacggttccaacatccatataggccgcaatttttatgcaaatttcaatttaaccattgtcgatgactacacggtaacaatcggtgataacgtactgattgcacccaacgttactctttccgttacgggacaccctgtacaccatgaattgagaaaaaacggcgagatgtactcttttccgataacgattggcaataacgtctggatcggaagtcatgtggttattaatccaggcgtcaccatcggggataattctgttattggcgcgggtagtatcgtcacaaaagacattccaccaaacgtcgtggcggctggcgttccttgtcgggttattcgcgaaataaacgaccgggataagcactattatttcaaagattataaagttgaatcgtcagtttaa

SEQ ID NO: 275 - DNA, 1801 bp, Artificial Sequence
  Synthetic construct GFP_kanR fragment used for integrating GFP into pathways as reporter gene with selectable marker  BP_DNA_158
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttg

SEQ ID NO: 276 - DNA, 2654 bp, Artificial Sequence
  Synthetic construct GFP_kanR  with homology arms (lacZ, version 2)   Fragment used to integrate GFP_kanR behind lacZ gene in E. coli (version 2)  BP_DNA_159
gtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagcc

SEQ ID NO: 277 - DNA, 2675 bp, Artificial Sequence
  Synthetic construct GFP_kanR with homology arms (lacY)  Fragment used to integrate GFP_kanR behind lacY gene in E. coli BP_DNA_160
gctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgcggcgcgagcgccttatccgaccaacatatcataacgatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttggagtgatcgcattgaacatgccaatgaccgaaagaataagagcaggcaagctatttaccgatatgtgcgaaggcttaccggaaaaaagacttcgtgggaaaacgttaatgtatgagtttaatcactcgcatccatcagaagttgaaaaaagagaaagcctgattaaagaaatgtttgccacggtaggggaaaacgcctgggtagaaccgcctgtctatttctcttacggttccaacatccatataggccgcaatttttatgcaaatttcaatttaaccattgtcgatgactacacggtaacaatcggtgataacgtactgattgcacccaacgttactctttccgttacgggacaccctgtacaccatgaattgagaaaaaacggcgagatgtactcttttccgataacgattggcaataacgtctggatcggaagtcatgtgg

SEQ ID NO: 278 - DNA, 4955 bp, Artificial Sequence
  Synthetic construct B-gal(St. therm)_kanR with homology arms (lacZ)  Fragment used to integrate B-gal (St. therm)_kanR behind lacZ gene in E. coli BP_DNA_161
gtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgatgaacacaacagaaaagatccagacatacttaaacgatccaaaaatcgtatcggttaacacagtagacgcccatagcgatcataagtattttgaatctttggctgaattctctgaaggcgagatgaaattgcgccaaagtcttaatggtaagtggaaaatccactacgcacaaaatacgaaccaggtattaaaggacttttataagacggaatttgacgaaacggaccttaactttatcaacgttcctggccaccttgaattccaaggtttcggctccccgcaatacgttaatacccagtatccgtgggacggcaaggaattcttacgccccccacaggtgccccaagagtctaatgcagtagcctcttacgtcaaacatttcactcttaacgacgcgcttaaagataaaaaggtattcatcagctttcagggagtggctacatcgatcttcgtctgggttaacggaaacttcgtgggctactctgaagactcattcactccttctgagttcgaaatctccgattaccttgttgaaggagataacaaattggccgttgctgtctaccgttactcaacagcttcgtggttggaggatcaagacttttggcgtctgtatgggatttttcgtgacgtgtacttatacgccattcccaaagtgcatgtacaagacttgtttgttaaaggagattatgattatcagaccaagaccggacagcttgatatcgatcttaaaacagtaggtgattacgaggacaagaaaatcaagtacgtgctgtccgatcacgagggtatcgtcacagaaggtgacgcctctgttaatggagatggagagctttctgttagccttgagaacttaaagattaagccttggtccgccgagtctcccaagctttataacctgactctgcatgttcttgatgatgatcaggtagtagaagttgttcccgtaaaggtaggcttccgtcacttcgagatcaaagacaagcttatgttgttaaatggaaagcgcatcgtttttaagggagtaaaccgccatgagtttaacgctcgtacaggccgctgtattacagaagaggacatgttgtgggacatcaaagtcatgaagcagcacaacatcaacgccgttcgtaccagtcattacccgaaccaaacacgttggtatgagctgtgcgacgagtatggactgtatgtgatcgatgaggctaaccttgagactcatggaacatggcaaaagttgggactgagtgaaccttcctggaatatcccagcctcggaaccagaatggctgcccgcatgcttagatcgtgctaataatatgtttcagcgcgacaaaaaccatgcttcggtgatcatctggtcttgtggcaacgagagttacgcgggcaaggacattgcggatatggcggattacttccgttccgtcgacaacacacgtcccgtccactacgagggtgtgacttggtgtcgcgagttcgactatatcaccgatatcgagtcacgcatgtacgcgaaacctgccgatattgaagagtatcttacaaacaacccgcagaaaccgtatatcagttgtgagtacatgcatacaatgggcaattcaggaggaggtctgaagttatatacagatttggagaagtatccggaataccaaggcggttttatttgggatttcatcgatcaagcaatctataagccccttcccaacggctccgaatttttgtcgtatggcggcgactggcatgaccgtccatcggattacgagttctgtggcaatggcattgtcttcgcggaccgcacattgacgccaaaacttcagacagtcaaacatctttattctaacattaaaattgcggtggatgagaaatccgtaacaattaacaatgacaatatttttgaagatctttccgcttatacatttttagcccgtgtctacgaggacggacgcaaggtttctgagtccgagtatcacttcgatgtcaaaccgggggaagaagctacgttcccagttgaattcgccgtaggagcatccaacgcggaacgtatctatgaagtggtttgcgtgcagaaggaagctacggaatgggcgccaaaaggtcacgagattgtacgtggacaatatgtagcggaaaaaatttctaccgaaacgcccgtcaaagctccactgaatgtggtggaaggtgatttcaatattgggatccagggacagaatttttctattcttttaagccgtgcacaaaacacgctggtctcagctaaatataacggtgttgagttcattgaaaaaggcccaaaattaaattttacgcgtgcttatacagacaacgatcgtggggccggctacccgtttgaaatggcggggtggaaagtggcgggaaattattccaaggttacggatacccagatccaaatcgaggacgattcggttaaggtcacttatgtccatgaacttccaggtctgagtgatgtggaagtcaaagtaacgtaccaggtggactataaagggcgtatttttgtcaccgcgaattacgatggaaaagcggggcttccaaatttccccgaattcgggcttgaatttgccattggatcacagttcacaaacctgtcctactatggatatggtgcggaggagtcttacctggataaattgcctggtgcatacctgggacgttatgagacgtcggtggaaaaaacttttgcaccgtatcttatgcctcaggaaagtggcaatcattatggaacacgcgagtttaccgtatcggatgataatcacaatggcctgaagtttactgctcttaacaaagcttttgaattttccgctctgcgtaactccactgagcagatcgaaaacgcccgtcaccagtatgaattgcagcaatcggatgcaacttggatcaaagtattagctgcgcaaatgggtgtaggcggtgacgactcgtggggcgcacccgtacatgacgaatttcttttgagtagcgcggactcttaccagttgggctttatgatcgagcccttatcataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagcc

SEQ ID NO: 279 - DNA, 4976 bp, Artificial Sequence
  Synthetic construct B-gal(St. therm)_kanR with homology arms (lacY)  Fragment used to integrate B-gal (St. therm)_kanR behind lacY gene in E. coli  BP_DNA_162
gctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgcggcgcgagcgccttatccgaccaacatatcataacgatgaacacaacagaaaagatccagacatacttaaacgatccaaaaatcgtatcggttaacacagtagacgcccatagcgatcataagtattttgaatctttggctgaattctctgaaggcgagatgaaattgcgccaaagtcttaatggtaagtggaaaatccactacgcacaaaatacgaaccaggtattaaaggacttttataagacggaatttgacgaaacggaccttaactttatcaacgttcctggccaccttgaattccaaggtttcggctccccgcaatacgttaatacccagtatccgtgggacggcaaggaattcttacgccccccacaggtgccccaagagtctaatgcagtagcctcttacgtcaaacatttcactcttaacgacgcgcttaaagataaaaaggtattcatcagctttcagggagtggctacatcgatcttcgtctgggttaacggaaacttcgtgggctactctgaagactcattcactccttctgagttcgaaatctccgattaccttgttgaaggagataacaaattggccgttgctgtctaccgttactcaacagcttcgtggttggaggatcaagacttttggcgtctgtatgggatttttcgtgacgtgtacttatacgccattcccaaagtgcatgtacaagacttgtttgttaaaggagattatgattatcagaccaagaccggacagcttgatatcgatcttaaaacagtaggtgattacgaggacaagaaaatcaagtacgtgctgtccgatcacgagggtatcgtcacagaaggtgacgcctctgttaatggagatggagagctttctgttagccttgagaacttaaagattaagccttggtccgccgagtctcccaagctttataacctgactctgcatgttcttgatgatgatcaggtagtagaagttgttcccgtaaaggtaggcttccgtcacttcgagatcaaagacaagcttatgttgttaaatggaaagcgcatcgtttttaagggagtaaaccgccatgagtttaacgctcgtacaggccgctgtattacagaagaggacatgttgtgggacatcaaagtcatgaagcagcacaacatcaacgccgttcgtaccagtcattacccgaaccaaacacgttggtatgagctgtgcgacgagtatggactgtatgtgatcgatgaggctaaccttgagactcatggaacatggcaaaagttgggactgagtgaaccttcctggaatatcccagcctcggaaccagaatggctgcccgcatgcttagatcgtgctaataatatgtttcagcgcgacaaaaaccatgcttcggtgatcatctggtcttgtggcaacgagagttacgcgggcaaggacattgcggatatggcggattacttccgttccgtcgacaacacacgtcccgtccactacgagggtgtgacttggtgtcgcgagttcgactatatcaccgatatcgagtcacgcatgtacgcgaaacctgccgatattgaagagtatcttacaaacaacccgcagaaaccgtatatcagttgtgagtacatgcatacaatgggcaattcaggaggaggtctgaagttatatacagatttggagaagtatccggaataccaaggcggttttatttgggatttcatcgatcaagcaatctataagccccttcccaacggctccgaatttttgtcgtatggcggcgactggcatgaccgtccatcggattacgagttctgtggcaatggcattgtcttcgcggaccgcacattgacgccaaaacttcagacagtcaaacatctttattctaacattaaaattgcggtggatgagaaatccgtaacaattaacaatgacaatatttttgaagatctttccgcttatacatttttagcccgtgtctacgaggacggacgcaaggtttctgagtccgagtatcacttcgatgtcaaaccgggggaagaagctacgttcccagttgaattcgccgtaggagcatccaacgcggaacgtatctatgaagtggtttgcgtgcagaaggaagctacggaatgggcgccaaaaggtcacgagattgtacgtggacaatatgtagcggaaaaaatttctaccgaaacgcccgtcaaagctccactgaatgtggtggaaggtgatttcaatattgggatccagggacagaatttttctattcttttaagccgtgcacaaaacacgctggtctcagctaaatataacggtgttgagttcattgaaaaaggcccaaaattaaattttacgcgtgcttatacagacaacgatcgtggggccggctacccgtttgaaatggcggggtggaaagtggcgggaaattattccaaggttacggatacccagatccaaatcgaggacgattcggttaaggtcacttatgtccatgaacttccaggtctgagtgatgtggaagtcaaagtaacgtaccaggtggactataaagggcgtatttttgtcaccgcgaattacgatggaaaagcggggcttccaaatttccccgaattcgggcttgaatttgccattggatcacagttcacaaacctgtcctactatggatatggtgcggaggagtcttacctggataaattgcctggtgcatacctgggacgttatgagacgtcggtggaaaaaacttttgcaccgtatcttatgcctcaggaaagtggcaatcattatggaacacgcgagtttaccgtatcggatgataatcacaatggcctgaagtttactgctcttaacaaagcttttgaattttccgctctgcgtaactccactgagcagatcgaaaacgcccgtcaccagtatgaattgcagcaatcggatgcaacttggatcaaagtattagctgcgcaaatgggtgtaggcggtgacgactcgtggggcgcacccgtacatgacgaatttcttttgagtagcgcggactcttaccagttgggctttatgatcgagcccttatcataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttggagtgatcgcattgaacatgccaatgaccgaaagaataagagcaggcaagctatttaccgatatgtgcgaaggcttaccggaaaaaagacttcgtgggaaaacgttaatgtatgagtttaatcactcgcatccatcagaagttgaaaaaagagaaagcctgattaaagaaatgtttgccacggtaggggaaaacgcctgggtagaaccgcctgtctatttctcttacggttccaacatccatataggccgcaatttttatgcaaatttcaatttaaccattgtcgatgactacacggtaacaatcggtgataacgtactgattgcacccaacgttactctttccgttacgggacaccctgtacaccatgaattgagaaaaaacggcgagatgtactcttttccgataacgattggcaataacgtctggatcggaagtcatgtgg

SEQ ID NO: 280 - DNA, 4102 bp, Artificial Sequence
  Synthetic construct B-gal_kanR (Strep therm, IDT codon opt for E. coli)  Fragment used to integrate an E. coli codon optimized B-gal from Strep therm into the E. coli genome BP_DNA_163
atgaacacaacagaaaagatccagacatacttaaacgatccaaaaatcgtatcggttaacacagtagacgcccatagcgatcataagtattttgaatctttggctgaattctctgaaggcgagatgaaattgcgccaaagtcttaatggtaagtggaaaatccactacgcacaaaatacgaaccaggtattaaaggacttttataagacggaatttgacgaaacggaccttaactttatcaacgttcctggccaccttgaattccaaggtttcggctccccgcaatacgttaatacccagtatccgtgggacggcaaggaattcttacgccccccacaggtgccccaagagtctaatgcagtagcctcttacgtcaaacatttcactcttaacgacgcgcttaaagataaaaaggtattcatcagctttcagggagtggctacatcgatcttcgtctgggttaacggaaacttcgtgggctactctgaagactcattcactccttctgagttcgaaatctccgattaccttgttgaaggagataacaaattggccgttgctgtctaccgttactcaacagcttcgtggttggaggatcaagacttttggcgtctgtatgggatttttcgtgacgtgtacttatacgccattcccaaagtgcatgtacaagacttgtttgttaaaggagattatgattatcagaccaagaccggacagcttgatatcgatcttaaaacagtaggtgattacgaggacaagaaaatcaagtacgtgctgtccgatcacgagggtatcgtcacagaaggtgacgcctctgttaatggagatggagagctttctgttagccttgagaacttaaagattaagccttggtccgccgagtctcccaagctttataacctgactctgcatgttcttgatgatgatcaggtagtagaagttgttcccgtaaaggtaggcttccgtcacttcgagatcaaagacaagcttatgttgttaaatggaaagcgcatcgtttttaagggagtaaaccgccatgagtttaacgctcgtacaggccgctgtattacagaagaggacatgttgtgggacatcaaagtcatgaagcagcacaacatcaacgccgttcgtaccagtcattacccgaaccaaacacgttggtatgagctgtgcgacgagtatggactgtatgtgatcgatgaggctaaccttgagactcatggaacatggcaaaagttgggactgagtgaaccttcctggaatatcccagcctcggaaccagaatggctgcccgcatgcttagatcgtgctaataatatgtttcagcgcgacaaaaaccatgcttcggtgatcatctggtcttgtggcaacgagagttacgcgggcaaggacattgcggatatggcggattacttccgttccgtcgacaacacacgtcccgtccactacgagggtgtgacttggtgtcgcgagttcgactatatcaccgatatcgagtcacgcatgtacgcgaaacctgccgatattgaagagtatcttacaaacaacccgcagaaaccgtatatcagttgtgagtacatgcatacaatgggcaattcaggaggaggtctgaagttatatacagatttggagaagtatccggaataccaaggcggttttatttgggatttcatcgatcaagcaatctataagccccttcccaacggctccgaatttttgtcgtatggcggcgactggcatgaccgtccatcggattacgagttctgtggcaatggcattgtcttcgcggaccgcacattgacgccaaaacttcagacagtcaaacatctttattctaacattaaaattgcggtggatgagaaatccgtaacaattaacaatgacaatatttttgaagatctttccgcttatacatttttagcccgtgtctacgaggacggacgcaaggtttctgagtccgagtatcacttcgatgtcaaaccgggggaagaagctacgttcccagttgaattcgccgtaggagcatccaacgcggaacgtatctatgaagtggtttgcgtgcagaaggaagctacggaatgggcgccaaaaggtcacgagattgtacgtggacaatatgtagcggaaaaaatttctaccgaaacgcccgtcaaagctccactgaatgtggtggaaggtgatttcaatattgggatccagggacagaatttttctattcttttaagccgtgcacaaaacacgctggtctcagctaaatataacggtgttgagttcattgaaaaaggcccaaaattaaattttacgcgtgcttatacagacaacgatcgtggggccggctacccgtttgaaatggcggggtggaaagtggcgggaaattattccaaggttacggatacccagatccaaatcgaggacgattcggttaaggtcacttatgtccatgaacttccaggtctgagtgatgtggaagtcaaagtaacgtaccaggtggactataaagggcgtatttttgtcaccgcgaattacgatggaaaagcggggcttccaaatttccccgaattcgggcttgaatttgccattggatcacagttcacaaacctgtcctactatggatatggtgcggaggagtcttacctggataaattgcctggtgcatacctgggacgttatgagacgtcggtggaaaaaacttttgcaccgtatcttatgcctcaggaaagtggcaatcattatggaacacgcgagtttaccgtatcggatgataatcacaatggcctgaagtttactgctcttaacaaagcttttgaattttccgctctgcgtaactccactgagcagatcgaaaacgcccgtcaccagtatgaattgcagcaatcggatgcaacttggatcaaagtattagctgcgcaaatgggtgtaggcggtgacgactcgtggggcgcacccgtacatgacgaatttcttttgagtagcgcggactcttaccagttgggctttatgatcgagcccttatcataagtacccaggaaacagctatgaccatgtaatacgactcactatacggggatatcgtcggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgagagctcgcttggactcctgttgatagatccagtaatgacctcagaactccatctggatttgttcagaacgctcggttg